(19) United States
(12) Patent Application Publication
(10) Pub. No.: US 2008/0269072 A1
(43) Pub. Date: Oct. 30, 2008

(54) Rational Probe Optimization for Detection of MicroRNAs

(76) Inventors: Ronald P. Hart, Cranford, NJ (US); Loyal A. Goff, Belle Mead, NJ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/577,581

(22) PCT Filed: Oct. 21, 2005
(86) PCT No.: PCT/US05/38261

§ 371 Date: Jun. 12, 2008

Related U.S. Application Data
(60) Provisional application No. 60/620,343, filed on Oct. 21, 2004.

(51) Int. Cl. C40B 40/06; C07H 21/02; G06F 19/00

(52) U.S. Cl. 506/16; 536/24.3; 702/19

(57) ABSTRACT

A method for the rational optimization of probes for the detection of miRNAs from different species is provided.

This application claims priority to U.S. provisional Application 60/620,343 filed Oct. 21, 2004, the entire contents of which are incorporated by reference herein.

### FIELD OF THE INVENTION

This invention relates to the fields of molecular biology and the regulation of gene expression. More specifically, the invention provides an improved method for designing oligonucleotide probes for use in nucleic acid detection technologies, including the creation of DNA microarrays for the detection of biologically important microRNA molecules.

### BACKGROUND OF THE INVENTION

Several publications and patent documents are cited throughout the specification in order to describe the state of the art to which this invention pertains. Each of these citations is incorporated by reference herein as though set forth in full.

MiRNAs represent a class of small (˜18-25 nt), endogenous, non-coding RNA molecules that function in post-transcriptional regulation of specific target mRNAs (1-5). While several hundred miRNAs have been identified to date, the functions of only a few have been described in detail. This has been hindered in part by their small size and imperfect base pairing to target mRNAs, although several computational methods have been proposed to identify miRNA-target mRNA interactions (6-9). The functions of miRNAs that have been elucidated indicate that these miRNAs influence a wide range of biological activities and cellular processes. miRNAs have been implicated in developmental patterning and timing (1), restriction of differentiation potential (10, 11), maintenance of pluripotency, hematopoietic cell lineage differentiation (10), regulation of insulin secretion (12), adipocyte differentiation (11), proliferation of differentiated cell types (13), genomic rearrangements (14), and carcinogenesis (14-17).

The recent discovery of miRNAs has led to the development of several species specific, high-throughput detection methods. In several reports, spotted oligonucleotide microarray technology has proven to be effective (11, 15, 16, 18-26). However, design of spotted oligonucleotide probes for mature miRNAs presents several challenges. For example, strong conservation between miRNA family members makes it difficult to design probes that are specific at the level of a single nucleotide out of a 20 nucleotide sequence. Thus, it is an object of the invention to provide an improved design strategy for the generation of highly specific probes for miRNA detection.

### SUMMARY OF THE INVENTION

In accordance with the present invention, an algorithm for the design of highly selective probes for the detection of miRNAs has been developed. Probes have been designed and validated for miRNAs from six species, thereby providing the means by which to identify novel miRNAs with homologous probes from other species. These methods are useful for high-throughput analysis of micro RNAs from various sources, and allow analysis with limiting quantities of RNA. The system design can also be extended for use on Luminex beads or on 96-well plates in an ELISA-style assay. We optimized hybridization temperatures using sequence variations on 20 of the probes and determined that all probes distinguish wild-type from 2 nt mutations, and most probes distinguish a 1 nt mutation, producing good selectivity between closely-related small RNA sequences. Results of tissue comparisons on our microarrays created using probes designed using the algorithm of the invention reveal patterns of hybridization that agree with results from Northern blots and other methods.

Thus, in one embodiment of the invention, a computer assisted method for optimizing design of probes which selectively hybridize to target miRNAs obtained from a database using a programmed computer, including a processor, an input device and an output device is provided. An exemplary computer assisted method entails inputting into the computer, miRNA sequence data, upper and lower ranges of sequence length and upper and lower ranges of Tm and determining, using the processor, those probes which satisfy the inputted Tm parameters and sequence length following truncation of the sequences at either the 3′ or 5′ end of said sequence. Once such sequences are identified they are then outputted by the program. Also provided in the present invention is a computer program for implementing the method described above. In one aspect of the method, the sequences are truncated at the 5′ end only. In yet another approach, sequences are truncated at the 3′ end only, although truncation at the 5′ end is preferred.

Also encompassed within the invention is a computer-readable medium having recorded thereon a program that provides at least one miRNA probe which specifically hybridizes to the target miRNA according to the method set forth above. A computational analysis system comprising a computer-readable medium described above is also provided.

In yet another aspect, a kit for identifying a sequence of a nucleic acid that is suitable for use as an probe for a target miRNA is disclosed. An exemplary kit comprises (a) an algorithm that identifies a sequence of a nucleic acid that is suitable for use as a probe according to the methods provided herein, wherein said algorithm is present on a computer readable medium; and (b) instructions for using said algorithm to identify said sequence of a nucleic acid that is suitable for use as a probe for said miRNA target nucleic acid.

The invention also provides a method for rational probe optimization for detection of Mi RNA molecules comprising: a) providing a database of known miRNA sequences; b) performing the miRMAX algorithm on said sequences to identify probes having enhanced sequence specificity, substantially similar hybridization temperatures and sequence length; and c) obtaining the probe sequences identified in step b) and optionally synthesizing the same. The method of the invention may also comprise generating the reverse complement of the sequences obtained using the MiRMAX algorithm and preparing concatamers of said probe sequences. Such multimeric probe sequences are useful in a variety of different detection platforms.

In a preferred embodiment, the probes so identified are affixed to a solid support. Exemplary solid supports include, without limitation, glass slides, magnetic beads, glass beads, latex beads, luminex beads, filters, multiwell plates and microarrays.

Finally, the invention also provides an oligonucleotide array comprising an array of multiple oligonucleotides with different base sequences fixed onto known and separate positions on a support substrate, said oligonucleotides being synthesized using the outputted sequences identified using the MiRMAX algorithm of the invention, wherein said oligonucleotides specifically hybridize to miRNA sequences or the complement thereof, and the said oligonucleotides are classified according to their sequence of origin, wherein the fixation region on the support substrate is divided into the said classification.

### BRIEF DESCRIPTION OF THE DRAWINGS

FIG. 1—Probe design algorithm FIG. 1A shows evaluation of probe design algorithms. Test microarrays were printed with various versions of oligonucleotide probes to compare hybridization signals (sequences of numbered probes are shown in Table 1 hereinbelow). Results show the median intensity values of hybridization to synthetic miR-9 and miR-103, for each of several different probe design truncation patterns. The numbers following the hyphen are codes for various versions of the probe using different design strategies. The patterns chosen by our final probe design algorithm are indicated in bold italics and show hybridization levels equivalent to or, in most cases, stronger than that of the wt (unaltered) probe sequences while retaining appropriate hybridization results. FIG. 1B shows the selected probe design algorithm. A flow chart shows the steps in the selected design algorithm.

FIG. 2—Sequence selectivity by hybridization temperature. Control probe median intensity values (background subtracted) were obtained from hybridization to a pool of synthetic miRNAs, each ˜700 pg. Probes spotted onto the microarray for each control set included a wild-type, anti-sense monomer oligo (Monomer), a designed probe (miRMAX), the designed probe with one nucleotide mismatch (Mut1) or two nucleotides of mismatch (Mut2), a reverse complement probe (Rev) and a randomly shuffled sequence (Shuf). Individual lines indicate values obtained at various hybridization temperatures (see legend). The two predominant patterns of results obtained are demonstrated by the hybridization of (FIG. 2A) miR-16, in which the Mut1 intensities are decreased regardless of hybridization temperature, and (FIG. 2B) miR-152 in which the Mut1 probe showed comparable or slightly greater hybridization to the synthetic miRNA. This greater hybridization was almost entirely removed if more stringent hybridization temperatures were utilized. In an attempt to find if specific mutation types affect the selective hybridization to our designed probes, we plotted the percentage ratio of Mut1 median intensities (mm; mismatch) to probe (pm; perfect match) intensities against the calculated melting temperatures of the miRNA:probe dimer. Individual points are keyed by type of mutation (see legend). While a general trend was observed for all data, no obvious patterns emerged when comparisons were made between relative position of the mutation within the miRNA sequence (C) or type of nucleotide change that was made (D).

FIG. 3—Northern validation of microarray results. (FIG. 3A) Northern blots of three mature miRNA species, miR-191, miR-16, and miR-93, from liver (L) and brain (B) LMW RNA samples are shown. Probes for Northern and dot blots consisted of traditional antisense oligo probes coupled with StarFire detection sequences (IDT). Mean intensity values from the three liver/brain microarray hybridizations are shown in (FIG. 3B) for liver (grey) and brain (black). The integrated volume for each of the Northern images (FIG. 3C) shows similar patterns of relative miRNA levels between the two tissues for each of the three miRNAs. (FIG. 3D) Dot blots compared sequence specificity of synthetic miRNAs spotted on nylon membranes using traditional oligo probes. Synthetic miR-191 miRNA (wt), or a single mutation (mut1) or double-mutation (mut2) RNAs were spotted and detected with probes matching mut1 or wt sequence. Each probe detected its perfect complement as well as a 1 nt mismatch. Interestingly, the mut1 probe hybridized primarily with mut2 RNA over wt RNA, even though both synthetic RNAs were 1 nt different from probe.

FIG. 4—Tissue-specific hybridization. Scatterplot depicts average log₂ fluorescence intensity values for each rat and mouse miRNA probe for three liver and brain miRMAX hybridizations.

FIG. 5—Hierarchical clustering of miRNA expression levels in neural stem cell clones. A hierarchical clustering heat map shows rat and mouse miRNA expression levels in various stem cell lines as well as in adult liver and brain LMW RNA. Several miRNAs appear to be expressed more intensely in the stem cell lines as compared to the adult tissue (expanded region), including members of a previously identified “ES-cell specific” miRNA cluster (42).

FIG. 6 shows the MiRMAX algorithm of the invention.

### DETAILED DESCRIPTION OF THE INVENTION

We have designed and validated a method for designing oligonucleotide probes for a DNA microarray specific for micro RNAs (miRNA). miRNAs are short (18-22 nt) molecules processed from longer cellular precursors that inhibit translation of mRNA into protein, apparently under tissue-specific and other regulatory control. Using fluorescent labeling technologies developed by Genisphere Inc. (3DNA dendrimers) we have labeled miRNA mixtures directly with large numbers of fluorescent dyes. This method, since it directly labels the miRNA, requires an “anti-sense” DNA probe for construction of a microarray. Others have suggested merely synthesizing trimeric repeated sequences for designing oligo probes. We found that dimeric sequences were adequate, and possibly more sensitive than trimeric sequences. Furthermore, since most of the specificity of the miRNA for target mRNA is near the 5′ terminus, we have developed an algorithm for selecting sequence subsets. Our method optimizes melting temperature for uniform hybridization, retains sequences thought to be relevant for target mRNA binding, and removes nucleotides as needed to produce uniform-sized probes. We tested our algorithm by synthesizing several variations of our design, spotting them onto microarrays and hybridizing them with fluorescence-tagged synthetic miRNAs. Results of this hybridization were used to validate the optimal design algorithm.

Our method provides a straightforward way to produce anti-sense oligonucleotide probe sequences for constructing a microarray specific for miRNAs. The resulting microarray is uniquely suited to the labeling technologies developed by Genisphere, Inc.

The following definitions are provided to facilitate an understanding of the present invention.

The term “micro RNA” refers to small (approximately 18-25 nucleotide), endogenous, non-coding RNA molecules that function in post-transcriptional regulation of specific target mRNAs.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The phrase “consisting essentially of” when referring to a particular nucleotide or amino acid means a sequence having the properties of a given SEQ ID NO:. For example, when used in reference to a nucleic acid sequence, the phrase includes the sequence per se and molecular modifications that would not affect the basic and novel functional characteristics of the sequence.

The phrase “solid support” as used herein refers to any surface to which a nucleic acid may be affixed. Such supports include, without limitation, glass slides, magnetic, glass and latex beads, multiwell plates, filters and microarrays.

The term “probe” as used herein refers to an oligonucleotide; polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and the method used. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. Such probes must, therefore, be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. Most preferably, the probes of the invention are selected using the algorithm provided herein which generates probes having annealing characteristics within a specified range by reducing the length of the probe at one or both ends.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

For example, hybridizations may be performed, according to the method of Sambrook et al. using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is as follows:

Tm=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° below the calculated Tm of the hybrid. Wash conditions should be as stringent as possible for the degree of identity of the probe for the target. In general, wash conditions are selected to be approximately 12-20° C. below the Tm of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

A “specific binding pair” comprises a specific binding member (sbm) and a binding partner (bp) which have a particular specificity for each other and which in normal conditions bind to each other in preference to other molecules. Examples of specific binding pairs are nucleotide sequences and nucleotide sequence-binding proteins, antigens and antibodies, ligands and receptors and complementary nucleotide sequences. The skilled person is aware of many other examples and they do not need to be listed here. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

The term “substantially pure” refers to a preparation comprising at least 50-60% by weight of a given material (e.g., nucleic acid, oligonucleotide, polypeptide etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “dendrimer” as used herein refers to a branched macromolecule useful for the detection of nucleic acid molecules. See for Example U.S. Patent Applications 20020051981, 20040185470, and 20050003366.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. Chemical tag moieties include such molecules as biotin, which may be added to either nucleic acids or proteins and facilitate isolation or detection by interaction with avidin reagents, and the like. Numerous tag moieties are known to, and can be envisioned by, the trained artisan, and are contemplated to be within the scope of this definition.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### Labeling Methods/Strategies

In a preferred embodiment, the interaction of specific binding pairs (e.g., nucleic acid complexes), are detected by assessing one or more labels attached to the sample nucleic acids, polypeptides, or probes. In a particularly preferred embodiment, the interaction of hybridized nucleic acids is detected by assessing one or more labels attached to the sample nucleic acids or probes. The labels may be incorporated by any of a number of means well known to those of skill in the art. In one approach, the label is simultaneously incorporated during the amplification step in the preparation of the sample nucleic acids or probes. For example, polymerase chain reaction (PCR) with labeled primers or labeled nucleotides will provide a labeled amplification product. The nucleic acid (e.g., DNA) may be amplified, for example, in the presence of labeled deoxynucleotide triphosphates (dNTPs). For some applications, the amplified nucleic acid may be fragmented prior to incubation with an oligonoucleotide array, and the extent of hybridization determined by the amount of label now associated with the array. In a preferred embodiment, transcription amplification, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Such labeling can result in the increased yield of amplification products and reduce the time required for the amplification reaction. Means of attaching labels to nucleic acids include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., see below and, e.g., Molecular Probes, Eugene, Oreg., USA), radiolabels (e.g., .sup.32P, .sup.33P, .sup.35S, .sup.125I, and the like), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, which are incorporated by reference herein.

Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy3 and Cy5, macrocyclic chelates of lanthanide ions, e.g. quantum dye™, fluorescent energy transfer dyes, such as thiazole orange-ethidium heterodimer, TOTAB, ALEXA etc. As mentioned above, labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. For each sample of RNA, one can generate labeled oligos with the same labels.

Alternatively, one can use different labels for each physiological source, which provides for additional assay configuration possibilities.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another utilizing the methods of the present invention.

Suitable chromogens which may be employed include those molecules and compounds which absorb light in a distinctive range of wavelengths so that a color can be observed or, alternatively, which emit light when irradiated with radiation of a particular wave length or wave length range, e.g., fluorescers.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers may be employed either alone or, alternatively, in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene: 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4(3′pyrenyl)butyrate; d-3-aminodesoxy-equilenin; 12-(9′anthroyl)stearate; 2-methylanthracene; 9-vinylanthracene; 2,2′(vinylene-p-phenylene)bisbenzoxazole; p-bis[2-(4-methyl-5-phenyl-oxaz-olyl)]benzene; 6-dimethylamino-1,2-benzophenazin; retinol; bis(3′-aminopyridinium) 1,10-decandiyl diiodide; sulfonaphthylhydrazone of hellibrienin; chlorotetracycline; N(7-dimethylamino-4-methyl-2-oxo-3-chro-menyl)maleimide; N-[p-(2-benzimidazolyl)-phenyl]maleimide; N-(4-fluoranthyl)maleimide; bis(homovanillic acid); resazarin; 4-chloro-7-nitro-2,1,3benzooxadiazole; merocyanine 540; resorufin; rose bengal; and 2,4-diphenyl-3(2H)-furanone.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectible signal or donates energy to a fluorescent acceptor. A diverse number of families of compounds have been found to provide chemiluminescence under a variety or conditions. One family of compounds is 2,3-dihydro-1,-4-phthalazinedione. The must popular compound is luminol, which is the 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence can also be obtained with oxalates, usually oxalyl active esters, e.g., p-nitrophenyl and a peroxide, e.g., hydrogen peroxide, under basic conditions. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

A label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are preferred and easily added during an in vitro transcription reaction. In a preferred embodiment, fluorescein labeled UTP and CTP are incorporated into the RNA produced in an in vitro transcription reaction as described above.

The labels may be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. For example, labels may be attached at positions on the ribose ring or the ribose can be modified and even eliminated as desired. The base moieties of useful labeling reagents can include those that are naturally occurring or modified in a manner that does not interfere with their function. Modified bases include but are not limited to 7-deaza A and G, 7-deaza-8-aza A and G, and other heterocyclic moieties.

In a preferred embodiment, miRNAs may be detected using the dendrimer based labeling technology of Genisphere, Inc.

Aspects of the invention may be implemented in hardware or software, or a combination of both. However, preferably, the algorithms and processes of the invention are implemented in one or more computer programs executing on programmable computers each comprising at least one processor, at least one data storage system (including volatile and non-volatile memory and/or storage elements), at least one input device, and at least one output device. Program code is applied to input data to perform the functions described herein and generate output information. The output information is applied to one or more output devices, in known fashion.

Each program may be implemented in any desired computer language (including machine, assembly, high level procedural, or object oriented programming languages) to communicate with a computer system. In any case, the language may be a compiled or interpreted language.

Each such computer program is preferably stored on a storage medium or device (e.g., ROM, CD-ROM, tape, or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

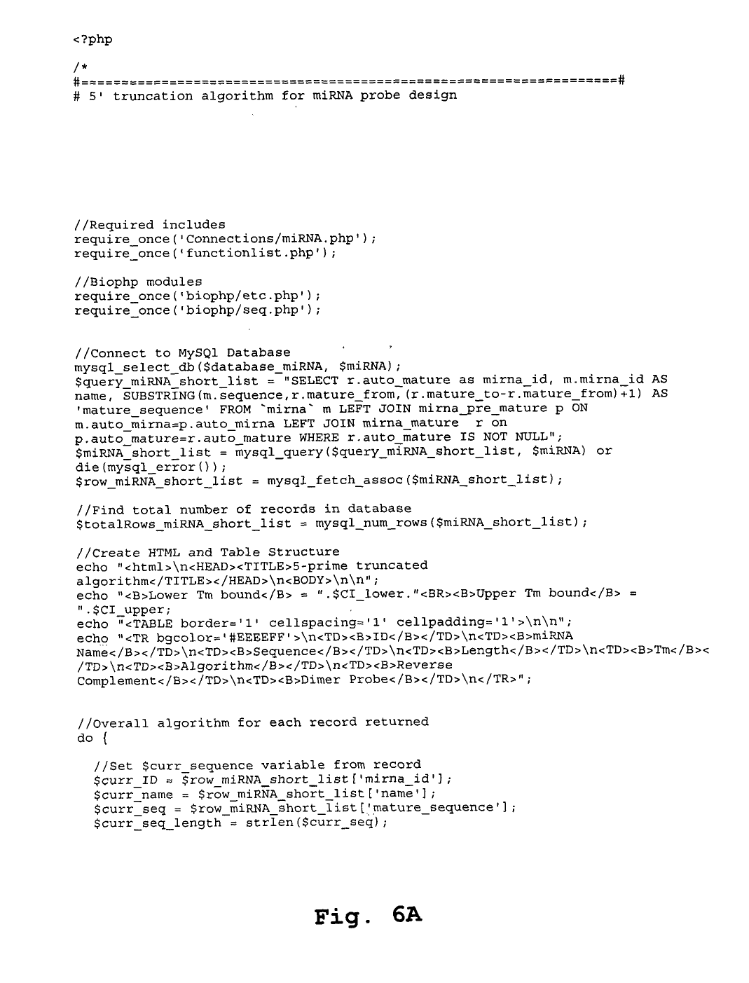

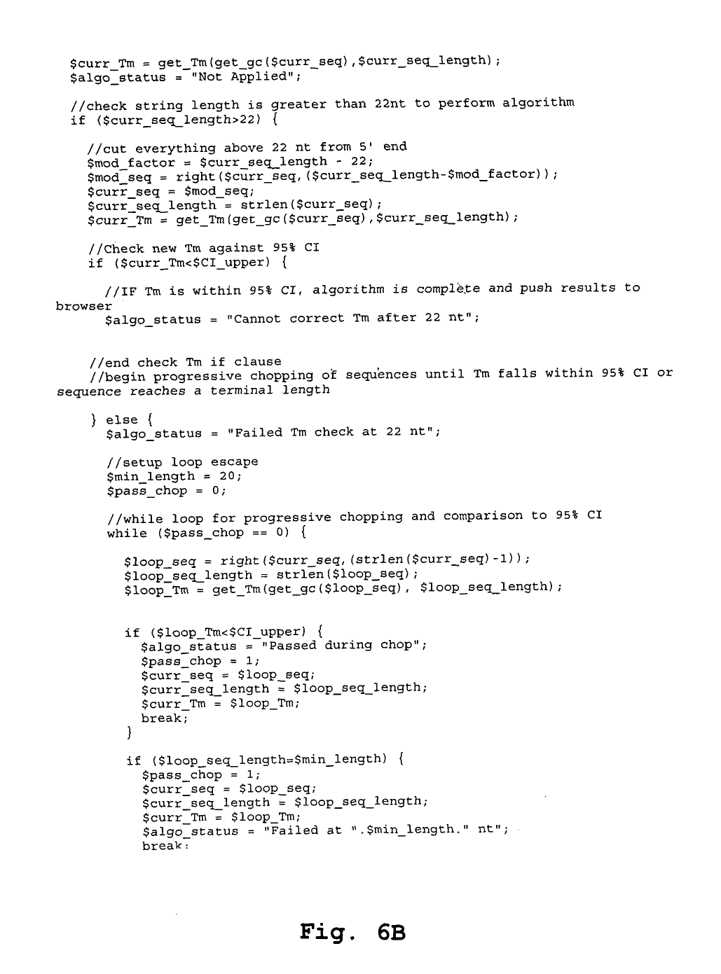

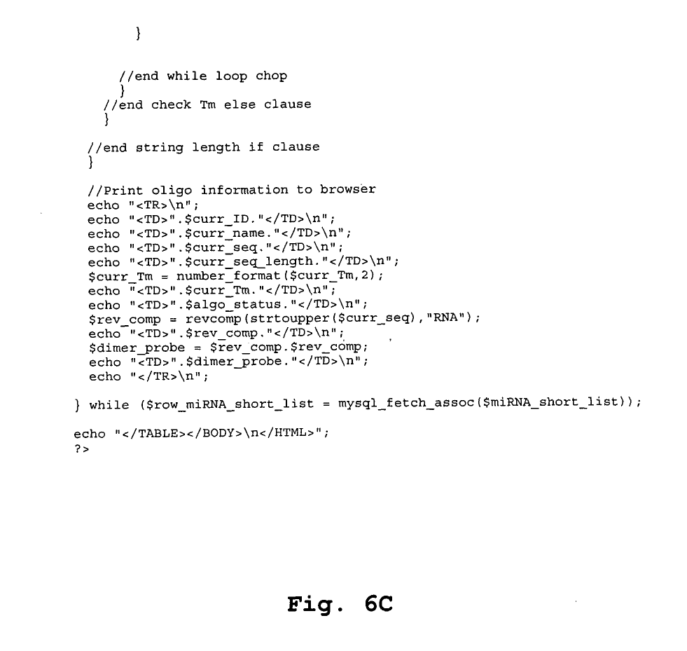

Thus, in another embodiment, the invention provides a computer program, stored on a computer-readable medium, for generating optimal probes for the detection of miRNAs from a variety of species and tissue types. The computer program includes instructions for causing a computer system to: 1) assemble and record known miRNA sequences; 2) inputting upper and lower parameters of sequence length and Tm; 3) selectively truncating the sequences at either the 3′ or 5′ end or both; and 4) outputting those probes that satisfy the inputted Tm parameters. The computer program will contain the algorithm shown in FIG. 6.

The following example is provided to illustrate various embodiments of the invention. It is not intended to limit the invention in any way.

### EXAMPLE I

We report here the development of miRMAX (MicroRNA MicroArray X-species), a cross-species, sensitive, and specific microarray platform for the detection of mature miRNAs. To facilitate detection of the miRNA we have employed a technique which sequence-tags mature miRNAs directly so that they may be detected with high specific-activity fluorescent dendrimers (27). Using these techniques, we identify and validate selected tissue-specific differences in miRNA expression in rat liver and brain tissues, as well as a limited number of embryonic and neural stem tissues.

The following materials and methods are provided to facilitate the practice of the present invention.

### Probe Oligo Design

A local MySQL database was developed and populated with mature miRNA sequences obtained from miRBase (http://microrna.sanger.ac.uk, formerly known as the Sanger Registry). While use of this particular database is exemplified herein, other databases are available to the skilled person. All known and categorized sequences for H. sapiens, M. musculus, R. Norvegicus, C. elegans, D. rerio, and D. melanogaster were utilized to create reverse-complementary microarray probes. Probes identified and verified using the miRMAX algorithm are set forth in Table 2 at the end of the specification.

Probe sequences were trimmed as described in Results to balance the Tₘ of each of the sequences. Several negative control probes were created for each species, with C→A or G→C mutations introduced to create mismatches. A 1 nt mismatch, a 2 nt mismatch, a random sequence, a shuffled sequence, and a monomer probe were generated for each selected control spot to serve as control. Shuffled sequences were randomized using the same base composition and tested for a lack of matches in GenBank by BLAST (28). Artificial miRNAs were synthesized (IDT, Inc., Coralville, Iowa) for each of the 20 miRNAs exemplified hereinto act as positive controls.

Probe sequences were synthesized by IDT, Inc., and suspended in Pronto Glymo Buffer (Coming Life Sciences, Acton, Mass.) at a concentration of 30 μM. Each control spot was printed in duplicate onto the array using an OmniGrid 100 (Genomic Solutions, Ann Arbor, Mich.) and Stealth SMP2 pins (Telechem, Inc., Sunnyvale, Calif.). Probes were arranged by species into different sub-arrays and were printed using an arraying robot on Coming Epoxide slides. Slides were dried overnight in nitrogen, and then placed in a humid chamber for 3 hours to complete coupling. Slides were then washed sequentially in 0.1% Triton-X100, 0.1 M HCl, and 0.1 M KCl, water, and then unreacted groups were blocked with 50 mM ethanolamine in 100 mM Tris-HCl pH 9.0 and 0.1% SDS, followed by water washes. The arrays were then allowed to dry overnight prior to hybridization.

### RNA Preparation and Labelling

Individual liver and brain tissue samples were obtained from three adult Long-Evans rats. Low molecular weight (LMW) RNA was extracted from each sample using the mirVana™ miRNA extraction kit (Ambion, Austin, Tex.). LMW RNA was quantified using the RiboGreen™ kit (Invitrogen, Carlsbad, Calif.) high-range assay. 100 ng of LMW RNA was typically used as input for the labelling reaction. Quality of LMW RNA was judged indirectly by running the high molecular weight fraction from the same preparation on an Agilent Bioanalyzer. We observed that low quality high molecular weight RNA produced poor hybridization results on arrays (not shown).

miRNAs were labelled using the Array900 miRNA Direct kit (Genisphere Inc, Hatfield, Pa.). Briefly, 100 ng of enriched miRNA was polyadenylated using poly(A) polymerase (2 U) and ATP (8 μM final concentration) in the provided reaction buffer (1× reaction buffer: 10 mM Tris-HCl, pH 8.0, 10 mM MgCl₂, 2.5 mM MnCl₂) in 25 μl for 15 minutes at 37° C. Polyadenylated miRNAs were sequence tagged by adding 6 μl of 6× Cy3 or Cy5 ligation mix and 2 μl of T4 DNA Ligase (1 U/μl) and incubating at 20° C. for 30 min in a final volume of 36 μl. For these experiments, 6× Ligation Mix consists of two prehybridized oligonucleotides, a Cy3 or Cy5 capture sequence tag and the appropriate bridging oligonucleotide, in 6× concentrated ligation buffer diluted from 10× Ligation Buffer (Roche). The capture sequence tag is a 31 base oligonucleotide complementary to an oligonucleotide attached to a 3DNA dendrimer labeled with either Cy3 or Cy5. The bridging oligonucleotide (19 nt) consists of 9 nt that are complementary to the capture sequence tag and 10 nt complementary to the added poly A tail (dT₁₀). After terminating the ligation reaction by adding 4 μl of 0.5 M EDTA, the tagged miRNAs were purified a MinElute PCR Purification kit (Qiagen) according to the manufacture's protocol for DNA cleanup.

### Array Hybridization

Sequence-tagged LMW RNA was hybridized to the miRNA microarrays using the Ventana Discovery System (Ventana Medical Systems, Tuscon Ariz.) as described below. Tagged miRNA samples were hybridized for 12 hours in ChipHyb buffer (Ventana) containing 8% formamide. After 12 hours, slides were washed with 2×SSC at 37° C. for 10 min; and then with 0.5×SSC at 37° C. for 2 min. After this initial hybridization, a mixture of Cy3 and Cy5 labelled 3DNA dendrimers was applied to each microarray and a second hybridization proceeded for 2 hours at 45° C. Arrays were washed with 2×SSC at 42° C. for 10 min and then removed from the hybridization system. Slides were then manually washed (1 min each) twice in Reaction Buffer (Ventana) and a final, room temperature wash in 2×SSC. Arrays were dried and coated with DyeSaver (Genisphere) to preserve Cy5 intensities. Arrays were scanned using an Axon GenePix 4000B scanner (Molecular Devices, Union City, Calif.) and median spot intensities collected using Axon GenePix 4.0 (Molecular Devices). Data analysis and manipulation were conducted in either GeneSpring 7.0 (Agilent, Redwood City, Calif.), or GeneTraffic Duo (Stratagene, La Jolla, Calif.).

### Northern Blots

For each Northern blot, 3 μg of LMW rat brain or rat liver RNA was electrophoretically separated in a 15% urea-polyacrylamide gel. RNAs were again electroblotted onto Hybond-N⁺ membrane, UV-crosslinked and baked for one hour at 80° C. StarFire probes (29) against miR-93 (5′-CTACCTGCACGAACAGCACTTT-3′), miR-16 (5′-CGCCAATATTTACGTGCTGCTA-3′), and miR- 191 (5′-AGCTGCTTTTGGGATTCCGTTG-3′) were radio-labelled with [α-P³²]-dATP at 6000 Ci/mmol. Membranes were probed with one of the StarFire Probes overnight for 50° C.

For the dot blot series of Northern hybridizations, 2 ng of either synthetic wt miR-191 RNA (5′-caacggaaucccaaaagcagcu-3′), a 1 nt mismatch miR-191 RNA (5′-caacgCaaucccaaaagcagcu-3′; mismatch underlined), or a 2 nt mismatch miR-191 (5′-caacgCaaucccaaaagAagcu-3′), was spotted to Hybond-N⁺ membrane followed by UV-crosslinking and baking at 80° C. for 1 hour. The quantity of synthetic miRNA was determined by comparing a serial dilution to 3 μg of LMW RNA (not shown). The membranes were then probed with StarFire probes (IDT) for either the miRMAX probe sequence for miR-191 or the mut-1 control probe for miR-191 that were radioactively labelled with [α-P³²]-dATP 6000 Ci/mmol following the vendor's recommendation. The membranes were probed overnight at 55° C. Dot intensities were recorded using a PhosphorImager (GE Biosciences, Niskayuna, N.Y.) and dot volume was measured using ImageQuant (GE Biosciences) software.

### Neural Stem Cell Culture

Neural stem cell cultures were created and maintained as described previously (30, 31). The N01 NS clone was prepared from rat fetal blood and grown as neurospheres using similar methods (D. Sun, unpublished). For comparison, tissues were prepared from adult rat olfactory bulb, brain or liver.

### RESULTS

### Probe Oligo Design

The initial probe design incorporated several concepts, including: (1) trimming of miRNA sequences to adjust for an inherently wide variance in melting temperatures, (2) constructing reverse-complement probes to allow direct hybridization to labelled miRNAs, and (3) comparing monomer, dimer, and trimer probe sequences to maximize sensitivity.

We decided to truncate miRNA sequences in an attempt to reduce the large range of Tₘ values across all known miRNA sequences. Several different miRNA truncation algorithms were evaluated to determine the effect on hybridization to a labelled extract. Initially, we judged hybridization intensity with reverse-complement dimer probes using several variations in probe sequence content. Initial truncation algorithms removed 1 nt from 3′ or 5′ ends in alternating succession from probes with high Tₘ. Further refinement of our approach involved calculating which end of the miRNA allowed for the most precise adjustment of Tₘ during truncation. Additionally, it has been shown that the 5′ “seed” region of a miRNA is conserved among miRNA family members (7, 32-34). Additional weight and preference was therefore given to truncation at the 5′ end, so as to preserve the more variable 3′ sequence, and allow for better discrimination between closely related miRNAs. The final adopted design algorithm created probe sequences with a mean Tₘ of 66.72° C. with a 95% CI ranging from 66.47 to 66.97° C., as compared to the wider distribution of the original miRNA sequences (mean 68.07° C., 95% CI 67.75 to 68.39° C.). This adjustment in melting temperature is expected to allow more uniform hybridization among different probe sequences with minimal loss of selectivity.

Previous methods for spotting probes for miRNAs have demonstrated the efficacy of constructing multimeric probe sequences to maximize the availability of a complementary sequence for hybridization (18, 20). One potential method would be to add a terminal amine group for attachment to epoxy groups on the glass slides, but since all oligos also contain internal amine groups that would compete for this reaction, we chose to eliminate the use of terminal amines. Using unmodified oligos also greatly reduces the cost of manufacture. We reasoned that multimers of probe sequence would covalently attach to epoxy groups via internal bases with primary amines without significantly affecting hybridization efficiency. With this in mind, we constructed monomer, dimer, and trimer probe sequences for comparison. While both dimer and trimer probes showed enhanced hybridization signal intensity as compared to the monomer sequence, there was no significant advantage to trimer sequences over dimer sequences as both yielded comparable intensities (not shown). For this reason, dimer probe sequences were utilized.

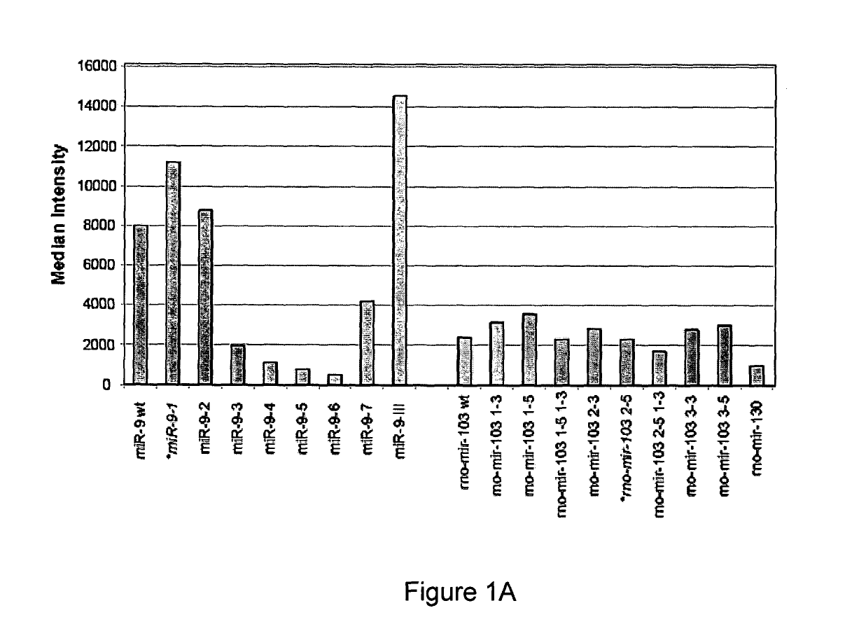

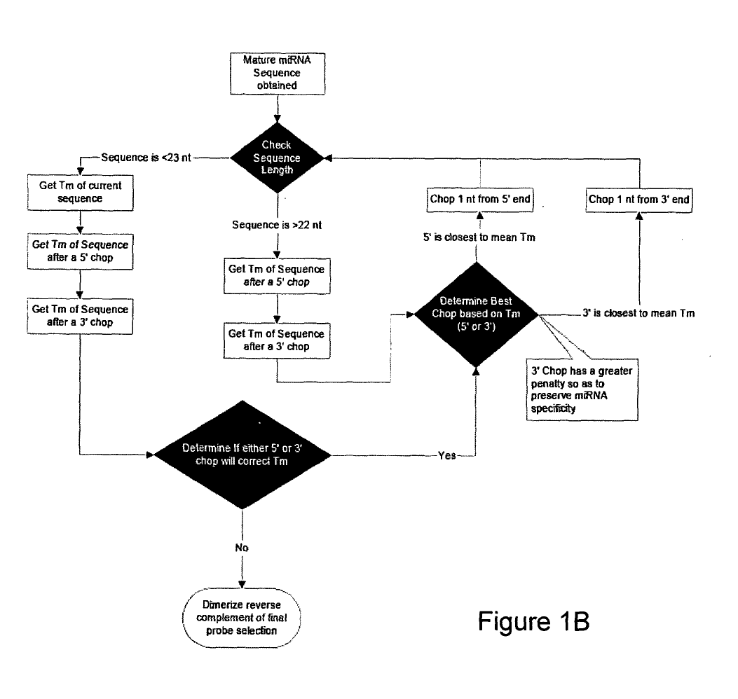

Low molecular weight (LMW) rat brain RNA extracts, hybridized to microarrays with probes of various truncation patterns (Table 1), indicated that our final probe design algorithm provides comparable intensities to wt (full-length, reverse-complement dimer) probe sequences (FIG. 1). In all but a few test cases, the designed probe showed an intensity equal to or greater than that of the wild-type probe. Those with weaker intensities than the wt probe showed only slight variation across different truncation patterns as well, indicating a minimal threshold of intensity for that given miRNA. We conclude that our probe design algorithm produces hybridization results that are indistinguishable from unaltered sequences. Furthermore, dimer probes produce improved hybridization over monomer probes and are similar to trimer probes. Probes were created for each mature miRNA from Homo sapiens, Rattus norvegicus, Mus musculus, Caenorhabditis elegans, and Drosophila melanogaster in the Sanger miRNA Registry (35). We designed a total of 457 unique probe sequences targeting 225 human, 198 rat, 229 mouse, 85 fly, and 117 worm miRNAs. See Table 2 at the end of the specification.

TABLE 1

Sequences of oligo probes used in FIG.1A. All sequences are
5′ to 3′, left to right.

| Target | | |
|---|---|---|
| miRNA | Variant | Printed Probe |
| miR-9 | Wt | TCATACAGCTAGATAACCAAAGATCATACAGCTAGATAACCAAAGA |
| | 1 | TCATACAGCTAGATAACCAAAGTCATACAGCTAGATAACCAAAG |
| | 2 | CATACAGCTAGATAACCAAAGCATACAGCTAGATAACCAAAG |
| | 3 | TCATACAGCTAGATAACCAATCATACAGCTAGATAACCAA |
| | 4 | CATACAGCTAGATAACCAAACATACAGCTAGATAACCAAA |
| | 5 | TCATACAGCTAGATAACCATCATACAGCTAGATAACCA |
| | 6 | TCATACAGCTAGATAACCTCATACAGCTAGATAACC |
| | 7 | TCATACAGCTAGATAACCAAATCATACAGCTAGATAACCAAA |
| | Tri | TCATACAGCTAGATAACCAAAGATCATACAGCTAGATAACCAAA |
| | | GATCATACAGCTAGATAACCAAAGA |
| miR-103 | Wt | TCATAGCCCTGTACAATGCTGTCATAGCCCTGTACAATGCTG |
| | 1 | TCATAGCCCTGTACAATGCTTCATAGCCCTGTACAATGCT |
| | 2 | CATAGCCCTGTACAATGCTGCATAGCCCTGTACAATGCTG |
| | 3 | CATAGCCCTGTACAATGCTCATAGCCCTGTACAATGCT |
| | 4 | TCATAGCCCTGTACAATGCTCATAGCCCTGTACAATGC |
| | 5 | ATAGCCCTGTACAATGCTGATAGCCCTGTACAATGCTG |
| | 6 | ATAGCCCTGTACAATGCTATAGCCCTGTACAATGCT |
| | 7 | TCATAGCCCTGTACAATGTCATAGCCCTGTACAATG |
| | 8 | TAGCCCTGTACAATGCTGTAGCCCTGTACAATGCTG |
| | 9 | TCATAGCCCTGTACAATTCATAGCCCTGTACAAT |

As compared with traditional microarrays, the miRNA labelling method faces unique limitations and challenges. Importantly, mature miRNAs are not normally polyadenylated, so traditional methods of priming with oligo d(T) will not work. Furthermore, since miRNAs are so small, either reverse transcription into labelled cDNA or direct coupling of fluorescent dyes to miRNAs often produces relatively low specific activities and may also tend to interfere with sequence-specific hybridization. Finally, reverse transcription might label precursors to miRNAs with more dye molecules, enhancing hybridization signals disproportionately from non-mature species.

Parallel to the testing of our probe design algorithm, a direct miRNA labelling reaction developed by Genisphere, Inc., was utilized. In this reaction, LMW RNA is 3′ extended with poly(A) polymerase and then ligated to a “capture” sequence tag via a bridging oligo. The sequence-tagged miRNA is hybridized directly to the anti-sense oligo probes and detected by hybridization to a complementary capture sequence on a fluorescent dendrimer. This protocol allows detection of a single molecule of miRNA with as many as 900 molecules of fluorescent dye, greatly amplifying the signal. While this protocol is designed to label mature miRNA we did not evaluate relative labelling efficiency of mature miRNA versus precursor species. After testing a series of diluted RNA samples, we chose to routinely begin with 100-200 ng of LMW RNA per sample, corresponding to 1 μg of total cellular RNA or less, since this gave median hybridization intensities near the center of our fluorescence detection range (not shown). Using 50-fold less input RNA produced essentially undetectable hybridization, and using 50-fold more RNA produced strong hybridization signals for mismatch probes. Other miRNA microarray labelling methods require 5-7 μg (16, 19, 21) or much more (22, 36).

### Optimization of Hybridization

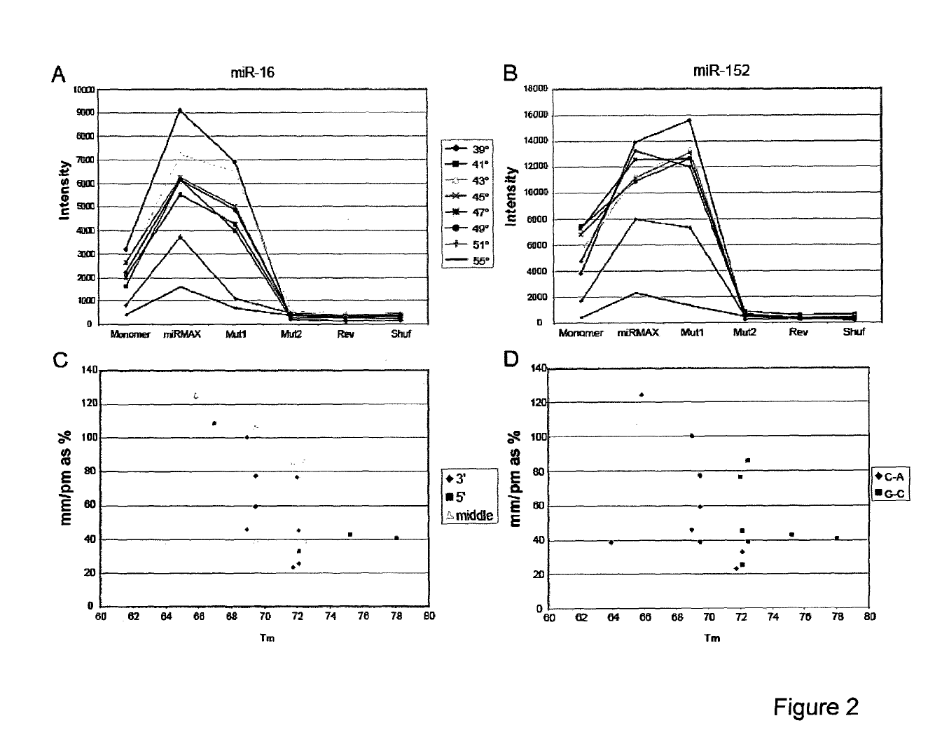

After validation of our probe design algorithm, we examined the ability to select specific miRNA sequences over different hybridization temperatures. Of the probes designed, a subset of 20 was chosen and additional control probes were designed to test sequence selectivity. The control probes included a 1 nt mismatch, 2 nt mismatch, reverse complement, shuffled sequence and monomer probe. The 1 and 2 nt mismatch control probes allowed for determination of the specificity and selectivity of our probes. An equimolar mix of synthetic miRNAs corresponding to the 20 control probe miRNAs was labelled and hybridized to the array. Median signal intensities were calculated for each of the wt probes, 1 nt mutant, 2 nt mutant, reverse complement, shuffled, and monomer sequences and compared for each of the 20 control miRNAs (example results in FIG. 2A and B). As anticipated, signal intensities for the 2 nt mismatch, reverse complement, and shuffled control probes were all but abolished in each case. As in earlier results, monomer probe sequences were also significantly less intense than the dimer sequence. Two distinct patterns emerged from the 1 nt mismatch results. In the majority of the 1 nt mismatch sequences, the intensity was only slightly reduced compared to the miRMAX probe (FIG. 2A). In a few instances however, at less stringent hybridization temperatures, the 1 nt mismatch probe yielded a slightly greater intensity than that obtained from the miRMAX probe (FIG. 2B). This signal was always, however, completely abolished in the 2 nt mutant probe. However, this reduced sensitivity is not due to the probe sequences per se but rather to the assay platform employed.

For each of the 1 nt mutant probes, a ratio of median intensities of the mismatch/perfect match probes (MM/PM) was determined and analyzed to discover what effect, if any, specific mutation types (C→A or G→C; FIG. 2D) or positions within the miRNA sequence (FIG. 2C) had on observed signal intensity. No obvious correlations were identified between sequence transversions or mutation position and signal intensity between the miRMAX probe and the 1 nt mismatches, although a wide range of MM/PM ratios was observed. These observations indicate that our miRNA detection system was quite capable of distinguishing between miRNAs with as few as 2 different nucleotides.

Interpreting the temperature data for all control probes, we selected 47° C. as the best trade-off between sequence specificity and signal intensity. Increasing the temperature to 49° C. slightly reduced the mismatch hybridization signal, but immediately above 49° C. the full-length probe intensity decreased substantially (by 35% from 49-51° C.). We selected 47° C. to reduce the chance of losing signal due to minor changes in temperature. All subsequent data were collected at 47° C.

Our design of control miRNA probes also provides methods for normalizing hybridization results between microarrays. If one sample is assayed per microarray, the second fluorescent channel can be used to label the mixture of 20 synthetic miRNAs as an internal standard. This standard can be used to adjust the fluorescence signal among different microarrays within an experiment. Alternatively, the use of many cross-reacting miRNA probes from other species increases the number of observed hybridization events so that Lowess normalization (37) can be applied to two-color experiments with a more valid number of spots. Experiments can therefore be designed to take advantage of internal standards (one sample per array) or more hybridization results for traditional two-color designs (38).

Validation of miRNA Expression

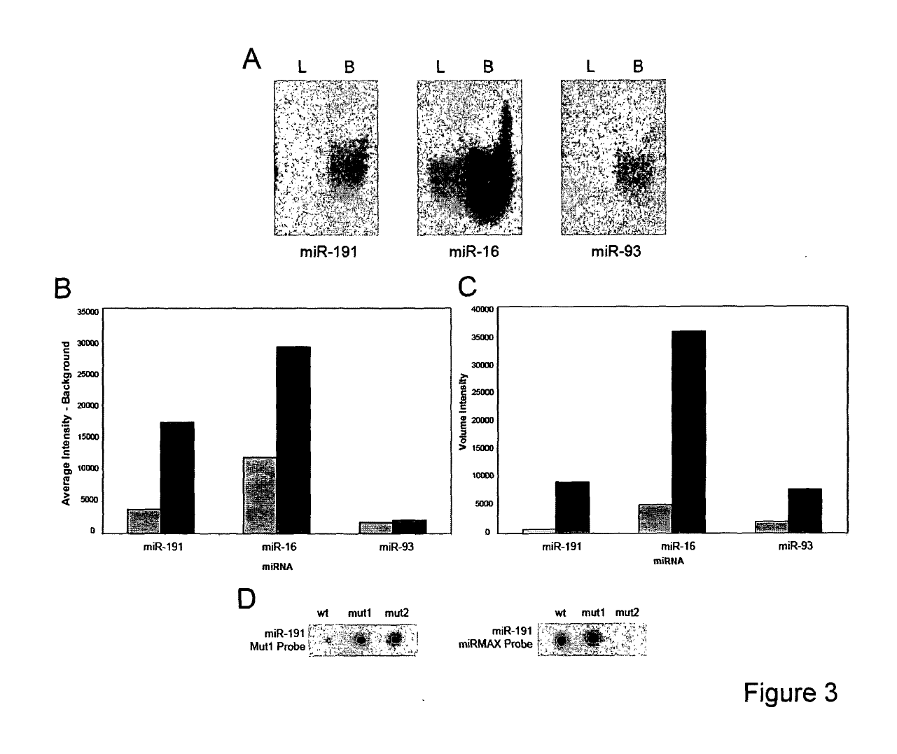

Northern blots were used to validate relative hybridization signals for three miRNAs, miR-191, miR-16, and miR-93. These miRNAs were chosen among the miRNAs for which control sequences had been made so as to facilitate analysis of sensitivity and selectivity (FIG. 3A). For Northern blots, probes were composed of complementary, monomer sequence modified to use the StarFire labelling system (IDT, Inc.). While none of these three miRNAs was expressed at high levels in either adult rat liver or brain, a similar order of hybridization signals was obtained from both Northerns and miRMAX microarrays. The background-subtracted median intensities from the microarray hybridizations matched the pattern observed for the Northern blots between liver and brain samples across all three miRNAs (FIG. 3B and C), indicating that our miRNA detection method was able to mimic results obtained via traditional Northern blot methods. In addition, observable signals of weakly-expressing miRNAs (miR-191 and miR-16 in liver as examples) were relatively greater (as compared to background levels) in the miRMAX system than in the Northern assay. Furthermore, Northern blots generally required 30-fold more input RNA than the microarrays.

To assess the selectivity of our microarray probes, we performed a dot blot comparing hybridization of wt, 1 nt mutated, and 2 nt mutated miR-191 to both the miRMAX probe as well as a probe with a complementary mutation to the 1 nt mutated miR-191 sequence (FIG. 3D). As anticipated, the miRMAX probe for miR-191 strongly hybridized to the wt miR-191, was slightly weaker in hybridizing to the mut1 RNA, and showed only minimal hybridization to the 2 nt mutated RNA. This indicates that the standard Northern assay is no more selective than our microarray assay in distinguishing between miRNA species with only 1 nt difference. The probe design has also been validated and demonstrated to be effective on other assay systems. The Luminex bead assay system has been used previously to detect miRNAs with a LNA labelling technology (20). We synthesized several terminally-aminated probes, using sequences identical to those found on our microarrays. Using the Luminex assay system with the same labelling system as our microarrays, we were able to reproduce the rank order of detection of mir-1, mir-122 and mir-124a in rat heart, liver and brain LMW RNAs, respectively (not shown). These three probes were chosen from microarray results because of their clear tissue-specific expression patterns. Similarly, using these probes in an ELISA-like well-based hybridization system also replicated the microarray results (not shown). These alternative assays further demonstrate the utility of our probe design and sensitive detection system in methods that may be more applicable for high-throughput assay of limited numbers of miRNAs with optimized sequence selectivity.

Comparison of miRNA Levels in Rat Brain and Liver

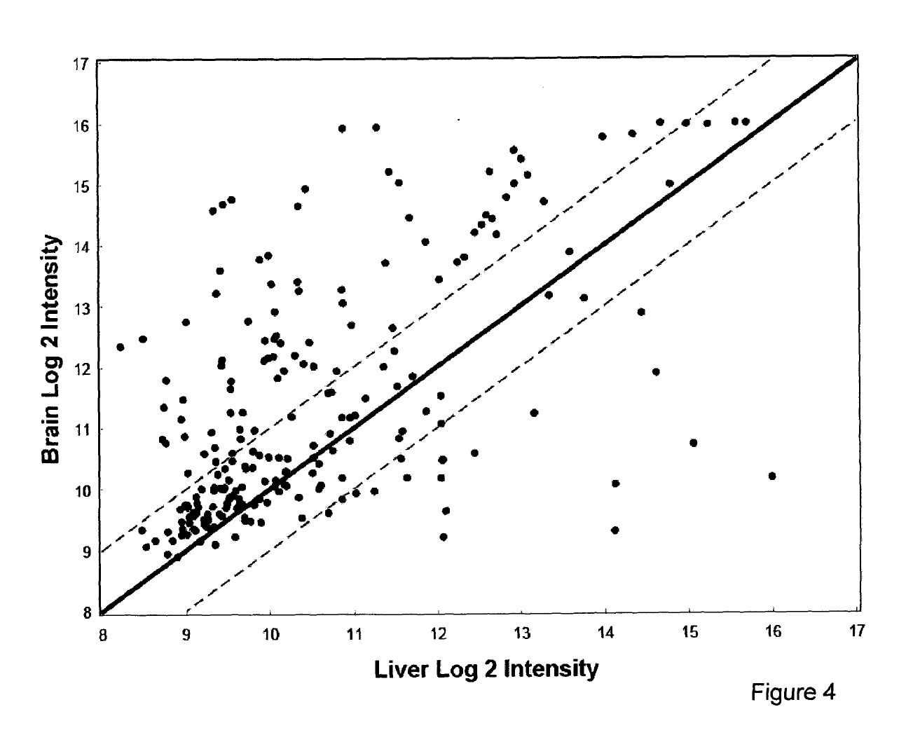

To test and validate the new platform, we chose to examine miRNAs in rat brain and liver, where there exists data for comparison. Three adult rat brain LMW RNA samples (Cy3) and three liver LMW RNA samples (Cy5) were labelled and hybridized to our custom chips. A wide range of log₂ ratios was observed (FIG. 4) indicating a distinct expression profile in each of the two tissues. Using a 2-fold expression level cutoff, it is interesting to note that there are more miRNAs preferentially expressed in brain than in liver. Expression of brain and liver specific miRNAs was well correlated with previously published data regarding. miR-124a, miR-125a & b, miR-128, miR-181, and miR-9, all previously shown to be enriched in brain tissue (18, 22, 39, 40), were also very highly expressed in the brain tissues in our assay. miR-122, miR-192, miR-194, and miR-337 were expressed at levels much higher in liver than brain in our study which again correlates with other studies (19, 26, 39-41).

miRNA Expression in Neural Stem Cells

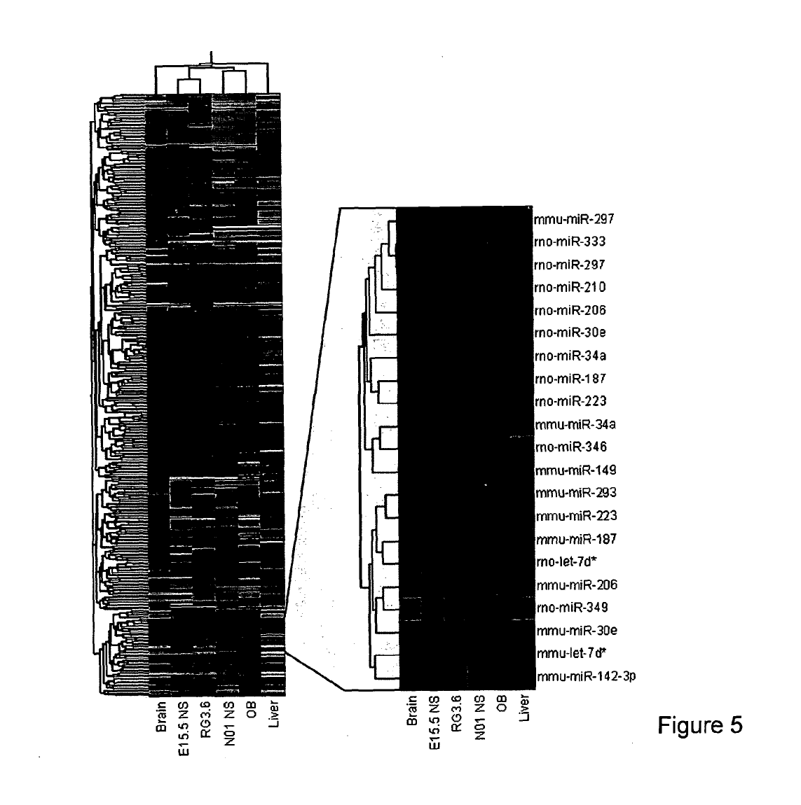

Several studies have indicated that miRNAs may play an important role in stem cell maintenance and differentiation (10, 11, 42, 43). As a broad comparative study, several available rat stem cell populations were assayed using the miRMAX microarray system (FIG. 5). While some miRNAs had similar profiles across all stem cell lines and adult tissues, the vast majority showed dramatic differences in expression between the stem cell lines and the adult tissues. Among the samples tested and clustered, the relationships appear to make sense. Liver is the least related sample. The most similar samples are E15.5 neurospheres and RG3.6 cells, which were derived from E15.5 neurospheres (44). RG3.6 is transfected with v-myc to stabilize a radial glial phenotype. The next most similar samples were neurospheres of N01 clones, derived from rat fetal blood, and olfactory bulb. Among the miRNAs that are enriched compared to brain or liver was a member of the “ES”-specific cluster (42), mir-293. Others (mir-223 and 142s) have been identified for expression in hematopoietic cell lines (10). Interestingly, none of these miRNAs correlates with a list found in human embryonic stem cells or embryonic carcinoma cells (43). In many cases, homologous probes from the two selected species hybridized similarly across all samples. We conclude that rat neural stem cell preparations express distinct populations of miRNAs, as has been observed in other species.

### DISCUSSION

We have developed an optimized miRNA microarray platform, including rationally-designed probes for multiple species printed on a single microarray as well as a high specific-activity labelling method. Our design reduced the predicted variability of miRNA melting temperatures, but retained hybridization intensities similar to unmodified sequence. Using a subset of probes with specific mutations, we find that all probes are specific within 2 nt, and many are detected selectively within 1 nt. Using a detailed hybridization temperature series, we selected the appropriate hybridization temperature (47° C.), a step that is crucial for optimizing sequence specificity. The labelling method employed herein is straightforward, producing directly-labelled miRNA, which allows use of minimal quantities of input RNA and takes advantage of more stable RNA-DNA hybridization properties. Results are similar to Northern blots performed with 30-fold more RNA. Using this platform, we have performed hundreds of arrays with validated and reproducible results, including the detection of tissue-specific expression in rat brain vs. liver, characterization of miRNA expression in several stem cell clones available in our laboratory, and a comparison of brain-specific miRNAs across all five species present on our chip. The latter study highlights the value of including probes for multiple species on a single microarray. Furthermore, the validation of a rational probe design algorithm is expected to be important for extending miRNA assays to high-throughput experiments as the numbers of miRNAs per genome is predicted to increase from 200 up to 1,000 (34). Efficient miRNA microarray platforms will be valuable in identifying miRNAs regulating biological systems and in predicting interactions with specific target mRNAs.

TABLE 2

| Probe | | |
|---|---|---|
| ID | mRNA Probe Name | Probe Sequence |
| 1514 | 1514-mut1-mo-mir- | TGTAAACCATGATGTTCTGCTATGTAAACCATGATGTTCTGCTA |
| | 15b | |
| 1516 | 1515-mut2-mo-mir- | TGTAAAGCATGATGTTCTGCTATGTAAAGCATGATGTTCTGCTA |
| | 15b | |
| 1516 | 1516-rev-mo-mir-15b | TAGCAGCACATCATGGTTTACATAGCAGCACATCATGGTTTACA |
| 1517 | 1517-shuf-mo-mir- | TCATATATTCGGCGATAGAGCTTCATATATTCGGCGATAGAGCT |
| | 15b | |
| 1518 | 1518-mut1-mo-mir-16 | CGCCAATATTTACGTGCTGGTACGCCAATATTTACGTGCTGGTA |
| 1519 | 1519-mut2-mo-mir-16 | CGCCAATATTTAGGTGCTGGTACGCCAATATTTAGGTGCTGGTA |
| 1520 | 1520-rev-mo-mir-16 | TAGCAGCACGTAAATATTGGCGTAGCAGCACGTAAATATTGGCG |
| 1521 | 1521-shuf-mo-mir-16 | CCCAGCATTTATCCGTGGTATACCCAGCATTTATCCGTGGTATA |
| 1522 | 1522-mut1-cel-mir- | AGCTCCTACCCGAAAGATGTAAAGCTCCTACCCGAAAGATGTAA |
| | 246 | |
| 1523 | 1523-mut2-cel-mir- | AGCTCCTACCCGAAAGATTTAAAGCTCCTACCCGAAAGATTTAA |
| | 246 | |
| 1524 | 1524-rev-cel-mir-246 | TTACATGTTTCGGGTAGGAGCTTTACATGTTTCGGGTAGGAGCT |
| 1525 | 1525-shuf-cel-mir-246 | CTAAGCAAAATAGCCGTTACCCCTAAGCAAAATAGCCGTTACCC |
| 1526 | 1526-mut1-has-mir- | CTACCTTCACGAACAGCACTTCTACCTTCACGAACAGCACTT |
| | 93 | |
| 1527 | 1527-mut2-has-mir- | CTACCTTCACGAACAGCAGTTCTACCTTCACGAACAGCAGTT |
| | 93 | |
| 1528 | 1528-rev-has-mir-93 | AAGTGCTGTTCGTGCAGGTAGAAGTGCTGTTCGTGCAGGTAG |
| 1529 | 1529-shuf-has-mir-93 | AATCCCTCCCGAAGTCGCTAAAATCCCTCCCGAAGTCGCTAA |
| 1530 | 1530-mut1-mir-150 | ACTGGTACAAGGGTTGTGAGAACTGGTACAAGGGTTGTGAGA |
| 1531 | 1531-mut2-mir-150 | ACTGGTAGAAGGGTTGTGAGAACTGGTAGAAGGGTTGTGAGA |
| 1532 | 1532-rev-mir-150 | TCTCCCAACCCTTGTACCAGTTCTCCCAACCCTTGTACCAGT |
| 1533 | 1533-shuf-mir-150 | AGCATGGTGTGAACGGAAGGTAGCATGGTGTGAACGGAAGGT |
| 1534 | 1534-mut1-has-mir- | GCGGAACTTAGGCACTGTGAAGCGGAACTTAGGCACTGTGAA |
| | 27a | |
| 1535 | 1535-mut2-has-mir- | GCGGAAGTTAGGCACTGTGAAGCGGAAGTTAGGCACTGTGAA |
| | 27a | |
| 1536 | 1536-rev-has-mir-27a | TTCACAGTGGCTAAGTTCCGCTTCACAGTGGCTAAGTTCCGC |
| 1537 | 1537-shuf-has-mir- | TAGCGAACGAGCCACTGTAGTTAGCGAACGAGCCACTGTAGT |
| | 27a | |
| 1538 | 1538-muti-mir-200c | TCCATCATTACCCGGCATTATTTCCATCATTACCCGGCATTATT |
| 1539 | 1539-mut2-mir-200c | TCCATCATTACCCTGCATTATTTCCATCATTACCCTGCATTATT |
| 1540 | 1540-rev-mir-200c | AATACTGCCGGGTAATGATGGAAATACTGCCGGGTAATGATGGA |
| 1541 | 1541-shuf-mir-200c | TTGCCAACCTTCCTCAGGATATTTGCCAACCTTCCTCAGGATAT |
| 1542 | 1542-mut1-mmu-mir- | AGCTGCTTTTGGGATTGCGTTAGCTGCTTTTGGGATTGCGTT |
| | 191 | |
| 1543 | 1543-mut2-mmu-mir- | AGCTTCTTTTGGGATTGCGTTAGCTTCTTTTGGGATTGCGTT |
| | 191 | |
| 1544 | 1544-rev-mmu-mir- | AACGGAATCCCAAAAGCAGCTAACGGAATCCCAAAAGCAGCT |
| | 191 | |
| 1545 | 1545-shuf-mmu-mir- | CTGTCTGCGGATTTGGTTTCACTGTCTGCGGATTTGGTTTCA |
| | 191 | |
| 1546 | 1546-mut1-cel-mir- | CATACGACTTTGTACAACCAAACATACGACTTTGTACAACCAAA |
| | 244 | |
| 1547 | 1547-mut2-cel-mir- | CATACGACTTTGTAGAACCAAACATACGACTTTGTAGAACCAAA |
| | 244 | |
| 1548 | 1548-rev-cel-mir-244 | TTTGGTTGTACAAAGTGGTATGTTTGGTTGTACAAAGTGGTATG |
| 1549 | 1549-shuf-cel-mir-244 | TAAACCCAGACATTACTATCACTAAACCCAGACATTACTATCAC |
| 1550 | 1550-mut1-mmu-mir- | ACACTCAAAACCTGGCGGGACTACACTCAAAACCTGGCGGGACT |
| | 292 | |
| 1551 | 1551-mut2-mmu-mir- | ACACTCAAAAGCTGGCGGGACTACACTCAAAAGCTGGCGGGACT |
| | 292 | |
| 1552 | 1552-rev-mmu-mir- | AGTGCCGCCAGGTTTTGAGTGTAGTGCCGCCAGGTTTTGAGTGT |
| | 292 | |
| 1553 | 1553-shuf-mmu-mir- | TAAGACCGACGACGACCTCTACTAAGACCGACGACGACCTCTAC |
| | 292 | |
| 1554 | 1554-mut1-mir-324 | ACACGAATGCCCTAGGGGATACACGAATGCCCTAGGGGAT |
| 1555 | 1555-mut2-mir-324 | ACACGAATGCGCTAGGGGATACACGAATGCGCTAGGGGAT |
| 1556 | 1556-rev-mir-324 | ATCCCGTAGGGCATTGGTGTATCCCCTAGGGCATTGGTGT |
| 1557 | 1557-shuf-mir-324 | ACAACTAGGGTACCGCCAGTACAACTAGGGTACCGCCAGT |
| 1558 | 1558-mut1-mo-mir- | CTTCAGCTATCACAGTACTTTACTTCAGCTATCACAGTACTTTA |
| | 101b | |
| 1559 | 1559-mut2-mo-mir- | CTTGAGCTATCACAGTACTTTACTTGAGCTATCACAGTACTTTA |
| | 101b | |
| 1560 | 1560-rev-mo-mir- | TACAGTACTGTGATAGCTGAAGTACAGTACTGTGATAGCTGAAG |
| | 101b | |
| 1561 | 1561-shuf-mo-mir- | TAGCCAGACTATTAGATCTCCTTAGCCAGACTATTAGATCTCCT |
| | 101b | |
| 1562 | 1562-mut1-mir-34c | CAATCAGCTAAGTACACTGCCTCAATCAGCTAAGTACACTGCCT |
| 1563 | 1563-mut2-mir-34c | CAATCAGCTAAGTAGACTGCCTCAATCAGCTAAGTAGACTGCCT |
| 1564 | 1564-rev-mir-34c | AGGCAGTGTAGTTAGCTGATTGAGGCAGTGTAGTTAGCTGATTG |
| 1565 | 1565-shuf-mir-34c | GGATCTAACCTCACAATACTCCGGATCTAACCTCACAATACTCC |
| 1566 | 1566-mut1-mmu-mir- | ACACTTACTGAGGACCTACTAGACACTTACTGAGGACCTACTAG |
| | 325 | |
| 1567 | 1567-mut2-mmu-mir- | ACAGTTACTGAGGACCTACTAGACAGTTACTGAGGACCTACTAG |
| | 325 | |
| 1568 | 1568-rev-mmu-mir- | CTAGTAGGTGCTCAGTAAGTGTCTAGTAGGTGCTCAGTAAGTGT |
| | 325 | |
| 1569 | 1569-shuf-mmu-mir- | CAAACCATTGGTCAAACCGCTTCAAACCATTGGTCAAACCGCTT |
| | 325 | |
| 1570 | 1570-mutt-has-mir- | CCAAGTTCTGTCATGCACTCACCAAGTTCTGTCATGCACTCA |
| | 152 | |
| 1571 | 1571-mut2-has-mir- | CCAATTTCTGTCATGCACTCACCAATTTCTGTCATGCACTCA |
| | 152 | |
| 1572 | 1572-rev-has-mir-152 | TCAGTGCATGACAGAACTTGGTCAGTGCATGACAGAACTTGG |
| 1573 | 1573-shuf-has-mir- | GGAGATATTCCTTCCGTAACCGGAGATATTCCTTCCGTAACC |
| | 152 | |
| 1574 | 1574-mut1-dme-mir- | ACTGGATAGCACCAGCTGTGTACTGGATAGCACCAGCTGTGT |
| | 317 | |
| 1575 | 1575-mut2-dme-mir- | ACTGGATAGCACCAGCTTTGTACTGGATAGCACCAGCTTTGT |
| | 317 | |
| 1576 | 1576-rev-dme-mir- | ACACAGCTGGTGGTATCCAGTACACAGCTGGTGGTATCCAGT |
| | 317 | |
| 1577 | 1577-shuf-dme-mir- | CATACTGTGTTCAGGCGCACACATACTGTGTTCAGGCGCACA |
| | 317 | |
| 1578 | 1578-mut1-dme-mir- | GCAAGAACTCAGACTTTGATGGCAAGAACTCAGACTTTGATG |
| | 11 | |
| 1579 | 1579-mut2-dme-mir- | GCAAGAAGTCAGACTTTGATGGCAAGAAGTCAGACTTTGATG |
| | 11 | |
| 1580 | 1580-rev-dme-mir-11 | CATCACAGTCTGAGTTCTTGCCATCACAGTCTGAGTTCTTGC |
| 1581 | 1581-shuf-dme-mir- | AGAGGGAGCTGTAAACCTTCAAGAGGGAGCTGTAAACCTTCA |
| | 11 | |
| 1582 | 1582-mut1-dme-mir-7 | ACAACAAAATCACTATTCTTCCACAACAAAATCACTATTCTTCC |
| 1583 | 1583-mut2-dme-mir-7 | ACAACAAAATGACTATTCTTCCACAACAAAATGACTATTCTTCC |
| 1584 | 1584-rev-dme-mir-7 | GGAAGACTAGTGATTTTGTTGTGGAAGACTAGTGATTTTGTTGT |
| 1585 | 1585-shuf-dme-mir-7 | TGCCAAACAATACCCATATCTATGCCAAACAATACCCATATCTA |
| 1586 | 1586-mut1-cel-mir-40 | TTAGCTGATGTACACGCGGTGTTAGCTGATGTACACGCGGTG |
| 1587 | 1587-mut2-cel-mir-40 | TTAGCTGATTTACACGCGGTGTTAGCTGATTTACACGCGGTG |
| 1588 | 1588-rev-cel-mir-40 | CACCGGGTGTACATCAGCTAACACCGGGTGTACATCAGCTAA |
| 1589 | 1589-shuf-cel-mir-40 | TTACCTGTGGGTACCCGATAGTTACCTGTGGGTACCCGATAG |
| 1666 | 1666-1mer-cel-mir-40 | TTAGCTGATGTACACCGGGTG |
| 1667 | 1667-1mer-hsa-mir- | GCGGAACTTAGCCACTGTGAA |
| | 27a | |
| 1668 | 1668-1mer-hsa-mir- | CTACCTGCACGAACAGCACTT |
| | 93 | |
| 1669 | 1669-1mer-dme-mir-7 | ACAACAAAATCACTAGTCTTCC |
| 1670 | 1670-1mer-dme-mir- | GCAAGAACTCAGACTGTGATG |
| | 11 | |
| 1671 | 1671-1mer-mmu-mir- | AGCTGCTTTTGGGATTCCGTT |
| | 191 | |
| 1672 | 1672-1mer-cel-mir- | CATACCACTTTGTACAACCAAA |
| | 244 | |
| 1673 | 1673-1mer-cel-mir- | AGCTCCTACCCGAAACATGTAA |
| | 246 | |
| 1674 | 1674-1mer-mmu-mir- | ACACTCAAAACCTGGCGGCACT |
| | 292 | |
| 1675 | 1675-1mer-dme-mir- | ACTGGATACCACCAGCTGTGT |
| | 317 | |
| 1676 | 1676-1mer-hsa-mir- | CCAAGTTCTGTCATGCACTGA |
| | 152 | |
| 1677 | 1677-1mer-hsa-mir- | ACTGGTACAAGGGTTGGGAGA |
| | 150 | |
| 1678 | 1678-1mer-mmu-mir- | ACACCAATGCCCTAGGGGAT |
| | 324 | |
| 1679 | 1679-1mer-mmu-mir- | ACACTTACTGAGCACCTACTAG |
| | 325 | |
| 1680 | 1680-1mer-mo-mir- | CTTCAGCTATCACAGTACTGTA |
| | 101b | |
| 1681 | 1681-1mer-mmu-mir- | TCCATCATTACCCGGCAGTATT |
| | 200c | |
| 1682 | 1682-1mer-hsa-mir- | CAATCAGCTAACTACACTGCCT |
| | 34c | |
| 1683 | 1683-1mer-mo-mir- | TGTAAACCATGATGTGCTGCTA |
| | 15b | |
| 1684 | 1684-1mer-mo-mir-16 | CGCCAATATTTACGTGCTGCTA |
| 1685 | 1685-1mer-mo-mir- | CAATCAGCTAACTACACTGCCT |
| | 34c | |
| 2001 | hsa-let-7a | AACTATACAACCTACTACCTCAAACTATACAACCTACTACCTCA |
| 2002 | hsa-let-7b | AACCACACAACCTACTACCTCAAACCACACAACCTACTACCTCA |
| 2003 | hsa-let-7c | AACCATACAACCTACTACCTCAAACCATACAACCTACTACCTCA |
| 2004 | hsa-let-7d | ACTATGCAACCTACTACCTCTACTATGCAACCTACTACCTCT |
| 2005 | hsa-let-7e | ACTATACAACCTCCTACCTCAACTATACAACCTCCTACCTCA |
| 2006 | hsa-let-7f | AACTATACAATCTACTACCTCAAACTATACAATCTACTACCTCA |
| 2007 | hsa-let-7g | ACTGTACAAACTACTACCTCAACTGTACAAACTACTACCTCA |
| 2008 | hsa-let-7i | ACAGCACAAACTACTACCTCAACAGCACAAACTACTACCTCA |
| 2009 | hsa-miR-1 | TACATACTTCTTTACATTCCATACATACTTCTTTACATTCCA |
| 2010 | hsa-miR-100 | CACAAGTTCGGATCTACGGGTCACAAGTTCGGATCTACGGGT |
| 2011 | hsa-miR-101 | CTTCAGTTATCACAGTACTGTACTTCAGTTATCACAGTACTGTA |
| 2012 | hsa-miR-103 | TCATAGCCCTGTACAATGCTGTCATAGCCCTGTACAATGCTG |
| 2013 | hsa-miR-105 | ACAGGAGTCTGAGCATTTGAACAGGAGTCTGAGCATTTGA |
| 2014 | hsa-miR-106a | CTACCTGCACTGTAAGCACTTTCTACCTGCACTGTAAGCACTTT |
| 2015 | hsa-miR-106b | ATCTGCACTGTCAGCACTTTAATCTGCACTGTCAGCACTTTA |
| 2016 | hsa-miR-107 | TGATAGCCCTGTACAATGCTGTGATAGCCCTGTACAATGCTG |
| 2017 | hsa-miR-10a | CACAAATTCGGATCTACAGGGTCACAAATTCGGATCTACAGGGT |
| 2018 | hsa-miR-10b | ACAAATTCGGTTCTACAGGGTAACAAATTCGGTTCTACAGGGTA |
| 2019 | hsa-miR-122a | ACAAACACCATTGTCACACTCCACAAACACCATTGTCACACTCC |
| 2020 | hsa-miR-124a | TGGCATTCACCGCGTGCCTTAATGGCATTCACCGCGTGCCTTAA |
| 2021 | hsa-miR-125a | CACAGGTTAAAGGGTCTCAGGCACAGGTTAAAGGGTCTCAGG |
| 2022 | hsa-miR-125b | TCACAAGTTAGGGTCTCAGGGTCACAAGTTAGGGTCTCAGGG |
| 2023 | hsa-miR-126 | GCATTATTACTCACGGTACGAGCATTATTACTCACGGTACGA |
| 2024 | hsa-miR-126* | CGCGTACCAAAAGTAATAATGCGCGTACCAAAAGTAATAATG |
| 2025 | hsa-miR-127 | AGCCAAGCTCAGACGGATCCGAAGCCAAGCTCAGACGGATCCGA |
| 2026 | hsa-miR-128a | AAAAGAGACCGGTTCACTGTGAAAAAGAGACCGGTTCACTGTGA |
| 2027 | hsa-miR-128b | GAAAGAGACCGGTTCACTGTGGAAAGAGACCGGTTCACTGTG |
| 2028 | hsa-miR-129 | GCAAGCCCAGACCGCAAAAAGCAAGCCCAGACCGCAAAAA |
| 2029 | hsa-miR-130a | ATGCCCTTTTAACATTGCACTGATGCCCTTTTAACATTGCACTG |
| 2030 | hsa-miR-130b | ATGCCCTTTCATCATTGCACTGATGCCCTTTCATCATTGCACTG |
| 2031 | hsa-miR-132 | CGACCATGGCTGTAGACTGTTCGACCATGGCTGTAGACTGTT |
| 2032 | hsa-miR-133a | ACAGCTGGTTGAAGGGGACCAAACAGCTGGTTGAAGGGGACCAA |
| 2033 | hsa-miR-133b | TAGCTGGTTGAAGGGGACCAATAGCTGGTTGAAGGGGACCAA |
| 2034 | hsa-miR-134 | CCTCTGGTCAACCAGTCACACCTCTGGTCAACCAGTCACA |
| 2035 | hsa-miR-135a | TCACATAGGAATAAAAAGCCATTCACATAGGAATAAAAAGCCAT |
| 2036 | hsa-miR-135b | CACATAGGAATGAAAAGCCATACACATAGGAATGAAAAGCCATA |
| 2037 | hsa-miR-136 | TCCATCATCAAAACAAATGGAGTCCATCATCAAAACAAATGGAG |
| 2038 | hsa-miR-137 | CTACGCGTATTCTTAAGCAATACTACGCGTATTCTTAAGCAATA |
| 2039 | hsa-miR-138 | GATTCACAACACCAGCTGATTCACAACACCAGCT |
| 2040 | hsa-miR-139 | AGACACGTGCACTGTAGAAGACACGTGCACTGTAGA |
| 2041 | hsa-miR-140 | CTACCATAGGGTAAAACCACTCTACCATAGGGTAAAACCACT |
| 2042 | hsa-miR-141 | CCATCTTTACCAGACAGTGTTACCATCTTTACCAGACAGTGTTA |
| 2043 | hsa-miR-142-3p | TCCATAAAGTAGGAAACACTACTCGATAAAGTAGGAAACACTAC |
| 2044 | hsa-miR-142-5p | GTAGTGCTTTCTACTTTATGGTAGTGCTTTCTACTTTATG |
| 2045 | hsa-miR-143 | TGAGCTACAGTGCTTCATCTCATGAGCTACAGTGCTTCATCTCA |
| 2046 | hsa-miR-144 | CTAGTACATCATCTATACTGTACTAGTACATCATCTATACTGTA |
| 2047 | hsa-miR-145 | AAGGGATTCCTGGGAAAACTGAAGGGATTCCTGGGAAAACTG |
| 2048 | hsa-miR-146a | AACCCATGGAATTCAGTTCTCAAACCCATGGAATTCAGTTCTCA |
| 2049 | hsa-miR-146b | AGCCTATGGAATTCAGTTCTCAAGCCTATGGAATTCAGTTCTCA |
| 2050 | hsa-miR-147 | GCAGAAGCATTTCCACACACGCAGAAGCATTTCCACACAC |
| 2051 | hsa-miR-148a | ACAAAGTTCTGTAGTGCACTGAACAAAGTTCTGTAGTGCACTGA |
| 2052 | hsa-miR-148b | ACAAAGTTCTGTGATGCACTGAACAAAGTTCTGTGATGCACTGA |
| 2053 | hsa-miR-149 | AGTGAAGACACGGAGCCAGAAGTGAAGACACGGAGCCAGA |
| 2054 | hsa-miR-150 | ACTGGTACAAGGGTTGGGAGAACTGGTACAAGGGTTGGGAGA |
| 2055 | hsa-miR-151 | CCTCAAGGAGCTTCAGTCTAGCCTCAAGGAGCTTCAGTCTAG |
| 2056 | hsa-miR-152 | CCCAAGTTCTGTCATGCACTGCCCAAGTTCTGTCATGCACTG |
| 2057 | hsa-miR-153 | TCACTTTTGTGACTATGCAATCACTTTTGTGACTATGCAA |
| 2058 | hsa-miR-154 | CGAAGGCAACACGGATAACCTCGAAGGCAACACGGATAACCT |
| 2059 | hsa-miR-154* | AATAGGTCAACCGTGTATGATVAATAGGTCAACCGTGTATGATT |
| 2060 | hsa-miR-155 | CCCCTATCACGATTAGCATTAACCCCTATCACGATTAGCATTAA |
| 2061 | hsa-miR-15a | CACAAACCATTATGTGCTGCTACACAAACCATTATGTGCTGCTA |
| 2062 | hsa-miR-15b | TGTAAACCATGATGTGCTGCTATGTAAACCATGATGTGCTGCTA |
| 2063 | hsa-miR-16 | CGCCAATATTTACGTGCTGCTACGCCAATATTTACGTGCTGCTA |
| 2064 | hsa-miR-17-3p | ACAAGTGCCTTCACTGCAGTACAAGTGCCTTCACTGCAGT |
| 2065 | hsa-miR-17-5p | ACTACCTGCACTGTAAGCACTTACTACCTGCACTGTAAGCACTT |
| 2066 | hsa-miR-181a | ACTCACCGACAGCGTTGAATGACTCACCGACAGCGTTGAATG |
| 2067 | hsa-miR-181b | CCCACCGACAGCAATGAATGTCCCACCGACAGCAATGAATGT |
| 2068 | hsa-miR-181c | ACTCACCGACAGGTTGAATGTTACTCACCGACAGGTTGAATGTT |
| 2069 | hsa-miR-181d | AACCCACCGACAACAATGAATGAACCCACCGACAACAATGAATG |
| 2070 | hsa-miR-182 | TGTGAGTTCTACCATTGCCAAATGTGAGTTCTACCATTGCCAAA |
| 2071 | hsa-miR-182* | TAGTTGGCAAGTCTAGAACCATAGTTGGCAAGTCTAGAACCA |
| 2072 | hsa-miR-183 | CAGTGAATTCTACCAGTGCCATCAGTGAATTCTACCAGTGCCAT |
| 2073 | hsa-miR-184 | ACCCTTATCAGTTCTCCGTCCACCCTTATCAGTTCTCCGTCC |
| 2074 | hsa-miR-185 | GAACTGCCTTTCTCTCCAGAACTGCCTTTCTCTCCA |
| 2075 | hsa-miR-186 | AGCCCAAAAGGAGAATTCTTTGAGCCCAAAAGGAGAATTCTTTG |
| 2076 | hsa-miR-187 | GGCTGCAACACAAGACACGAGGCTGCAACACAAGACACGA |
| 2077 | hsa-miR-188 | ACCCTCCACCATGCAAGGGATACCCTCCACCATGCAAGGGAT |
| 2078 | hsa-miR-189 | ACTGATATCAGCTCAGTAGGCAACTGATATCAGCTCAGTAGGCA |
| 2079 | hsa-miR-18a | TATCTGCACTAGATGCACCTTATATCTGCACTAGATGCACCTTA |
| 2080 | hsa-miR-18b | TAACTGCACTAGATGCACCTTATAACTGCACTAGATGCACCTTA |
| 2081 | hsa-miR-190 | ACCTAATATATCAAACATATCAACCTAATATATCAAACATATCA |
| 2082 | hsa-miR-191 | AGCTGCTTTTGGGATTCCGTTAGCTGCTTTTGGGATTCCGTT |
| 2083 | hsa-miR-191* | GGGACGAAATCCAAGCGCAGGGACGAAATCCAAGCGCA |
| 2084 | hsa-miR-192 | GGCTGTCAATTCATAGGTCAGGGCTGTCAATTCATAGGTCAG |
| 2085 | hsa-miR-193a | CTGGGACTTTGTAGGCCAGTTCTGGGACTTTGTAGGCCAGTT |
| 2086 | hsa-miR-193b | AAAGCGGGACTTTGAGGGCCAAAAGCGGGACTTTGAGGGCCA |
| 2087 | hsa-miR-194 | TCCACATGGAGTTGCTGTTACATCCACATGGAGTTGCTGTTACA |
| 2088 | hsa-miR-195 | GCCAATATTTCTGTGCTGCTAGCCAATATTTCTGTGCTGCTA |
| 2089 | hsa-miR-196a | CCAACAACATGAAACTACCTACCAACAACATGAAACTACCTA |
| 2090 | hsa-miR-196b | CCAACAACAGGAAACTACCTACCAACAACAGGAAACTACCTA |
| 2091 | hsa-miR-197 | TGGGTGGAGAAGGTGGTGAATGGGTGGAGAAGGTGGTGAA |
| 2092 | hsa-miR-198 | CCTATCTCCCCTCTGGACCCTATCTCCCCTCTGGAC |
| 2093 | hsa-miR-199a | GAACAGGTAGTCTGAACACTGGAACAGGTAGTCTGAACACTG |
| 2094 | hsa-miR-199a* | AACCAATGTGCAGACTACTGTAAACCAATGTGCAGACTACTGTA |
| 2095 | hsa-miR-199b | GAACAGATAGTCTAAACACTGGGAACAGATAGTCTAAACACTGG |
| 2096 | hsa-miR-19a | TCAGTTTTGCATAGATTTGCACTCAGTTTTGCATAGATTTGCAC |
| 2097 | hsa-miR-19b | TCAGTTTTGCATGGATTTGCACTCAGTTTTGCATGGATTTGCAC |
| 2098 | hsa-miR-200a | ACATCGTTACCAGACAGTGTTAACATCGTTACCAGACAGTGTTA |
| 2099 | hsa-miR-200a* | TCCAGCACTGTCCGGTAAGATTCCAGCACTGTCCGGTAAGAT |
| 2100 | hsa-miR-200b | GTCATCATTACCAGGCAGTATTGTCATCATTACCAGGCAGTATT |
| 2101 | hsa-miR-200c | CCATCATTACCCGGCAGTATTACCATCATTACCCGGCAGTATTA |
| 2102 | hsa-miR-202 | TTTTCCCATGCCCTATACCTCTTTTTCCCATGCCCTATACCTCT |
| 2103 | hsa-miR-202* | AAAGAAGTATATGCATAGGAAAAAAGAAGTATATGCATAGGAAA |
| 2104 | hsa-miR-203 | CTAGTGGTCCTAAACATTTCACCTAGTGGTCCTAAACATTTCAC |
| 2105 | hsa-miR-204 | AGGCATAGGATGACAAAGGGAAAGGCATAGGATGACAAAGGGAA |
| 2106 | hsa-miR-205 | AGACTCCGGTGGAATGAAGGAAGACTCCGGTGGAATGAAAGGA |
| 2107 | hsa-miR-206 | CCACACACTTCCTTACATTCCACCACACACTTCCTTACATTCCA |
| 2108 | hsa-miR-208 | ACAAGCTTTTTGCTCGTCTTATACAAGCTTTTTGCTCGTCTTAT |
| 2109 | hsa-miR-20a | CTACCTGCACTATAAGCACTTTCTACCTGCACTATAAGCACTTT |
| 2110 | hsa-miR-20b | CTACCTGCACTATGAGCACTTTCTACCTGCACTATGAGCACTTT |
| 2111 | hsa-miR-21 | TCAACATCAGTCTGATAAGCTATCAACATCAGTCTGATAAGCTA |
| 2112 | hsa-miR-210 | TCAGCCGCTGTCACACGCACATCAGCCGCTGTCACACGCACA |
| 2113 | hsa-miR-211 | AGGCGAAGGATGACAAAGGGAAGGCGAAGGATGACAAAGGGA |
| 2114 | hsa-miR-212 | GCCGTGACTGGAGACTGTTAGCCGTGACTGGAGACTGTTA |
| 2115 | hsa-miR-213 | GGTACAATCAACGGTCGATGGGGTACAATCAACGGTCGATGG |
| 2116 | hsa-miR-214 | TGCCTGTCTGTGCCTGCTGTTGCCTGTCTGTGCCTGCTGT |
| 2117 | hsa-miR-215 | GTCTGTCAATTCATAGGTCATGTCTGTCAATTCATAGGTCAT |
| 2118 | hsa-miR-216 | CACAGTTGCCAGCTGAGATTACACAGTTGCCAGCTGAGATTA |
| 2119 | hsa-miR-217 | ATCCAATCAGTTCCTGATGCAGATCCAATCAGTTCCTGATGCAG |
| 2120 | hsa-miR-218 | ACATGGTTAGATCAAGCACAAACATGGTTAGATCAAGCACAA |
| 2121 | hsa-miR-219 | AGAATTGCGTTTGGACAATCAAGAATTGCGTTTGGACAATCA |
| 2122 | hsa-miR-22 | ACAGTTCTTCAACTGGCAGCTTACAGTTCTTCAACTGGCAGCTT |
| 2123 | hsa-miR-220 | AAAGTGTCAGATACGGTGTGGAAAGTGTCAGATACGGTGTGG |
| 2124 | hsa-miR-221 | AAACCCAGCAGACAATGTAGCTAAACCCAGCAGACAATGTAGCT |
| 2125 | hsa-miR-222 | AGACCCAGTAGCCAGATGTAGAGACCCAGTAGCCAGATGTAG |
| 2126 | hsa-miR-223 | GGGGTATTTGACAAACTGACAGGGGTATTTGACAAACTGACA |
| 2127 | hsa-miR-224 | TAAACGGAACCACTAGTGACTTTAAACGGAACCACTAGTGACTT |
| 2128 | hsa-miR-23a | GGAAATCCCTGGCAATGTGATGGAAATCCCTGGCAATGTGAT |
| 2129 | hsa-miR-23b | GGTAATCCCTGGCAATGTGATGGTAATCCCTGGCAATGTGAT |
| 2130 | hsa-miR-24 | TGTTCCTGCTGAACTGAGCCATGTTCCTGCTGAACTGAGCCA |
| 2131 | hsa-miR-25 | TCAGACCGAGACAAGTGCAATTCAGACCGAGACAAGTGCAAT |
| 2132 | hsa-miR-26a | GCCTATCCTGGATTACTTGAAGCCTATCCTGGATTACTTGAA |
| 2133 | hsa-miR-26b | AACCTATCCTGAATTACTTGAAAACCTATCCTGAATTACTTGAA |
| 2134 | hsa-miR-27a | GCGGAACTTAGCCACTGTGAAGCGGAACTTAGCCACTGTGAA |
| 2135 | hsa-miR-27b | GCAGAACTTAGCCACTGTGAAGCAGAACTTAGCCACTGTGAA |
| 2136 | hsa-miR-28 | CTCAATAGACTGTGAGCTCCTTCTCAATAGACTGTGAGCTCCTT |
| 2137 | hsa-miR-296 | ACAGGATTGAGGGGGGGCCCTACAGGATTGAGGGGGGGCCCT |
| 2138 | hsa-miR-299-3p | AAGCGGTTTACCATCCCACATAAAGCGGTTTACCATCCCACATA |
| 2139 | hsa-miR-29a | AACCGATTTCAGATGGTGCTAAACCGATTTCAGATGGTGCTA |
| 2140 | hsa-miR-29b | AACACTGATTTCAAATGGTGCTAACACTGATTTCAAATGGTGCT |
| 2141 | hsa-miR-29c | ACCGATTTCAAATGGTGCTAACCGATTTCAAATGGTGCTA |
| 2142 | hsa-miR-301 | GCTTTGACAATACTATTGCACTGCTTTGACAATACTATTGCACT |
| 2143 | hsa-miR-302a | TCACCAAAACATGGAAGCACTTTCACCAAAACATGGAAGCACTT |
| 2144 | hsa-miR-302a* | AAAGCAAGTACATCCACGTTTAAAAGCAAGTACATCCACGTTTA |
| 2145 | hsa-miR-302b | CTACTAAAACATGGAAGCACTTCTACTAAAACATGGAAGCACTT |
| 2146 | hsa-miR-302b* | AGAAAGCACTTCCATGTTAAAGAGAAAGCACTTCCATGTTAAAG |
| 2147 | hsa-miR-302c | CCACTGAAACATGGAAGCACTTCCACTGAAACATGGAAGCACTT |
| 2148 | hsa-miR-302c* | CAGCAGGTACCCCCATGTTAACAGCAGGTACCCCCATGTTAA |
| 2149 | hsa-miR-302d | ACACTCAAACATGGAAGCACTTACACTCAAACATGGAAGCACTT |
| 2150 | hsa-miR-30a-3p | GCTGCAAACATCCGACTGAAAGCTGCAAACATCCGACTGAAA |
| 2151 | hsa-miR-30a-5p | CTTCCAGTCGAGGATGTTTACACTTCCAGTCGAGGATGTTTACA |
| 2152 | hsa-miR-30b | AGCTGAGTGTAGGATGTTTACAAGCTGAGTGTAGGATGTTTACA |
| 2153 | hsa-miR-30c | GCTGAGAGTGTAGGATGTTTACGCTGAGAGTGTAGGATGTTTAC |
| 2154 | hsa-miR-30d | CTTCCAGTCGGGGATGTTTACCTTCCAGTCGGGGATGTTTAC |
| 2155 | hsa-miR-30e-3p | GCTGTAAACATCCGACTGAAAGGCTGTAAACATCCGACTGAAAG |
| 2156 | hsa-miR-30e-5p | TCCAGTCAAGGATGTTTACATCCAGTCAAGGATGTTTACA |
| 2157 | hsa-miR-31 | CAGCTATGCCAGCATCTTGCCAGCTATGCCAGCATCTTGC |
| 2158 | hsa-miR-32 | GCAACTTAGTAATGTGCAATGCAACTTAGTAATGTGCAAT |
| 2159 | hsa-miR-320 | TTCGCCCTCTCAACCCAGCTTTTTCGCCCTCTCAACCCAGCTTT |
| 2160 | hsa-miR-323 | AGAGGTCGACCGTGTAATGTGAGAGGTCGACCGTGTAATGTG |
| 2161 | hsa-miR-324-3p | AGCAGCACCTGGGGCAGTAGCAGCACCTGGGGCAGT |
| 2162 | hsa-miR-324-5p | ACACCAATGCCCTAGGGGATACACCAATGCCCTAGGGGAT |
| 2163 | hsa-miR-325 | ACACTTACTGGACACCTACTAGACACTTACTGGACACCTACTAG |
| 2164 | hsa-miR-326 | TGGAGGAAGGGCCCAGATGGAGGAAGGGCCCAGA |
| 2165 | hsa-miR-328 | ACGGAAGGGCAGAGAGGGCCAACGGAAGGGCAGAGAGGGCCA |
| 2166 | hsa-miR-329 | AAAGAGGTTAACCAGGTGTGTTAAAGAGGTTAACCAGGTGTGTT |
| 2167 | hsa-miR-33 | CAATGCAACTACAATGCACCAATGCAACTACAATGCAC |
| 2168 | hsa-miR-330 | TCTCTGCAGGCCGTGTGCTTTTCTCTGCAGGCCGTGTGCTTT |
| 2169 | hsa-miR-331 | TTCTAGGATAGGCCCAGGGTTCTAGGATAGGCCCAGGG |
| 2170 | hsa-miR-335 | ACATTTTTCGTTATTGCTCTTGACATTTTTCGTTATTGCTCTTG |
| 2171 | hsa-miR-337 | AAAGGCATCATATAGGAGCTGGAAAGGCATCATATAGGAGCTGG |
| 2172 | hsa-miR-338 | TCAACAAAATCACTGATGCTGGTCAACAAAATCACTGATGCTGG |
| 2173 | hsa-miR-339 | TGAGCTCCTGGAGGACAGGGATGAGCTCCTGGAGGACAGGGA |
| 2174 | hsa-miR-340 | GGCTATAAAGTAACTGAGACGGGGCTATAAAGTAACTGAGACGG |
| 2175 | hsa-miR-342 | ACGGGTGCGATTTCTGTGTGAACGGGTGCGATTTCTGTGTGA |
| 2176 | hsa-miR-345 | CCTGGACTAGGAGTCAGCACCTGGACTAGGAGTCAGCA |
| 2177 | hsa-miR-346 | AGAGGCAGGCATGCGGGCAGAAGAGGCAGGCATGCGGGCAGA |
| 2178 | hsa-miR-34a | AACAACCAGCTAAGACACTGCAACAACCAGCTAAGACACTGC |
| 2179 | hsa-miR-34b | CAATCAGCTAATGACACTGCCTCAATCAGCTAATGACACTGCCT |
| 2180 | hsa-miR-34c | CAATCAGCTAACTACACTGCCTCAATCAGCTAACTACACTGCCT |
| 2181 | hsa-miR-361 | GTACCCCTGGAGATTCTGATAAGTACCCCTGGAGATTCTGATAA |
| 2182 | hsa-miR-362 | TCACACCTAGGTTCCAAGGATTTCACACCTAGGTTCCAAGGATT |
| 2183 | hsa-miR-363 | TTACAGATGGATACCGTGCAATTTACAGATGGATACCGTGCAAT |
| 2184 | hsa-miR-365 | ATAAGGATTTTTAGGGGCATTAATAAGGATTTTTAGGGGCATTA |
| 2185 | hsa-miR-367 | TCACCATTGCTAAAGTGCAATTTCACCATTGCTAAAGTGCAATT |
| 2186 | hsa-miR-368 | AAACGTGGAATTTCCTCTATGTAAACGTGGAATTTCCTCTATGT |
| 2187 | hsa-miR-369-3p | AAAGATCAACCATGTATTATTAAAGATCAACCATGTATTATT |
| 2188 | hsa-miR-369-5p | GCGAATATAACACGGTCGATCTGCGAATATAACACGGTCGATCT |
| 2189 | hsa-miR-370 | CAGGTTCCACCCCAGCACAGGTTCCACCCCAGCA |
| 2190 | hsa-miR-371 | ACACTCAAAAGATGGCGGCACACACTCAAAAGATGGCGGCAC |
| 2191 | hsa-miR-372 | ACGCTCAAATGTCGCAGCACTACGCTCAAATGTCGCAGCACT |
| 2192 | hsa-miR-373 | ACACCCCAAAATCGAAGCACTTACACCCCAAAATCGAAGCACTT |
| 2193 | hsa-miR-373* | GAAAGCGCCCCCATTTTGAGTGAAAGCGCCCCCATTTTGAGT |
| 2194 | hsa-miR-374 | CACTTATCAGGTTGTATTATAACACTTATCAGGTTGTATTATAA |
| 2195 | hsa-miR-375 | TCACGCGAGCCGAACGAACAAATCACGCGAGCCGAACGAACAAA |
| 2196 | hsa-miR-376a | ACGTGGATTTTCCTCTATGATACGTGGATTTTCCTCTATGAT |
| 2197 | hsa-miR-376b | AACATGGATTTTCCTCTATGATAACATGGATTTTCCTCTATGAT |
| 2198 | hsa-miR-377 | ACAAAAGTTGCCTTTGTGTGATACAAAAGTTGCCTTTGTGTGAT |
| 2199 | hsa-miR-378 | ACACAGGACCTGGAGTCAGGAACACAGGACCTGGAGTCAGGA |
| 2200 | hsa-miR-379 | TACGTTCCATAGTCTACCATACGTTCCATAGTCTACCA |
| 2201 | hsa-miR-380-3p | AAGATGTGGACCATATTACATAAAGATGTGGACCATATTACATA |
| 2202 | hsa-miR-380-5p | GCGCATGTTCTATGGTCAACCGCGCATGTTCTATGGTCAACC |
| 2203 | hsa-miR-381 | ACAGAGAGCTTGCCCTTGTATAACAGAGAGCTTGCCCTTGTATA |
| 2204 | hsa-miR-382 | CGAATCCACCACGAACAACTTCGAATCCACCACGAACAACTT |
| 2205 | hsa-miR-383 | AGCCACAATCACCTTCTGATCTAGCCACAATCACCTTCTGATCT |
| 2206 | hsa-miR-384 | TATGAACAATTTCTAGGAATTATGAACAATTTCTAGGAAT |
| 2207 | hsa-miR-409-3p | AGGGGTTCACCGAGCAACATTAGGGGTTCACCGAGCAACATT |
| 2208 | hsa-miR-409-5p | TGCAAAGTTGCTCGGGTAACCTGCAAAGTTGCTCGGGTAACC |
| 2209 | hsa-miR-410 | AACAGGCCATCTGTGTTATATTAACAGGCCATCTGTGTTATATT |
| 2210 | hsa-miR-412 | ACGGCTAGTGGACCAGGTGAAACGGCTAGTGGACCAGGTGAA |
| 2211 | hsa-miR-422a | GCCTTCTGACCCTAAGTCCAGCCTTCTGACCCTAAGTCCA |
| 2212 | hsa-miR-422b | GCCTTCTGACTCCAAGTCCAGCC1TCTGACTCCAAGTCCA |
| 2213 | hsa-miR-423 | TGAGGGGCCTCAGACCGAGCTTGAGGGGCCTCAGACCGAGCT |
| 2214 | hsa-miR-424 | TTCAAAACATGAATTGCTGCTGTTCAAAACATGAATTGCTGCTG |
| 2215 | hsa-miR-425 | CGGACACGACATTCCCGATCGGACACGACATVCCCGAT |
| 2216 | hsa-miR-429 | ACGGTTTTACCAGACAGTATTAACGGTTTTACCAGACAGTATTA |
| 2217 | hsa-miR-431 | TGCATGACGGCCTGCAAGACATGCATGACGGCCTGCAAGACA |
| 2218 | hsa-miR-432 | CCACCCAATGACCTACTCCAACCACCCAATGACCTACTCCAA |
| 2219 | hsa-miR-432* | AGACATGGAGGAGCCATCCAAGACATGGAGGAGCCATCCA |
| 2220 | hsa-miR-433 | ACACCGAGGAGCCCATCATGATACACCGAGGAGCCCATCATGAT |
| 2221 | hsa-miR-448 | ATGGGACATCCTACATATGCAAATGGGACATCCTACATATGCAA |
| 2222 | hsa-miR-449 | ACCAGCTAACAATACACTGCCAACCAGCTAACAATACACTGCCA |
| 2223 | hsa-miR-450 | TATTAGGAACACATCGCAAAAATATTAGGAACACATCGCAAAAA |
| 2224 | hsa-miR-451 | AAACTCAGTAATGGTAACGGTTAAACTCAGTAATGGTAACGGTT |
| 2225 | hsa-miR-452 | GTCTCAGTTTCCTCTGCAAACAGTCTCAGTTTCCTCTGCAAACA |
| 2226 | hsa-miR-452* | CTTCTTTGCAGATGAGACTGACTTCTTTGCAGATGAGACTGA |
| 2227 | hsa-miR-453 | GAACTCACCACGGACAACCTGAACTCACCACGGACAACCT |
| 2228 | hsa-miR-485-3p | AGAGGAGAGCCGTGTATGACAGAGGAGAGCCGTGTATGAC |
| 2229 | hsa-miR-485-5p | AATTCATCACGGCCAGCCTCTAATTCATCACGGCCAGCCTCT |
| 2230 | hsa-miR-488 | TTGAGAGTGCCATTATCTGGGTTGAGAGTGCCATTATCTGGG |
| 2231 | hsa-miR-489 | CTGCCGTATATGTGATGTCACTCTGCCGTATATGTGATGTCACT |
| 2232 | hsa-miR-490 | AGCATGGAGTCCTCCAGGTTAGCATGGAGTCCTCCAGGTT |
| 2233 | hsa-miR-491 | TCCTCATGGAAGGGTTCCCCATCCTCATGGAAGGGTTCCCCA |
| 2234 | hsa-miR-492 | AAGAATCTTGTCCCGCAGGTCAAGAATCTTGTCCCGCAGGTC |
| 2235 | hsa-miR-493 | AATGAAAGCCTACCATGTACAAAATGAAAGCCTACCATGTACAA |
| 2236 | hsa-miR-494 | AAGAGGTTTCCCGTGTATGTTTAAGAGGTTTCCCGTGTATGTTT |
| 2237 | hsa-miR-495 | AAAGAAGTGCACCATGTTTGTTAAAGAAGTGCACCATGTTTGTT |
| 2238 | hsa-miR-496 | GAGATTGGCCATGTAATGAGATTGGCCATGTAAT |
| 2239 | hsa-miR-497 | ACAAACCACAGTGTGCTGCTGACAAACCACAGTGTGCTGCTG |
| 2240 | hsa-miR-498 | AAAAACGCCCCCTGGCTTGAAAAAAACGCCCCCTGGCTTGAA |
| 2241 | hsa-miR-499 | TTAAACATCACTGCAAGTCTTATTAAACATCACTGCAAGTCTTA |
| 2242 | hsa-miR-500 | AGAATCCTTGCCCAGGTGCATAGAATCCTTGCCCAGGTGCAT |
| 2243 | hsa-miR-501 | TCTCACCCAGGGACAAAGGATTCTCACCCAGGGAGAAAGGAT |
| 2244 | hsa-miR-502 | TAGCACCCAGATAGCAAGGATTAGCACCCAGATAGCAAGGAT |
| 2245 | hsa-miR-503 | TGCAGAACTGTTCCCGCTGCTATGCAGAACTGTTCCCGCTGCTA |
| 2246 | hsa-miR-504 | ATAGAGTGCAGACCAGGGTCTATAGAGTGCAGACCAGGGTCT |
| 2247 | hsa-miR-505 | GAGGAAACCAGCAAGTGTTGAGAGGAAACCAGCAAGTGTTGA |
| 2248 | hsa-miR-506 | TCTACTCAGAAGGGTGCCTTATCTACTCAGAAGGGTGCCTTA |
| 2249 | hsa-miR-507 | TTCACTCCAAAAGGTGCAAAATTCACTCCAAAAGGTGCAAAA |
| 2250 | hsa-miR-508 | TCTACTCCAAAAGGCTACAATCTCTACTCCAAAAGGCTACAATC |
| 2251 | hsa-miR-509 | TCTACCCACAGACGTACCAATTCTACCCACAGACGTACCAAT |
| 2252 | hsa-miR-510 | TGTGATTGCCACTCTCCTGAGTGTGATTGCCACTCTCCTGAG |
| 2253 | hsa-miR-511 | TGACTGCAGAGCAAAAGACACTGACTGCAGAGCAAAAGACAC |
| 2254 | hsa-miR-512-3p | GACCTCAGCTATGACAGCACTGACCTCAGCTATGACAGCACT |
| 2255 | hsa-miR-512-5p | AAAGTGCCCTCAAGGCTGAGTAAAGTGCCCTCAAGGCTGAGT |
| 2256 | hsa-miR-513 | ATAAATGACACCTCCCTGTGAAATAAATGACACCTCCCTGTGAA |
| 2257 | hsa-miR-514 | CTACTCACAGAAGTGTCAATCTACTCACAGAAGTGTCAAT |
| 2258 | hsa-miR-515-3p | ACGCTCCAAAAGAAGGCACTCACGCTCCAAAAGAAGGCACTC |
| 2259 | hsa-miR-515-5p | CAGAAAGTGCTTTCTTTTGGAGCAGAAAGTGCTTTCTTTTGGAG |
| 2260 | hsa-miR-516-3p | ACCCTCTGAAAGGAAGCAACCCTCTGAAAGGAAGCA |
| 2261 | hsa-miR-516-5p | AAAGTGCTTCTTACCTCCAGATAAAGTGCTTCTTACCTCCAGAT |
| 2262 | hsa-miR-517* | AGACAGTGCTTCCATCTAGAGAGACAGTGCTTCCATCTAGAG |
| 2263 | hsa-miR-517a | AACACTCTAAAGGGATGCACGAAACACTCTAAAGGGATGCACGA |
| 2264 | hsa-miR-517b | AACACTCTAAAGGGATGCACGAAACACTCTAAAGGGATGCACGA |
| 2265 | hsa-miR-517c | ACACTCTAAAAGGATGCACGATACACTCTAAAAGGATGCACGAT |
| 2266 | hsa-miR-518a | TCCAGCAAAGGGAAGCGCTTTTCCAGCAAAGGGAAGCGCTTT |
| 2267 | hsa-miR-518a-2* | AAAGGGCTTCCCTTTGCAGAAAAGGGCTTCCCTTTGCAGA |
| 2268 | hsa-miR-518b | ACCTCTAAAGGGGAGCGCTTTACCTCTAAAGGGGAGCGCTTT |
| 2269 | hsa-miR-518c | CACTCTAAAGAGAAGCGCTTTGCACTCTAAAGAGAAGCGCTTTG |
| 2270 | hsa-miR-518c* | CAGAAAGTGCTTCCCTCCAGACAGAAAGTGCTTCCCTCCAGA |
| 2271 | hsa-miR-518d | GCTCCAAAGGGAAGCGCTTTGCTCCAAAGGGAAGCGCTTT |
| 2272 | hsa-miR-518e | ACACTCTGAAGGGAAGCGCTTACACTCTGAAGGGAAGCGCTT |
| 2273 | hsa-miR-518f | TCCTCTAAAGAGAAGCGCTTTTCCTCTAAAGAGAAGCGCTTT |
| 2274 | hsa-miR-518f* | AGAGAAAGTGCTTCCCTCTAGAAGAGAAAGTGCTTCCCTCTAGA |
| 2275 | hsa-miR-519a | GTAACACTCTAAAAGGATGCACGTAACACTCTAAAAGGATGCAC |
| 2276 | hsa-miR-519b | AAACCTCTAAAAGGATGCACTTAAACCTCTAAAAGGATGCACTT |
| 2277 | hsa-miR-519c | ATCCTCTAAAAAGATGCACTTTATCCTCTAAAAAGATGCACTTT |
| 2278 | hsa-miR-519d | ACACTCTAAAGGGAGGCACTTTACACTCTAAAGGGAGGCACTTT |
| 2279 | hsa-miR-519e | ACACTCTAAAAGGAGGCACTTTACACTCTAAAAGGAGGCACTTT |
| 2280 | hsa-miR-519e* | GAAAGTGCTCCCTTTTGGAGAAGAAAGTGCTCCCTTTTGGAGAA |
| 2281 | hsa-miR-520a | ACAGTCCAAAGGGAAGCACTTTACAGTCCAAAGGGAAGCACTTT |
| 2282 | hsa-miR-520a* | AGAAAGTACTTCCCTCTGGAGAGAAAGTACTTCCCTCTGGAG |
| 2283 | hsa-miR-520b | CCCTCTAAAAGGAAGCACTTTCCCTCTAAAAGGAAGCACTTT |
| 2284 | hsa-miR-520c | AACCCTCTAAAAGGAAGCACTTAACCCTCTAAAAGGAAGCACTT |
| 2285 | hsa-miR-520d | AACCCACCAAAGAGAAGCACTTAACCCACCAAAGAGAAGCACTT |
| 2286 | hsa-miR-520d* | AGAAAGGGCTTCCCTTTGTAGAAGAAAGGGCTTCCCTTTGTAGA |
| 2287 | hsa-miR-520e | CCCTCAAAAAGGAAGCACTTTCCCTCAAAAAGGAAGCACTTT |
| 2288 | hsa-miR-520f | AACCCTCTAAAAGGAAGCACTTAACCCTCTAAAAGGAAGCACTT |
| 2289 | hsa-miR-520g | ACACTCTAAAGGGAAGCACTTTACACTCTAAAGGGAAGCACTTT |
| 2290 | hsa-miR-520h | ACTCTAAAGGGAAGCACTTTGTACTCTAAAGGGAAGCACTTTGT |
| 2291 | hsa-miR-521 | ACACTCTAAAGGGAAGTGCGTTACACTCTAAAGGGAAGTGCGTT |
| 2292 | hsa-miR-522 | AACACTCTAAAGGGAACCATTTAACACTCTAAAGGGAACCATTT |
| 2293 | hsa-miR-523 | CCTCTATAGGGAAGCGCGTTCCTCTATAGGGAAGCGCGTT |
| 2294 | hsa-miR-524 | ACTCCAAAGGGAAGCGCCTTACTCCAAAGGGAAGCGCCTT |
| 2295 | hsa-miR-524* | GAGAAAGTGCTTCCCTTTGTAGGAGAAAGTGCTTCCCTTTGTAG |
| 2296 | hsa-miR-525 | AGAAAGTGCATCCCTCTGGAGAGAAAGTGCATCCCTCTGGAG |
| 2297 | hsa-miR-525* | GCTCTAAAGGGAAGCGCCTTGCTCTAAAGGGAAGCGCCTT |
| 2298 | hsa-miR-526a | AGAAAGTGCTTCCCTCTAGAGAGAAAGTGCTTCCCTCTAGAG |
| 2299 | hsa-miR-526b | AACAGAAAGTGCTTCCCTCAAGAACAGAAAGTGCTTCCCTCAAG |
| 2300 | hsa-miR-526b* | GCCTCTAAAAGGAAGCACTTTGCCTCTAAAAGGAAGCACTTT |
| 2301 | hsa-miR-526c | AACAGAAAGCGCTTCCCTCTAAACAGAAAGCGCTTCCCTCTA |
| 2302 | hsa-miR-527 | AGAAAGGGCTTCCCTTTGCAGAGAAAGGGCTTCCCTTTGCAG |
| 2303 | hsa-miR-7 | CAACAAAATCACTAGTCTTCCACAACAAAATCACTAGTCTTCCA |
| 2304 | hsa-miR-9 | TCATACAGCTAGATAACCAAAGTCATACAGCTAGATAACCAAAG |
| 2305 | hsa-miR-9* | ACTTTCGGTTATCTAGCTTTACTTTCGGTTATCTAGCTTT |
| 2306 | hsa-miR-92 | AGGCCGGGACAAGTGCAATAAGGCCGGGACAAGTGCAATA |
| 2307 | hsa-miR-93 | CTACCTGCACGAACAGCACTTCTACCTGCACGAACAGCACTT |
| 2308 | hsa-miR-95 | TGCTCAATAAATACCCGTTGAATGCTCAATAAATACCCGTTGAA |
| 2309 | hsa-miR-96 | GCAAAAATGTGCTAGTGCCAAAGCAAAAATGTGCTAGTGCCAAA |
| 2310 | hsa-miR-98 | AACAATACAACTTACTACCTCAAACAATACAACTTACTACCTCA |
| 2311 | hsa-miR-99a | CACAAGATCGGATCTACGGGTCACAAGATCGGATCTACGGGT |
| 2312 | hsa-miR-99b | CAAGGTCGGTTCTACGGGTCAAGGTCGGTTCTACGGGT |
| 2313 | mo-miR-322 | TGTTGCAGCGCTTCATGTTTTGTTGCAGCGCTTCATGTTT |
| 2314 | mo-miR-323 | AGAGGTCGACCGTGTAATGTGAGAGGTCGACCGTGTAATGTG |
| 2315 | mo-miR-301 | GCTTTGACAATACTATTGCACTGCTTTGACAATACTATTGCACT |
| 2316 | mo-miR-324-5p | ACACCAATGCCCTAGGGGATACACCAATGCCCTAGGGGAT |
| 2317 | mo-miR-324-3p | AGCAGCACCTGGGGCAGTAGCAGCACCTGGGGCAGT |
| 2318 | mo-miR-325 | ACACTTACTGAGCACCTACTAGACACTTACTGAGCACCTACTAG |
| 2319 | mo-miR-326 | ACTGGAGGAAGGGCCCAGAACTGGAGGAAGGGCCCAGA |
| 2320 | mo-miR-327 | ACCCTCATGCCCCTCAAGACCCTCATGCCCCTCAAG |
| 2321 | mo-let-7d | ACTATGCAACCTACTACCTCTACTATGCAACCTACTACCTCT |
| 2322 | mo-let-7d* | AGAAAGGCAGCAGGTCGTATAAGAAAGGCAGCAGGTCGTATA |
| 2323 | mo-miR-328 | ACGGAAGGGCAGAGAGGGCCAACGGAAGGGCAGAGAGGGCCA |
| 2324 | mo-miR-329 | AAAAAGGTTAGCTGGGTGTGTTAAAAAGGTTAGCTGGGTGTGTT |
| 2325 | mo-miR-330 | TCTCTGCAGGCCCTGTGCTTTTCTCTGCAGGCCCTGTGCTTT |
| 2326 | mo-miR-331 | TTCTAGGATAGGCCCAGGGTTCTAGGATAGGCCCAGGG |
| 2327 | mo-miR-333 | AAAAGTAACTAGCACACCACAAAAGTAACTAGCACACCAC |
| 2328 | mo-miR-140 | CTACCATAGGGTAAAACCACTCTACCATAGGGTAAAACCACT |
| 2329 | mo-miR-140* | TGTCCGTGGTTCTACCCTGTTGTCCGTGGTTCTACCCTGT |
| 2330 | mo-miR-335 | ACATTTTTCGTTATTGCTCTTGACATTTTTCGTTATTGCTCTTG |
| 2331 | mo-miR-336 | AGACTAGATATGGAAGGGTGAAGACTAGATATGGAAGGGTGA |
| 2332 | mo-miR-337 | AAAGGCATCATATAGGAGCTGAAAAGGCATCATATAGGAGCTGA |
| 2333 | mo-miR-148b | ACAAAGTTCTGTGATGCACTGAACAAAGTTCTGTGATGCACTGA |
| 2334 | mo-miR-338 | TCAACAAAATCACTGATGCTGGTCAACAAAATCACTGATGCTGG |
| 2335 | mo-miR-339 | TGAGCTCCTGGAGGACAGGGATGAGCTCCTGGAGGACAGGGA |
| 2336 | mo-miR-340 | GGCTATAAAGTAACTGAGACGGGGCTATAAAGTAACTGAGACGG |
| 2337 | mo-miR-341 | ACTGACCGACCGACCGATCGAACTGACCGACCGACCGATCGA |
| 2338 | mo-miR-342 | ACGGGTGCGATTTCTGTGTGAACGGGTGCGATTTCTGTGTGA |
| 2339 | mo-miR-343 | TCTGGGCACACGGAGGGAGATCTGGGCACACGGAGGGAGA |
| 2340 | mo-miR-344 | ACGGTCAGGCTTTGGCTAGATACGGTCAGGCTTTGGCTAGAT |
| 2341 | mo-miR-345 | ACTGGACTAGGGGTCAGCAACTGGACTAGGGGTCAGCA |
| 2342 | mo-miR-346 | AGAGGCAGGCACTCAGGCAGAAGAGGCAGGCACTCAGGCAGA |
| 2343 | mo-miR-347 | TGGGCGACCCAGAGGGACATGGGCGACCCAGAGGGACA |
| 2344 | mo-miR-349 | AGAGGTTAAGACAGCAGGGCTAGAGGTTAAGACAGCAGGGCT |
| 2345 | mo-miR-129 | AGCAAGCCCAGACCGCAAAAAAGCAAGCCCAGACCGCAAAAA |
| 2346 | mo-miR-129* | ATGCTTTTTGGGGTAAGGGCTTATGCTTTTTGGGGTAAGGGCTT |
| 2347 | mo-miR-20 | CTACCTGCACTATAAGCACTTTCTACCTGCACTATAAGCACTTT |
| 2348 | mo-miR-20* | TGTAAGTGCTCGTAATGCAGTTGTAAGTGCTCGTAATGCAGT |
| 2349 | mo-miR-350 | GTGAAAGTGTATGGGCTTTGTGGTGAAAGTGTATGGGCTTTGTG |
| 2350 | mo-miR-7 | AACAAAATCACTAGTCTTCCAACAAAATCACTAGTCTTCC |
| 2351 | mo-miR-7* | TATGGCAGACTGTGATTTGTTGTATGGCAGACTGTGATTTGTTG |
| 2352 | mo-miR-351 | AGGCTCAAAGGGCTCCTCAAGGCTCAAAGGGCTCCTCA |
| 2353 | mo-miR-352 | TACTATGCAACCTACTACTCTTACTATGCAACCTACTACTCT |
| 2354 | mo-miR-135b | CACATAGGAATGAAAAGCCATACACATAGGAATGAAAAGCCATA |
| 2355 | mo-miR-151* | TACTAGACTGTGAGCTCCTCGTACTAGACTGTGAGCTCCTCG |
| 2356 | mo-miR-151 | CTCAAGGAGCCTCAGTCTAGTCTCAAGGAGCCTCAGTCTAGT |
| 2357 | mo-miR-101b | CTTCAGCTATCACAGTACTGTACTTCAGCTATCACAGTACTGTA |
| 2358 | mo-let-7a | AACTATACAACCTACTACCTCAAACTATACAACCTACTACCTCA |
| 2359 | mo-let-7b | AACCACACAACCTACTACCTCAAACCACACAACCTACTACCTCA |
| 2360 | mo-let-7c | AACCATACAACCTACTACCTCAAACCATACAACCTACTACCTCA |
| 2361 | mo-let-7e | ACTATACAACCTCCTACCTCAACTATACAACCTCCTACCTCA |
| 2362 | mo-let-7f | AACTATACAATCTACTACCTCAAACTATACAATCTACTACCTCA |
| 2363 | mo-let-7i | ACAGCACAAACTACTACCTCAACAGCACAAACTACTACCTCA |
| 2364 | mo-miR-7b | AACAAAATCACAAGTCTTCCAACAAAATCACAAGTCTTCC |
| 2365 | mo-miR-9 | TCATACAGCTAGATAACCAAAGTCATACAGCTAGATAACCAAAG |
| 2366 | mo-miR-10a | CACAAATTCGGATCTACAGGGTCACAAATTCGGATCTACAGGGT |
| 2367 | mo-miR-10b | ACACAAATTCGGTTCTACAGGGACACAAATTCGGTTCTACAGGG |
| 2368 | mo-miR-15b | TGTAAACCATGATGTGCTGCTATGTAAACCATGATGTGCTGCTA |
| 2369 | mo-miR-16 | CGCCAATATTTACGTGCTGCTACGCCAATATTTACGTGCTGCTA |
| 2370 | mo-miR-17 | ACTACCTGCACTGTAAGCACTTACTACCTGCACTGTAAGCACTT |
| 2371 | mo-miR-18 | TATCTGCACTAGATGCACCTTATATCTGCACTAGATGCACCTTA |
| 2372 | mo-miR-19b | TCAGTTTTGCATGGATTTGCACTCAGTTTTGCATGGATTTGCAC |
| 2373 | mo-miR-19a | TCAGTTTTGCATAGATTTGCACTCAGTTTTGCATAGATTTGCAC |
| 2374 | mo-miR-21 | TCAACATCAGTCTGATAAGCTATCAACATCAGTCTGATAAGCTA |
| 2375 | mo-miR-22 | ACAGTTCTTCAACTGGCAGCTTACAGTTCTTCAACTGGCAGCTT |
| 2376 | mo-miR-23a | GGAAATCCCTGGCAATGTGATGGAAATCCCTGGCAATGTGAT |
| 2377 | mo-miR-23b | GGTAATCCCTGGCAATGTGATGGTAATCCCTGGCAATGTGAT |
| 2378 | mo-miR-24 | TGTTCCTGCTGAACTGAGCCATGTTCCTGCTGAACTGAGCCA |
| 2379 | mo-miR-25 | TCAGACCGAGACAAGTGCAATTCAGACCGAGACAAGTGCAAT |
| 2380 | mo-miR-26a | GCCTATCCTGGATTACTTGAAGCCTATCCTGGATTACTTGAA |
| 2381 | mo-miR-26b | AACCTATCCTGAATTACTTGAAAACCTATCCTGAATTACTTGAA |
| 2382 | mo-miR-27b | GCAGAACTTAGCCACTGTGAAGCAGAACTTAGCCACTGTGAA |
| 2383 | mo-miR-27a | GCGGAACTTAGCCACTGTGAAGCGGAACTTAGCCACTGTGAA |
| 2384 | mo-miR-28 | CTCAATAGACTGTGAGCTCCTTCTCAATAGACTGTGAGCTCCTT |
| 2385 | mo-miR-29b | AACACTGATTTCAAATGGTGCTAACACTGATTTCAAATGGTGCT |
| 2386 | mo-miR-29a | AACCGATTTCAGATGGTGCTAAACCGATTTCAGATGGTGCTA |
| 2387 | mo-miR-29c | ACCGATTTCAAATGGTGCTAACCGATTTCAAATGGTGCTA |
| 2388 | mo-miR-30c | GCTGAGAGTGTAGGATGTTTACGCTGAGAGTGTAGGATGTTTAC |
| 2389 | mo-miR-30e | TCCAGTCAAGGATGTTTACATCCAGTCAAGGATGTTTACA |
| 2390 | mo-miR-30b | AGCTGAGTGTAGGATGTTTACAAGCTGAGTGTAGGATGTTTACA |
| 2391 | mo-miR-30d | CTTCCAGTCGGGGATGTTTACCTTCCAGTCGGGGATGTTTAC |
| 2392 | mo-miR-30a-5p | CTTCCAGTCGAGGATGTTTACACTTCCAGTCGAGGATGTTTACA |
| 2393 | mo-miR-30a-3p | GCTGCAAACATCCGACTGAAAGCTGCAAACATCCGACTGAAA |
| 2394 | mo-miR-31 | AGCTATGCCAGCATCTTGCCTAGCTATGCCAGCATCTTGCCT |
| 2395 | mo-miR-32 | GCAACTTAGTAATGTGCAATGCAACTTAGTAATGTGCAAT |
| 2396 | mo-miR-33 | CAATGCAACTACAATGCACCAATGCAACTACAATGCAC |
| 2397 | mo-miR-34b | CAATCAGCTAATTACACTGCCTCAATCAGCTAATTACACTGCCT |
| 2398 | mo-miR-34c | CAATCAGCTAACTACACTGCCTCAATCAGCTAACTACACTGCCT |
| 2399 | mo-miR-34a | AACAACCAGCTAAGACACTGCAACAACCAGCTAAGACACTGC |
| 2400 | mo-miR-92 | AGGCCGGGACAAGTGCAATAAGGCCGGGACAAGTGCAATA |
| 2401 | mo-miR-93 | CTACCTGCACGAACAGCACTTCTACCTGCACGAACAGCACTT |
| 2402 | mo-miR-96 | AGCAAAAATGTGCTAGTGCCAAAGCAAAAATGTGCTAGTGCCAA |
| 2403 | mo-miR-98 | AACAATACAACTTACTACCTCAAACAATACAACTTACTACCTCA |
| 2404 | mo-miR-99a | CACAAGATCGGATCTACGGGTCACAAGATCGGATCTACGGGT |
| 2405 | mo-miR-99b | CAAGGTCGGTTCTACGGGTCAAGGTCGGTTCTACGGGT |
| 2406 | mo-miR-100 | CACAAGTTCGGATCTACGGGTCACAAGTTCGGATCTACGGGT |
| 2407 | mo-miR-101 | CTTCAGTTATCACAGTACTGTACTTCAGTTATCACAGTACTGTA |
| 2408 | mo-miR-103 | TCATAGCCCTGTACAATGCTGTCATAGCCCTGTACAATGCTG |
| 2409 | mo-miR-106b | ATCTGCACTGTCAGCACTTTAATCTGCACTGTCAGCACTTTA |
| 2410 | mo-miR-107 | TGATAGCCCTGTACAATGCTGTGATAGCCCTGTACAATGCTG |
| 2411 | mo-miR-122a | ACAAACACCATTGTCACACTCCACAAACACCATTGTCACACTCC |
| 2412 | mo-miR-124a | TGGCATTCACCGCGTGCCTTAATGGCATTCACCGCGTGCCTTAA |
| 2413 | mo-miR-125a | CACAGGTTAAAGGGTCTCAGGCACAGGTTAAAGGGTCTCAGG |
| 2414 | mo-miR-125b | TCACAAGTTAGGGTCTCAGGGTCACAAGTTAGGGTCTCAGGG |
| 2415 | mo-miR-126* | CGCGTACCAAAAGTAATAATGCGCGTACCAAAAGTAATAATG |
| 2416 | mo-miR-126 | GCATTATTACTCACGGTACGAGCATTATTACTCACGGTACGA |
| 2417 | mo-miR-127 | AGCCAAGCTCAGACGGATCCGAAGCCAAGCTCAGACGGATCCGA |
| 2418 | mo-miR-128a | AAAAGAGACCGGTTCACTGTGAAAAAGAGACCGGTTCACTGTGA |
| 2419 | mo-miR-128b | GAAAGAGACCGGTTCACTGTGGAAAGAGACCGGTTCACTGTG |
| 2420 | mo-miR-130a | ATGCCCTTTTAACATTGCACTGATGCCCTTTTAACATTGCACTG |
| 2421 | mo-miR-130b | ATGCCCTTTCATCATTGCACTGATGCCCTTTCATCATTGCACTG |
| 2422 | mo-miR-132 | CGACCATGGCTGTAGACTGTTCGACCATGGCTGTAGACTGTT |
| 2423 | mo-miR-133a | ACAGCTGGTTGAAGGGGACCAAACAGCTGGTTGAAGGGGACCAA |
| 2424 | mo-miR-134 | CCTCTGGTCAACCAGTCACACCTCTGGTCAACCAGTCACA |
| 2425 | mo-miR-135a | TCACATAGGAATAAAAAGCCATTCACATAGGAATAAAAAGCCAT |
| 2426 | mo-miR-136 | TCCATCATCAAAACAAATGGAGTCCATCATCAAAACAAATGGAG |
| 2427 | mo-miR-137 | CTACGCGTATTCTTAAGCAATACTACGCGTATTCTTAAGCAATA |
| 2428 | mo-miR-138 | GATTCACAACACCAGCTGATTCACAACACCAGCT |
| 2429 | mo-miR-139 | AGACACGTGCACTGTAGAAGACACGTGCACTGTAGA |
| 2430 | mo-miR-141 | CCATCTTTACCAGACAGTGTTACCATCTTTACCAGACAGTGTTA |
| 2431 | mo-miR-142-5p | GTAGTGCTTTCTACTTTATGGTAGTGCTTTCTACTTTATG |
| 2432 | mo-miR-142-3p | TCCATAAAGTAGGAAACACTACTCCATAAAGTAGGAAACACTAC |
| 2433 | mo-miR-143 | TGAGCTACAGTGCTTCATCTCATGAGCTACAGTGCTTCATCTCA |
| 2434 | mo-miR-144 | CTAGTACATCATCTATACTGTACTAGTACATCATCTATACTGTA |
| 2435 | mo-miR-145 | AAGGGATTCCTGGGAAAACTGAAGGGATTCCTGGGAAAACTG |
| 2436 | mo-miR-146 | AACCCATGGAATTCAGTTCTCAAACCCATGGAATTCAGTTGTCA |
| 2437 | mo-miR-150 | ACTGGTACAAGGGTTGGGAGAACTGGTACAAGGGTTGGGAGA |
| 2438 | mo-miR-152 | CCCAAGTTCTGTCATGCACTGCCCAAGTTCTGTCATGCACTG |
| 2439 | mo-miR-153 | TCACTTTTGTGACTATGCAATCACTTTTGTGACTATGCAA |
| 2440 | mo-miR-154 | CGAAGGCAACACGGATAACCTCGAAGGCAACACGGATAACCT |
| 2441 | mo-miR-181c | ACTCACCGACAGGTTGAATGTTACTCACCGACAGGTTGAATGTT |
| 2442 | mo-miR-181a | ACTCACCGACAGCGTTGAATGACTCACCGACAGCGTTGAATG |
| 2443 | mo-miR-181b | CCCACCGACAGCAATGAATGTCCCACCGACAGCAATGAATGT |
| 2444 | mo-miR-183 | CAGTGAATTCTACCAGTGCCATCAGTGAATTCTACCAGTGCCAT |
| 2445 | mo-miR-184 | ACCCTTATCAGTTCTCCGTCCACCCTTATCAGTTCTCCGTCC |
| 2446 | mo-miR-185 | GAACTGCCTTTCTCTCCAGAACTGCCTTTCTCTCCA |
| 2447 | mo-miR-186 | AGCCCAAAAGGAGAATTCTTTGAGCCCAAAAGGAGAATTCTTTG |
| 2448 | mo-miR-187 | GGCTGCAACACAAGACACGAGGCTGCAACACAAGACACGA |
| 2449 | mo-miR-190 | ACCTAATATATCAAACATATCAACCTAATATATCAAACATATCA |
| 2450 | mo-miR-191 | AGCTGCTTTTGGGATTCCGTTAGCTGCTTTTGGGATTCCGTT |
| 2451 | mo-miR-192 | GGCTGTCAATTCATAGGTCAGGGCTGTCAATTCATAGGTCAG |
| 2452 | mo-miR-193 | CTGGGACTTTGTAGGCCAGTTCTGGGACTTTGTAGGCCAGTT |
| 2453 | mo-miR-194 | TCCACATGGAGTTGCTGTTACATCCACATGGAGTTGCTGTTACA |
| 2454 | mo-miR-195 | GCCAATATTTCTGTGCTGCTAGCCAATATTTCTGTGCTGCTA |
| 2455 | mo-miR-196a | CCAACAACATGAAACTACCTACCAACAACATGAAACTACCTA |
| 2456 | mo-miR-199a | GAACAGGTAGTCTGAACACTGGAACAGGTAGTCTGAACACTG |
| 2457 | mo-miR-200c | CCATCATTACCCGGCAGTATTACCATCATTACCCGGCAGTATTA |
| 2458 | mo-miR-200a | ACATCGTTACCAGACAGTGTTAACATCGTTACCAGACAGTGTTA |
| 2459 | mo-miR-200b | GTCATCATTACCAGGCAGTATTGTCATCATTACCAGGCAGTATT |
| 2460 | mo-miR-203 | CTAGTGGTCCTAAACATTTCACCTAGTGGTCCTAAACATTTCAC |
| 2461 | mo-miR-204 | AGGCATAGGATGACAAAGGGAAAGGCATAGGATGACAAAGGGAA |
| 2462 | mo-miR-205 | AGACTCCGGTGGAATGAAGGAAGACTCCGGTGGAATGAAGGA |
| 2463 | mo-miR-206 | CCACACACTTCCTTACATTCCACCACACACTTCCTTACATTCCA |
| 2464 | mo-miR-208 | ACAAGCTTTTTGCTCGTCTTATACAAGCTTTTTGCTCGTCTTAT |
| 2465 | mo-miR-210 | TCAGCCGCTGTCACACGCACATCAGCCGCTGTCACACGCACA |
| 2466 | mo-miR-211 | AGGCAAAGGATGACAAAGGGAAAGGCAAAGGATGACAAAGGGAA |
| 2467 | mo-miR-212 | GCCGTGACTGGAGACTGTTAGCCGTGACTGGAGACTGTTA |
| 2468 | mo-miR-213 | GGTACAATCAACGGTCGATGGGGTACAATCAACGGTCGATGG |
| 2469 | mo-miR-214 | TGCCTGTCTGTGCCTGCTGTTGCCTGTCTGTGCCTGCTGT |
| 2470 | mo-miR-216 | CACAGTTGCCAGCTGAGATTACACAGTTGCCAGCTGAGATTA |
| 2471 | mo-miR-217 | ATCCAGTCAGTTCCTGATGCAATCCAGTCAGTTCCTGATGCA |
| 2472 | mo-miR-218 | ACATGGTTAGATCAAGCACAAACATGGTTAGATCAAGCACAA |
| 2473 | mo-miR-219 | AGAATTGCGTTTGGACAATCAAGAATTGCGTTTGGACAATCA |
| 2474 | mo-miR-221 | AAACCCAGCAGACAATGTAGCTAAACCCAGCAGACAATGTAGCT |
| 2475 | mo-miR-222 | AGACCCAGTAGCCAGATGTAGAGACCCAGTAGCCAGATGTAG |
| 2476 | mo-miR-223 | GGGGTATTTGACAAACTGACAGGGGTATTTGACAAACTGACA |
| 2477 | mo-miR-290 | AAAAAGTGCCCCCATAGTTTGAAAAAAGTGCCCCCATAGTTTGA |
| 2478 | mo-miR-291-5p | AGAGAGGGCCTCCACTTTGATAGAGAGGGCCTCCACTTTGAT |
| 2479 | mo-miR-291-3p | GCACACAAAGTGGAAGCACTTTGCACACAAAGTGGAAGCACTTT |
| 2480 | mo-miR-292-5p | CAAAAGAGCCCCCAGTTTGAGCAAAAGAGCCCCCAGTTTGAG |
| 2481 | mo-miR-292-3p | ACACTCAAAACCTGGCGGCACTACACTCAAAACCTGGCGGCACT |
| 2482 | mo-miR-296 | ACAGGATTGAGGGGGGGCCCTACAGGATTGAGGGGGGGCCCT |
| 2483 | mo-miR-297 | CATGCATACATGCACACATACACATGCATACATGCACACATACA |
| 2484 | mo-miR-298 | GGAAGAACAGCCCTCCTCTGGAAGAACAGCCCTCCTCT |
| 2485 | mo-miR-299 | ATGTATGTGGGACGGTAAACCAATGTATGTGGGACGGTAAACCA |
| 2486 | mo-miR-300 | GAAGAGAGCTTGCCCTTGCATGAAGAGAGCTTGCCCTTGCAT |
| 2487 | mo-miR-320 | TTCGCCCTCTCAACCCAGCTTTTTCGCCCTCTCAACCCAGCTTT |
| 2488 | mo-miR-196b | CCAACAACAGGAAACTACCTACCAACAACAGGAAACTACCTA |
| 2489 | mo-miR-421 | CAACAAACATTTAATGAGGCCCAACAAACATTTAATGAGGCC |
| 2490 | mo-miR-448 | ATGGGACATCCTACATATGCAAATGGGACATCCTACATATGCAA |
| 2491 | mo-miR-429 | ACGGCATTACCAGACAGTATTAACGGCATTACCAGACAGTATTA |
| 2492 | mo-miR-449 | ACCAGCTAACAATACACTGCCAACCAGCTAACAATACACTGCCA |
| 2493 | mo-miR-450 | CATTAGGAACACATCGCAAAAACATTAGGAACACATCGCAAAAA |
| 2494 | mo-miR-365 | ATAAGGATTTTTAGGGGCATTAATAAGGATTTTTAGGGGCATTA |
| 2495 | mo-miR-424 | TCCAAAACATGAATTGCTGCTGTCCAAAACATGAATTGCTGCTG |
| 2496 | mo-miR-431 | TGCATGACGGCCTGCAAGACATGCATGACGGCCTGCAAGACA |
| 2497 | mo-miR-433 | ACACCGAGGAGCCCATCATGATACACCGAGGAGCCCATCATGAT |
| 2498 | mo-miR-451 | AACTCAGTAATGGTAACGGTTTAACTCAGTAATGGTAACGGTTT |
| 2499 | mmu-let-7g | ACTGTACAAACTACTACCTCAACTGTACAAACTACTACCTCA |
| 2500 | mmu-let-7i | ACAGCACAAACTACTACCTCAACAGCACAAACTACTACCTCA |
| 2501 | mmu-miR-1 | TACATACTTCTTTACATTCCATACATACTTCTTTACATTCCA |
| 2502 | mmu-miR-15b | TGTAAACCATGATGTGCTGCTATGTAAACCATGATGTGCTGCTA |
| 2503 | mmu-miR-23b | GGTAATCCCTGGCAATGTGATGGTAATCCCTGGCAATGTGAT |
| 2504 | mmu-miR-27b | GCAGAACTTAGCCACTGTGAAGCAGAACTTAGCCACTGTGAA |
| 2505 | mmu-miR-29b | AACACTGATTTCAAATGGTGCTAACACTGATTTCAAATGGTGCT |
| 2506 | mmu-miR-30a-5p | CTTCCAGTCGAGGATGTTTACACTTCCAGTCGAGGATGTTTACA |
| 2507 | mmu-miR-30a-3p | GCTGCAAACATCCGACTGAAAGCTGCAAACATCCGACTGAAA |
| 2508 | mmu-miR-30b | AGCTGAGTGTAGGATGTTTACAAGCTGAGTGTAGGATGTTTACA |
| 2509 | mmu-miR-99a | ACAAGATCGGATCTACGGGTACAAGATCGGATCTACGGGT |
| 2510 | mmu-miR-99b | CAAGGTCGGTTCTACGGGTCAAGGTCGGTTCTACGGGT |
| 2511 | mmu-miR-101a | CTTCAGTTATCACAGTACTGTACTTCAGTTATCACAGTACTGTA |
| 2512 | mmu-miR-124a | GCATTCACCGCGTGCCTTAGCATTCACCGCGTGCCTTA |
| 2513 | mmu-miR-125a | CACAGGTTAAAGGGTCTCAGGCACAGGTTAAAGGGTCTCAGG |
| 2514 | mmu-miR-125b | TCACAAGTTAGGGTCTCAGGGTCACAAGTTAGGGTCTCAGGG |
| 2515 | mmu-miR-126-5p | CGCGTACCAAAAGTAATAATGCGCGTACCAAAAGTAATAATG |
| 2516 | mmu-miR-126-3p | GCATTATTACTCACGGTACGAGCATTATTACTCACGGTACGA |
| 2517 | mmu-miR-127 | CAAGCTCAGACGGATCCGACAAGCTCAGACGGATCCGA |
| 2518 | mmu-miR-128a | AAAAGAGACCGGTTCACTGTGAAAAAGAGACCGGTTCACTGTGA |
| 2519 | mmu-miR-130a | ATGCCCTTTTAACATTGCAGTGATGCCCTTTTAACATTGCACTG |
| 2520 | mmu-miR-9 | CATACAGCTAGATAACCAAAGACATACAGCTAGATAACCAAAGA |
| 2521 | mmu-miR˜9* | ACTTTCGGTTATCTAGCTTTACTTTCGGTTATCTAGCTTT |
| 2522 | mmu-miR-132 | CGACCATGGCTGTAGACTGTTCGACCATGGCTGTAGACTGTT |
| 2523 | mmu-miR-133a | ACAGCTGGTTGAAGGGGACCAAACAGCTGGTTGAAGGGGACCAA |
| 2524 | mmu-miR-134 | CCTCTGGTCAACCAGTCACACCTCTGGTCAACCAGTCACA |
| 2525 | mmu-miR-135a | TCACATAGGAATAAAAAGCCATTCACATAGGAATAAAAAGCCAT |
| 2526 | mmu-miR-136 | TCCATCATCAAAACAAATGGAGTCCATCATCAAAACAAATGGAG |
| 2527 | mmu-miR-137 | CTACGCGTATTCTTAAGCAATACTACGCGTATTCTTAAGCAATA |
| 2528 | mmu-miR-138 | GATTCACAACACCAGCTGATTCACAACACCAGCT |
| 2529 | mmu-miR-140 | CTACCATAGGGTAAAACCACTGCTACCATAGGGTAAAACGACTG |
| 2530 | mmu-miR-140* | TCCGTGGTTCTACCCTGTGGTATCCGTGGTTCTACCCTGTGGTA |
| 2531 | mmu-miR-141 | CCATCTTTACCAGACAGTGTTACCATCTTTACCAGACAGTGTTA |
| 2532 | mmu-miR-142-5p | GTAGTGCTTTCTACTTTATGGTAGTGCTTTCTACTTTATG |
| 2533 | mmu-miR-142-3p | CCATAAAGTAGGAAACACTACACCATAAAGTAGGAAACACTACA |
| 2534 | mmu-miR-144 | CTAGTACATCATCTATACTGTACTAGTACATCATCTATACTGTA |
| 2535 | mmu-miR-145 | AAGGGATTCCTGGGAAAACTGAAGGGATTCCTGGGAAAACTG |
| 2536 | mmu-miR-146 | AACCCATGGAATTCAGTTCTCAAACCCATGGAATTCAGTTCTCA |
| 2537 | mmu-miR-149 | AGTGAAGACACGGAGCCAGAAGTGAAGACACGGAGCCAGA |
| 2538 | mmu-miR-150 | ACTGGTACAAGGGTTTGGGAGAACTGGTACAAGGGTTGGGAGA |
| 2539 | mmu-miR-151 | CCTCAAGGAGCCTCAGTCTACCTCAAGGAGCCTCAGTCTA |
| 2540 | mmu-miR-152 | CCCAAGTTCTGTCATGCACTGCCCAAGTTCTGTCATGCACTG |
| 2541 | mmu-miR-153 | GATCACTTTTGTGACTATGCAAGATCACTTTTGTGACTATGCAA |
| 2542 | mmu-miR-154 | CGAAGGCAACACGGATAACCTCGAAGGCAACACGGATAACCT |
| 2543 | mmu-miR-155 | CCCCTATCACAATTAGCATTAACCCCTATCACAATTAGCATTAA |
| 2544 | mmu-miR-10b | ACACAAATTCGGTTCTACAGGGACACAAATTCGGTTCTACAGGG |
| 2545 | mmu-miR-129-5p | AGCAAGCCCAGACCGCAAAAAAGCAAGCCCAGACCGCAAAAA |
| 2546 | mmu-miR-181a | ACTCACCGACAGCGTTGAATGACTCACCGACAGCGTTGAATG |
| 2547 | mmu-miR-182 | TGTGAGTTCTACCATTGCCAAATGTGAGTTCTACCATTGCCAAA |
| 2548 | mmu-miR-183 | CAGTGAATTCTACCAGTGCCATCAGTGAATTCTACCAGTGCCAT |
| 2549 | mmu-miR-184 | ACCCTTATCAGTTCTCCGTCCACCCTTATCAGTTCTCCGTCC |
| 2550 | mmu-miR-185 | GAACTGCCTTTCTCTCCAGAACTGCCTTTCTCTCCA |
| 2551 | mmu-miR-186 | AGCCCAAAAGGAGAATTCTTTGAGCCCAAAAGGAGAATTCTTTG |
| 2552 | mmu-miR-187 | GGCTGCAACACAAGACACGAGGCTGCAACACAAGACACGA |
| 2553 | mmu-miR-188 | ACCCTCCACCATGCAAGGGATACCCTCCACCATGCAAGGGAT |
| 2554 | mmu-miR-189 | ACTGATATCAGCTCAGTAGGCAACTGATATCAGCTCAGTAGGCA |
| 2555 | mmu-miR-24 | TGTTCCTGCTGAACTGAGCCATGTTCCTGCTGAACTGAGCCA |
| 2556 | mmu-miR-190 | ACCTAATATATCAAACATATCAACCTAATATATCAAACATATCA |
| 2557 | mmu-miR-191 | AGCTGCTTTTGGGATTCCGTTAGCTGCTTTTGGGATTCCGTT |
| 2558 | mmu-miR-193 | CTGGGACTTTGTAGGCCAGTTCTGGGACTTTGTAGGCCAGTT |
| 2559 | mmu-miR-194 | TCCACATGGAGTTGCTGTTACATCCACATGGAGTTGCTGTTACA |
| 2560 | mmu-miR-195 | GCCAATATTTCTGTGCTGCTAGCCAATATTTCTGTGCTGCTA |
| 2561 | mmu-miR-199a | GAACAGGTAGTCTGAACACTGGAACAGGTAGTCTGAACACTG |
| 2562 | mmu-miR-199a* | AACCAATGTGCAGACTACTGTAAACCAATGTGCAGACTACTGTA |
| 2563 | mmu-miR-200b | GTCATCATTACCAGGCAGTATTGTCATCATTACCAGGCAGTATT |
| 2564 | mmu-miR-201 | AGAACAATGCCTTACTGAGTAAGAACAATGCCTTACTGAGTA |
| 2565 | mmu-miR-202 | TCTTCCCATGCGCTATACCTCTCTTCCCATGCGCTATACCTC |
| 2566 | mmu-miR-203 | CTAGTGGTCCTAAACATTTCACTAGTGGTCCTAAACATTTCA |
| 2567 | mmu-miR-204 | AGGCATAGGATGACAAAGGGAAAGGCATAGGATGACAAAGGGAA |
| 2568 | mmu-miR-205 | AGACTCCGGTGGAATGAAGGAAGACTCCGGTGGAATGAAGGA |
| 2569 | mmu-miR-206 | CCACACACTTCCTTACATTCCACCACACACTTCCTTACATTCCA |
| 2570 | mmu-miR-207 | AGGGAGGAGAGCCAGGAGAAAGGGAGGAGAGCCAGGAGAA |
| 2571 | mmu-miR-122a | ACAAACACCATTGTCACACTCCACAAACACCATTGTCACACTCC |
| 2572 | mmu-miR-143 | TGAGCTACAGTGCTTCATCTCATGAGCTACAGTGCTTCATCTCA |
| 2573 | mmu-miR-30e | TCCAGTCAAGGATGTTTACATCCAGTCAAGGATGTTTACA |
| 2574 | mmu-miR-30e* | CTGTAAACATCCGACTGAAAGCTGTAAACATCCGACTGAAAG |
| 2575 | mmu-miR-290 | AAAAAGTGCCCCCATAGTTTGAAAAAAGTGCCCCCATAGTTTGA |
| 2576 | mmu-miR-291-5p | AGAGAGGGCCTCCACTTTGATAGAGAGGGCCTCCACTTTGAT |
| 2577 | mmu-miR-291-3p | GCACACAAAGTGGAAGCACTTTGCACACAAAGTGGAAGCACTTT |
| 2578 | mmu-miR-292-5p | CAAAAGAGCCCCCAGTTTGAGCAAAAGAGCCCCCAGTTTGAG |
| 2579 | mmu-miR-292-3p | ACACTCAAAACCTGGCGGCACTACACTCAAAACCTGGCGGCACT |
| 2580 | mmu-miR-293 | ACACTACAAACTCTGCGGCACACACTACAAACTCTGCGGCAC |
| 2581 | mmu-miR-294 | ACACACAAAAGGGAAGCACTTTACACACAAAAGGGAAGCACTTT |
| 2582 | mmu-miR-295 | AGACTCAAAAGTAGTAGCACTTAGACTCAAAAGTAGTAGCACTT |
| 2583 | mmu-miR-296 | ACAGGATTGAGGGGGGGCCCTACAGGATTGAGGGGGGGCCCT |
| 2584 | mmu-miR-297 | CATGCACATGCACACATACATCATGCACATGCACACATACAT |
| 2585 | mmu-miR-298 | GGAAGAACAGCCCTCCTCTGGAAGAACAGCCCTCCTCT |
| 2586 | mmu-miR-299 | ATGTATGTGGGACGGTAAACCAATGTATGTGGGACGGTAAACCA |
| 2587 | mmu-miR-300 | GAAGAGAGCTTGCCCTTGCATGAAGAGAGCTTGCCCTTGCAT |
| 2588 | mmu-miR-301 | GCTTTGACAATACTATTGCACTGCTTTGACAATACTATTGCACT |
| 2589 | mmu-miR-302 | TCACCAAAACATGGAAGCACTTTCACCAAAACATGGAAGCACTT |
| 2590 | mmu-miR-34c | CAATCAGCTAACTACACTGCCTCAATCAGCTAACTACACTGCCT |
| 2591 | mmu-miR-34b | CAATCAGCTAATTACACTGCCTCAATCAGCTAATTACACTGCCT |
| 2592 | mmu-let-7d | ACTATGCAACCTACTACCTCTACTATGCAACCTACTACCTCT |
| 2593 | mmu-let-7d* | AGAAAGGCAGCAGGTCGTATAAGAAAGGCAGCAGGTCGTATA |
| 2594 | mmu-miR-106a | TACCTGCACTGTTAGCACTTTGTACCTGCACTGTTAGCACTTTG |
| 2595 | mmu-miR-106b | ATCTGCAGTGTCAGCACTTTAATCTGCACTGTCAGCACTTTA |
| 2596 | mmu-miR-130b | ATGCCCTTTCATCATTGCACTGATGCCC1TTCATCATTGCACTG |
| 2597 | mmu-miR-19b | TCAGTTTTGCATGGATTTGCACTCAGTTTTGCATGGATTTGCAC |
| 2598 | mmu-miR-30c | GCTGAGAGTGTAGGATGTTTACGCTGAGAGTGTAGGATGTTTAC |
| 2599 | mmu-miR-30d | CTTCCAGTCGGGGATGTTTACCTTCCAGTCGGGGATGTTTAC |
| 2600 | mmu-miR-148a | ACAAAGTTCTGTAGTGCACTGAACAAAGTTCTGTAGTGCACTGA |
| 2601 | mmu-miR-192 | TGTCAATTCATAGGTCAGTGTCAATTCATAGGTCAG |
| 2602 | mmu-miR-196a | CCAACAACATGAAACTACCTACCAACAACATGAAACTACCTA |
| 2603 | mmu-miR-200a | ACATCGTTACCAGACAGTGTTAACATCGTTACCAGACAGTGTTA |
| 2604 | mmu-miR-208 | ACAAGCTTTTTGCTCGTCTTATACAAGCTTTTTGCTCGTCTTAT |
| 2605 | mmu-let-7a | ACTATACAACCTACTACCTCAACTATACAACCTACTACCTCA |
| 2606 | mmu-let-7b | AACCACACAACCTACTACCTCAAACCACACAACCTACTACCTCA |
| 2607 | mmu-let-7c | AACCATACAACCTACTACCTCAAACCATACAACCTACTACCTCA |
| 2608 | mmu-let-7e | ACTATACAACCTCCTACCTCAACTATACAACCTCCTACCTCA |
| 2609 | mmu-let-7f | ACTATACAATCTACTACCTCACTATACAATCTACTACCTC |
| 2610 | mmu-miR-15a | CACAAACCATTATGTGCTGCTACACAAACCATTATGTGCTGCTA |
| 2611 | mmu-miR-16 | CGCCAATATTTACGTGCTGCTACGCCAATA1TTACGTGCTGCTA |
| 2612 | mmu-miR-18 | TATCTGCACTAGATGCACCTTATATCTGCACTAGATGCACCTTA |
| 2613 | mmu-miR-20 | CTACCTGCACTATAAGCACTTTCTACCTGCACTATAAGCACTTT |
| 2614 | mmu-miR-21 | TCAACATCAGTCTGATAAGCTATCAACATCAGTCTGATAAGCTA |
| 2615 | mmu-miR-22 | ACAGTTCTTCAACTGGCAGCTTACAGTTCTTCAACTGGCAGCTT |
| 2616 | mmu-miR-23a | GGAAATCCCTGGCAATGTGATGGAAATCCCTGGCAATGTGAT |
| 2617 | mmu-miR-26a | GCCTATCCTGGATTACTTGAAGCCTATCCTGGATTACTTGAA |
| 2618 | mmu-miR-26b | AACCTATCCTGAATTACTTGAAAACCTATCCTGAATTACTTGAA |
| 2619 | mmu-miR-29a | AACCGATTTCAGATGGTGCTAAACCGATTTCAGATGGTGCTA |
| 2620 | mmu-miR-29c | ACCGATTTCAAATGGTGCTAACCGATTTCAAATGGTGCTA |
| 2621 | mmu-miR-27a | GCGGAACTTAGCCACTGTGAAGCGGAACTTAGCCACTGTGAA |
| 2622 | mmu-miR-31 | AGCTATGCCAGCATCTTGCCTAGCTATGCCAGCATCTTGCCT |
| 2623 | mmu-miR-92 | AGGCCGGGACAAGTGCAATAAGGCCGGGACAAGTGCAATA |
| 2624 | mmu-miR-93 | CTACCTGCACGAACAGCACTTCTACCTGCACGAACAGCACTT |
| 2625 | mmu-miR-96 | AGCAAAAATGTGCTAGTGCCAAAGCAAAAATGTGCTAGTGCCAA |
| 2626 | mmu-miR-34a | AACAACCAGCTAAGACACTGCAACAACCAGCTAAGACACTGC |
| 2627 | mmu-miR-129-3p | ATGCTTTTTGGGGTAAGGGCTTATGCTTTTTGGGGTAAGGGCTT |
| 2628 | mmu-miR-98 | AACAATACAACTTACTACCTCAAACAATACAACTTACTACCTCA |
| 2629 | mmu-miR-103 | TCATAGCCCTGTACAATGCTGTCATAGCCCTGTACAATGCTG |
| 2630 | mmu-miR-424 | TCCAAAACATGAATTGCTGCTGTCCAAAACATGAATTGCTGCTG |
| 2631 | mmu-miR-322 | TGTTGCAGCGCTTCATGTTTTGTTGCAGCGCTTCATGTTT |
| 2632 | mmu-miR-323 | AGAGGTCGACCGTGTAATGTGAGAGGTCGACCGTGTAATGTG |
| 2633 | mmu-miR-324-5p | CACCAATGCCCTAGGGGATCACCAATGCCCTAGGGGAT |
| 2634 | mmu-miR-324-3p | AGCAGCACCTGGGGCAGTAGCAGCACCTGGGGCAGT |
| 2635 | mmu-miR-325 | ACACTTACTGAGCACCTACTAGACACTTACTGAGCACCTACTAG |
| 2636 | mmu-miR-326 | ACTGGAGGAAGGGCCCAGAACTGGAGGAAGGGCCCAGA |
| 2637 | mmu-miR-328 | ACGGAAGGGCAGAGAGGGCCAACGGAAGGGCAGAGAGGGCCA |
| 2638 | mmu-miR-329 | AAAAAGGTTAGCTGGGTGTGTTAAAAAGGTTAGCTGGGTGTGTT |
| 2639 | mmu-miR-330 | TCTCTGCAGGCCCTGTGCTTTTCTCTGCAGGCCCTGTGCTTT |
| 2640 | mmu-miR-331 | TTCTAGGATAGGCCCAGGGTTCTAGGATAGGCCCAGGG |
| 2641 | mmu-miR-337 | AAAGGCATCATATAGGAGCTGAAAAGGCATCATATAGGAGCTGA |
| 2642 | mmu-miR-148b | ACAAAGTTCTGTGATGCACTGAACAAAGTTCTGTGATGCACTGA |
| 2643 | mmu-miR-338 | TCAACAAAATCACTGATGCTGGTCAACAAAATCACTGATGCTGG |
| 2644 | mmu-miR-339 | TGAGCTCCTGGAGGACAGGGATGAGCTCCTGGAGGACAGGGA |
| 2645 | mmu-miR-340 | GGCTATAAAGTAACTGAGACGGGGCTATAAAGTAACTGAGACGG |
| 2646 | mmu-miR-341 | ACTGACCGACCGACCGATCGAACTGACCGACCGACCGATCGA |
| 2647 | mmu-miR-342 | ACGGGTGCGATTTCTGTGTGAACGGGTGCGATTTCTGTGTGA |
| 2648 | mmu-miR-344 | ACAGTCAGGCTTTGGCTAGATACAGTCAGGCTTTGGCTAGAT |
| 2649 | mmu-miR-345 | ACTGGACTAGGGGTCAGCAACTGGACTAGGGGTCAGCA |
| 2650 | mmu-miR-346 | AGAGGCAGGCACTCGGGCAGAAGAGGCAGGCACTCGGGCAGA |
| 2651 | mmu-miR-350 | TGAAAGTGTATGGGCTTTGTGATGAAAGTGTATGGGCTTTGTGA |
| 2652 | mmu-miR-351 | AGGCTCAAAGGGCTCCTCAAGGCTCAAAGGGCTCCTCA |
| 2653 | mmu-miR-135b | CACATAGGAATGAAAAGCCATACACATAGGAATGAAAAGCCATA |
| 2654 | mmu-miR-101b | CTTCAGCTATCACAGTACTGTACTTCAGCTATCACAGTACTGTA |
| 2655 | mmu-miR-107 | TGATAGCCCTGTACAATGCTGTGATAGCCCTGTACAATGCTG |
| 2656 | mmu-miR-10a | CACAAATTCGGATCTACAGGGTCACAAATTCGGATCTACAGGGT |
| 2657 | mmu-miR-17-5p | ACTACCTGCACTGTAAGCACTTACTACCTGCACTGTAAGCACTT |
| 2658 | mmu-miR-17-3p | TACAAGTGCCCTCACTGCAGTTACAAGTGCCCTCACTGCAGT |
| 2659 | mmu-miR-19a | TCAGTTTTGCATAGATTTGCACTCAGTTTTGCATAGATTTGCAC |
| 2660 | mmu-miR-25 | TCAGACCGAGACAAGTGCAATTCAGACCGAGACAAGTGCAAT |
| 2661 | mmu-miR-28 | CTCAATAGACTGTGAGCTCCTTCTCAATAGACTGTGAGCTCCTT |
| 2662 | mmu-miR-32 | GCAACTTAGTAATGTGCAATGCAACTTAGTAATGTGCAAT |
| 2663 | mmu-miR-100 | CACAAGTTCGGATCTACGGGTCACAAGTTCGGATCTACGGGT |
| 2664 | mmu-miR-139 | AGACACGTGCACTGTAGAAGACACGTGCACTGTAGA |
| 2665 | mmu-miR-200c | CCATCATTACCCGGCAGTATTACCATCATTACCCGGCAGTATTA |
| 2666 | mmu-miR-210 | TCAGCCGCTGTCACACGCACATCAGCCGCTGTCACACGCACA |
| 2667 | mmu-miR-212 | GCCGTGACTGGAGACTGTTAGCCGTGACTGGAGACTGTTA |
| 2668 | mmu-miR-213 | GGTACAATCAACGGTCGATGGGGTACAATCAACGGTCGATGG |
| 2669 | mmu-miR-214 | TGCCTGTCTGTGCCTGCTGTTGCCTGTCTGTGCCTGCTGT |
| 2670 | mmu-miR-216 | CACAGTTGCCAGCTGAGATTACACAGTTGCCAGCTGAGATTA |
| 2671 | mmu-miR-218 | ACATGGTTAGATCAAGCACAAACATGGTTAGATCAAGCACAA |
| 2672 | mmu-miR-219 | AGAATTGCGTTTGGACAATCAAGAATTGCGTTTGGACAATCA |
| 2673 | mmu-miR-223 | GGGGTATTTGACAAACTGACAGGGGTATTTGACAAACTGACA |
| 2674 | mmu-miR-320 | TTCGCCCTCTCAACCCAGCTTTTTCGCCCTCTCAACCCAGCTTT |
| 2675 | mmu-miR-33 | CAATGCAACTACAATGCACCAATGCAACTACAATGCAC |
| 2676 | mmu-miR-211 | AGGCAAAGGATGACAAAGGGAAAGGCAAAGGATGACAAAGGGAA |
| 2677 | mmu-miR-221 | AAACCCAGCAGACAATGTAGCTAAACCCAGCAGACAATGTAGCT |
| 2678 | mmu-miR-222 | AGACCCAGTAGCCAGATGTAGAGACCCAGTAGCCAGATGTAG |
| 2679 | mmu-miR-224 | TAAACGGAACCACTAGTGACTTTAAACGGAACCACTAGTGACTT |
| 2680 | mmu-miR-199b | GAACAGGTAGTCTAAACACTGGGAACAGGTAGTCTAAACACTGG |
| 2681 | mmu-miR-181b | CCCACCGACAGCAATGAATGTCCCACCGACAGCAATGAATGT |
| 2682 | mmu-miR-181c | ACTCACCGACAGGTTGAATGTTACTCACCGACAGGTTGAATGTT |
| 2683 | mmu-miR-128b | GAAAGAGACCGGTTCACTGTGGAAAGAGACCGGTTCACTGTG |
| 2684 | mmu-miR-7 | CAACAAAATCACTAGTCTTCCACAACAAAATCACTAGTCTTCCA |
| 2685 | mmu-miR-7b | AACAAAATCACAAGTCTTCCAACAAAATCACAAGTCTTCC |
| 2686 | mmu-miR-217 | ATCCAGTCAGTTCCTGATGCAATCCAGTCAGTTCCTGATGCA |
| 2687 | mmu-miR-361 | GTACCCCTGGAGATTCTGATAAGTACCCCTGGAGATTCTGATAA |
| 2688 | mmu-miR-363 | TTACAGATGGATACCGTGCAATTTACAGATGGATACCGTGCAAT |
| 2689 | mmu-miR-365 | ATAAGGATTTTTAGGGGCATTAATAAGGATTTTTAGGGGCATTA |
| 2690 | mmu-miR-375 | TCACGCGAGCCGAACGAACAAATCACGCGAGCCGAACGAACAAA |
| 2691 | mmu-miR-376a | ACGTGGATTTTCCTCTACGATACGTGGATTTTCCTCTACGAT |
| 2692 | mmu-miR-377 | ACAAAAGTTGCCTTTGTGTGATACAAAAGTTGCCTTTGTGTGAT |
| 2693 | mmu-miR-378 | ACACAGGACCTGGAGTCAGGAACACAGGACCTGGAGTCAGGA |
| 2694 | mmu-miR-379 | CCTACGTTCCATAGTCTACCACCTACGTTCCATAGTCTACCA |
| 2695 | mmu-miR-380-5p | GCGCATGTTCTATGGTCAACCGCGCATGTTCTATGGTCAACC |
| 2696 | mmu-miR-380-3p | AAGATGTGGACCATACTACATAAAGATGTGGACCATACTACATA |
| 2697 | mmu-miR-381 | ACAGAGAGCTTGCCCTTGTATAACAGAGAGCTTGCCCTTGTATA |
| 2698 | mmu-miR-382 | CGAATCCACCACGAACAACTTCGAATCCACCACGAACAACTT |
| 2699 | mmu-miR-383 | AGCCACAGTCACCTTCTGATCAGCCACAGTCACCTTCTGATC |
| 2700 | mmu-miR-335 | ACATTTTTCGTTATTGCTCTTGACATTTTTCGTTATTGCTCTTG |
| 2701 | mmu-miR-133b | TAGCTGGTTGAAGGGGACCAATAGCTGGTTGAAGGGGACCAA |
| 2702 | mmu-miR-215 | GTCTGTCAAATCATAGGTCATGTCTGTCAAATCATAGGTCAT |
| 2703 | mmu-miR-384 | TGTGAACAATTTCTAGGAATTGTGAACAATTTCTAGGAAT |
| 2704 | mmu-miR-196b | CCAACAACAGGAAACTACCTACCAACAACAGGAAACTACCTA |
| 2705 | mmu-miR-409 | AAGGGGTTCACCGAGCAACATAAGGGGTTCACCGAGCAACAT |
| 2706 | mmu-miR-410 | AACAGGCCATCTGTGTTATATTAACAGGCCATCTGTGTTATATT |
| 2707 | mmu-miR-376b | AAAGTGGATGTTCCTCTATGATAAAGTGGATGTTCCTCTATGAT |
| 2708 | mmu-miR-411 | ACTGAGGGTTAGTGGACCGTGTACTGAGGGTTAGTGGACCGTGT |
| 2709 | mmu-miR-412 | ACGGCTAGTGGACCAGGTGAAACGGCTAGTGGACCAGGTGAA |
| 2710 | mmu-miR-370 | AACCAGGTTCCACCCCAGCAAACCAGGTTCCACCCCAGCA |
| 2711 | mmu-miR-425 | CGGACACGACATTCCCGATCGGACACGACATTCCCGAT |
| 2712 | mmu-miR-431 | TGCATGACGGCCTGCAAGACATGCATGACGGCCTGCAAGACA |
| 2713 | mmu-miR-433-5p | GAATAATGACAGGCTCACCGTAGAATAATGACAGGCTCACCGTA |
| 2714 | mmu-miR-433-3p | ACACCGAGGAGCCCATCATGATACACCGAGGAGCCCATCATGAT |
| 2715 | mmu-miR-434-5p | GGTTCAAACCATGAGTCGAGCGGTTCAAACCATGAGTCGAGC |
| 2716 | mmu-miR-434-3p | GGAGTCGAGTGATGGTTCAAAGGAGTCGAGTGATGGTTCAAA |
| 2717 | mmu-miR-448 | ATGGGACATCCTACATATGCAAATGGGACATCCTACATATGCAA |
| 2718 | mmu-miR-429 | ACGGCATTACCAGACAGTATTAACGGCATTACCAGACAGTATTA |
| 2719 | mmu-miR-449 | ACCAGCTAACAATACACTGCCAACCAGCTAACAATACACTGCCA |
| 2720 | mmu-miR-450 | TATTAGGAACACATCGCAAAAATATTAGGAACACATCGCAAAAA |
| 2721 | mmu-miR-451 | AACTCAGTAATGGTAACGGTTTAACTCAGTAATGGTAACGGTTT |
| 2722 | mmu-miR-452 | GTCTCAGTTTCCTCTGCAAACAGTCTCAGTTTCCTCTGCAAACA |
| 2723 | mmu-miR-463 | TGATGGACAACAAATTAGGTTGATGGACAACAAATTAGGT |
| 2724 | mmu-miR-464 | TATCTCACAGAATAAACTTGGTTATCTCACAGAATAAACTTGGT |
| 2725 | mmu-miR-465 | TCACATCAGTGCCATTCTAAATTCACATCAGTGCCATTCTAAAT |
| 2726 | mmu-miR-466 | GTCTTATGTGTGCGTGTATGTAGTCTTATGTGTGCGTGTATGTA |
| 2727 | mmu-miR-467 | GTGTAGGTGTGTGTATGTATATGTGTAGGTGTGTGTATGTATAT |
| 2728 | mmu-miR-468 | AGACACACGCACATCAGTCATAAGACACACGCACATCAGTCATA |
| 2729 | mmu-miR-469 | ACACCAAGATCAATGAAAGAGGACACCAAGATCAATGAAAGAGG |
| 2730 | mmu-miR-470 | TCACCAGTGCCAGTCCAAGAATCACCAGTGCCAGTCCAAGAA |
| 2731 | mmu-miR-471 | TGTGAAAAGCACTATACTACGTTGTGAAAAGCACTATACTACGT |
| 2732 | dme-miR-1 | CTCCATACTTCTTTACATTCCACTCCATACTTCTTTACATTCCA |
| 2733 | dme-miR-2a | CTCATCAAAGCTGGCTGTGATACTCATCAAAGCTGGCTGTGATA |
| 2734 | dme-miR-2b | CTCCTCAAAGCTGGCTGTGATCTCCTCAAAGCTGGCTGTGAT |
| 2735 | dme-miR-3 | TGAGACACACTTTGCCCAGTGTGAGACACACTTTGCCCAGTG |
| 2736 | dme-miR-4 | TCAATGGTTGTCTAGCTTTATCAATGGTTGTCTAGCTTTA |
| 2737 | dme-miR-5 | CATATCACAACGATCGTTCCTTCATATCACAACGATCGTTCCTT |
| 2738 | dme-miR-6 | AAAAAGAACAGCCACTGTGATAAAAAAGAACAGCCACTGTGATA |
| 2739 | dme-miR-7 | ACAACAAAATCACTAGTCTTCCACAACAAAATCACTAGTCTTCC |
| 2740 | dme-miR-8 | GACATCTTTACCTGACAGTATTGACATCTTTACCTGACAGTATT |
| 2741 | dme-miR-9a | TCATACAGCTAGATAACCAAAGTCATACAGCTAGATAACCAAAG |
| 2742 | dme-miR-10 | ACAAATTCGGATCTACAGGGTACAAATTCGGATCTACAGGGT |
| 2743 | dme-miR-11 | GCAAGAACTCAGACTGTGATGGCAAGAACTCAGACTGTGATG |
| 2744 | dme-miR-12 | ACCAGTACCTGATGTAATACTCACCAGTACCTGATGTAATACTC |
| 2745 | dme-miR-13a | ACTCATCAAAATGGCTGTGATAACTCATCAAAATGGCTGTGATA |
| 2746 | dme-miR-13b | ACTCGTCAAAATGGCTGTGATAACTCGTCAAAATGGCTGTGATA |
| 2747 | dme-miR-14 | TAGGAGAGAGAAAAAGACTGATAGGAGAGAGAAAAAGACTGA |
| 2748 | dme-miR-263a | GTGAATTCTTCCAGTGCCATTAGTGAATTCTTCCAGTGCCATTA |
| 2749 | dme-miR-184* | CGGGGCGAGAGAATGATAAGCGGGGCGAGAGAATGATAAG |
| 2750 | dme-miR-184 | CCCTTATCAGTTCTCCGTCCACCCTTATCAGTTCTCCGTCCA |
| 2751 | dme-miR-274 | ATTACCCGTTAGTGTCGGTCAATTACCCGTTAGTGTCGGTCA |
| 2752 | dme-miR-275 | GCGCTACTTCAGGTACCTGAGCGCTACTTCAGGTACCTGA |
| 2753 | dme-miR-92a | ATAGGCCGGGACAAGTGCAATATAGGCCGGGACAAGTGCAAT |
| 2754 | dme-miR-219 | CAAGAATTGCGTTTGGACAATCCAAGAATTGCGTTTGGACAATC |
| 2755 | dme-miR-276* | CGTAGGAACTCTATACCTCGCCGTAGGAACTCTATACCTCGC |
| 2756 | dme-miR-276a | AGAGCACGGTATGAAGTTCCTAAGAGCACGGTATGAAGTTCCTA |
| 2757 | dme-miR-277 | TGTCGTACCAGATAGTGCATTTTGTCGTACCAGATAGTGCATTT |
| 2758 | dme-miR-278 | AAACGGACGAAAGTCCCACGGAAAACGGACGAAAGTCCCACCGA |
| 2759 | dme-miR-133 | ACAGCTGGTTGAAGGGGACCAAACAGCTGGTTGAAGGGGACCAA |
| 2760 | dme-miR-279 | TTAATGAGTGTGGATCTAGTCATTAATGAGTGTGGATCTAGTCA |
| 2761 | dme-miR-33 | CAATGCGACTACAATGCACCTCAATGCGACTACAATGCACCT |
| 2762 | dme-miR-280 | CATTTCATATGCAACGTAAATACA1TTCATATGCAACGTAAATA |
| 2763 | dme-miR-281-1* | ACTGTCGACGGACAGCTCTCTTACTGTCGACGGACAGCTCTCTT |
| 2764 | dme-miR-281 | ACAAAGAGAGCAATTCCATGACACAAAGAGAGCAATTCCATGAC |
| 2765 | dme-miR-282 | ACAGACAAAGCCTAGTAGAGGACAGACAAAGCCTAGTAGAGG |
| 2766 | dme-miR-283 | AGAATTACCAGCTGATATTTAAGAATTACGAGCTGATATTTA |
| 2767 | dme-miR-284 | AATTGCTGGAATCAAGTTGCTGAATTGCTGGAATCAAGTTGCTG |
| 2768 | dme-miR-281-2* | ACTGTCGACGGATAGCTCTCTACTGTCGACGGATAGCTCTCT |
| 2769 | dme-miR-34 | AACCAGCTAACCACACTGCCAAACCAGCTAACCACACTGCCA |
| 2770 | dme-miR-124 | TTGGCATTCACCGCGTGCCTTATTGGCATTCACCGCGTGCCTTA |
| 2771 | dme-miR-79 | ATGCTTTGGTAATCTAGCTTTAATGCTTTGGTAATCTAGCTTTA |
| 2772 | dme-miR-276b | AGAGCACGGTATTAAGTTCCTAAGAGCACGGTATTAAGTTCCTA |
| 2773 | dme-miR-210 | TAGCCGCTGTCACACGCACAATAGCCGCTGTCACACGCACAA |
| 2774 | dme-miR-285 | GCACTGATTTCGAATGGTGCTAGCACTGATTTCGAATGGTGCTA |
| 2775 | dme-miR-100 | CACAAGTTCGGATTTACGGGTTCACAAGTTCGGATTTACGGGTT |
| 2776 | dme-miR-92b | AGGCCGGGACTAGTGCAATTAGGCCGGGACTAGTGCAATT |
| 2777 | dme-miR-286 | AGCACGAGTGTTCGGTCTAGTAGCACGAGTGTTCGGTCTAGT |
| 2778 | dme-miR-287 | GTGCAAACGATTTTCAACACAGTGCAAACGATTTTCAACACA |
| 2779 | dme-miR-87 | CACACCTGAAATTTTGCTCAACACACCTGAAATTTTGCTCAA |
| 2780 | dme-miR-263b | GTGAATTCTCCCAGTGCCAAGGTGAATTCTCCCAGTGCCAAG |
| 2781 | dme-miR-288 | CATGAAATGAAATCGACATGAACATGAAATGAAATCGACATGAA |
| 2782 | dme-miR-289 | AGTCGCAGGCTCCACTTAAATAAGTCGCAGGCTCCACTTAAATA |
| 2783 | dine-bantam | AATCAGCTTTCAAAATGATCTCAATCAGCTTTCAAAATGATCTC |
| 2784 | dme-miR-303 | ACCAGTTTCCTGTGAAACCTAAACCAGTTTCCTGTGAAACCTAA |
| 2785 | dme-miR-31b | CAGCTATTCCGACATCTTGCCCAGCTATTCCGACATCTTGCC |
| 2786 | dme-miR-304 | CTCACATTTACAAATTGAGATTCTCACATTTACAAATTGAGATT |
| 2787 | dme-miR-305 | CAGAGCACCTGATGAAGTACAACAGAGCACCTGATGAAGTACAA |
| 2788 | dme-miR-9c | TCTACAGCTAGAATACCAAAGATCTACAGCTAGAATACCAAAGA |
| 2789 | dme-miR-306 | TTGAGAGTCACTAAGTACCTGATTGAGAGTCACTAAGTACCTGA |
| 2790 | dme-miR-306* | GCACAGGCACAGAGTGACGCACAGGCACAGAGTGAC |
| 2791 | dme-miR-9b | CATACAGCTAAAATCACCAAAGCATACAGCTAAAATCACCAAAG |
| 2792 | dme-let-7 | ACTATACAACCTACTACCTCAACTATACAACCTACTACCTCA |
| 2793 | dme-miR-125 | TCACAAGTTAGGGTCTCAGGGTCACAAGTTAGGGTCTCAGGG |
| 2794 | dme-miR-307 | CTCACTCAAGGAGGTVGTGACTCACTCAAGGAGGTTGTGA |
| 2795 | dme-miR-308 | CTCACAGTATAATCCTGTGATTCTCACAGTATAATCCTGTGATT |
| 2796 | dme-miR-31a | TCAGCTATGCCGACATCTTGCTCAGCTATGCCGACATCTTGC |
| 2797 | dme-miR-309 | TAGGACAAACTTTACCCAGTGCTAGGACAAACTTTACCCAGTGC |
| 2798 | dme-miR-310 | AAAGGCCGGGAAGTGTGCAATAAAGGCCGGGAAGTGTGCAAT |
| 2799 | dme-miR-311 | TCAGGCCGGTGAATGTGCAATTCAGGCCGGTGAATGTGCAAT |
| 2800 | dme-miR-312 | TCAGGCCGTCTCAAGTGCAATTCAGGCCGTCTCAAGTGCAAT |
| 2801 | dme-miR-313 | TCGGGCTGTGAAAAGTGCAATATCGGGCTGTGAAAAGTGCAATA |
| 2802 | dme-miR-314 | CCGAACTTATTGGCTCGAATACCGAACTTATTGGCTCGAATA |
| 2803 | dme-miR-315 | GCTTTCTGAGCAACAATCAAAAGCTTTCTGAGCAACAATCAAAA |
| 2804 | dme-miR-316 | CGCCAGTAAGCGGAAAAAGACCGCCAGTAAGCGGAAAAAGAC |
| 2805 | dme-miR-317 | ACTGGATACCACCAGCTGTGTACTGGATACCACCAGCTGTGT |
| 2806 | dme-miR-318 | TGAGATAAACAAAGCCCAGTGATGAGATAAACAAAGCCCAGTGA |
| 2807 | dme-miR-2c | CCCATCAAAGCTGGCTGTGATCCCATCAAAGCTGGCTGTGAT |
| 2808 | dme-miR-iab-4-5p | TCAGGATACATTCAGTATACGTTCAGGATACATTCAGTATACGT |
| 2809 | dme-miR-iab-4-3p | GTTACGTATACTGAAGGTATACGTTACGTATACTGAAGGTATAC |
| 2810 | cel-let-7 | AACTATACAACCTACTACCTCAAACTATACAACCTACTACCTCA |
| 2811 | cel-lin-4 | TCACACTTGAGGTCTCAGGGATCACACTTGAGGTCTCAGGGA |
| 2812 | cel-miR-1 | TACATACTTCTTTACATTCCATACATACTTCTTTACATTCCA |
| 2813 | cei-miR-2 | CACATCAAAGCTGGCTGTGATACACATCAAAGCTGGCTGTGATA |
| 2814 | cel-miR-34 | AACCAGCTAACCACACTGCCTAACCAGCTAACCACACTGCCT |
| 2815 | cel-miR-35 | ACTGCTAGTTTCCACCCGGTGAACTGCTAGTTTCCACCCGGTGA |
| 2816 | cel-miR-36 | CATGCGAATTTTCACCCGGTGCATGCGAATTTTCACCCGGTG |
| 2817 | cel-miR-37 | ACTGCAAGTGTTCACCCGGTGAACTGCAAGTGTTCACCCGGTGA |
| 2818 | cel-miR-38 | ACTCCAGTTTTTCTCCCGGTGACTCCAGTTTTTCTCCCGGTG |
| 2819 | cel-miR-39 | CAAGCTGATTTACACCCGGTGCAAGCTGATTTACACCCGGTG |
| 2820 | cel-miR-40 | TTAGCTGATGTACACCCGGTGTTAGCTGATGTACACCCGGTG |
| 2821 | cel-miR-41 | TAGGTGATTTTTCACCCGGTGATAGGTGATTTTTCACCCGGTGA |
| 2822 | cel-miR-42 | CTGTAGATGTTAACCCGGTGCTGTAGATGTTAACCCGGTG |
| 2823 | cel-miR-43 | GCGACAGCAAGTAAACTGTGATGCGACAGCAAGTAAACTGTGAT |
| 2824 | cei-miR-44 | AGCTGAATGTGTCTCTAGTCAAGCTGAATGTGTCTCTAGTCA |
| 2825 | cel-miR-45 | AGCTGAATGTGTCTCTAGTCAAGCTGAATGTGTCTCTAGTCA |
| 2826 | cel-miR-46 | TGAAGAGAGCGACTCCATGACTGAAGAGAGCGACTCCATGAC |
| 2827 | cel-miR-47 | TGAAGAGAGCGCCTCCATGACATGAAGAGAGCGCCTCCATGACA |
| 2828 | cel-miR-48 | TCGCATCTACTGAGCCTACCTTCGCATCTACTGAGCCTACCT |
| 2829 | cel-miR-49 | TCTGCAGCTTCTCGTGGTGCTTTCTGCAGCTTCTCGTGGTGCTT |
| 2830 | cel-miR-50 | ACCCAAGAATACCAGACATATCACCCAAGAATACCAGACATATC |
| 2831 | cel-miR-51 | AACATGGATAGGAGCTACGGGAACATGGATAGGAGCTACGGG |
| 2832 | cel-miR-52 | AGCACGGAAACATATGTACGGAGCACGGAAACATATGTACGG |
| 2833 | cel-miR-53 | AGCACGGAAACAAATGTACGGAGCACGGAAACAAATGTACGG |
| 2834 | cel-miR-54 | CTCGGATTATGAAGATTACGGGCTCGGATTATGAAGATTACGGG |
| 2835 | cel-miR-55 | CTCAGCAGAAACTTATACGGGTCTCAGCAGAAACTTATACGGGT |
| 2836 | cel-miR-56* | TACAACCCAAAATGGATCCGCTACAACCCAAAATGGATCCGC |
| 2837 | cel-miR-56 | CTCAGCGGAAACATTACGGGTCTCAGCGGAAACATTACGGGT |
| 2838 | cel-miR-57 | ACACACAGCTCGATCTACAGGACACACAGCTCGATCTACAGG |
| 2839 | cel-miR-58 | ATTGCCGTACTGAACGATCTCAATTGCCGTACTGAACGATCTCA |
| 2840 | cel-miR-59 | CATCATCCTGATAAACGATTCGCATCATCCTGATAAACGATTCG |
| 2841 | cel-miR-60 | TGAACTAGAAAATGTGCATAATTGAACTAGAAAATGTGCATAAT |
| 2842 | cel-miR-61 | GAGATGAGTAACGGTTCTAGTCGAGATGAGTAACGGTTCTAGTC |
| 2843 | cel-miR-62 | CTGTAAGCTAGATTACATATCACTGTAAGCTAGATTACATATCA |
| 2844 | cel-miR-63 | TTTCCAACTCGCTTCAGTGTCATTTCCAACTCGCTTCAGTGTCA |
| 2845 | cel-miR-64 | TTCGGTAACGCTTCAGTGTCATTTCGGTAACGCTTCAGTGTCAT |
| 2846 | cel-miR-65 | TTCGGTTACGCTTCAGTGTCATTTCGGTTACGCTTCAGTGTCAT |
| 2847 | cel-miR-66 | TCACATCCCTAATCAGTGTCATTCACATCCCTAATCAGTGTCAT |
| 2848 | cel-miR-67 | TCTACTCTTTCTAGGAGGTTGTTCTACTCTTTCTAGGAGGTTGT |
| 2849 | cel-miR-70 | ATGGAAACACCAACGACGTATTATGGAAACACCAACGACGTATT |
| 2850 | cel-miR-71 | TCACTACCCATGTCTTTCATCACTACCCATGTCTTTCA |
| 2851 | cei-miR-72 | GCTATGCCAACATCTTGCCTGCTATGCCAACATCTTGCCT |
| 2852 | cel-miR-73 | ACTGAACTGCCTACATCTTGCACTGAACTGCCTACATCTTGC |
| 2853 | cel-miR-74 | TGTAGACTGCCATTTCTTGCCATGTAGACTGCCATTTCTTGCCA |
| 2854 | cel-miR-75 | TGAAGCCGGTTGGTAGCTTTAATGAAGCCGGTTGGTAGCTTTAA |
| 2855 | cel-miR-76 | TCAAGGCTTCATCAACAACGAATCAAGGCTTCATCAACAACGAA |
| 2856 | cel-miR-77 | TGGACAGCTATGGCCTGATGATGGACAGCTATGGCCTGATGA |
| 2857 | cel-miR-78 | CACAAACAACCAGGCCTCCACACAAACAACCAGGCCTCCA |
| 2858 | cel-miR-79 | AGCTTTGGTAACCTAGCTTTATAGCTTTGGTAACCTAGCTTTAT |
| 2859 | cel-miR-227 | GTTCAGAATCATGTCGAAAGCTGTTCAGAATCATGTCGAAAGCT |
| 2860 | cel-miR-80 | TCGGCTTTCAACTAATGATCTCTCGGCTTTCAACTAATGATCTC |
| 2861 | cel-miR-81 | ACTAGCTTTCACGATGATCTCAACTAGCTTTCACGATGATCTCA |
| 2862 | cel-miR-82 | ACTGGCTTTCACGATGATCTCAACTGGCTTTCACGATGATCTCA |
| 2863 | cel-miR-83 | TTACTGAATTTATATGGTGCTATTACTGAATTTATATGGTGCTA |
| 2864 | cel-miR-84 | TACAATATTACATACTACCTCATACAATATTACATACTACCTCA |
| 2865 | cel-miR-85 | GCACGACTTTTCAAATACTTTGGCACGACTTTTCAAATACTTTG |
| 2866 | cel-miR-86 | GACTGTGGCAAAGCATTCACTTGACTGTGGCAAAGCATTCACTT |
| 2867 | cel-miR-87 | ACACCTGAAACTTTGCTCACACACCTGAAACTTTGCTCAC |
| 2868 | cel-miR-90 | GGGGCATTCAAACAACATATCAGGGGCATTCAAACAACATATCA |
| 2869 | cel-miR-124 | TGGCATTCACCGCGTGCCTTATGGCATTCACCGCGTGCCTTA |
| 2870 | cel-miR-228 | CGTGAATTCATGCAGTGCCATTCGTGAATTCATGCAGTGCCATT |
| 2871 | cel-miR-229 | ACGATGGAAAAGATAACCAGTGACGATGGAAAAGATAACCAGTG |
| 2872 | cel-miR-230 | TCTCCTGGTCGCACAACTAATATCTCCTGGTCGCACAACTAATA |
| 2873 | cel-miR-231 | TTCTGCCTGTTGATCACGAGCTTCTGCCTGTTGATCACGAGC |
| 2874 | cel-miR-232 | TCACCGCAGTTAAGATGCATTTTCACCGCAGTTAAGATGCATTT |
| 2875 | cel-miR-233 | TCCCGCACATGCGCATTGCTCATCCCGCACATGCGCATTGCTCA |
| 2876 | cel-miR-234 | AAGGGTATTCTCGAGCAATAAAAGGGTATTCTCGAGCAATAA |
| 2877 | cel-miR-235 | TCAGGCCGGGGAGAGTGCAATATCAGGCCGGGGAGAGTGCAATA |
| 2878 | cel-miR-236 | AGCGTCATTACCTGACAGTATTAGCGTCATTACCTGACAGTATT |
| 2879 | cel-miR-237 | AAGCTGTTCGAGAATTCTCAGGAAGCTGTTCGAGAATTCTCAGG |
| 2880 | cel-miR-238 | TCTGAATGGCATCGGAGTACAATCTGAATGGCATCGGAGTACAA |
| 2881 | cel-miR-239a | CCAGTACCTATGTGTAGTACAACCAGTACCTATGTGTAGTACAA |
| 2882 | cel-miR-239b | CAGTACTTTTGTGTAGTACACAGTACTTTTGTGTAGTACA |
| 2883 | cel-miR-240 | AGCGAAGATTTGGGGGCCAGTAAGCGAAGATTTGGGGGCCAGTA |
| 2884 | cel-miR-241 | TCATTTCTCGCACCTACCTCATCATTTCTCGCACCTACCTCA |
| 2885 | cel-miR-242 | TCGAAGCAAAGGCCTACGCAATCGAAGCAAAGGCCTACGCAA |
| 2886 | cel-miR-243 | ATATCCCGCCGCGATCGTAATATCCCGCCGCGATCGTA |
| 2887 | cel-miR-244 | CATACCACTTTGTACAACCAAACATACCACTTTGTACAACCAAA |
| 2888 | cel-miR-245 | AGCTACTTGGAGGGGACCAATAGCTACTTGGAGGGGACCAAT |
| 2889 | cel-miR-246 | AGCTCCTACCCGAAACATGTAAAGCTCCTACCCGAAACATGTAA |
| 2890 | cel-miR-247 | AAGAAGAGAATAGGCTCTAGTCAAGAAGAGAATAGGCTCTAGTC |
| 2891 | cel-miR-248 | TGAGCGTTATCCGTGCACGTGTTGAGCGTTATCCGTGCACGTGT |
| 2892 | cel-miR-249 | GCAACGCTCAAAAGTCCTGTGGCAACGCTCAAAAGTCCTGTG |
| 2893 | cel-miR-250 | CCATGCCAACAGTTGACTGTGCCATGCCAACAGTTGACTGTG |
| 2894 | cel-miR-251 | AATAAGAGCGGCACCACTACTTAATAAGAGCGGCACCACTACTT |
| 2895 | cel-miR-252 | TTACCTGCGGCACTACTACTTATTACCTGCGGCACTACTACTTA |
| 2896 | cel-miR-253 | GGTCAGTGTTAGTGAGGTGTGGGTCAGTGTTAGTGAGGTGTG |
| 2897 | cel-miR-254 | CTACAGTCGCGAAAGATTTGCACTACAGTCGCGAAAGATTTGCA |
| 2898 | cel-miR-256 | TACAGTCTTCTATGCATTCCATACAGTCTTCTATGCATTCCA |
| 2899 | cel-miR-257 | TCACTGGGTACTCCTGATACTTCACTGGGTACTCCTGATACT |
| 2900 | cel-miR-258 | AAAAGGATTCCTCTCAAAACCAAAAGGATTCCTCTCAAAACC |
| 2901 | cel-miR-259 | TACCAGATTAGGATGAGATTTACCAGATTAGGATGAGATT |
| 2902 | cel-miR-260 | CTACAAGAGTTCGACATCACCTACAAGAGTTCGACATCAC |
| 2903 | cel-miR-261 | CGTGAAAACTAAAAAGCTACGTGAAAACTAAAAAGCTA |
| 2904 | cel-miR-262 | ATCAGAAAACATCGAGAAACATCAGAAAACATCGAGAAAC |
| 2905 | cel-miR-264 | CATAACAACAACCACCCGCCCATAACAACAACCACCCGCC |
| 2906 | cel-miR-265 | ATACCACCCTTCCTCCCTCAATACCACCCTTCCTCCCTCA |
| 2907 | cel-miR-266 | GCTTTGCCAAAGTCTTGCCTGCTTTGCCAAAGTCTTGCCT |
| 2908 | cel-miR-267 | TGCAGCAGACACTTCACGGTGCAGCAGACACTTCACGG |
| 2909 | cel-miR-268 | CCAAACTGCTTCTAATTCTTGCCCAAACTGCTTCTAATTCTTGC |
| 2910 | cel-miR-269 | AGTTTTGCCAGAGTCTTGCCAGTTTTGCCAGAGTCTTGCC |
| 2911 | cel-miR-270 | CTCCACTGCTACATCATGCCCTCCACTGCTACATCATGCC |
| 2912 | cel-miR-271 | AATGCTTTCCCACCCGGCGAAATGCTTTCCCACCCGGCGA |
| 2913 | cel-miR-272 | CAAACACCCATGCCTACACAAACACCCATGCCTACA |
| 2914 | cel-miR-273 | AGCCGACACAGTACGGGCAAGCCGACACAGTACGGGCA |
| 2915 | cel-miR-353 | AATACCAACACATGGCAATTGAATACCAACACATGGCAATTG |
| 2916 | cel-miR-354 | AGGAGCAGCAACAAACAAGGTAGGAGCAGCAACAAACAAGGT |
| 2917 | cel-miR-355 | CATAGCTCAGGCTAAAACAAACATAGCTCAGGCTAAAACAAA |
| 2918 | cel-miR-356 | TGATTTGTTCGCGTTGCTCAATGATTTGTTCGCGTTGCTCAA |
| 2919 | cel-miR-357 | TCCTGCAACGACTGGCATTTATCCTGCAACGACTGGCATTTA |
| 2920 | cel-miR-358 | CCTTGACAGGGATACCAATTGCCTTGACAGGGATACCAATTG |
| 2921 | cel-miR-359 | TCGTGAGAGAAAGACCAGTGATCGTCAGAGAAAGACCAGTGA |
| 2922 | cel-miR-360 | TTGTGAACGGGATTACGGTCATTGTGAACGGGATTACGGTCA |
| 2923 | cel-Isy-6 | CGAAATGCGTCTCATACAAAACGAAATGCGTCTCATACAAAA |
| 2924 | cel-miR-392 | TCATCACACGTGATCGATGATATCATCACACGTGATCGATGATA |
| 2925 | dre-miR-7b | AACAAAATCACAAGTCTTCCAACAAAATCACAAGTCTTCC |
| 2926 | dre-miR-7a | ACAACAAAATCACTAGTCTTCCACAACAAAATCACTAGTCTTCC |
| 2927 | dre-miR-10a | ACAAATTCGGATCTACAGGGTAACAAATTCGGATCTACAGGGTA |
| 2928 | dre-miR-10b | CACAAATTCGGTTCTACAGGGTCACAAATTCGGTTCTACAGGGT |
| 2929 | dre-miR-34 | ACAACCAGCTAAGACACTGCCACAACCAGCTAAGACACTGCC |
| 2930 | dre-miR-181b | CCCACCGACAGCAATGAATGTCCCACCGACAGCAATGAATGT |
| 2931 | dre-miR-182 | TGTGAGTTCTACCATTGCCAAATGTGAGTTCTACCATTGCCAAA |
| 2932 | dre-miR-182* | TAGTTGGCAAGTCTAGAACCATAGTTGGCAAGTCTAGAACCA |
| 2933 | dre-miR-183 | CAGTGAATTCTACCAGTGCCATCAGTGAATTCTACCAGTGCCAT |
| 2934 | dre-miR-187 | GGCTGGAACACAAGACACGAGGCTGCAACACAAGACACGA |
| 2935 | dre-miR-192 | GGCTGTCAATTCATAGGTCATGGCTGTCAATTCATAGGTCAT |
| 2936 | dre-miR-196a | CCCAACAACATGAAACTACCTACCCAACAACATGAAACTACCTA |
| 2937 | dre-miR-199 | GAACAGGTAGTCTGAACACTGGAACAGGTAGTCTGAACACTG |
| 2938 | dre-miR-203a | CAAGTGGTCCTAAACATTTCACCAAGTGGTCCTAAACATTTCAC |
| 2939 | dre-miR-204 | AGGCATAGGATGACAAAGGGAAAGGCATAGGATGACAAAGGGAA |
| 2940 | dre-miR-205 | AGACTCCGGTGGAATGAAGGAAGACTCCGGTGGAATGAAGGA |
| 2941 | dre-miR-210 | TTAGCCGCTGTCACACGCACATTAGCCGCTGTCACACGCACA |
| 2942 | dre-miR-213 | GGTACAATCAACGGTCAATGGTGGTACAATCAACGGTCAATGGT |
| 2943 | dre-miR-214 | TGCCTGTCTGTGCCTGCTGTTGCCTGTCTGTGCCTGCTGT |
| 2944 | dre-miR-216a | TCACAGTTGCCAGCTGAGATTATCACAGTTGCCAGCTGAGATTA |
| 2945 | dre-miR-217 | CCAATCAGTTCCTGATGCAGTACCAATCAGTTCCTGATGCAGTA |
| 2946 | dre-miR-219 | AAGAATTGCGTTTGGACAATCAAAGAATTGCGTTTGGACAATCA |
| 2947 | dre-miR-220 | AAGTGTCCGATACGGTTGTGGAAGTGTCCGATACGGTTGTGG |
| 2948 | dre-miR-221 | AAACCCAGCAGACAATGTAGCTAAACCCAGCAGACAATGTAGCT |
| 2949 | dre-miR-222 | AGACCCAGTAGCCAGATGTAGAGACCCAGTAGCCAGATGTAG |
| 2950 | dre-miR-223 | GGGGTATTTGACAAACTGACAGGGGTATTTGACAAACTGACA |
| 2951 | dre-miR-430a | CTACCCCAACAAATAGCACTTACTACCCCAACAAATAGCACTTA |
| 2952 | dre-miR-430b | CTACCCCAACTTGATAGCACTTCTACCCCAACTTGATAGCACTT |
| 2953 | dre-miR-430c | CTACCCCAAAGAGAAGCACTTACTACCCCAAAGAGAAGCACTTA |
| 2954 | dre-miR-181a | ACTCACCGACAGCGTTGAATGACTCACCGACAGCGTTGAATG |
| 2955 | dre-miR-429 | ACGGCATTACCAGACAGTATTAACGGCATTACCAGACAGTATTA |
| 2956 | dre-miR-451 | AACTCAGTAATGGTAACGGTTTAACTCAGTAATGGTAACGGTTT |
| 2957 | dre-let-7a | AACTATACAACCTACTACCTCAAACTATACAACCTACTACCTCA |
| 2958 | dre-let-7b | AACCACACAACCTACTACCTCAAACCACACAACCTACTACCTCA |
| 2959 | dre-let-7c | AACCATACAACCTACTACCTCAAACCATACAACCTACTACCTCA |
| 2960 | dre-let-7d | AACCATACAACCAACTACCTCAAACCATACAACCAACTACCTCA |
| 2961 | dre-let-7e | AACTATTCAATCTACTACCTCAAACTATTCAATCTAGTACCTCA |
| 2962 | dre-let-7f | AACTATACAATCTACTACCTCAAACTATACAATCTACTACCTCA |
| 2963 | dre-let-7g | AACTATACAAACTACTACCTCAAACTATACAAACTACTACCTCA |
| 2964 | dre-let-7h | AACAACACAACTTACTACCTCAAACAACACAACTTACTACCTCA |
| 2965 | dre-let-7i | AACAGCACAAACTACTACCTCAAACAGCACAAACTACTACCTCA |
| 2966 | dre-miR-1 | ATACATACTTCTTTACATTCCAATACATACTTCTTTACATTCCA |
| 2967 | dre-miR-9 | TCATACAGCTAGATAACCAAAGTCATACAGCTAGATAACCAAAG |
| 2968 | dre-miR-10c | ACAAATCCGGATCTACAGGGTAACAAATCCGGATCTACAGGGTA |
| 2969 | dre-miR-10d | ACACATTCGGTTCTACAGGGTAACACATTCGGTTCTACAGGGTA |
| 2970 | dre-miR-15a | CACAAACCATTCTGTGCTGCTACACAAACCATTCTGTGCTGCTA |
| 2971 | dre-miR-15b | TACAAACCATGATGTGCTGCTATACAAACCATGATGTGCTGCTA |
| 2972 | dre-miR-16a | CACCAATATTTACGTGCTGCTACACCAATATTTACGTGCTGCTA |
| 2973 | dre-miR-16b | CTCCAATATTTACGTGCTGCTACTCCAATATTTACGTGCTGCTA |
| 2974 | dre-miR-16c | CTCCAATATTTACATGCTGCTACTCCAATATTTACATGCTGCTA |
| 2975 | dre-miR-17a | TACCTGCACTGTAAGCACTTTGTACCTGCACTGTAAGCACTTTG |
| 2976 | dre-miR-20b | CTACCTGCACTGTGAGCACTTCTACCTGCACTGTGAGCACTT |
| 2977 | dre-miR-18a | TATCTGCACTAGATGCACCTTATATCTGCACTAGATGCACCTTA |
| 2978 | dre-miR-18b | TATCTGCACTAAATGCACCTTATATCTGCACTAAATGCACCTTA |
| 2979 | dre-miR-18c | TAACTACACAAGATGCACCTTATAACTACACAAGATGCACCTTA |
| 2980 | dre-miR-19a | TCAGTTTTGCATAGATTTGCACTCAGTTTTGCATAGATTTGCAC |
| 2981 | dre-miR-19b | TCAGTTTTGCATGGATTTGCACTCAGTTTTGCATGGATTTGCAC |
| 2982 | dre-miR-19c | CGAGTTTTGCATGGATTTGCACCGAGTTTTGCATGGATTTGCAC |
| 2983 | dre-miR-19d | TCAGTTTTGCATGGGTTTGCACTCAGTTTTGCATGGGTTTGCAC |
| 2984 | dre-miR-20a | CTACCTGCACTATAAGCACTTTCTACCTGCACTATAAGCACTTT |
| 2985 | dre-miR-21 | CCAACACCAGTCTGATAAGCTACCAACACCAGTCTGATAAGCTA |
| 2986 | dre-miR-22a | ACAGTTCTTCAGCTGGCAGCTACAGTTCTTCAGCTGGCAGCT |
| 2987 | dre-miR-22b | ACAGCTCTTCAACTGGCAGCTACAGCTCTTCAACTGGCAGCT |
| 2988 | dre-miR-23a | TGGAAATCCCTGGCAATGTGATTGGAAATCCCTGGCAATGTGAT |
| 2989 | dre-miR-23b | TGGTAATCCCTGGCAATGTGATTGGTAATCCCTGGCAATGTGAT |
| 2990 | dre-miR-24 | TGTTCCTGCTGAACTGAGCCATGTTCCTGCTGAACTGAGCCA |
| 2991 | dre-miR-25 | TCAGACCGAGACAAGTGCAATTCAGACCGAGACAAGTGCAAT |
| 2992 | dre-miR-26a | AGCCTATCCTGGATTACTTGAAAGCCTATCCTGGATTACTTGAA |
| 2993 | dre-miR-26b | AACCTATCCTGGATTACTTGAAAACCTATCCTGGATTACTTGAA |
| 2994 | dre-miR-27a | AGCGGAACTTAGCCACTGTGAAGCGGAACTTAGCCACTGTGA |
| 2995 | dre-miR-27b | TGCAGAACTTAGCCACTGTGAATGCAGAACTTAGCCACTGTGAA |
| 2996 | dre-miR-27c | GCAGAACTTAACCACTGTGAAGCAGAACTTAACCACTGTGAA |
| 2997 | dre-miR-27d | TGAAGAACTTAGCCACTGTGAATGAAGAACTTAGCCACTGTGAA |
| 2998 | dre-miR-27e | CACTGAACTTAGCCACTGTGAACACTGAACTTAGCCACTGTGAA |
| 2999 | dre-miR-29b | ACACTGATTTCAAATGGTGCTAACACTGATTTCAAATGGTGCTA |
| 3000 | dre-miR-29a | TAACCGATTTCAAATGGTGCTATAACCGATTTCAAATGGTGCTA |
| 3001 | dre-miR-30a | CTTCCAGTCGGGAATGTTTACACTTCCAGTCGGGAATGTTTACA |
| 3002 | dre-miR-30b | AGCTGAGTGTAGGATGTTTACAAGCTGAGTGTAGGATGTTTACA |
| 3003 | dre-miR-30c | CTGAGAGTGTAGGATGTTTACACTGAGAGTGTAGGATGTTTACA |
| 3004 | dre-miR-30d | CTTCCAGTCGGGGATGTTTACCTTCCAGTCGGGGATGTTTAC |
| 3005 | dre-miR-30e | CTTCCAGTCAAGGATGTTTACACTTCCAGTCAAGGATGTTTACA |
| 3006 | dre-miR-92a | ACAGGCCGGGACAAGTGCAATAACAGGCCGGGACAAGTGCAATA |
| 3007 | dre-miR-92b | AGGCCGGGACGAGTGCAATAAGGCCGGGACGAGTGCAATA |
| 3008 | dre-miR-93 | TACCTGCACAAACAGCACTTTTTACCTGCACAAACAGCACTTTT |
| 3009 | dre-miR-96 | AGCAAAAATGTGCTAGTGCCAAAGCAAAAATGTGCTAGTGCCAA |
| 3010 | dre-miR-99 | CACAAGATCGGATCTACGGGTCACAAGATCGGATCTACGGGT |
| 3011 | dre-miR-100 | CACAAGTTCGGATCTACGGGTCACAAGTTCGGATCTACGGGT |
| 3012 | dre-miR-101a | CTTCAGTTATCACAGTACTGTACTTCAGTTATCACAGTACTGTA |
| 3013 | dre-miR-101b | CTTCAGTTATCATAGTACTGTACTTCAGTTATCATAGTACTGTA |
| 3014 | dre-miR-103 | TCATAGCCCTGTACAATGCTGTCATAGCCCTGTACAATGCTG |
| 3015 | dre-miR-107 | TGATAGCCCTGTACAATGCTGTGATAGCCCTGTACAATGCTG |
| 3016 | dre-miR-122 | CAAACACCATTGTCACACTCCACAAACACCATTGTCACACTCCA |
| 3017 | dre-miR-124 | TTGGCATTCACCGCGTGCCTTATTGGCATTCACCGCGTGCCTTA |
| 3018 | dre-miR-125a | ACAGGTTAAGGGTCTCAGGGAACAGGTTAAGGGTCTCAGGGA |
| 3019 | dre-miR-125b | TCACAAGTTAGGGTCTCAGGGTCACAAGTTAGGGTCTCAGGG |
| 3020 | dre-miR-125c | TCACGAGTTAGGGTCTCAGGGATCACGAGTTAGGGTCTCAGGGA |
| 3021 | dre-miR-126 | GCATTATTACTCACGGTACGAGCATTATTACTCACGGTACGA |
| 3022 | dre-miR-128 | AAAAGAGACCGGTTCACTGTGAAAAAGAGACCGGTTCACTGTGA |
| 3023 | dre-miR-129 | AGCAAGCCCAGACCGCAAAAAAGCAAGCCCAGACCGCAAAAA |
| 3024 | dre-miR-130a | ATGCCCTTTTAACATTGCACTGATGCCCTTTTAACATTGCACTG |
| 3025 | dre-miR-130b | ATGCCCTTTCATTATTGCACTGATGCCCTTTCATTATTGCACTG |
| 3026 | dre-miR-130c | ATGCCCTTTTAATATTGCACTGATGCCCT1TTAATATTGCACTG |
| 3027 | dre-miR-132 | CGACCATGGCTGTAGACTGTTCGACCATGGCTGTAGACTGTT |
| 3028 | dre-miR-133a | AGCTGGTTGAAGGGGACCAAAAGCTGGTTGAAGGGGACCAAA |
| 3029 | dre-miR-133b | TAGCTGGTTGAAGGGGACCAATAGCTGGTTGAAGGGGACCAA |
| 3030 | dre-miR-133c | TAGCTGGTTGAAAGGGACCAAATAGCTGGTTGAAAGGGACCAAA |
| 3031 | dre-miR-135 | CACATAGGAATAGAAAGCCATACACATAGGAATAGAAAGCCATA |
| 3032 | dre-miR-137 | TACGCGTATTCTTAAGCAATAATACGCGTATTCTTAAGCAATAA |
| 3033 | dre-miR-138 | GCCTGATTCACAACACCAGCTGCCTGATTCACAACACCAGCT |
| 3034 | dre-miR-140 | CTACCATAGGGTAAAACCACTGCTACCATAGGGTAAAACCACTG |
| 3035 | dre-miR-141 | GCATCGTTACCAGACAGTGTTAGCATCGTTACCAGACAGTGTTA |
| 3036 | dre-miR-142a-5p | GTAGTGCTTTCTACTTTATGGTAGTGCTTTCTACTTTATG |
| 3037 | dre-miR-142b-5p | TAGTAGTGCTGTCTACTTTATGTAGTAGTGCTGTCTACTTTATG |
| 3038 | dre-miR-143 | GAGCTACAGTGCTTCATCTCAGAGCTACAGTGCTTCATCTCA |
| 3039 | dre-miR-144 | AGTACATCATCTATACTGTAAGTACATCATCTATACTGTA |
| 3040 | dre-miR-145 | GGGATTCCTGGGAAAACTGGAGGGATTCCTGGGAAAACTGGA |
| 3041 | dre-miR-146a | CCATCTATGGAATTCAGTTCTCCCATCTATGGAATTCAGTTCTC |
| 3042 | dre-miR-146b | CACCCTTGGAATTCAGTTCTCACACCCTTGGAATTCAGTTCTCA |
| 3043 | dre-miR-148 | ACAAAGTTCTGTAATGCACTGAACAAAGTTCTGTAATGCACTGA |
| 3044 | dre-miR-150 | CACTGGTACAAGGATTGGGAGCACTGGTACAAGGATTGGGAG |
| 3045 | dre-miR-152 | CCAAAGTTCTGTCATGCACTGACCAAAGTTCTGTCATGCACTGA |
| 3046 | dre-miR-153b | GCTCATTTTTGTGACTATGCAAGCTCATTTTTGTGACTATGCAA |
| 3047 | dre-miR-153a | GATCACTTTTGTGACTATGCAAGATCACTTTTGTGACTATGCAA |
| 3048 | dre-miR-153c | GATCATTTTTGTGACTATGCAAGATCATTTTTGTGACTATGCAA |
| 3049 | dre-miR-155 | CCCCTATCACGATTAGCATTAACCCCTATCACGATTAGCATTAA |
| 3050 | dre-miR-181c | CCCACCGACAGCAATGAATGTCCCACCGACAGCAATGAATGT |
| 3051 | dre-miR-184 | CCCTTATCAGTTCTCCGTCCACCCTTATCAGTTCTCCGTCCA |
| 3052 | dre-miR-190 | ACCTAATATATCAAACATATCAACCTAATATATCAAACATATCA |
| 3053 | dre-miR-462 | AGCTGCATTATGGGTTCCGTTAAGCTGCATTATGGGTTCCGTTA |
| 3054 | dre-miR-193a | ACTGGGACTTTGTAGGCCAGTACTGGGACTTTGTAGGCCAGT |
| 3055 | dre-miR-193b | AGCGGGACTTTGCGGGCCAGTTAGCGGGACTTTGCGGGCCAGTT |
| 3056 | dre-miR-194a | CCACATGGAGTTGCTGTTACACCACATGGAGTTGCTGTTACA |
| 3057 | dre-miR-194b | TCCACATGGAGCGGCTGTTACATCCACATGGAGCGGCTGTTACA |
| 3058 | dre-miR-196b | CCCAACAACTTGAAACTACCTACCCAACAACTTGAAACTACCTA |
| 3059 | dre-miR-200a | ACATCGTTACCAGACAGTGTTAACATCGTTACCAGACAGTGTTA |
| 3060 | dre-miR-200b | TCATCATTACCAGGCAGTATTATCATCATTACCAGGCAGTATTA |
| 3061 | dre-miR-200c | GCATCATTACCAGGCAGTATTAGCATCATTACCAGGCAGTATTA |
| 3062 | dre-miR-202 | TTTTCCCATGCCCTATGCCTCTTTTCCCATGCCCTATGCCTC |
| 3063 | dre-miR-203b | CAAGTGGTCCTGAACATTTCACCAAGTGGTCCTGAACATTTCAC |
| 3064 | dre-miR-206 | CCACACACTTCCTTACATTCCACCACACACTTCCTTACATTCCA |
| 3065 | dre-miR-216b | TCACAGTTGCCTGCAGAGATTATCACAGTTGCCTGCAGAGATTA |
| 3066 | dre-miR-218a | CACATGGTTAGATCAAGCACAACACATGGTTAGATCAAGCACAA |
| 3067 | dre-miR-218b | TGCATGGTTAGATCAAGCACAATGCATGGTTAGATCAAGCACAA |
| 3068 | dre-miR-301a | CTTTGACAATACTATTGCACTGCTTTGACAATACTATTGCACTG |
| 3069 | dre-miR-301b | CAATGACAATACTATTGCACTGCAATGACAATACTATTGCACTG |
| 3070 | dre-miR-301c | CTATGACAATACTATTGCACTGCTATGACAATACTATTGCACTG |
| 3071 | dre-miR-338 | CAACAAAATCACTGATGCTGGACAACAAAATCACTGATGCTGGA |
| 3072 | dre-miR-363 | TACAGATGGATACCGTGCAATTTACAGATGGATACCGTGCAATT |
| 3073 | dre-miR-365 | ATAAGGATTTTTAGGGGCATTAATAAGGATTTTTAGGGGCATTA |
| 3074 | dre-miR-375 | TAACGCGAGCCGAACGAACAATAACGCGAGCCGAACGAACAA |
| 3075 | dre-miR-454a | CCCTATTAGCAATATTGCACTACCCTATTAGCAATATTGCACTA |
| 3076 | dre-miR-454b | CCCTATAAGCAATATTGCACTACCCTATAAGCAATATTGCACTA |
| 3077 | dre-miR-455 | CGATGTAGTCCAAGGGCACATCGATGTAGTCCAAGGGCACAT |
| 3078 | dre-miR-430i | CTACGCCAACAAATAGCACTTACTACGCCAACAAATAGCACTTA |
| 3079 | dre-miR-430j | TACCCCAATTTGATAGCACTTTTACCCCAATTTGATAGCACTTT |
| 3080 | dre-miR-456 | TGACAACCATCTAACCAGCCTTGACAACCATCTAACCAGCCT |
| 3081 | dre-miR-457a | TGCCAATATTGATGTGCTGCTTTGCCAATATTGATGTGCTGCTT |
| 3082 | dre-miR-457b | CTCCAGTATTTATGTGCTGCTTCTCCAGTATTTATGTGCTGCTT |
| 3083 | dre-miR-458 | GCAGTACCATTCAAAGAGCTATGCAGTACCATTCAAAGAGCTAT |
| 3084 | dre-miR-459 | CAGGATGAATCCTTGTTACTGACAGGATGAATCCTTGTTACTGA |
| 3085 | dre-miR-460-5p | CGCACAGTGTGTACAATGCAGCGCACAGTGTGTACAATGCAG |
| 3086 | dre-miR-460-3p | CATCCACATTGTATGCGCTGTCATCCACATTGTATGCGCTGT |
| 3087 | dre-miR-461 | TTGGCATTTAGCCCATTCCTGATTGGCATTTAGCCCATTCCTGA |
| 3088 | PREDICTED_MIR12 | AAACATCACTGCAAGTCTTAACAAACATCACTGCAAGTCTTAAC |
| 3089 | PREDICTED_MIR23 | AGAGGAGAGCCGTGTATGACTAGAGGAGAGCCGTGTATGACT |
| 3090 | PREDICTED_MIR26 | ACAGGCCATCTGTGTTATATTCACAGGCCATCTGTGTTATATTC |
| 3091 | PREDICTED_MIR30 | AGGCCGGGACGAGTGCAATAGGCCGGGACGAGTGCAAT |
| 3092 | PREDICTED_MIR43 | GTACAAACCACAGTGTGCTGCGTACAAACCACAGTGTGCTGC |
| 3093 | PREDICTED_MIR52 | AATGAAAGCCTACCATGTACAAAATGAAAGCCTACCATGTACAA |
| 3094 | PREDICTED_MIR54 | ACCAGCTAACAATACACTGCCAACCAGCTAACAATACACTGCCA |
| 3095 | PREDICTED_MIR56 | AAAATCTCTGCAGGCAAATGTGAAAATCTCTGCAGGCAAATGTG |
| 3096 | PREDICTED_MIR61 | AAGAGGTTTCCCGTGTATGTTTAAGAGGTTTCCCGTGTATGTTT |
| 3097 | PREDICTED_MIR64 | ATGGGACATCCTACATATGCAAATGGGACATCCTACATATGCAA |
| 3098 | PREDICTED_MIR65 | AGAGAACCATTACCATTACTAAAGAGAACCATTACCATTACTAA |
| 3099 | PREDICTED_MIR74 | CCCACCGACAACAATGAATGTTCCCACCGACAACAATGAATGTT |
| 3100 | PREDICTED_MIR78 | GCTCCAGGCAGCCCAAAGCTCCAGGCAGCCCAAA |
| 3101 | PREDICTED_MIR88 | CCCACGCACCAGGGTAACCCACGCACCAGGGTAA |
| 3102 | PREDICTED_MIR89 | ATGTTCAAATAAGCTTTTGTAAATGTTCAAATAAGCTTTTGTAA |
| 3103 | PREDICTED_MIR90 | TTTTTTTTCAACTTGTTACAGCTTTTTTTTCAACTTGTTACAGC |
| 3104 | PREDICTED_MIR92 | AAACAAAGCACCTCTCCAAAAAAAACAAAGCACCTCTCCAAAAA |
| 3105 | PREDICTED_MIR93 | GCTAACAAGGAATGCTGCCAAAGCTAACAAGGAATGCTGCCAAA |
| 3106 | PREDICTED_MIR100 | GAGAAATTTTCAGGGCTACTGAGAGAAATTTTCAGGGCTACTGA |
| 3107 | PREDICTED_MIR102 | TGAATCCTTGCCCAGGTGCATTGAATCCTTGCCCAGGTGCAT |
| 3108 | PREDICTED_MIR103 | GAGCTGAGTGGAGCACAAACAGAGCTGAGTGGAGCACAAACA |
| 3109 | PREDICTED_MIR104 | TTGTTCAACCAGTTACTAATCTTTGTTCAACCAGTTACTAATCT |
| 3110 | PREDICTED_MIR105 | AGCTGCCGGCATTAAAGGGCTAAGCTGCCGGCATTAAAGGGCTA |
| 3111 | PREDICTED_MIR108 | CCAAATTAGCTTTTTAAATAGACCAAATTAGCTTTTTAAATAGA |
| 3112 | PREDICTED_MIR109 | AACCCAATATCAAACATATCACAACCCAATATCAAACATATCAC |
| 3113 | PREDICTED_MIR110 | CCAAGAAATAGCCTTTCAAACACCAAGAAATAGCCTTTCAAACA |
| 3114 | PREDICTED_MIR112 | ACCCCGTGCCACTGTGTACCCCGTGCCACTGTGT |
| 3115 | PREDICTED_MIR113 | CATGTCATAAGCCATTTATTTCCATGTCATAAGCCATTTATTTC |
| 3116 | PREDICTED_MIR114 | TTGGGAGACCCTGGTCTGCACTTTGGGAGACCCTGGTCTGCACT |
| 3117 | PREDICTED_MIR119 | CTAATGACCGCAGAAAGCCATTCTAATGACCGCAGAAAGCCATT |
| 3118 | PREDICTED_MIR120 | CATTCAACAAACATTTAATGAGCATTCAACAAACATTTAATGAG |
| 3119 | PREDICTED_MIR121 | AGCCTATGGAATTCAGTTCTCAAGCCTATGGAATTCAGTTCTCA |
| 3120 | PREDICTED_MIR124 | AAGAAGTGCACCATGTTTGTTTAAGAAGTGCACCATGTTTGTTT |
| 3121 | PREDICTED_MIR127 | TGCCTGGCACCTACACACTAATGCCTGGCACCTACACACTAA |
| 3122 | PREDICTED_MIR128 | TGCTAAATGATCCCCTGGTGCTGCTAAATGATCCCCTGGTGC |
| 3123 | PREDICTED_MIR129 | CCAATTAAGTCTTTTAAATAAACCAATTAAGTCTTTTAAATAAA |
| 3124 | PREDICTED_MIR131 | CACTTCACTGCCTGCAGACAACACTTCACTGCCTGCAGACAA |
| 3125 | PREDICTED_MIR132 | CGTTCCTGATAAGTGAATAAAACGTTCCTGATAAGTGAATAAAA |
| 3126 | PREDICTED_MIR135 | GCAGTTCAGAAAATTAAATAGAGCAGTTCAGAAAATTAAATAGA |
| 3127 | PREDICTED_MIR137 | GTTCTCCAATACCTAGGCACAAGTTCTCCAATACCTAGGCACAA |
| 3128 | PREDICTED_MIR138 | TATTAGGAACACATCGCAAAAATATTAGGAACACATCGCAAAAA |
| 3129 | PREDICTED_MIR139 | TAGGGTCACACAGGATGTGAATTAGGGTCACACAGGATGTGAAT |
| 3130 | PREDICTED_MIR140 | ACAAGGATGAATCTTTGTTACTACAAGGATGAATCTTTGTTACT |
| 3131 | PREDICTED_MIR141 | CAGAACTGTTCCCGCTGCTACAGAACTGTTCCCGCTGCTA |
| 3132 | PREDICTED_MIR142 | AGGTTACCCGAGCAACTTTGCAGGTTACCCGAGCAACTTTGC |
| 3133 | PREDICTED_MIR143 | GAGGGGAGTTTTCTTTCAAAAGGAGGGGAGTTTTCTTTCAAAAG |
| 3134 | PREDICTED_MIR144 | ATCCTTGAATAGGTGTGTTGCAATCCTTGAATAGGTGTGTTGCA |
| 3135 | PREDICTED_MIR145 | TTTACAGGGTGGCCCATTTAAATTTACAGGGTGGCCCATTTAAA |
| 3136 | PREDICTED_MIR146 | CAAAGAGCATGATATTTGACAGCAAAGAGCATGATATTTGACAG |
| 3137 | PREDICTED_MIR149 | GGTCAATATTTACCTCTCAGGTGGTCAATATTTACCTCTCAGGT |
| 3138 | PREDICTED_MIR150 | TCAGGCCATCAGCAGCTGCTA1TCAGGCCATCAGCAGCTGCTAT |
| 3139 | PREDICTED_MIR151 | CCAGGAATTGATGACCAGCTGCCAGGAATTGATGACCAGCTG |
| 3140 | PREDICTED_MIR152 | AGGACCCAGAGAACAACTCAGAGGACCCAGAGAACAACTCAG |
| 3141 | PREDICTED_MIR153 | ACCTAGGGATCGTCAAAGGGAACCTAGGGATCGTCAAAGGGA |
| 3142 | PREDICTED_MIR154 | TTTCCTCTGCAAACAGTTGTAATTTCCTCTGCAAACAGTTGTAA |
| 3143 | PREDICTED_MIR155 | TTTAGTCAATATCAAGATTTATTTTAGTCAATATCAAGATTTAT |
| 3144 | PREDICTED_MIR156 | AAGCTTCCCGGGCAGCTAAGCTTCCCGGGCAGCT |
| 3145 | PREDICTED_MIR157 | TGCCCATGGACTGCATGGTGCTTGCCCATGGACTGCATGGTGCT |
| 3146 | PREDICTED_MIR158 | GCTGATTGCCTCTGTGCCAATGCTGATTGCCTCTGTGCCAAT |
| 3147 | PREDICTED_MIR160 | AACGCCGGGGCCACGTTGCTAAAACGCCGGGGCCACGTTGCTAA |
| 3148 | PREDICTED_MIR161 | CGAAAGGAGATTGGCCATGTAACGAAAGGAGATTGGCCATGTAA |
| 3149 | PREDICTED_MIR162 | TTCCTACTGAAATCTGACAATCTTCCTACTGAAATCTGACAATC |
| 3150 | PREDICTED_MIR163 | GAAAGACCCCATTTAACTTGAAGAAAGACCCCATTTAACTTGAA |
| 3151 | PREDICTED_MIR164 | TGAACAATCCAGATAATTGCTTTGAACAATCCAGATAATTGCTT |
| 3152 | PREDICTED_MIR165 | TCCCCTGCAAGTGGTGCTTCCCCTGCAAGTGGTGCT |
| 3153 | PREDICTED_MIR166 | TCCCACACCCAAGGCTTGCATCCCACACCCAAGGCTTGCA |
| 3154 | PREDICTED_MIR167 | GAAACCAAGTATGGGTCGCCTGAAACCAAGTATGGGTCGCCT |
| 3155 | PREDICTED_MIR168 | TGTGTGCAATTACCCATTTTATTGTGTGCAATTACCCATTTTAT |
| 3156 | PREDICTED_MIR170 | ATTTAAAAGGCTTTTAAATGATATTTAAAAGGCTTTTAAATGAT |
| 3157 | PREDICTED_MIR171 | ATAGTAGACCGTATAGCGTACGATAGTAGACCGTATAGCGTACG |
| 3158 | PREDICTED_MIR172 | ACTGGGGCTGCATGCTGCTCAACTGGGGCTGCATGCTGCTCA |
| 3159 | PREDICTED_MIR173 | CTACTGTTAATGACCTATTTCTCTACTGTTAATGACCTATTTCT |
| 3160 | PREDICTED_MIR174 | CCTAAATACCTGGTATTTGAGACCTAAATACCTGGTATTTGAGA |
| 3161 | PREDICTED_MIR176 | CTTTGACAGCATTTTAATTATACTTTGACAGCATTTTAATTATA |
| 3162 | PREDICTED_MIR177 | GAACACACCAAGGATAATTTCTGAACACACCAAGGATAATTTCT |
| 3163 | PREDICTED_MIR179 | AGTTATGAAATGTCATCAATAAAGTTATGAAATGTCATCAATAA |
| 3164 | PREDICTED_MIR180 | CACAGGAAGTGGCCTTCAATACACAGGAAGTGGCCTTCAATA |
| 3165 | PREDICTED_MIR181 | ATTGTTTGCACTCTGCCAGTTTATTGTTTGCACTCTGCCAGTTT |
| 3166 | PREDICTED_MIR182 | GAGCTGAACTCAAAACCAAATGGAGCTGAACTCAAAACCAAATG |
| 3167 | PREDICTED_MIR183 | TCTTTATTGCAAAGTCAGTATGTCTTTATTGCAAAGTCAGTATG |
| 3168 | PREDICTED_MIR184 | AACCCTAGGAGAGGGTGCCATTAACCCTAGGAGAGGGTGCCATT |
| 3169 | PREDICTED_MIR186 | ATTCTGCCCCTGGATATGCATATTCTGCCCCTGGATATGCAT |
| 3170 | PREDICTED_MIR187 | AACCAAGCAGCCGGGCAGTAACCAAGCAGCCGGGCAGT |
| 3171 | PREDICTED_MIR189 | AGCAGGGCTCCCTCACCAGCAAGCAGGGCTCCCTCACCAGCA |
| 3172 | PREDICTED_MIR190 | ATAAGGATTTTTAGGGGCATTAATAAGGATTTTTAGGGGCATTA |
| 3173 | PREDICTED_MIR191 | CGCCGCCCCGCACCTGCTGCCGCCGCCCCGCACCTGCTGC |
| 3174 | PREDICTED_MIR192 | ACATCTCGGGGATCATCATGTACATCTCGGGGATCATCATGT |
| 3175 | PREDICTED_MIR194 | GGGCCCTATATTAATGGACCAAGGGCCCTATATTAATGGACCAA |
| 3176 | PREDICTED_MIR196 | AGTAAAGCCAAGTAGTGCATGAAGTAAAGCCAAGTAGTGCATGA |
| 3177 | PREDICTED_MIR197 | AAGAAGGACCTTGTAATAAATAAAGAAGGACCTTGTAATAAATA |
| 3178 | PREDICTED_MIR198 | CCAGATGCTAAGCACTGGAAGCCAGATGCTAAGCACTGGAAG |
| 3179 | PREDICTED_MIR199 | TAACCACTCTCCAAGTACCAAATAACCACTCTCCAAGTACCAAA |
| 3180 | PREDICTED_MIR200 | TTAACAGGCAGTTCTGCTGCTATTAACAGGCAGTTCTGCTGCTA |
| 3181 | PREDICTED_MIR201 | ACGGTTTTACCAGACAGTATTAACGGTTTTACCAGACAGTATTA |
| 3182 | PREDICTED_MIR202 | AGAAGTGCACCGCGAATGTTTAGAAGTGCACCGCGAATGTTT |
| 3183 | PREDICTED_MIR203 | TTAAGAGCCCGGCTTTGCCTTTAAGAGCCCGGCTTTGCCT |
| 3184 | PREDICTED_MIR205 | ATCCACGTTTTAAATACCAAAGATCCACGTTTTAAATACCAAAG |
| 3185 | PREDICTED_MIR206 | TGCCTCCCACACACAGCTTTATGCCTCCCACACACAGCTTTA |
| 3186 | PREDICTED_MIR207 | TTCCCCGGCACCAGCACAAAGTTTCCCCGGCACCAGCACAAAGT |
| 3187 | PREDICTED_MIR208 | CAATCAGAGGCAATCAAGCACACAATCAGAGGCAATCAAGCACA |
| 3188 | PREDICTED_MIR209 | TAATTCTAAAGACAAAGCACAATAATTCTAAAGACAAAGCACAA |
| 3189 | PREDICTED_MIR210 | GGTTGTCAGGAACAGAAGTGCGGTTGTCAGGAACAGAAGTGC |
| 3190 | PREDICTED_MIR211 | TACAGATGGATACCGTGCAATTTACAGATGGATACCGTGCAATT |
| 3191 | PREDICTED_MIR212 | ACTTGATCAAACAGAGCACAACACTTGATCAAACAGAGCACAAC |
| 3192 | PREDICTED_MIR213 | TTTTCTCCTGACTGATTGCACTTTTTCTCCTGACTGATTGCACT |
| 3193 | PREDICTED_MIR214 | TTAAAATGACATGGATAATGCATTAAAATGACATGGATAATGCA |
| 3194 | PREDICTED_MIR215 | AGAAGCGCCTTTGGCAGCTAAGAAGCGCCTTTGGCAGCTA |
| 3195 | PREDICTED_MIR216 | TACCTGCACTATGAGCACTTTGTACCTGCACTATGAGCACTTTG |
| 3196 | PREDICTED_MIR218 | GTCATGATCATCCCACACTAATGTCATGATCATCCCACACTAAT |
| 3197 | PREDICTED_MIR219 | TGGCACCTATGCCCACCAGCATGGCACCTATGCCCACCAGCA |
| 3198 | PREDICTED_MIR220 | GCTTTGACAATATCATTGCACTGCTTTGACAATATCATTGCACT |
| 3199 | PREDICTED_MIR222 | GTCGGCATCTACACTTGCACTGTCGGCATCTACACTTGCACT |
| 3200 | PREDICTED_MIR223 | ACCTGCTGCCACTGGCACTTAACCTGCTGCCACTGGCACTTA |
| 3201 | PREDICTED_MIR224 | GGCATGAATTTATTGTGCAATAGGCATGAATTTATTGTGCAATA |
| 3202 | PREDICTED_MIR225 | GCTGGCAGGGAAGTAGTGGCTGGCAGGGAAGTAGTG |
| 3203 | PREDICTED_MIR226 | ATAACACCTACGAGCACTGCCATAACACCTACGAGCACTGCC |
| 3204 | PREDICTED_MIR227 | AGTCACAGCATCCATTAATAAAAGTCACAGCATCCATTAATAAA |
| 3205 | PREDICTED_MIR228 | ATGAGAAGACTGTCACAATCAAATGAGAAGACTGTCACAATCAA |
| 3206 | PREDICTED_MIR229 | CTGCCAAACCAATTAATACCTCCTGCCAAACCAATTAATACCTC |
| 3207 | PREDICTED_MIR230 | TCATATTTTAGTTCTGCACTGATCATATTTTAGTTCTGCACTGA |
| 3208 | PREDICTED_MIR231 | CACATAACAGGTGCTCAAATAACACATAACAGGTGCTCAAATAA |
| 3209 | PREDICTED_MIR232 | TAGAGATTGTTTCAACACTGAATAGAGATTGTTTCAACACTGAA |
| 3210 | PREDICTED_MIR234 | GTCTCCACAGAAACTTTTGTCCGTCTCCACAGAAACTTTTGTCC |
| 3211 | PREDICTED_MIR235 | ACCCGGTCTGCCAGAAGCTGCTACCCGGTCTGCCAGAAGCTGCT |
| 3212 | PREDICTED_MIR236 | TTCAATAGGGCATAGGTGCCAATTCAATAGGGCATAGGTGCCAA |
| 3213 | PREDICTED_MIR237 | CTCCAAAGAACATTACTGTGATCTCCAAAGAACATTACTGTGAT |
| 3214 | PREDICTED_MIR238 | TATTAGGAACACATCGCAAAAATATTAGGAACACATCGCAAAAA |
| 3215 | PREDICTED_MIR239 | ATCAATGCTATGTGATCTGCATATCAATGCTATGTGATCTGCAT |
| 3216 | PREDICTED_MIR240 | TCACCCCAAAGTTGTGGCAATATCACCCCAAAGTTGTGGCAATA |
| 3217 | PREDICTED_MIR241 | ATGTGACAGAGCCAAGCACAAAATGTGACAGAGCCAAGCACAAA |
| 3218 | PREDICTED_MIR242 | ACCTACACTGAAACTGCCAAAAACCTACACTGAAACTGCCAAAA |
| 3219 | PREDICTED_MIR243 | TTACCAAGGGCGACTCGCATTTACCAAGGGCGACTCGCAT |
| 3220 | PREDICTED_MIR245 | ATAAGGATTTTTAGGGGCATTAATAAGGATTTTTAGGGGCATTA |
| 3221 | PREDICTED_MIR246 | CCCGTATGTAATAAATGTGCTACCCGTATGTAATAAATGTGCTA |
| 3222 | PREDICTED_MIR247 | TTAAGTTTTGAAAAGTACATAGTTAAGTTTTGAAAAGTACATAG |
| 3223 | PREDICTED_MIR249 | AAAGCATACCAGCTGAACCAAAAAAGCATACCAGCTGAACCAAA |
| 3224 | PREDICTED_MIR250 | CACAAGTTCCTGCAAATGCACACACAAGTTCCTGCAAATGCACA |
| 3225 | PREDICTED_MIR252 | AAAAGAGACCTTCATATGCAAAAAAAGAGACCTTCATATGCAAA |
| 3226 | PREDICTED_MIR253 | TAACTGCACTAGATGCACCTTATAACTGCACTAGATGCACCTTA |
| 3227 | PREDICTED_MIR254 | AAGCATATTTCTCCCACTGTGAAAGCATATTTCTCCCACTGTGA |
| 3228 | PREDICTED_MIR255 | TCCTGATGGTCGAAGTGCCAATCCTGATGGTCGAAGTGCCAA |
| 3229 | PREDICTED_MIR256 | CATAATTACAGAAAATTGCACTCATAATTACAGAAAATTGCACT |
| 3230 | PREDICTED_MIR257 | ACACTTAGCAGGTTGTATTATAACACTTAGCAGGTTGTATTATA |
| 3231 | PREDICTED_MIR258 | TCACCCGAGGCGCACTTATCACCCGAGGCGCACTTA |

### REFERENCES

1. Ambros V. The functions of animal microRNAs. Nature 2004; 431(7006):350-5.
2. Ambros V, Lee R C, Lavanway A, Williams P T, Jewell D. MicroRNAs and other tiny endogenous RNAs in C. elegans. Curr Biol 2003; 13(10):807-18.
3. Bartel D P. MicroRNAs: genomics, biogenesis, mechanism, and function. Cell 2004; 116(2):281-97.
4. Nakahara K, Carthew R W. Expanding roles for miRNAs and siRNAs in cell regulation. Curr Opin Cell Biol 2004; 16(2):127-33.
5. Yang M, Li Y, Padgett R W. MicroRNAs: Small regulators with a big impact. Cytokine Growth Factor Rev 2005.
6. Stark A, Brennecke J, Russell R B, Cohen S M. Identification of Drosophila MicroRNA Targets. PLoS Biol 2003; 1(3):E60.
7. Lewis B P, Shih I H, Jones-Rhoades M W, Bartel D P, Burge C B. Prediction of mammalian microRNA targets. Cell 2003; 115(7):787-98.
8. John B, Enright A J, Aravin A, Tuschl T, Sander C, Marks D S. Human MicroRNA Targets. PLoS Biol 2004; 2(11):e363.
9. Robins H, Li Y, Padgett R W. Incorporating structure to predict microRNA targets. Proc Natl Acad Sci USA 2005; 102(11):4006-9.
10. Chen C Z, Li L, Lodish H F, Bartel D P. MicroRNAs modulate hematopoietic lineage differentiation. Science 2004; 303(5654):83-6.
11. Esau C, Kang X, Peralta E, Hanson E, Marcusson E G, Ravichandran L V, Sun Y, Koo S, Perera R J, Jain R, Dean N M, Freier S M, Bennett C F, Lollo B, Griffey R. MicroRNA-143 regulates adipocyte differentiation. J Biol Chem 2004.
12. Poy M N, Eliasson L, Krutzfeldt J, Kuwajima S, Ma X, Macdonald P E, Pfeffer S, Tuschl T, Rajewsky N, Rorsman P, Stoffel M. A pancreatic islet-specific microRNA regulates insulin secretion. Nature 2004; 432(7014):226-30.
13. Lee Y S, Kim H K, Chung S, Kim K S, Dutta A. Depletion of human microRNA miR-125b reveals that it is critical for the proliferation of differentiated cells but not for the down-regulation of putative targets during differentiation. J Biol Chem 2005.
14. Calin G A, Sevignani C, Dumitru C D, Hyslop T, Noch E, Yendamuri S, Shimizu M, Rattan S, Bullrich F, Negrini M, Croce C M. Human microRNA genes are frequently located at fragile sites and genomic regions involved in cancers. Proc Natl Acad Sci USA 2004; 101(9):2999-3004.
15. Calin G A, Dumitru C D, Shimizu M, Bichi R, Zupo S, Noch E, Aldler H, Rattan S, Keating M, Rai K, Rassenti L, Kipps T, Negrini M, Bullrich F, Croce C M. Frequent deletions and down-regulation of micro-RNA genes miR15 and miR16 at 13q14 in chronic lymphocytic leukemia. Proc Natl Acad Sci USA 2002; 99(24):15524-9.
16. Calin G A, Liu C G, Sevignani C, Ferracin M, Felli N, Dumitru C D, Shimizu M, Cimmino A, Zupo S, Dono M, Dell'Aquila M L, Alder H, Rassenti L, Kipps T J, Bullrich F, Negrini M, Croce C M. MicroRNA profiling reveals distinct signatures in B cell chronic lymphocytic leukemias. Proc Natl Acad Sci USA 2004.
17. Michael M Z, SM O C, van Holst Pellekaan N G, Young G P, James R J. Reduced accumulation of specific microRNAs in colorectal neoplasia. Mol Cancer Res 2003; 1(12):882-91.
18. Krichevsky A M, King K S, Donahue C P, Khrapko K, Kosik K S. A microRNA array reveals extensive regulation of microRNAs during brain development. Rna 2003; 9(10):1274-81.
19. Babak T, Zhang W, Morris Q, Blencowe B J, Hughes T R. Probing microRNAs with microarrays: Tissue specificity and functional inference. Rna 2004; 10(11):1813-9.
20. Barad O, Meiri E, Avniel A, Aharonov R, Barzilai A, Bentwich I, Einav U, Gilad S, Hurban P, Karov Y, Lobenhofer E K, Sharon E, Shiboleth Y M, Shtutman M, Bentwich Z, Einat P. MicroRNA expression detected by oligonucleotide microarrays: system establishment and expression profiling in human tissues. Genome Res 2004; 14(12):2486-94.
21. Liu C G, Calin G A, Meloon B, Gamliel N, Sevignani C, Ferracin M, Dumitru C D, Shimizu M, Zupo S, Dono M, Alder H, Bullrich F, Negrini M, Croce C M. An oligonucleotide microchip for genome-wide microRNA profiling in human and mouse tissues. Proc Natl Acad Sci USA 2004; 101(26):9740-4.
22. Miska E A, Alvarez-Saavedra E, Townsend M, Yoshii A, Sestan N, Rakic P, Constantine-Paton M, Horvitz H R. Microarray analysis of microRNA expression in the developing mammalian brain. Genome Biol 2004; 5(9):R68.
23. Sun Y, Koo S, White N, Peralta E, Esau C, Dean N M, Perera R J. Development of a micro-array to detect human and mouse microRNAs and characterization of expression in human organs. Nucleic Acids Res 2004; 32(22):e188.
24. Baskerville S, Bartel D P. Microarray profiling of microRNAs reveals frequent coexpression with neighboring miRNAs and host genes. Rna 2005; 11(3):241-7.
25. Liang R Q, Li W, Li Y, Tan C Y, Li J X, Jin Y X, Ruan K C. An oligonucleotide microarray for microRNA expression analysis based on labeling RNA with quantum dot and nanogold probe. Nucleic Acids Res 2005; 33(2):e17.
26. Lim L P, Lau N C, Garrett-Engele P, Grimson A, Schelter J M, Castle J, Bartel D P, Linsley P S, Johnson J M. Microarray analysis shows that some microRNAs downregulate large numbers of target mRNAs. Nature 2005.
27. Stears R L, Getts R C, Gullans S R. A novel, sensitive detection system for high-density microarrays using dendrimer technology. Physiol Genomics 2000; 3(2):93-9.
28. Altschul S F, Gish W, Miller W, Myers E W, Lipman D J. Basic local alignment search tool. J Mol Biol 1990; 215(3):403-10.
29. Behlke M A, Dames S A, McDonald W H, Gould K L, Devor E J, Walder J A. Use of high specific activity StarFire oligonucleotide probes to visualize low-abundance pre-mRNA splicing intermediates in S. pombe. Biotechniques 2000; 29(4):892-7.
30. Hasegawa K, Chang Y W, Li H, Berlin Y, Ikeda O, Kane-Goldsmith N, Grumet M. Embryonic radial glia bridge spinal cord lesions and promote functional recovery following spinal cord injury. Exp Neurol 2005; 193(2):394-410.
31. Li H, Babiarz J, Woodbury J, Kane-Goldsmith N, Grumet M. Spatiotemporal heterogeneity of CNS radial glial cells and their transition to restricted precursors. Dev Biol 2004; 271(2):225-38.
32. Brennecke J, Stark A, Russell R B, Cohen S M. Principles of MicroRNA-Target Recognition. PLoS Biol 2005; 3(3):e85.
33. Saxena S, Jonsson Z O, Dutta A. Small RNAs with imperfect match to endogenous mRNA repress translation. Implications for off-target activity of small inhibitory RNA in mammalian cells. J Biol Chem 2003;

278(45):44312-9.
34. Lewis B P, Burge C B, Bartel D P. Conserved seed pairing, often flanked by adenosines, indicates that thousands of human genes are microRNA targets. Cell 2005; 120(1):15-20.
35. Griffiths-Jones S. The microRNA Registry. Nucleic Acids Res 2004; 32 Database issue:D 109-11.
36. Thomson J M, Parker J, Perou C M, Hammond S M. A custom microarray platform for analysis of microRNA gene expression. 2004; 1(1):47.
37. Yang Y H, Dudoit S, Luu P, Lin D M, Peng V, Ngai J, Speed T P. Normalization for cDNA microarray data: a robust composite method addressing single and multiple slide systematic variation. Nucleic Acids Res. 2002; 30(4):e15.
38. Yang Y H, Speed T. Design issues for cDNA microarray experiments. Nat. Rev. Genet. 2002; 3(8):579.
39. Lagos-Quintana M, Rauhut R, Yalcin A, Meyer J, Lendeckel W, Tuschl T. Identification of tissue-specific microRNAs from mouse. Curr Biol 2002; 12(9):735-9.
40. Sempere L F, Freemantle S, Pitha-Rowe I, Moss E, Dmitrovsky E, Ambros V. Expression profiling of mammalian microRNAs uncovers a subset of brain-expressed microRNAs with possible roles in murine and human neuronal differentiation. Genome Biol 2004; 5(3):R13.
41. Lagos-Quintana M, Rauhut R, Meyer J, Borkhardt A, Tuschl T. New microRNAs from mouse and human. Rna 2003; 9(2):175-9.
42. Houbaviy H B, Murray M F, Sharp P A. Embryonic stem cell-specific MicroRNAs. Dev Cell 2003; 5(2):351-8.
43. Suh M R, Lee Y, Kim J Y, Kim S K, Moon S H, Lee J Y, Cha K Y, Chung H M, Yoon H S, Moon S Y, Kim V N, Kim K S. Human embryonic stem cells express a unique set of microRNAs. Dev Biol 2004; 270(2):488-98.
44. Li H, Babiarz J, Woodbury J, Kane-Goldsmith N, Grumet M. Spatiotemporal heterogeneity of CNS radial glial cells and their transition to restricted precursors. Developmental Biology 2004.
45. Goff, L. A., Yang, M, Bowers, J, Getts, R. C., Padgett, R and Hart, R P. Rational Probe Optimization and Enhanced Detection Strategy for M icroRNAs using MicroArrays. RNA Biology 2:3 e9-e16 2005.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A computer assisted method for optimizing design of probes which selectively hybridize to target miRNAs obtained from a database using a programmed computer, including a processor, an input device and an output device comprising:
a) inputting into the programmed computer miRNA sequence data,
b) inputting upper and lower ranges of sequence length;
c) inputting upper and lower ranges of Tm;
d) determining using the processor those probes which satisfy the inputted Tm parameters and sequence length following truncation of the sequences at either the 3′ or 5′ end of said sequence; and
e) outputting those probes that satisfy the inputted Tm parameters.

2. A computer program for implementing the method of claim 1.

3. The method of claim 1, wherein said sequences are truncated at the 5′ end only.

4. The method of claim 1, wherein said sequence are truncated at the 3′ end only.

5. A computer-readable medium having recorded thereon a program that identifies a miRNA probe which specifically hybridizes to the target miRNA according to the method of claim 1.

6. A computational analysis system comprising a computer-readable medium according to claim 5.

7. A kit for identifying a sequence of a nucleic acid that is suitable for use as a immobilized probe for a target miRNA, said kit comprising: (a) an algorithm that identifies a sequence of a nucleic acid that is suitable for use as a probe according to the method according to claim 1, wherein said algorithm is present on a computer readable medium; and (b) instructions for using said algorithm to identify said sequence of a nucleic acid that is suitable for use as a probe for said miRNA target nucleic acid.

8. A method for rational probe optimization for detection of Mi RNA molecules comprising:
a) providing a database of known miRNA sequences;
b) performing the miRMAX algorithm on said sequences to identify probes having enhanced sequence specificity, substantially similar hybridization temperatures and sequence length; and
c) obtaining the probe sequences identified in step b) and optionally synthesizing the same.

9. The method of claim 8, comprising generating the reverse complement of the sequences of step c) and
d) preparing concatamers of said probe sequences.

10. The method of claim 9, wherein said concatamer is selected from the group consisting of a dimer, a trimer or a multimer.

11. The method of claim 8, wherein said probe sequences are affixed to a solid support.

12. The method of claim 11, wherein said solid support is selected from the group consisting of a glass slide, a magnetic bead, a glass bead, a latex bead, a luminex bead, a filter, a multiwell plate and a microarray.

13. The method of claim 8, wherein said miRNA molecules are mature miRNAs.

14. An oligonucleotide array comprising an array of multiple oligonucleotides with different base sequences fixed onto known and separate positions on a support substrate, said oligonucleotides being synthesized using the outputted sequences of claim 1, wherein said oligonucleotides specifically hybridize to miRNA sequences or the complement thereof, and the said oligonucleotides are classified according to their sequence of origin, wherein the fixation region on the support substrate is divided into the said classification.

15. The array of claim 14, wherein said sequences are further classified according to biological organism of origin.

16. The array of claim 14, wherein said sequences are further classified according to the function of the target gene modulated by said miRNA.

17. The array of claim 14, wherein said sequences are further classified according to their tissue of origin.

18. The array of claim 14, comprising at least one probe from Tables 1 or 2.

19. The method of claim 9, wherein said probe sequences are affixed to a solid support.


SEQUENCE LISTING (1352 sequences)

SEQ ID NO: 1 - DNA, 22 bp, Artificial Sequence
  Synthetic Sequence
ctacctgcacgaacagcacttt

SEQ ID NO: 2 - DNA, 22 bp, Artificial Sequence
  Synthetic Sequence
cgccaatatttacgtgctgcta

SEQ ID NO: 3 - DNA, 22 bp, Artificial Sequence
  Synthetic Sequence
agctgcttttgggattccgttg

SEQ ID NO: 4 - DNA, 22 bp, Artificial Sequence
  Synthetic Sequence
caacggaaucccaaaagcagcu

SEQ ID NO: 5 - DNA, 22 bp, Artificial Sequence
  Synthetic Sequence
caacgcaaucccaaaagcagcu

SEQ ID NO: 6 - DNA, 22 bp, Artificial Sequence
  Synthetic Sequence
caacgcaaucccaaaagaagcu

SEQ ID NO: 7 - DNA, 46 bp, Artificial Sequence
  Synthetic Sequence
tcatacagctagataaccaaagatcatacagctagataaccaaaga

SEQ ID NO: 8 - DNA, 44 bp, Artificial Sequence
  Synthetic Sequence
tcatacagctagataaccaaagtcatacagctagataaccaaag

SEQ ID NO: 9 - DNA, 42 bp, Artificial Sequence
  Synthetic Sequence
catacagctagataaccaaagcatacagctagataaccaaag

SEQ ID NO: 10 - DNA, 40 bp, Artificial Sequence
  Synthetic Sequence
tcatacagctagataaccaatcatacagctagataaccaa

SEQ ID NO: 11 - DNA, 40 bp, Artificial Sequence
  Synthetic Sequence
catacagctagataaccaaacatacagctagataaccaaa

SEQ ID NO: 12 - DNA, 38 bp, Artificial Sequence
  Synthetic Sequence
tcatacagctagataaccatcatacagctagataacca

SEQ ID NO: 13 - DNA, 36 bp, Artificial Sequence
  Synthetic Sequence
tcatacagctagataacctcatacagctagataacc

SEQ ID NO: 14 - DNA, 42 bp, Artificial Sequence
  Synthetic Sequence
tcatacagctagataaccaaatcatacagctagataaccaaa

SEQ ID NO: 15 - DNA, 69 bp, Artificial Sequence
  Synthetic Sequence
tcatacagctagataaccaaagatcatacagctagataaccaaagatcatacagctagataaccaaaga

SEQ ID NO: 16 - DNA, 42 bp, Artificial Sequence
  Synthetic Sequence
tcatagccctgtacaatgctgtcatagccctgtacaatgctg

SEQ ID NO: 17 - DNA, 40 bp, Artificial Sequence
  Synthetic Sequence
tcatagccctgtacaatgcttcatagccctgtacaatgct

SEQ ID NO: 18 - DNA, 40 bp, Artificial Sequence
  Synthetic Sequence
catagccctgtacaatgctgcatagccctgtacaatgctg

SEQ ID NO: 19 - DNA, 38 bp, Artificial Sequence
  Synthetic Sequence
catagccctgtacaatgctcatagccctgtacaatgct

SEQ ID NO: 20 - DNA, 38 bp, Artificial Sequence
  Synthetic Sequence
tcatagccctgtacaatgctcatagccctgtacaatgc

SEQ ID NO: 21 - DNA, 38 bp, Artificial Sequence
  Synthetic Sequence
atagccctgtacaatgctgatagccctgtacaatgctg

SEQ ID NO: 22 - DNA, 36 bp, Artificial Sequence
  Synthetic Sequence
atagccctgtacaatgctatagccctgtacaatgct

SEQ ID NO: 23 - DNA, 36 bp, Artificial Sequence
  Synthetic Sequence
tcatagccctgtacaatgtcatagccctgtacaatg

SEQ ID NO: 24 - DNA, 36 bp, Artificial Sequence
  Synthetic Sequence
tagccctgtacaatgctgtagccctgtacaatgctg

SEQ ID NO: 25 - DNA, 34 bp, Artificial Sequence
  Synthetic Sequence
tcatagccctgtacaattcatagccctgtacaat

SEQ ID NO: 26 - DNA, 44 bp, Artificial Sequence
  Synthetic Sequence
tgtaaaccatgatgttctgctatgtaaaccatgatgttctgcta

SEQ ID NO: 27 - DNA, 44 bp, Artificial Sequence
  Synthetic Sequence
tgtaaagcatgatgttctgctatgtaaagcatgatgttctgcta

SEQ ID NO: 28 - DNA, 44 bp, Artificial Sequence
  Synthetic Sequence
tagcagcacatcatggtttacatagcagcacatcatggtttaca

SEQ ID NO: 29 - DNA, 44 bp, Artificial Sequence
  Synthetic Sequence
tcatatattcggcgatagagcttcatatattcggcgatagagct

SEQ ID NO: 30 - DNA, 44 bp, Artificial Sequence
  Synthetic Sequence
cgccaatatttacgtgctggtacgccaatatttacgtgctggta

SEQ ID NO: 31 - DNA, 44 bp, Artificial Sequence
  Synthetic Sequence
cgccaatatttaggtgctggtacgccaatatttaggtgctggta

SEQ ID NO: 32 - DNA, 44 bp, Artificial Sequence
  Synthetic Sequence
tagcagcacgtaaatattggcgtagcagcacgtaaatattggcg

SEQ ID NO: 33 - DNA, 44 bp, Artificial Sequence
  Synthetic Sequence
cccagcatttatccgtggtatacccagcatttatccgtggtata

SEQ ID NO: 34 - DNA, 44 bp, Artificial Sequence
  Synthetic Sequence
agctcctacccgaaagatgtaaagctcctacccgaaagatgtaa

SEQ ID NO: 35 - DNA, 44 bp, Artificial Sequence
  Synthetic Sequence
agctcctacccgaaagatttaaagctcctacccgaaagatttaa

SEQ ID NO: 36 - DNA, 44 bp, Artificial Sequence
  Synthetic Sequence
ttacatgtttcgggtaggagctttacatgtttcgggtaggagct

SEQ ID NO: 37 - DNA, 44 bp, Artificial Sequence
  Synthetic Sequence
ctaagcaaaatagccgttacccctaagcaaaatagccgttaccc

SEQ ID NO: 38 - DNA, 42 bp, Artificial Sequence
  Synthetic Sequence
ctaccttcacgaacagcacttctaccttcacgaacagcactt

SEQ ID NO: 39 - DNA, 42 bp, Artificial Sequence
  Synthetic Sequence
ctaccttcacgaacagcagttctaccttcacgaacagcagtt

SEQ ID NO: 40 - DNA, 42 bp, Artificial Sequence
  Synthetic Sequence
aagtgctgttcgtgcaggtagaagtgctgttcgtgcaggtag

SEQ ID NO: 41 - DNA, 42 bp, Artificial Sequence
  Synthetic Sequence
aatccctcccgaagtcgctaaaatccctcccgaagtcgctaa

SEQ ID NO: 42 - DNA, 42 bp, Artificial Sequence
  Synthetic Sequence
actggtacaagggttgtgagaactggtacaagggttgtgaga

SEQ ID NO: 43 - DNA, 42 bp, Artificial Sequence
  Synthetic Sequence
actggtagaagggttgtgagaactggtagaagggttgtgaga

SEQ ID NO: 44 - DNA, 42 bp, Artificial Sequence
  Synthetic Sequence
tctcccaacccttgtaccagttctcccaacccttgtaccagt

SEQ ID NO: 45 - DNA, 42 bp, Artificial Sequence
  Synthetic Sequence
agcatggtgtgaacggaaggtagcatggtgtgaacggaaggt

SEQ ID NO: 46 - DNA, 42 bp, Artificial Sequence
  Synthetic Sequence
gcggaacttaggcactgtgaagcggaacttaggcactgtgaa

SEQ ID NO: 47 - DNA, 42 bp, Artificial Sequence
  Synthetic Sequence
gcggaagttaggcactgtgaagcggaagttaggcactgtgaa

SEQ ID NO: 48 - DNA, 42 bp, Artificial Sequence
  Synthetic Sequence
ttcacagtggctaagttccgcttcacagtggctaagttccgc

SEQ ID NO: 49 - DNA, 42 bp, Artificial Sequence
  Synthetic Sequence
tagcgaacgagccactgtagttagcgaacgagccactgtagt

SEQ ID NO: 50 - DNA, 44 bp, Artificial Sequence
  Synthetic Sequence
tccatcattacccggcattatttccatcattacccggcattatt

SEQ ID NO: 51 - DNA, 44 bp, Artificial Sequence
  Synthetic Sequence
tccatcattaccctgcattatttccatcattaccctgcattatt

SEQ ID NO: 52 - DNA, 44 bp, Artificial Sequence
  Synthetic Sequence
aatactgccgggtaatgatggaaatactgccgggtaatgatgga

SEQ ID NO: 53 - DNA, 44 bp, Artificial Sequence
  Synthetic Sequence
ttgccaaccttcctcaggatatttgccaaccttcctcaggatat

SEQ ID NO: 54 - DNA, 42 bp, Artificial Sequence
  Synthetic Sequence
agctgcttttgggattgcgttagctgcttttgggattgcgtt

SEQ ID NO: 55 - DNA, 42 bp, Artificial Sequence
  Synthetic Sequence
agcttcttttgggattgcgttagcttcttttgggattgcgtt

SEQ ID NO: 56 - DNA, 42 bp, Artificial Sequence
  Synthetic Sequence
aacggaatcccaaaagcagctaacggaatcccaaaagcagct

SEQ ID NO: 57 - DNA, 42 bp, Artificial Sequence
  Synthetic Sequence
ctgtctgcggatttggtttcactgtctgcggatttggtttca

SEQ ID NO: 58 - DNA, 44 bp, Artificial Sequence
  Synthetic Sequence
catacgactttgtacaaccaaacatacgactttgtacaaccaaa

SEQ ID NO: 59 - DNA, 44 bp, Artificial Sequence
  Synthetic Sequence
catacgactttgtagaaccaaacatacgactttgtagaaccaaa

SEQ ID NO: 60 - DNA, 44 bp, Artificial Sequence
  Synthetic Sequence
tttggttgtacaaagtggtatgtttggttgtacaaagtggtatg

SEQ ID NO: 61 - DNA, 44 bp, Artificial Sequence
  Synthetic Sequence
taaacccagacattactatcactaaacccagacattactatcac

SEQ ID NO: 62 - DNA, 44 bp, Artificial Sequence
  Synthetic Sequence
acactcaaaacctggcgggactacactcaaaacctggcgggact

SEQ ID NO: 63 - DNA, 44 bp, Artificial Sequence
  Synthetic Sequence
acactcaaaagctggcgggactacactcaaaagctggcgggact

SEQ ID NO: 64 - DNA, 44 bp, Artificial Sequence
  Synthetic Sequence
agtgccgccaggttttgagtgtagtgccgccaggttttgagtgt

SEQ ID NO: 65 - DNA, 44 bp, Artificial Sequence
  Synthetic Sequence
taagaccgacgacgacctctactaagaccgacgacgacctctac

SEQ ID NO: 66 - DNA, 40 bp, Artificial Sequence
  Synthetic Sequence
acacgaatgccctaggggatacacgaatgccctaggggat

SEQ ID NO: 67 - DNA, 40 bp, Artificial Sequence
  Synthetic Sequence
acacgaatgcgctaggggatacacgaatgcgctaggggat

SEQ ID NO: 68 - DNA, 40 bp, Artificial Sequence
  Synthetic Sequence
atcccctagggcattggtgtatcccctagggcattggtgt

SEQ ID NO: 69 - DNA, 40 bp, Artificial Sequence
  Synthetic Sequence
acaactagggtaccgccagtacaactagggtaccgccagt

SEQ ID NO: 70 - DNA, 44 bp, Artificial Sequence
  Synthetic Sequence
cttcagctatcacagtactttacttcagctatcacagtacttta

SEQ ID NO: 71 - DNA, 44 bp, Artificial Sequence
  Synthetic Sequence
cttgagctatcacagtactttacttgagctatcacagtacttta

SEQ ID NO: 72 - DNA, 44 bp, Artificial Sequence
  Synthetic Sequence
tacagtactgtgatagctgaagtacagtactgtgatagctgaag

SEQ ID NO: 73 - DNA, 44 bp, Artificial Sequence
  Synthetic Sequence
tagccagactattagatctccttagccagactattagatctcct

SEQ ID NO: 74 - DNA, 44 bp, Artificial Sequence
  Synthetic Sequence
caatcagctaagtacactgcctcaatcagctaagtacactgcct

SEQ ID NO: 75 - DNA, 44 bp, Artificial Sequence
  Synthetic Sequence
caatcagctaagtagactgcctcaatcagctaagtagactgcct

SEQ ID NO: 76 - DNA, 44 bp, Artificial Sequence
  Synthetic Sequence
aggcagtgtagttagctgattgaggcagtgtagttagctgattg

SEQ ID NO: 77 - DNA, 44 bp, Artificial Sequence
  Synthetic Sequence
ggatctaacctcacaatactccggatctaacctcacaatactcc

SEQ ID NO: 78 - DNA, 44 bp, Artificial Sequence
  Synthetic Sequence
acacttactgaggacctactagacacttactgaggacctactag

SEQ ID NO: 79 - DNA, 44 bp, Artificial Sequence
  Synthetic Sequence
acagttactgaggacctactagacagttactgaggacctactag

SEQ ID NO: 80 - DNA, 44 bp, Artificial Sequence
  Synthetic Sequence
ctagtaggtgctcagtaagtgtctagtaggtgctcagtaagtgt

SEQ ID NO: 81 - DNA, 44 bp, Artificial Sequence
  Synthetic Sequence
caaaccattggtcaaaccgcttcaaaccattggtcaaaccgctt

SEQ ID NO: 82 - DNA, 42 bp, Artificial Sequence
  Synthetic Sequence
ccaagttctgtcatgcactcaccaagttctgtcatgcactca

SEQ ID NO: 83 - DNA, 42 bp, Artificial Sequence
  Synthetic Sequence
ccaatttctgtcatgcactcaccaatttctgtcatgcactca

SEQ ID NO: 84 - DNA, 42 bp, Artificial Sequence
  Synthetic Sequence
tcagtgcatgacagaacttggtcagtgcatgacagaacttgg

SEQ ID NO: 85 - DNA, 42 bp, Artificial Sequence
  Synthetic Sequence
ggagatattccttccgtaaccggagatattccttccgtaacc

SEQ ID NO: 86 - DNA, 42 bp, Artificial Sequence
  Synthetic Sequence
actggatagcaccagctgtgtactggatagcaccagctgtgt

SEQ ID NO: 87 - DNA, 42 bp, Artificial Sequence
  Synthetic Sequence
actggatagcaccagctttgtactggatagcaccagctttgt

SEQ ID NO: 88 - DNA, 42 bp, Artificial Sequence
  Synthetic Sequence
acacagctggtggtatccagtacacagctggtggtatccagt

SEQ ID NO: 89 - DNA, 42 bp, Artificial Sequence
  Synthetic Sequence
catactgtgttcaggcgcacacatactgtgttcaggcgcaca

SEQ ID NO: 90 - DNA, 42 bp, Artificial Sequence
  Synthetic Sequence
gcaagaactcagactttgatggcaagaactcagactttgatg

SEQ ID NO: 91 - DNA, 42 bp, Artificial Sequence
  Synthetic Sequence
gcaagaagtcagactttgatggcaagaagtcagactttgatg

SEQ ID NO: 92 - DNA, 42 bp, Artificial Sequence
  Synthetic Sequence
catcacagtctgagttcttgccatcacagtctgagttcttgc

SEQ ID NO: 93 - DNA, 42 bp, Artificial Sequence
  Synthetic Sequence
agagggagctgtaaaccttcaagagggagctgtaaaccttca

SEQ ID NO: 94 - DNA, 44 bp, Artificial Sequence
  Synthetic Sequence
acaacaaaatcactattcttccacaacaaaatcactattcttcc

SEQ ID NO: 95 - DNA, 44 bp, Artificial Sequence
  Synthetic Sequence
acaacaaaatgactattcttccacaacaaaatgactattcttcc

SEQ ID NO: 96 - DNA, 44 bp, Artificial Sequence
  Synthetic Sequence
ggaagactagtgattttgttgtggaagactagtgattttgttgt

SEQ ID NO: 97 - DNA, 44 bp, Artificial Sequence
  Synthetic Sequence
tgccaaacaatacccatatctatgccaaacaatacccatatcta

SEQ ID NO: 98 - DNA, 42 bp, Artificial Sequence
  Synthetic Sequence
ttagctgatgtacacgcggtgttagctgatgtacacgcggtg

SEQ ID NO: 99 - DNA, 42 bp, Artificial Sequence
  Synthetic Sequence
ttagctgatttacacgcggtgttagctgatttacacgcggtg

SEQ ID NO: 100 - DNA, 42 bp, Artificial Sequence
  Synthetic Sequence
caccgggtgtacatcagctaacaccgggtgtacatcagctaa

SEQ ID NO: 101 - DNA, 42 bp, Artificial Sequence
  Synthetic Sequence
ttacctgtgggtacccgatagttacctgtgggtacccgatag

SEQ ID NO: 102 - DNA, 21 bp, Artificial Sequence
  Synthetic Sequence
ttagctgatgtacacccggtg

SEQ ID NO: 103 - DNA, 21 bp, Artificial Sequence
  Synthetic Sequence
gcggaacttagccactgtgaa

SEQ ID NO: 104 - DNA, 21 bp, Artificial Sequence
  Synthetic Sequence
ctacctgcacgaacagcactt

SEQ ID NO: 105 - DNA, 22 bp, Artificial Sequence
  Synthetic Sequence
acaacaaaatcactagtcttcc

SEQ ID NO: 106 - DNA, 21 bp, Artificial Sequence
  Synthetic Sequence
gcaagaactcagactgtgatg

SEQ ID NO: 107 - DNA, 21 bp, Artificial Sequence
  Synthetic Sequence
agctgcttttgggattccgtt

SEQ ID NO: 108 - DNA, 22 bp, Artificial Sequence
  Synthetic Sequence
cataccactttgtacaaccaaa

SEQ ID NO: 109 - DNA, 22 bp, Artificial Sequence
  Synthetic Sequence
agctcctacccgaaacatgtaa

SEQ ID NO: 110 - DNA, 22 bp, Artificial Sequence
  Synthetic Sequence
acactcaaaacctggcggcact

SEQ ID NO: 111 - DNA, 21 bp, Artificial Sequence
  Synthetic Sequence
actggataccaccagctgtgt

SEQ ID NO: 112 - DNA, 21 bp, Artificial Sequence
  Synthetic Sequence
ccaagttctgtcatgcactga

SEQ ID NO: 113 - DNA, 21 bp, Artificial Sequence
  Synthetic Sequence
actggtacaagggttgggaga

SEQ ID NO: 114 - DNA, 20 bp, Artificial Sequence
  Synthetic Sequence
acaccaatgccctaggggat

SEQ ID NO: 115 - DNA, 22 bp, Artificial Sequence
  Synthetic Sequence
acacttactgagcacctactag

SEQ ID NO: 116 - DNA, 22 bp, Artificial Sequence
  Synthetic Sequence
cttcagctatcacagtactgta

SEQ ID NO: 117 - DNA, 22 bp, Artificial Sequence
  Synthetic Sequence
tccatcattacccggcagtatt

SEQ ID NO: 118 - DNA, 22 bp, Artificial Sequence
  Synthetic Sequence
caatcagctaactacactgcct

SEQ ID NO: 119 - DNA, 22 bp, Artificial Sequence
  Synthetic Sequence
tgtaaaccatgatgtgctgcta

SEQ ID NO: 120 - DNA, 22 bp, Artificial Sequence
  Synthetic Sequence
cgccaatatttacgtgctgcta

SEQ ID NO: 121 - DNA, 22 bp, Artificial Sequence
  Synthetic Sequence
caatcagctaactacactgcct

SEQ ID NO: 122 - DNA, 44 bp, Artificial Sequence
  Synthetic Sequence
aactatacaacctactacctcaaactatacaacctactacctca

SEQ ID NO: 123 - DNA, 44 bp, Artificial Sequence
  Synthetic Sequence
aaccacacaacctactacctcaaaccacacaacctactacctca

SEQ ID NO: 124 - DNA, 44 bp, Artificial Sequence
  Synthetic Sequence
aaccatacaacctactacctcaaaccatacaacctactacctca

SEQ ID NO: 125 - DNA, 42 bp, Artificial Sequence
  Synthetic Sequence
actatgcaacctactacctctactatgcaacctactacctct

SEQ ID NO: 126 - DNA, 42 bp, Artificial Sequence
  Synthetic Sequence
actatacaacctcctacctcaactatacaacctcctacctca

SEQ ID NO: 127 - DNA, 44 bp, Artificial Sequence
  Synthetic Sequence
aactatacaatctactacctcaaactatacaatctactacctca

SEQ ID NO: 128 - DNA, 42 bp, Artificial Sequence
  Synthetic Sequence
actgtacaaactactacctcaactgtacaaactactacctca

SEQ ID NO: 129 - DNA, 42 bp, Artificial Sequence
  Synthetic Sequence
acagcacaaactactacctcaacagcacaaactactacctca

SEQ ID NO: 130 - DNA, 42 bp, Artificial Sequence
  Synthetic Sequence
tacatacttctttacattccatacatacttctttacattcca

SEQ ID NO: 131 - DNA, 42 bp, Artificial Sequence
  Synthetic Sequence
cacaagttcggatctacgggtcacaagttcggatctacgggt

SEQ ID NO: 132 - DNA, 44 bp, Artificial Sequence
  Synthetic Sequence
cttcagttatcacagtactgtacttcagttatcacagtactgta

SEQ ID NO: 133 - DNA, 42 bp, Artificial Sequence; identical to SEQ ID NO: 16
  Synthetic Sequence

SEQ ID NO: 134 - DNA, 40 bp, Artificial Sequence
  Synthetic Sequence
acaggagtctgagcatttgaacaggagtctgagcatttga

SEQ ID NO: 135 - DNA, 44 bp, Artificial Sequence
  Synthetic Sequence
ctacctgcactgtaagcactttctacctgcactgtaagcacttt

SEQ ID NO: 136 - DNA, 42 bp, Artificial Sequence
  Synthetic Sequence
atctgcactgtcagcactttaatctgcactgtcagcacttta

SEQ ID NO: 137 - DNA, 42 bp, Artificial Sequence
  Synthetic Sequence
tgatagccctgtacaatgctgtgatagccctgtacaatgctg

SEQ ID NO: 138 - DNA, 44 bp, Artificial Sequence
  Synthetic Sequence
cacaaattcggatctacagggtcacaaattcggatctacagggt

SEQ ID NO: 139 - DNA, 44 bp, Artificial Sequence
  Synthetic Sequence
acaaattcggttctacagggtaacaaattcggttctacagggta

SEQ ID NO: 140 - DNA, 44 bp, Artificial Sequence
  Synthetic Sequence
acaaacaccattgtcacactccacaaacaccattgtcacactcc

SEQ ID NO: 141 - DNA, 44 bp, Artificial Sequence
  Synthetic Sequence
tggcattcaccgcgtgccttaatggcattcaccgcgtgccttaa

SEQ ID NO: 142 - DNA, 42 bp, Artificial Sequence
  Synthetic Sequence
cacaggttaaagggtctcaggcacaggttaaagggtctcagg

SEQ ID NO: 143 - DNA, 42 bp, Artificial Sequence
  Synthetic Sequence
tcacaagttagggtctcagggtcacaagttagggtctcaggg

SEQ ID NO: 144 - DNA, 42 bp, Artificial Sequence
  Synthetic Sequence
gcattattactcacggtacgagcattattactcacggtacga

SEQ ID NO: 145 - DNA, 42 bp, Artificial Sequence
  Synthetic Sequence
cgcgtaccaaaagtaataatgcgcgtaccaaaagtaataatg

SEQ ID NO: 146 - DNA, 44 bp, Artificial Sequence
  Synthetic Sequence
agccaagctcagacggatccgaagccaagctcagacggatccga

SEQ ID NO: 147 - DNA, 44 bp, Artificial Sequence
  Synthetic Sequence
aaaagagaccggttcactgtgaaaaagagaccggttcactgtga

SEQ ID NO: 148 - DNA, 42 bp, Artificial Sequence
  Synthetic Sequence
gaaagagaccggttcactgtggaaagagaccggttcactgtg

SEQ ID NO: 149 - DNA, 40 bp, Artificial Sequence
  Synthetic Sequence
gcaagcccagaccgcaaaaagcaagcccagaccgcaaaaa

SEQ ID NO: 150 - DNA, 44 bp, Artificial Sequence
  Synthetic Sequence
atgcccttttaacattgcactgatgcccttttaacattgcactg

SEQ ID NO: 151 - DNA, 44 bp, Artificial Sequence
  Synthetic Sequence
atgccctttcatcattgcactgatgccctttcatcattgcactg

SEQ ID NO: 152 - DNA, 42 bp, Artificial Sequence
  Synthetic Sequence
cgaccatggctgtagactgttcgaccatggctgtagactgtt

SEQ ID NO: 153 - DNA, 44 bp, Artificial Sequence
  Synthetic Sequence
acagctggttgaaggggaccaaacagctggttgaaggggaccaa

SEQ ID NO: 154 - DNA, 42 bp, Artificial Sequence
  Synthetic Sequence
tagctggttgaaggggaccaatagctggttgaaggggaccaa

SEQ ID NO: 155 - DNA, 40 bp, Artificial Sequence
  Synthetic Sequence
cctctggtcaaccagtcacacctctggtcaaccagtcaca

SEQ ID NO: 156 - DNA, 44 bp, Artificial Sequence
  Synthetic Sequence
tcacataggaataaaaagccattcacataggaataaaaagccat

SEQ ID NO: 157 - DNA, 44 bp, Artificial Sequence
  Synthetic Sequence
cacataggaatgaaaagccatacacataggaatgaaaagccata

SEQ ID NO: 158 - DNA, 44 bp, Artificial Sequence
  Synthetic Sequence
tccatcatcaaaacaaatggagtccatcatcaaaacaaatggag

SEQ ID NO: 159 - DNA, 44 bp, Artificial Sequence
  Synthetic Sequence
ctacgcgtattcttaagcaatactacgcgtattcttaagcaata

SEQ ID NO: 160 - DNA, 34 bp, Artificial Sequence
  Synthetic Sequence
gattcacaacaccagctgattcacaacaccagct

SEQ ID NO: 161 - DNA, 36 bp, Artificial Sequence
  Synthetic Sequence
agacacgtgcactgtagaagacacgtgcactgtaga

SEQ ID NO: 162 - DNA, 42 bp, Artificial Sequence
  Synthetic Sequence
ctaccatagggtaaaaccactctaccatagggtaaaaccact

SEQ ID NO: 163 - DNA, 44 bp, Artificial Sequence
  Synthetic Sequence
ccatctttaccagacagtgttaccatctttaccagacagtgtta

SEQ ID NO: 164 - DNA, 44 bp, Artificial Sequence
  Synthetic Sequence
tccataaagtaggaaacactactccataaagtaggaaacactac

SEQ ID NO: 165 - DNA, 40 bp, Artificial Sequence
  Synthetic Sequence
gtagtgctttctactttatggtagtgctttctactttatg

SEQ ID NO: 166 - DNA, 44 bp, Artificial Sequence
  Synthetic Sequence
tgagctacagtgcttcatctcatgagctacagtgcttcatctca

SEQ ID NO: 167 - DNA, 44 bp, Artificial Sequence
  Synthetic Sequence
ctagtacatcatctatactgtactagtacatcatctatactgta

SEQ ID NO: 168 - DNA, 42 bp, Artificial Sequence
  Synthetic Sequence
aagggattcctgggaaaactgaagggattcctgggaaaactg

SEQ ID NO: 169 - DNA, 44 bp, Artificial Sequence
  Synthetic Sequence
aacccatggaattcagttctcaaacccatggaattcagttctca

SEQ ID NO: 170 - DNA, 44 bp, Artificial Sequence
  Synthetic Sequence
agcctatggaattcagttctcaagcctatggaattcagttctca

SEQ ID NO: 171 - DNA, 40 bp, Artificial Sequence
  Synthetic Sequence
gcagaagcatttccacacacgcagaagcatttccacacac

SEQ ID NO: 172 - DNA, 44 bp, Artificial Sequence
  Synthetic Sequence
acaaagttctgtagtgcactgaacaaagttctgtagtgcactga

SEQ ID NO: 173 - DNA, 44 bp, Artificial Sequence
  Synthetic Sequence
acaaagttctgtgatgcactgaacaaagttctgtgatgcactga

SEQ ID NO: 174 - DNA, 40 bp, Artificial Sequence
  Synthetic Sequence
agtgaagacacggagccagaagtgaagacacggagccaga

SEQ ID NO: 175 - DNA, 42 bp, Artificial Sequence
  Synthetic Sequence
actggtacaagggttgggagaactggtacaagggttgggaga

SEQ ID NO: 176 - DNA, 42 bp, Artificial Sequence
  Synthetic Sequence
cctcaaggagcttcagtctagcctcaaggagcttcagtctag

SEQ ID NO: 177 - DNA, 42 bp, Artificial Sequence
  Synthetic Sequence
cccaagttctgtcatgcactgcccaagttctgtcatgcactg

SEQ ID NO: 178 - DNA, 40 bp, Artificial Sequence
  Synthetic Sequence
tcacttttgtgactatgcaatcacttttgtgactatgcaa

SEQ ID NO: 179 - DNA, 42 bp, Artificial Sequence
  Synthetic Sequence
cgaaggcaacacggataacctcgaaggcaacacggataacct

SEQ ID NO: 180 - DNA, 44 bp, Artificial Sequence
  Synthetic Sequence
aataggtcaaccgtgtatgattaataggtcaaccgtgtatgatt

SEQ ID NO: 181 - DNA, 44 bp, Artificial Sequence
  Synthetic Sequence
cccctatcacgattagcattaacccctatcacgattagcattaa

SEQ ID NO: 182 - DNA, 44 bp, Artificial Sequence
  Synthetic Sequence
cacaaaccattatgtgctgctacacaaaccattatgtgctgcta

SEQ ID NO: 183 - DNA, 44 bp, Artificial Sequence
  Synthetic Sequence
tgtaaaccatgatgtgctgctatgtaaaccatgatgtgctgcta

SEQ ID NO: 184 - DNA, 44 bp, Artificial Sequence
  Synthetic Sequence
cgccaatatttacgtgctgctacgccaatatttacgtgctgcta

SEQ ID NO: 185 - DNA, 40 bp, Artificial Sequence
  Synthetic Sequence
acaagtgccttcactgcagtacaagtgccttcactgcagt

SEQ ID NO: 186 - DNA, 44 bp, Artificial Sequence
  Synthetic Sequence
actacctgcactgtaagcacttactacctgcactgtaagcactt

SEQ ID NO: 187 - DNA, 42 bp, Artificial Sequence
  Synthetic Sequence
actcaccgacagcgttgaatgactcaccgacagcgttgaatg

SEQ ID NO: 188 - DNA, 42 bp, Artificial Sequence
  Synthetic Sequence
cccaccgacagcaatgaatgtcccaccgacagcaatgaatgt

SEQ ID NO: 189 - DNA, 44 bp, Artificial Sequence
  Synthetic Sequence
actcaccgacaggttgaatgttactcaccgacaggttgaatgtt

SEQ ID NO: 190 - DNA, 44 bp, Artificial Sequence
  Synthetic Sequence
aacccaccgacaacaatgaatgaacccaccgacaacaatgaatg

SEQ ID NO: 191 - DNA, 44 bp, Artificial Sequence
  Synthetic Sequence
tgtgagttctaccattgccaaatgtgagttctaccattgccaaa

SEQ ID NO: 192 - DNA, 42 bp, Artificial Sequence
  Synthetic Sequence
tagttggcaagtctagaaccatagttggcaagtctagaacca

SEQ ID NO: 193 - DNA, 44 bp, Artificial Sequence
  Synthetic Sequence
cagtgaattctaccagtgccatcagtgaattctaccagtgccat

SEQ ID NO: 194 - DNA, 42 bp, Artificial Sequence
  Synthetic Sequence
acccttatcagttctccgtccacccttatcagttctccgtcc

SEQ ID NO: 195 - DNA, 36 bp, Artificial Sequence
  Synthetic Sequence
gaactgcctttctctccagaactgcctttctctcca

SEQ ID NO: 196 - DNA, 44 bp, Artificial Sequence
  Synthetic Sequence
agcccaaaaggagaattctttgagcccaaaaggagaattctttg

SEQ ID NO: 197 - DNA, 40 bp, Artificial Sequence
  Synthetic Sequence
ggctgcaacacaagacacgaggctgcaacacaagacacga

SEQ ID NO: 198 - DNA, 42 bp, Artificial Sequence
  Synthetic Sequence
accctccaccatgcaagggataccctccaccatgcaagggat

SEQ ID NO: 199 - DNA, 44 bp, Artificial Sequence
  Synthetic Sequence
actgatatcagctcagtaggcaactgatatcagctcagtaggca

SEQ ID NO: 200 - DNA, 44 bp, Artificial Sequence
  Synthetic Sequence
tatctgcactagatgcaccttatatctgcactagatgcacctta

SEQ ID NO: 201 - DNA, 44 bp, Artificial Sequence
  Synthetic Sequence
taactgcactagatgcaccttataactgcactagatgcacctta

SEQ ID NO: 202 - DNA, 44 bp, Artificial Sequence
  Synthetic Sequence
acctaatatatcaaacatatcaacctaatatatcaaacatatca

SEQ ID NO: 203 - DNA, 42 bp, Artificial Sequence
  Synthetic Sequence
agctgcttttgggattccgttagctgcttttgggattccgtt

SEQ ID NO: 204 - DNA, 38 bp, Artificial Sequence
  Synthetic Sequence
gggacgaaatccaagcgcagggacgaaatccaagcgca

SEQ ID NO: 205 - DNA, 42 bp, Artificial Sequence
  Synthetic Sequence
ggctgtcaattcataggtcagggctgtcaattcataggtcag

SEQ ID NO: 206 - DNA, 42 bp, Artificial Sequence
  Synthetic Sequence
ctgggactttgtaggccagttctgggactttgtaggccagtt

SEQ ID NO: 207 - DNA, 42 bp, Artificial Sequence
  Synthetic Sequence
aaagcgggactttgagggccaaaagcgggactttgagggcca

SEQ ID NO: 208 - DNA, 44 bp, Artificial Sequence
  Synthetic Sequence
tccacatggagttgctgttacatccacatggagttgctgttaca

SEQ ID NO: 209 - DNA, 42 bp, Artificial Sequence
  Synthetic Sequence
gccaatatttctgtgctgctagccaatatttctgtgctgcta

SEQ ID NO: 210 - DNA, 42 bp, Artificial Sequence
  Synthetic Sequence
ccaacaacatgaaactacctaccaacaacatgaaactaccta

SEQ ID NO: 211 - DNA, 42 bp, Artificial Sequence
  Synthetic Sequence
ccaacaacaggaaactacctaccaacaacaggaaactaccta

SEQ ID NO: 212 - DNA, 40 bp, Artificial Sequence
  Synthetic Sequence
tgggtggagaaggtggtgaatgggtggagaaggtggtgaa

SEQ ID NO: 213 - DNA, 36 bp, Artificial Sequence
  Synthetic Sequence
cctatctcccctctggaccctatctcccctctggac

SEQ ID NO: 214 - DNA, 42 bp, Artificial Sequence
  Synthetic Sequence
gaacaggtagtctgaacactggaacaggtagtctgaacactg

SEQ ID NO: 215 - DNA, 44 bp, Artificial Sequence
  Synthetic Sequence
aaccaatgtgcagactactgtaaaccaatgtgcagactactgta

SEQ ID NO: 216 - DNA, 44 bp, Artificial Sequence
  Synthetic Sequence
gaacagatagtctaaacactgggaacagatagtctaaacactgg

SEQ ID NO: 217 - DNA, 44 bp, Artificial Sequence
  Synthetic Sequence
tcagttttgcatagatttgcactcagttttgcatagatttgcac

SEQ ID NO: 218 - DNA, 44 bp, Artificial Sequence
  Synthetic Sequence
tcagttttgcatggatttgcactcagttttgcatggatttgcac

SEQ ID NO: 219 - DNA, 44 bp, Artificial Sequence
  Synthetic Sequence
acatcgttaccagacagtgttaacatcgttaccagacagtgtta

SEQ ID NO: 220 - DNA, 42 bp, Artificial Sequence
  Synthetic Sequence
tccagcactgtccggtaagattccagcactgtccggtaagat

SEQ ID NO: 221 - DNA, 44 bp, Artificial Sequence
  Synthetic Sequence
gtcatcattaccaggcagtattgtcatcattaccaggcagtatt

SEQ ID NO: 222 - DNA, 44 bp, Artificial Sequence
  Synthetic Sequence
ccatcattacccggcagtattaccatcattacccggcagtatta

SEQ ID NO: 223 - DNA, 44 bp, Artificial Sequence
  Synthetic Sequence
ttttcccatgccctatacctctttttcccatgccctatacctct

SEQ ID NO: 224 - DNA, 44 bp, Artificial Sequence
  Synthetic Sequence
aaagaagtatatgcataggaaaaaagaagtatatgcataggaaa

SEQ ID NO: 225 - DNA, 44 bp, Artificial Sequence
  Synthetic Sequence
ctagtggtcctaaacatttcacctagtggtcctaaacatttcac

SEQ ID NO: 226 - DNA, 44 bp, Artificial Sequence
  Synthetic Sequence
aggcataggatgacaaagggaaaggcataggatgacaaagggaa

SEQ ID NO: 227 - DNA, 42 bp, Artificial Sequence
  Synthetic Sequence
agactccggtggaatgaaggaagactccggtggaatgaagga

SEQ ID NO: 228 - DNA, 44 bp, Artificial Sequence
  Synthetic Sequence
ccacacacttccttacattccaccacacacttccttacattcca

SEQ ID NO: 229 - DNA, 44 bp, Artificial Sequence
  Synthetic Sequence
acaagctttttgctcgtcttatacaagctttttgctcgtcttat

SEQ ID NO: 230 - DNA, 44 bp, Artificial Sequence
  Synthetic Sequence
ctacctgcactataagcactttctacctgcactataagcacttt

SEQ ID NO: 231 - DNA, 44 bp, Artificial Sequence
  Synthetic Sequence
ctacctgcactatgagcactttctacctgcactatgagcacttt

SEQ ID NO: 232 - DNA, 44 bp, Artificial Sequence
  Synthetic Sequence
tcaacatcagtctgataagctatcaacatcagtctgataagcta

SEQ ID NO: 233 - DNA, 42 bp, Artificial Sequence
  Synthetic Sequence
tcagccgctgtcacacgcacatcagccgctgtcacacgcaca

SEQ ID NO: 234 - DNA, 42 bp, Artificial Sequence
  Synthetic Sequence
aggcgaaggatgacaaagggaaggcgaaggatgacaaaggga

SEQ ID NO: 235 - DNA, 40 bp, Artificial Sequence
  Synthetic Sequence
gccgtgactggagactgttagccgtgactggagactgtta

SEQ ID NO: 236 - DNA, 42 bp, Artificial Sequence
  Synthetic Sequence
ggtacaatcaacggtcgatggggtacaatcaacggtcgatgg

SEQ ID NO: 237 - DNA, 40 bp, Artificial Sequence
  Synthetic Sequence
tgcctgtctgtgcctgctgttgcctgtctgtgcctgctgt

SEQ ID NO: 238 - DNA, 42 bp, Artificial Sequence
  Synthetic Sequence
gtctgtcaattcataggtcatgtctgtcaattcataggtcat

SEQ ID NO: 239 - DNA, 42 bp, Artificial Sequence
  Synthetic Sequence
cacagttgccagctgagattacacagttgccagctgagatta

SEQ ID NO: 240 - DNA, 44 bp, Artificial Sequence
  Synthetic Sequence
atccaatcagttcctgatgcagatccaatcagttcctgatgcag

SEQ ID NO: 241 - DNA, 42 bp, Artificial Sequence
  Synthetic Sequence
acatggttagatcaagcacaaacatggttagatcaagcacaa

SEQ ID NO: 242 - DNA, 42 bp, Artificial Sequence
  Synthetic Sequence
agaattgcgtttggacaatcaagaattgcgtttggacaatca

SEQ ID NO: 243 - DNA, 44 bp, Artificial Sequence
  Synthetic Sequence
acagttcttcaactggcagcttacagttcttcaactggcagctt

SEQ ID NO: 244 - DNA, 42 bp, Artificial Sequence
  Synthetic Sequence
aaagtgtcagatacggtgtggaaagtgtcagatacggtgtgg

SEQ ID NO: 245 - DNA, 44 bp, Artificial Sequence
  Synthetic Sequence
aaacccagcagacaatgtagctaaacccagcagacaatgtagct

SEQ ID NO: 246 - DNA, 42 bp, Artificial Sequence
  Synthetic Sequence
agacccagtagccagatgtagagacccagtagccagatgtag

SEQ ID NO: 247 - DNA, 42 bp, Artificial Sequence
  Synthetic Sequence
ggggtatttgacaaactgacaggggtatttgacaaactgaca

SEQ ID NO: 248 - DNA, 44 bp, Artificial Sequence
  Synthetic Sequence
taaacggaaccactagtgactttaaacggaaccactagtgactt

SEQ ID NO: 249 - DNA, 42 bp, Artificial Sequence
  Synthetic Sequence
ggaaatccctggcaatgtgatggaaatccctggcaatgtgat

SEQ ID NO: 250 - DNA, 42 bp, Artificial Sequence
  Synthetic Sequence
ggtaatccctggcaatgtgatggtaatccctggcaatgtgat

SEQ ID NO: 251 - DNA, 42 bp, Artificial Sequence
  Synthetic Sequence
tgttcctgctgaactgagccatgttcctgctgaactgagcca

SEQ ID NO: 252 - DNA, 42 bp, Artificial Sequence
  Synthetic Sequence
tcagaccgagacaagtgcaattcagaccgagacaagtgcaat

SEQ ID NO: 253 - DNA, 42 bp, Artificial Sequence
  Synthetic Sequence
gcctatcctggattacttgaagcctatcctggattacttgaa

SEQ ID NO: 254 - DNA, 44 bp, Artificial Sequence
  Synthetic Sequence
aacctatcctgaattacttgaaaacctatcctgaattacttgaa

SEQ ID NO: 255 - DNA, 42 bp, Artificial Sequence
  Synthetic Sequence
gcggaacttagccactgtgaagcggaacttagccactgtgaa

SEQ ID NO: 256 - DNA, 42 bp, Artificial Sequence
  Synthetic Sequence
gcagaacttagccactgtgaagcagaacttagccactgtgaa

SEQ ID NO: 257 - DNA, 44 bp, Artificial Sequence
  Synthetic Sequence
ctcaatagactgtgagctccttctcaatagactgtgagctcctt

SEQ ID NO: 258 - DNA, 42 bp, Artificial Sequence
  Synthetic Sequence
acaggattgagggggggccctacaggattgagggggggccct

SEQ ID NO: 259 - DNA, 44 bp, Artificial Sequence
  Synthetic Sequence
aagcggtttaccatcccacataaagcggtttaccatcccacata

SEQ ID NO: 260 - DNA, 42 bp, Artificial Sequence
  Synthetic Sequence
aaccgatttcagatggtgctaaaccgatttcagatggtgcta

SEQ ID NO: 261 - DNA, 44 bp, Artificial Sequence
  Synthetic Sequence
aacactgatttcaaatggtgctaacactgatttcaaatggtgct

SEQ ID NO: 262 - DNA, 40 bp, Artificial Sequence
  Synthetic Sequence
accgatttcaaatggtgctaaccgatttcaaatggtgcta

SEQ ID NO: 263 - DNA, 44 bp, Artificial Sequence
  Synthetic Sequence
gctttgacaatactattgcactgctttgacaatactattgcact

SEQ ID NO: 264 - DNA, 44 bp, Artificial Sequence
  Synthetic Sequence
tcaccaaaacatggaagcactttcaccaaaacatggaagcactt

SEQ ID NO: 265 - DNA, 44 bp, Artificial Sequence
  Synthetic Sequence
aaagcaagtacatccacgtttaaaagcaagtacatccacgttta

SEQ ID NO: 266 - DNA, 44 bp, Artificial Sequence
  Synthetic Sequence
ctactaaaacatggaagcacttctactaaaacatggaagcactt

SEQ ID NO: 267 - DNA, 44 bp, Artificial Sequence
  Synthetic Sequence
agaaagcacttccatgttaaagagaaagcacttccatgttaaag

SEQ ID NO: 268 - DNA, 44 bp, Artificial Sequence
  Synthetic Sequence
ccactgaaacatggaagcacttccactgaaacatggaagcactt

SEQ ID NO: 269 - DNA, 42 bp, Artificial Sequence
  Synthetic Sequence
cagcaggtacccccatgttaacagcaggtacccccatgttaa

SEQ ID NO: 270 - DNA, 44 bp, Artificial Sequence
  Synthetic Sequence
acactcaaacatggaagcacttacactcaaacatggaagcactt

SEQ ID NO: 271 - DNA, 42 bp, Artificial Sequence
  Synthetic Sequence
gctgcaaacatccgactgaaagctgcaaacatccgactgaaa

SEQ ID NO: 272 - DNA, 44 bp, Artificial Sequence
  Synthetic Sequence
cttccagtcgaggatgtttacacttccagtcgaggatgtttaca

SEQ ID NO: 273 - DNA, 44 bp, Artificial Sequence
  Synthetic Sequence
agctgagtgtaggatgtttacaagctgagtgtaggatgtttaca

SEQ ID NO: 274 - DNA, 44 bp, Artificial Sequence
  Synthetic Sequence
gctgagagtgtaggatgtttacgctgagagtgtaggatgtttac

SEQ ID NO: 275 - DNA, 42 bp, Artificial Sequence
  Synthetic Sequence
cttccagtcggggatgtttaccttccagtcggggatgtttac

SEQ ID NO: 276 - DNA, 44 bp, Artificial Sequence
  Synthetic Sequence
gctgtaaacatccgactgaaaggctgtaaacatccgactgaaag

SEQ ID NO: 277 - DNA, 40 bp, Artificial Sequence
  Synthetic Sequence
tccagtcaaggatgtttacatccagtcaaggatgtttaca

SEQ ID NO: 278 - DNA, 40 bp, Artificial Sequence
  Synthetic Sequence
cagctatgccagcatcttgccagctatgccagcatcttgc

SEQ ID NO: 279 - DNA, 40 bp, Artificial Sequence
  Synthetic Sequence
gcaacttagtaatgtgcaatgcaacttagtaatgtgcaat

SEQ ID NO: 280 - DNA, 44 bp, Artificial Sequence
  Synthetic Sequence
ttcgccctctcaacccagctttttcgccctctcaacccagcttt

SEQ ID NO: 281 - DNA, 42 bp, Artificial Sequence
  Synthetic Sequence
agaggtcgaccgtgtaatgtgagaggtcgaccgtgtaatgtg

SEQ ID NO: 282 - DNA, 36 bp, Artificial Sequence
  Synthetic Sequence
agcagcacctggggcagtagcagcacctggggcagt

SEQ ID NO: 283 - DNA, 40 bp, Artificial Sequence
  Synthetic Sequence
acaccaatgccctaggggatacaccaatgccctaggggat

SEQ ID NO: 284 - DNA, 44 bp, Artificial Sequence
  Synthetic Sequence
acacttactggacacctactagacacttactggacacctactag

SEQ ID NO: 285 - DNA, 34 bp, Artificial Sequence
  Synthetic Sequence
tggaggaagggcccagatggaggaagggcccaga

SEQ ID NO: 286 - DNA, 42 bp, Artificial Sequence
  Synthetic Sequence
acggaagggcagagagggccaacggaagggcagagagggcca

SEQ ID NO: 287 - DNA, 44 bp, Artificial Sequence
  Synthetic Sequence
aaagaggttaaccaggtgtgttaaagaggttaaccaggtgtgtt

SEQ ID NO: 288 - DNA, 38 bp, Artificial Sequence
  Synthetic Sequence
caatgcaactacaatgcaccaatgcaactacaatgcac

SEQ ID NO: 289 - DNA, 42 bp, Artificial Sequence
  Synthetic Sequence
tctctgcaggccgtgtgcttttctctgcaggccgtgtgcttt

SEQ ID NO: 290 - DNA, 38 bp, Artificial Sequence
  Synthetic Sequence
ttctaggataggcccagggttctaggataggcccaggg

SEQ ID NO: 291 - DNA, 44 bp, Artificial Sequence
  Synthetic Sequence
acatttttcgttattgctcttgacatttttcgttattgctcttg

SEQ ID NO: 292 - DNA, 44 bp, Artificial Sequence
  Synthetic Sequence
aaaggcatcatataggagctggaaaggcatcatataggagctgg

SEQ ID NO: 293 - DNA, 44 bp, Artificial Sequence
  Synthetic Sequence
tcaacaaaatcactgatgctggtcaacaaaatcactgatgctgg

SEQ ID NO: 294 - DNA, 42 bp, Artificial Sequence
  Synthetic Sequence
tgagctcctggaggacagggatgagctcctggaggacaggga

SEQ ID NO: 295 - DNA, 44 bp, Artificial Sequence
  Synthetic Sequence
ggctataaagtaactgagacggggctataaagtaactgagacgg

SEQ ID NO: 296 - DNA, 42 bp, Artificial Sequence
  Synthetic Sequence
acgggtgcgatttctgtgtgaacgggtgcgatttctgtgtga

SEQ ID NO: 297 - DNA, 38 bp, Artificial Sequence
  Synthetic Sequence
cctggactaggagtcagcacctggactaggagtcagca

SEQ ID NO: 298 - DNA, 42 bp, Artificial Sequence
  Synthetic Sequence
agaggcaggcatgcgggcagaagaggcaggcatgcgggcaga

SEQ ID NO: 299 - DNA, 42 bp, Artificial Sequence
  Synthetic Sequence
aacaaccagctaagacactgcaacaaccagctaagacactgc

SEQ ID NO: 300 - DNA, 44 bp, Artificial Sequence
  Synthetic Sequence
caatcagctaatgacactgcctcaatcagctaatgacactgcct

SEQ ID NO: 301 - DNA, 44 bp, Artificial Sequence
  Synthetic Sequence
caatcagctaactacactgcctcaatcagctaactacactgcct

SEQ ID NO: 302 - DNA, 44 bp, Artificial Sequence
  Synthetic Sequence
gtacccctggagattctgataagtacccctggagattctgataa

SEQ ID NO: 303 - DNA, 44 bp, Artificial Sequence
  Synthetic Sequence
tcacacctaggttccaaggatttcacacctaggttccaaggatt

SEQ ID NO: 304 - DNA, 44 bp, Artificial Sequence
  Synthetic Sequence
ttacagatggataccgtgcaatttacagatggataccgtgcaat

SEQ ID NO: 305 - DNA, 44 bp, Artificial Sequence
  Synthetic Sequence
ataaggatttttaggggcattaataaggatttttaggggcatta

SEQ ID NO: 306 - DNA, 44 bp, Artificial Sequence
  Synthetic Sequence
tcaccattgctaaagtgcaatttcaccattgctaaagtgcaatt

SEQ ID NO: 307 - DNA, 44 bp, Artificial Sequence
  Synthetic Sequence
aaacgtggaatttcctctatgtaaacgtggaatttcctctatgt

SEQ ID NO: 308 - DNA, 42 bp, Artificial Sequence
  Synthetic Sequence
aaagatcaaccatgtattattaaagatcaaccatgtattatt

SEQ ID NO: 309 - DNA, 44 bp, Artificial Sequence
  Synthetic Sequence
gcgaatataacacggtcgatctgcgaatataacacggtcgatct

SEQ ID NO: 310 - DNA, 34 bp, Artificial Sequence
  Synthetic Sequence
caggttccaccccagcacaggttccaccccagca

SEQ ID NO: 311 - DNA, 42 bp, Artificial Sequence
  Synthetic Sequence
acactcaaaagatggcggcacacactcaaaagatggcggcac

SEQ ID NO: 312 - DNA, 42 bp, Artificial Sequence
  Synthetic Sequence
acgctcaaatgtcgcagcactacgctcaaatgtcgcagcact

SEQ ID NO: 313 - DNA, 44 bp, Artificial Sequence
  Synthetic Sequence
acaccccaaaatcgaagcacttacaccccaaaatcgaagcactt

SEQ ID NO: 314 - DNA, 42 bp, Artificial Sequence
  Synthetic Sequence
gaaagcgcccccattttgagtgaaagcgcccccattttgagt

SEQ ID NO: 315 - DNA, 44 bp, Artificial Sequence
  Synthetic Sequence
cacttatcaggttgtattataacacttatcaggttgtattataa

SEQ ID NO: 316 - DNA, 44 bp, Artificial Sequence
  Synthetic Sequence
tcacgcgagccgaacgaacaaatcacgcgagccgaacgaacaaa

SEQ ID NO: 317 - DNA, 42 bp, Artificial Sequence
  Synthetic Sequence
acgtggattttcctctatgatacgtggattttcctctatgat

SEQ ID NO: 318 - DNA, 44 bp, Artificial Sequence
  Synthetic Sequence
aacatggattttcctctatgataacatggattttcctctatgat

SEQ ID NO: 319 - DNA, 44 bp, Artificial Sequence
  Synthetic Sequence
acaaaagttgcctttgtgtgatacaaaagttgcctttgtgtgat

SEQ ID NO: 320 - DNA, 42 bp, Artificial Sequence
  Synthetic Sequence
acacaggacctggagtcaggaacacaggacctggagtcagga

SEQ ID NO: 321 - DNA, 38 bp, Artificial Sequence
  Synthetic Sequence
tacgttccatagtctaccatacgttccatagtctacca

SEQ ID NO: 322 - DNA, 44 bp, Artificial Sequence
  Synthetic Sequence
aagatgtggaccatattacataaagatgtggaccatattacata

SEQ ID NO: 323 - DNA, 42 bp, Artificial Sequence
  Synthetic Sequence
gcgcatgttctatggtcaaccgcgcatgttctatggtcaacc

SEQ ID NO: 324 - DNA, 44 bp, Artificial Sequence
  Synthetic Sequence
acagagagcttgcccttgtataacagagagcttgcccttgtata

SEQ ID NO: 325 - DNA, 42 bp, Artificial Sequence
  Synthetic Sequence
cgaatccaccacgaacaacttcgaatccaccacgaacaactt

SEQ ID NO: 326 - DNA, 44 bp, Artificial Sequence
  Synthetic Sequence
agccacaatcaccttctgatctagccacaatcaccttctgatct

SEQ ID NO: 327 - DNA, 40 bp, Artificial Sequence
  Synthetic Sequence
tatgaacaatttctaggaattatgaacaatttctaggaat

SEQ ID NO: 328 - DNA, 42 bp, Artificial Sequence
  Synthetic Sequence
aggggttcaccgagcaacattaggggttcaccgagcaacatt

SEQ ID NO: 329 - DNA, 42 bp, Artificial Sequence
  Synthetic Sequence
tgcaaagttgctcgggtaacctgcaaagttgctcgggtaacc

SEQ ID NO: 330 - DNA, 44 bp, Artificial Sequence
  Synthetic Sequence
aacaggccatctgtgttatattaacaggccatctgtgttatatt

SEQ ID NO: 331 - DNA, 42 bp, Artificial Sequence
  Synthetic Sequence
acggctagtggaccaggtgaaacggctagtggaccaggtgaa

SEQ ID NO: 332 - DNA, 40 bp, Artificial Sequence
  Synthetic Sequence
gccttctgaccctaagtccagccttctgaccctaagtcca

SEQ ID NO: 333 - DNA, 40 bp, Artificial Sequence
  Synthetic Sequence
gccttctgactccaagtccagccttctgactccaagtcca

SEQ ID NO: 334 - DNA, 42 bp, Artificial Sequence
  Synthetic Sequence
tgaggggcctcagaccgagcttgaggggcctcagaccgagct

SEQ ID NO: 335 - DNA, 44 bp, Artificial Sequence
  Synthetic Sequence
ttcaaaacatgaattgctgctgttcaaaacatgaattgctgctg

SEQ ID NO: 336 - DNA, 38 bp, Artificial Sequence
  Synthetic Sequence
cggacacgacattcccgatcggacacgacattcccgat

SEQ ID NO: 337 - DNA, 44 bp, Artificial Sequence
  Synthetic Sequence
acggttttaccagacagtattaacggttttaccagacagtatta

SEQ ID NO: 338 - DNA, 42 bp, Artificial Sequence
  Synthetic Sequence
tgcatgacggcctgcaagacatgcatgacggcctgcaagaca

SEQ ID NO: 339 - DNA, 42 bp, Artificial Sequence
  Synthetic Sequence
ccacccaatgacctactccaaccacccaatgacctactccaa

SEQ ID NO: 340 - DNA, 40 bp, Artificial Sequence
  Synthetic Sequence
agacatggaggagccatccaagacatggaggagccatcca

SEQ ID NO: 341 - DNA, 44 bp, Artificial Sequence
  Synthetic Sequence
acaccgaggagcccatcatgatacaccgaggagcccatcatgat

SEQ ID NO: 342 - DNA, 44 bp, Artificial Sequence
  Synthetic Sequence
atgggacatcctacatatgcaaatgggacatcctacatatgcaa

SEQ ID NO: 343 - DNA, 44 bp, Artificial Sequence
  Synthetic Sequence
accagctaacaatacactgccaaccagctaacaatacactgcca

SEQ ID NO: 344 - DNA, 44 bp, Artificial Sequence
  Synthetic Sequence
tattaggaacacatcgcaaaaatattaggaacacatcgcaaaaa

SEQ ID NO: 345 - DNA, 44 bp, Artificial Sequence
  Synthetic Sequence
aaactcagtaatggtaacggttaaactcagtaatggtaacggtt

SEQ ID NO: 346 - DNA, 44 bp, Artificial Sequence
  Synthetic Sequence
gtctcagtttcctctgcaaacagtctcagtttcctctgcaaaca

SEQ ID NO: 347 - DNA, 42 bp, Artificial Sequence
  Synthetic Sequence
cttctttgcagatgagactgacttctttgcagatgagactga

SEQ ID NO: 348 - DNA, 40 bp, Artificial Sequence
  Synthetic Sequence
gaactcaccacggacaacctgaactcaccacggacaacct

SEQ ID NO: 349 - DNA, 40 bp, Artificial Sequence
  Synthetic Sequence
agaggagagccgtgtatgacagaggagagccgtgtatgac

SEQ ID NO: 350 - DNA, 42 bp, Artificial Sequence
  Synthetic Sequence
aattcatcacggccagcctctaattcatcacggccagcctct

SEQ ID NO: 351 - DNA, 42 bp, Artificial Sequence
  Synthetic Sequence
ttgagagtgccattatctgggttgagagtgccattatctggg

SEQ ID NO: 352 - DNA, 44 bp, Artificial Sequence
  Synthetic Sequence
ctgccgtatatgtgatgtcactctgccgtatatgtgatgtcact

SEQ ID NO: 353 - DNA, 40 bp, Artificial Sequence
  Synthetic Sequence
agcatggagtcctccaggttagcatggagtcctccaggtt

SEQ ID NO: 354 - DNA, 42 bp, Artificial Sequence
  Synthetic Sequence
tcctcatggaagggttccccatcctcatggaagggttcccca

SEQ ID NO: 355 - DNA, 42 bp, Artificial Sequence
  Synthetic Sequence
aagaatcttgtcccgcaggtcaagaatcttgtcccgcaggtc

SEQ ID NO: 356 - DNA, 44 bp, Artificial Sequence
  Synthetic Sequence
aatgaaagcctaccatgtacaaaatgaaagcctaccatgtacaa

SEQ ID NO: 357 - DNA, 44 bp, Artificial Sequence
  Synthetic Sequence
aagaggtttcccgtgtatgtttaagaggtttcccgtgtatgttt

SEQ ID NO: 358 - DNA, 44 bp, Artificial Sequence
  Synthetic Sequence
aaagaagtgcaccatgtttgttaaagaagtgcaccatgtttgtt

SEQ ID NO: 359 - DNA, 34 bp, Artificial Sequence
  Synthetic Sequence
gagattggccatgtaatgagattggccatgtaat

SEQ ID NO: 360 - DNA, 42 bp, Artificial Sequence
  Synthetic Sequence
acaaaccacagtgtgctgctgacaaaccacagtgtgctgctg

SEQ ID NO: 361 - DNA, 42 bp, Artificial Sequence
  Synthetic Sequence
aaaaacgccccctggcttgaaaaaaacgccccctggcttgaa

SEQ ID NO: 362 - DNA, 44 bp, Artificial Sequence
  Synthetic Sequence
ttaaacatcactgcaagtcttattaaacatcactgcaagtctta

SEQ ID NO: 363 - DNA, 42 bp, Artificial Sequence
  Synthetic Sequence
agaatccttgcccaggtgcatagaatccttgcccaggtgcat

SEQ ID NO: 364 - DNA, 42 bp, Artificial Sequence
  Synthetic Sequence
tctcacccagggacaaaggattctcacccagggacaaaggat

SEQ ID NO: 365 - DNA, 42 bp, Artificial Sequence
  Synthetic Sequence
tagcacccagatagcaaggattagcacccagatagcaaggat

SEQ ID NO: 366 - DNA, 44 bp, Artificial Sequence
  Synthetic Sequence
tgcagaactgttcccgctgctatgcagaactgttcccgctgcta

SEQ ID NO: 367 - DNA, 42 bp, Artificial Sequence
  Synthetic Sequence
atagagtgcagaccagggtctatagagtgcagaccagggtct

SEQ ID NO: 368 - DNA, 42 bp, Artificial Sequence
  Synthetic Sequence
gaggaaaccagcaagtgttgagaggaaaccagcaagtgttga

SEQ ID NO: 369 - DNA, 42 bp, Artificial Sequence
  Synthetic Sequence
tctactcagaagggtgccttatctactcagaagggtgcctta

SEQ ID NO: 370 - DNA, 42 bp, Artificial Sequence
  Synthetic Sequence
ttcactccaaaaggtgcaaaattcactccaaaaggtgcaaaa

SEQ ID NO: 371 - DNA, 44 bp, Artificial Sequence
  Synthetic Sequence
tctactccaaaaggctacaatctctactccaaaaggctacaatc

SEQ ID NO: 372 - DNA, 42 bp, Artificial Sequence
  Synthetic Sequence
tctacccacagacgtaccaattctacccacagacgtaccaat

SEQ ID NO: 373 - DNA, 42 bp, Artificial Sequence
  Synthetic Sequence
tgtgattgccactctcctgagtgtgattgccactctcctgag

SEQ ID NO: 374 - DNA, 42 bp, Artificial Sequence
  Synthetic Sequence
tgactgcagagcaaaagacactgactgcagagcaaaagacac

SEQ ID NO: 375 - DNA, 42 bp, Artificial Sequence
  Synthetic Sequence
gacctcagctatgacagcactgacctcagctatgacagcact

SEQ ID NO: 376 - DNA, 42 bp, Artificial Sequence
  Synthetic Sequence
aaagtgccctcaaggctgagtaaagtgccctcaaggctgagt

SEQ ID NO: 377 - DNA, 44 bp, Artificial Sequence
  Synthetic Sequence
ataaatgacacctccctgtgaaataaatgacacctccctgtgaa

SEQ ID NO: 378 - DNA, 40 bp, Artificial Sequence
  Synthetic Sequence
ctactcacagaagtgtcaatctactcacagaagtgtcaat

SEQ ID NO: 379 - DNA, 42 bp, Artificial Sequence
  Synthetic Sequence
acgctccaaaagaaggcactcacgctccaaaagaaggcactc

SEQ ID NO: 380 - DNA, 44 bp, Artificial Sequence
  Synthetic Sequence
cagaaagtgctttcttttggagcagaaagtgctttcttttggag

SEQ ID NO: 381 - DNA, 36 bp, Artificial Sequence
  Synthetic Sequence
accctctgaaaggaagcaaccctctgaaaggaagca

SEQ ID NO: 382 - DNA, 44 bp, Artificial Sequence
  Synthetic Sequence
aaagtgcttcttacctccagataaagtgcttcttacctccagat

SEQ ID NO: 383 - DNA, 42 bp, Artificial Sequence
  Synthetic Sequence
agacagtgcttccatctagagagacagtgcttccatctagag

SEQ ID NO: 384 - DNA, 44 bp, Artificial Sequence
  Synthetic Sequence
aacactctaaagggatgcacgaaacactctaaagggatgcacga

SEQ ID NO: 385 - DNA, 44 bp, Artificial Sequence; identical to SEQ ID NO: 384
  Synthetic Sequence

SEQ ID NO: 386 - DNA, 44 bp, Artificial Sequence
  Synthetic Sequence
acactctaaaaggatgcacgatacactctaaaaggatgcacgat

SEQ ID NO: 387 - DNA, 42 bp, Artificial Sequence
  Synthetic Sequence
tccagcaaagggaagcgcttttccagcaaagggaagcgcttt

SEQ ID NO: 388 - DNA, 40 bp, Artificial Sequence
  Synthetic Sequence
aaagggcttccctttgcagaaaagggcttccctttgcaga

SEQ ID NO: 389 - DNA, 42 bp, Artificial Sequence
  Synthetic Sequence
acctctaaaggggagcgctttacctctaaaggggagcgcttt

SEQ ID NO: 390 - DNA, 44 bp, Artificial Sequence
  Synthetic Sequence
cactctaaagagaagcgctttgcactctaaagagaagcgctttg

SEQ ID NO: 391 - DNA, 42 bp, Artificial Sequence
  Synthetic Sequence
cagaaagtgcttccctccagacagaaagtgcttccctccaga

SEQ ID NO: 392 - DNA, 40 bp, Artificial Sequence
  Synthetic Sequence
gctccaaagggaagcgctttgctccaaagggaagcgcttt

SEQ ID NO: 393 - DNA, 42 bp, Artificial Sequence
  Synthetic Sequence
acactctgaagggaagcgcttacactctgaagggaagcgctt

SEQ ID NO: 394 - DNA, 42 bp, Artificial Sequence
  Synthetic Sequence
tcctctaaagagaagcgcttttcctctaaagagaagcgcttt

SEQ ID NO: 395 - DNA, 44 bp, Artificial Sequence
  Synthetic Sequence
agagaaagtgcttccctctagaagagaaagtgcttccctctaga

SEQ ID NO: 396 - DNA, 44 bp, Artificial Sequence
  Synthetic Sequence
gtaacactctaaaaggatgcacgtaacactctaaaaggatgcac

SEQ ID NO: 397 - DNA, 44 bp, Artificial Sequence
  Synthetic Sequence
aaacctctaaaaggatgcacttaaacctctaaaaggatgcactt

SEQ ID NO: 398 - DNA, 44 bp, Artificial Sequence
  Synthetic Sequence
atcctctaaaaagatgcactttatcctctaaaaagatgcacttt

SEQ ID NO: 399 - DNA, 44 bp, Artificial Sequence
  Synthetic Sequence
acactctaaagggaggcactttacactctaaagggaggcacttt

SEQ ID NO: 400 - DNA, 44 bp, Artificial Sequence
  Synthetic Sequence
acactctaaaaggaggcactttacactctaaaaggaggcacttt

SEQ ID NO: 401 - DNA, 44 bp, Artificial Sequence
  Synthetic Sequence
gaaagtgctcccttttggagaagaaagtgctcccttttggagaa

SEQ ID NO: 402 - DNA, 44 bp, Artificial Sequence
  Synthetic Sequence
acagtccaaagggaagcactttacagtccaaagggaagcacttt

SEQ ID NO: 403 - DNA, 42 bp, Artificial Sequence
  Synthetic Sequence
agaaagtacttccctctggagagaaagtacttccctctggag

SEQ ID NO: 404 - DNA, 42 bp, Artificial Sequence
  Synthetic Sequence
ccctctaaaaggaagcactttccctctaaaaggaagcacttt

SEQ ID NO: 405 - DNA, 44 bp, Artificial Sequence
  Synthetic Sequence
aaccctctaaaaggaagcacttaaccctctaaaaggaagcactt

SEQ ID NO: 406 - DNA, 44 bp, Artificial Sequence
  Synthetic Sequence
aacccaccaaagagaagcacttaacccaccaaagagaagcactt

SEQ ID NO: 407 - DNA, 44 bp, Artificial Sequence
  Synthetic Sequence
agaaagggcttccctttgtagaagaaagggcttccctttgtaga

SEQ ID NO: 408 - DNA, 42 bp, Artificial Sequence
  Synthetic Sequence
ccctcaaaaaggaagcactttccctcaaaaaggaagcacttt

SEQ ID NO: 409 - DNA, 44 bp, Artificial Sequence; identical to SEQ ID NO: 405
  Synthetic Sequence

SEQ ID NO: 410 - DNA, 44 bp, Artificial Sequence
  Synthetic Sequence
acactctaaagggaagcactttacactctaaagggaagcacttt

SEQ ID NO: 411 - DNA, 44 bp, Artificial Sequence
  Synthetic Sequence
actctaaagggaagcactttgtactctaaagggaagcactttgt

SEQ ID NO: 412 - DNA, 44 bp, Artificial Sequence
  Synthetic Sequence
acactctaaagggaagtgcgttacactctaaagggaagtgcgtt

SEQ ID NO: 413 - DNA, 44 bp, Artificial Sequence
  Synthetic Sequence
aacactctaaagggaaccatttaacactctaaagggaaccattt

SEQ ID NO: 414 - DNA, 40 bp, Artificial Sequence
  Synthetic Sequence
cctctatagggaagcgcgttcctctatagggaagcgcgtt

SEQ ID NO: 415 - DNA, 40 bp, Artificial Sequence
  Synthetic Sequence
actccaaagggaagcgccttactccaaagggaagcgcctt

SEQ ID NO: 416 - DNA, 44 bp, Artificial Sequence
  Synthetic Sequence
gagaaagtgcttccctttgtaggagaaagtgcttccctttgtag

SEQ ID NO: 417 - DNA, 42 bp, Artificial Sequence
  Synthetic Sequence
agaaagtgcatccctctggagagaaagtgcatccctctggag

SEQ ID NO: 418 - DNA, 40 bp, Artificial Sequence
  Synthetic Sequence
gctctaaagggaagcgccttgctctaaagggaagcgcctt

SEQ ID NO: 419 - DNA, 42 bp, Artificial Sequence
  Synthetic Sequence
agaaagtgcttccctctagagagaaagtgcttccctctagag

SEQ ID NO: 420 - DNA, 44 bp, Artificial Sequence
  Synthetic Sequence
aacagaaagtgcttccctcaagaacagaaagtgcttccctcaag

SEQ ID NO: 421 - DNA, 42 bp, Artificial Sequence
  Synthetic Sequence
gcctctaaaaggaagcactttgcctctaaaaggaagcacttt

SEQ ID NO: 422 - DNA, 42 bp, Artificial Sequence
  Synthetic Sequence
aacagaaagcgcttccctctaaacagaaagcgcttccctcta

SEQ ID NO: 423 - DNA, 42 bp, Artificial Sequence
  Synthetic Sequence
agaaagggcttccctttgcagagaaagggcttccctttgcag

SEQ ID NO: 424 - DNA, 44 bp, Artificial Sequence
  Synthetic Sequence
caacaaaatcactagtcttccacaacaaaatcactagtcttcca

SEQ ID NO: 425 - DNA, 44 bp, Artificial Sequence; identical to SEQ ID NO: 8
  Synthetic Sequence

SEQ ID NO: 426 - DNA, 40 bp, Artificial Sequence
  Synthetic Sequence
actttcggttatctagctttactttcggttatctagcttt

SEQ ID NO: 427 - DNA, 40 bp, Artificial Sequence
  Synthetic Sequence
aggccgggacaagtgcaataaggccgggacaagtgcaata

SEQ ID NO: 428 - DNA, 42 bp, Artificial Sequence
  Synthetic Sequence
ctacctgcacgaacagcacttctacctgcacgaacagcactt

SEQ ID NO: 429 - DNA, 44 bp, Artificial Sequence
  Synthetic Sequence
tgctcaataaatacccgttgaatgctcaataaatacccgttgaa

SEQ ID NO: 430 - DNA, 44 bp, Artificial Sequence
  Synthetic Sequence
gcaaaaatgtgctagtgccaaagcaaaaatgtgctagtgccaaa

SEQ ID NO: 431 - DNA, 44 bp, Artificial Sequence
  Synthetic Sequence
aacaatacaacttactacctcaaacaatacaacttactacctca

SEQ ID NO: 432 - DNA, 42 bp, Artificial Sequence
  Synthetic Sequence
cacaagatcggatctacgggtcacaagatcggatctacgggt

SEQ ID NO: 433 - DNA, 38 bp, Artificial Sequence
  Synthetic Sequence
caaggtcggttctacgggtcaaggtcggttctacgggt

SEQ ID NO: 434 - DNA, 40 bp, Artificial Sequence
  Synthetic Sequence
tgttgcagcgcttcatgttttgttgcagcgcttcatgttt

SEQ ID NO: 435 - DNA, 42 bp, Artificial Sequence; identical to SEQ ID NO: 281
  Synthetic Sequence

SEQ ID NO: 436 - DNA, 44 bp, Artificial Sequence; identical to SEQ ID NO: 263
  Synthetic Sequence

SEQ ID NO: 437 - DNA, 40 bp, Artificial Sequence; identical to SEQ ID NO: 283
  Synthetic Sequence

SEQ ID NO: 438 - DNA, 36 bp, Artificial Sequence; identical to SEQ ID NO: 282
  Synthetic Sequence

SEQ ID NO: 439 - DNA, 44 bp, Artificial Sequence
  Synthetic Sequence
acacttactgagcacctactagacacttactgagcacctactag

SEQ ID NO: 440 - DNA, 38 bp, Artificial Sequence
  Synthetic Sequence
actggaggaagggcccagaactggaggaagggcccaga

SEQ ID NO: 441 - DNA, 36 bp, Artificial Sequence
  Synthetic Sequence
accctcatgcccctcaagaccctcatgcccctcaag

SEQ ID NO: 442 - DNA, 42 bp, Artificial Sequence; identical to SEQ ID NO: 125
  Synthetic Sequence

SEQ ID NO: 443 - DNA, 42 bp, Artificial Sequence
  Synthetic Sequence
agaaaggcagcaggtcgtataagaaaggcagcaggtcgtata

SEQ ID NO: 444 - DNA, 42 bp, Artificial Sequence; identical to SEQ ID NO: 286
  Synthetic Sequence

SEQ ID NO: 445 - DNA, 44 bp, Artificial Sequence
  Synthetic Sequence
aaaaaggttagctgggtgtgttaaaaaggttagctgggtgtgtt

SEQ ID NO: 446 - DNA, 42 bp, Artificial Sequence
  Synthetic Sequence
tctctgcaggccctgtgcttttctctgcaggccctgtgcttt

SEQ ID NO: 447 - DNA, 38 bp, Artificial Sequence; identical to SEQ ID NO: 290
  Synthetic Sequence

SEQ ID NO: 448 - DNA, 40 bp, Artificial Sequence
  Synthetic Sequence
aaaagtaactagcacaccacaaaagtaactagcacaccac

SEQ ID NO: 449 - DNA, 42 bp, Artificial Sequence; identical to SEQ ID NO: 162
  Synthetic Sequence

SEQ ID NO: 450 - DNA, 40 bp, Artificial Sequence
  Synthetic Sequence
tgtccgtggttctaccctgttgtccgtggttctaccctgt

SEQ ID NO: 451 - DNA, 44 bp, Artificial Sequence; identical to SEQ ID NO: 291
  Synthetic Sequence

SEQ ID NO: 452 - DNA, 42 bp, Artificial Sequence
  Synthetic Sequence
agactagatatggaagggtgaagactagatatggaagggtga

SEQ ID NO: 453 - DNA, 44 bp, Artificial Sequence
  Synthetic Sequence
aaaggcatcatataggagctgaaaaggcatcatataggagctga

SEQ ID NO: 454 - DNA, 44 bp, Artificial Sequence; identical to SEQ ID NO: 173
  Synthetic Sequence

SEQ ID NO: 455 - DNA, 44 bp, Artificial Sequence; identical to SEQ ID NO: 293
  Synthetic Sequence

SEQ ID NO: 456 - DNA, 42 bp, Artificial Sequence; identical to SEQ ID NO: 294
  Synthetic Sequence

SEQ ID NO: 457 - DNA, 44 bp, Artificial Sequence; identical to SEQ ID NO: 295
  Synthetic Sequence

SEQ ID NO: 458 - DNA, 42 bp, Artificial Sequence
  Synthetic Sequence
actgaccgaccgaccgatcgaactgaccgaccgaccgatcga

SEQ ID NO: 459 - DNA, 42 bp, Artificial Sequence; identical to SEQ ID NO: 296
  Synthetic Sequence

SEQ ID NO: 460 - DNA, 40 bp, Artificial Sequence
  Synthetic Sequence
tctgggcacacggagggagatctgggcacacggagggaga

SEQ ID NO: 461 - DNA, 42 bp, Artificial Sequence
  Synthetic Sequence
acggtcaggctttggctagatacggtcaggctttggctagat

SEQ ID NO: 462 - DNA, 38 bp, Artificial Sequence
  Synthetic Sequence
actggactaggggtcagcaactggactaggggtcagca

SEQ ID NO: 463 - DNA, 42 bp, Artificial Sequence
  Synthetic Sequence
agaggcaggcactcaggcagaagaggcaggcactcaggcaga

SEQ ID NO: 464 - DNA, 38 bp, Artificial Sequence
  Synthetic Sequence
tgggcgacccagagggacatgggcgacccagagggaca

SEQ ID NO: 465 - DNA, 42 bp, Artificial Sequence
  Synthetic Sequence
agaggttaagacagcagggctagaggttaagacagcagggct

SEQ ID NO: 466 - DNA, 42 bp, Artificial Sequence
  Synthetic Sequence
agcaagcccagaccgcaaaaaagcaagcccagaccgcaaaaa

SEQ ID NO: 467 - DNA, 44 bp, Artificial Sequence
  Synthetic Sequence
atgctttttggggtaagggcttatgctttttggggtaagggctt

SEQ ID NO: 468 - DNA, 44 bp, Artificial Sequence; identical to SEQ ID NO: 230
  Synthetic Sequence

SEQ ID NO: 469 - DNA, 42 bp, Artificial Sequence
  Synthetic Sequence
tgtaagtgctcgtaatgcagttgtaagtgctcgtaatgcagt

SEQ ID NO: 470 - DNA, 44 bp, Artificial Sequence
  Synthetic Sequence
gtgaaagtgtatgggctttgtggtgaaagtgtatgggctttgtg

SEQ ID NO: 471 - DNA, 40 bp, Artificial Sequence
  Synthetic Sequence
aacaaaatcactagtcttccaacaaaatcactagtcttcc

SEQ ID NO: 472 - DNA, 44 bp, Artificial Sequence
  Synthetic Sequence
tatggcagactgtgatttgttgtatggcagactgtgatttgttg

SEQ ID NO: 473 - DNA, 38 bp, Artificial Sequence
  Synthetic Sequence
aggctcaaagggctcctcaaggctcaaagggctcctca

SEQ ID NO: 474 - DNA, 42 bp, Artificial Sequence
  Synthetic Sequence
tactatgcaacctactactcttactatgcaacctactactct

SEQ ID NO: 475 - DNA, 44 bp, Artificial Sequence; identical to SEQ ID NO: 157
  Synthetic Sequence

SEQ ID NO: 476 - DNA, 42 bp, Artificial Sequence
  Synthetic Sequence
tactagactgtgagctcctcgtactagactgtgagctcctcg

SEQ ID NO: 477 - DNA, 42 bp, Artificial Sequence
  Synthetic Sequence
ctcaaggagcctcagtctagtctcaaggagcctcagtctagt

SEQ ID NO: 478 - DNA, 44 bp, Artificial Sequence
  Synthetic Sequence
cttcagctatcacagtactgtacttcagctatcacagtactgta

SEQ ID NO: 479 - DNA, 44 bp, Artificial Sequence; identical to SEQ ID NO: 122
  Synthetic Sequence

SEQ ID NO: 480 - DNA, 44 bp, Artificial Sequence; identical to SEQ ID NO: 123
  Synthetic Sequence

SEQ ID NO: 481 - DNA, 44 bp, Artificial Sequence; identical to SEQ ID NO: 124
  Synthetic Sequence

SEQ ID NO: 482 - DNA, 42 bp, Artificial Sequence; identical to SEQ ID NO: 126
  Synthetic Sequence

SEQ ID NO: 483 - DNA, 44 bp, Artificial Sequence; identical to SEQ ID NO: 127
  Synthetic Sequence

SEQ ID NO: 484 - DNA, 42 bp, Artificial Sequence; identical to SEQ ID NO: 129
  Synthetic Sequence

SEQ ID NO: 485 - DNA, 40 bp, Artificial Sequence
  Synthetic Sequence
aacaaaatcacaagtcttccaacaaaatcacaagtcttcc

SEQ ID NO: 486 - DNA, 44 bp, Artificial Sequence; identical to SEQ ID NO: 8
  Synthetic Sequence

SEQ ID NO: 487 - DNA, 44 bp, Artificial Sequence; identical to SEQ ID NO: 138
  Synthetic Sequence

SEQ ID NO: 488 - DNA, 44 bp, Artificial Sequence
  Synthetic Sequence
acacaaattcggttctacagggacacaaattcggttctacaggg

SEQ ID NO: 489 - DNA, 44 bp, Artificial Sequence; identical to SEQ ID NO: 183
  Synthetic Sequence

SEQ ID NO: 490 - DNA, 44 bp, Artificial Sequence; identical to SEQ ID NO: 184
  Synthetic Sequence

SEQ ID NO: 491 - DNA, 44 bp, Artificial Sequence; identical to SEQ ID NO: 186
  Synthetic Sequence

SEQ ID NO: 492 - DNA, 44 bp, Artificial Sequence; identical to SEQ ID NO: 200
  Synthetic Sequence

SEQ ID NO: 493 - DNA, 44 bp, Artificial Sequence; identical to SEQ ID NO: 218
  Synthetic Sequence

SEQ ID NO: 494 - DNA, 44 bp, Artificial Sequence; identical to SEQ ID NO: 217
  Synthetic Sequence

SEQ ID NO: 495 - DNA, 44 bp, Artificial Sequence; identical to SEQ ID NO: 232
  Synthetic Sequence

SEQ ID NO: 496 - DNA, 44 bp, Artificial Sequence; identical to SEQ ID NO: 243
  Synthetic Sequence

SEQ ID NO: 497 - DNA, 42 bp, Artificial Sequence; identical to SEQ ID NO: 249
  Synthetic Sequence

SEQ ID NO: 498 - DNA, 42 bp, Artificial Sequence; identical to SEQ ID NO: 250
  Synthetic Sequence

SEQ ID NO: 499 - DNA, 42 bp, Artificial Sequence; identical to SEQ ID NO: 251
  Synthetic Sequence

SEQ ID NO: 500 - DNA, 42 bp, Artificial Sequence; identical to SEQ ID NO: 252
  Synthetic Sequence

SEQ ID NO: 501 - DNA, 42 bp, Artificial Sequence; identical to SEQ ID NO: 253
  Synthetic Sequence

SEQ ID NO: 502 - DNA, 44 bp, Artificial Sequence; identical to SEQ ID NO: 254
  Synthetic Sequence

SEQ ID NO: 503 - DNA, 42 bp, Artificial Sequence; identical to SEQ ID NO: 256
  Synthetic Sequence

SEQ ID NO: 504 - DNA, 42 bp, Artificial Sequence; identical to SEQ ID NO: 255
  Synthetic Sequence

SEQ ID NO: 505 - DNA, 44 bp, Artificial Sequence; identical to SEQ ID NO: 257
  Synthetic Sequence

SEQ ID NO: 506 - DNA, 44 bp, Artificial Sequence; identical to SEQ ID NO: 261
  Synthetic Sequence

SEQ ID NO: 507 - DNA, 42 bp, Artificial Sequence; identical to SEQ ID NO: 260
  Synthetic Sequence

SEQ ID NO: 508 - DNA, 40 bp, Artificial Sequence; identical to SEQ ID NO: 262
  Synthetic Sequence

SEQ ID NO: 509 - DNA, 44 bp, Artificial Sequence; identical to SEQ ID NO: 274
  Synthetic Sequence

SEQ ID NO: 510 - DNA, 40 bp, Artificial Sequence; identical to SEQ ID NO: 277
  Synthetic Sequence

SEQ ID NO: 511 - DNA, 44 bp, Artificial Sequence; identical to SEQ ID NO: 273
  Synthetic Sequence

SEQ ID NO: 512 - DNA, 42 bp, Artificial Sequence; identical to SEQ ID NO: 275
  Synthetic Sequence

SEQ ID NO: 513 - DNA, 44 bp, Artificial Sequence; identical to SEQ ID NO: 272
  Synthetic Sequence

SEQ ID NO: 514 - DNA, 42 bp, Artificial Sequence; identical to SEQ ID NO: 271
  Synthetic Sequence

SEQ ID NO: 515 - DNA, 42 bp, Artificial Sequence
  Synthetic Sequence
agctatgccagcatcttgcctagctatgccagcatcttgcct

SEQ ID NO: 516 - DNA, 40 bp, Artificial Sequence; identical to SEQ ID NO: 279
  Synthetic Sequence

SEQ ID NO: 517 - DNA, 38 bp, Artificial Sequence; identical to SEQ ID NO: 288
  Synthetic Sequence

SEQ ID NO: 518 - DNA, 44 bp, Artificial Sequence
  Synthetic Sequence
caatcagctaattacactgcctcaatcagctaattacactgcct

SEQ ID NO: 519 - DNA, 44 bp, Artificial Sequence; identical to SEQ ID NO: 301
  Synthetic Sequence

SEQ ID NO: 520 - DNA, 42 bp, Artificial Sequence; identical to SEQ ID NO: 299
  Synthetic Sequence

SEQ ID NO: 521 - DNA, 40 bp, Artificial Sequence; identical to SEQ ID NO: 427
  Synthetic Sequence

SEQ ID NO: 522 - DNA, 42 bp, Artificial Sequence; identical to SEQ ID NO: 428
  Synthetic Sequence

SEQ ID NO: 523 - DNA, 44 bp, Artificial Sequence
  Synthetic Sequence
agcaaaaatgtgctagtgccaaagcaaaaatgtgctagtgccaa

SEQ ID NO: 524 - DNA, 44 bp, Artificial Sequence; identical to SEQ ID NO: 431
  Synthetic Sequence

SEQ ID NO: 525 - DNA, 42 bp, Artificial Sequence; identical to SEQ ID NO: 432
  Synthetic Sequence

SEQ ID NO: 526 - DNA, 38 bp, Artificial Sequence; identical to SEQ ID NO: 433
  Synthetic Sequence

SEQ ID NO: 527 - DNA, 42 bp, Artificial Sequence; identical to SEQ ID NO: 131
  Synthetic Sequence

SEQ ID NO: 528 - DNA, 44 bp, Artificial Sequence; identical to SEQ ID NO: 132
  Synthetic Sequence

SEQ ID NO: 529 - DNA, 42 bp, Artificial Sequence; identical to SEQ ID NO: 16
  Synthetic Sequence

SEQ ID NO: 530 - DNA, 42 bp, Artificial Sequence; identical to SEQ ID NO: 136
  Synthetic Sequence

SEQ ID NO: 531 - DNA, 42 bp, Artificial Sequence; identical to SEQ ID NO: 137
  Synthetic Sequence

SEQ ID NO: 532 - DNA, 44 bp, Artificial Sequence; identical to SEQ ID NO: 140
  Synthetic Sequence

SEQ ID NO: 533 - DNA, 44 bp, Artificial Sequence; identical to SEQ ID NO: 141
  Synthetic Sequence

SEQ ID NO: 534 - DNA, 42 bp, Artificial Sequence; identical to SEQ ID NO: 142
  Synthetic Sequence

SEQ ID NO: 535 - DNA, 42 bp, Artificial Sequence; identical to SEQ ID NO: 143
  Synthetic Sequence

SEQ ID NO: 536 - DNA, 42 bp, Artificial Sequence; identical to SEQ ID NO: 145
  Synthetic Sequence

SEQ ID NO: 537 - DNA, 42 bp, Artificial Sequence; identical to SEQ ID NO: 144
  Synthetic Sequence

SEQ ID NO: 538 - DNA, 44 bp, Artificial Sequence; identical to SEQ ID NO: 146
  Synthetic Sequence

SEQ ID NO: 539 - DNA, 44 bp, Artificial Sequence; identical to SEQ ID NO: 147
  Synthetic Sequence

SEQ ID NO: 540 - DNA, 42 bp, Artificial Sequence; identical to SEQ ID NO: 148
  Synthetic Sequence

SEQ ID NO: 541 - DNA, 44 bp, Artificial Sequence; identical to SEQ ID NO: 150
  Synthetic Sequence

SEQ ID NO: 542 - DNA, 44 bp, Artificial Sequence; identical to SEQ ID NO: 151
  Synthetic Sequence

SEQ ID NO: 543 - DNA, 42 bp, Artificial Sequence; identical to SEQ ID NO: 152
  Synthetic Sequence

SEQ ID NO: 544 - DNA, 44 bp, Artificial Sequence; identical to SEQ ID NO: 153
  Synthetic Sequence

SEQ ID NO: 545 - DNA, 40 bp, Artificial Sequence; identical to SEQ ID NO: 155
  Synthetic Sequence

SEQ ID NO: 546 - DNA, 44 bp, Artificial Sequence; identical to SEQ ID NO: 156
  Synthetic Sequence

SEQ ID NO: 547 - DNA, 44 bp, Artificial Sequence; identical to SEQ ID NO: 158
  Synthetic Sequence

SEQ ID NO: 548 - DNA, 44 bp, Artificial Sequence; identical to SEQ ID NO: 159
  Synthetic Sequence

SEQ ID NO: 549 - DNA, 34 bp, Artificial Sequence; identical to SEQ ID NO: 160
  Synthetic Sequence

SEQ ID NO: 550 - DNA, 36 bp, Artificial Sequence; identical to SEQ ID NO: 161
  Synthetic Sequence

SEQ ID NO: 551 - DNA, 44 bp, Artificial Sequence; identical to SEQ ID NO: 163
  Synthetic Sequence

SEQ ID NO: 552 - DNA, 40 bp, Artificial Sequence; identical to SEQ ID NO: 165
  Synthetic Sequence

SEQ ID NO: 553 - DNA, 44 bp, Artificial Sequence; identical to SEQ ID NO: 164
  Synthetic Sequence

SEQ ID NO: 554 - DNA, 44 bp, Artificial Sequence; identical to SEQ ID NO: 166
  Synthetic Sequence

SEQ ID NO: 555 - DNA, 44 bp, Artificial Sequence; identical to SEQ ID NO: 167
  Synthetic Sequence

SEQ ID NO: 556 - DNA, 42 bp, Artificial Sequence; identical to SEQ ID NO: 168
  Synthetic Sequence

SEQ ID NO: 557 - DNA, 44 bp, Artificial Sequence; identical to SEQ ID NO: 169
  Synthetic Sequence

SEQ ID NO: 558 - DNA, 42 bp, Artificial Sequence; identical to SEQ ID NO: 175
  Synthetic Sequence

SEQ ID NO: 559 - DNA, 42 bp, Artificial Sequence; identical to SEQ ID NO: 177
  Synthetic Sequence

SEQ ID NO: 560 - DNA, 40 bp, Artificial Sequence; identical to SEQ ID NO: 178
  Synthetic Sequence

SEQ ID NO: 561 - DNA, 42 bp, Artificial Sequence; identical to SEQ ID NO: 179
  Synthetic Sequence

SEQ ID NO: 562 - DNA, 44 bp, Artificial Sequence; identical to SEQ ID NO: 189
  Synthetic Sequence

SEQ ID NO: 563 - DNA, 42 bp, Artificial Sequence; identical to SEQ ID NO: 187
  Synthetic Sequence

SEQ ID NO: 564 - DNA, 42 bp, Artificial Sequence; identical to SEQ ID NO: 188
  Synthetic Sequence

SEQ ID NO: 565 - DNA, 44 bp, Artificial Sequence; identical to SEQ ID NO: 193
  Synthetic Sequence

SEQ ID NO: 566 - DNA, 42 bp, Artificial Sequence; identical to SEQ ID NO: 194
  Synthetic Sequence

SEQ ID NO: 567 - DNA, 36 bp, Artificial Sequence; identical to SEQ ID NO: 195
  Synthetic Sequence

SEQ ID NO: 568 - DNA, 44 bp, Artificial Sequence; identical to SEQ ID NO: 196
  Synthetic Sequence

SEQ ID NO: 569 - DNA, 40 bp, Artificial Sequence; identical to SEQ ID NO: 197
  Synthetic Sequence

SEQ ID NO: 570 - DNA, 44 bp, Artificial Sequence; identical to SEQ ID NO: 202
  Synthetic Sequence

SEQ ID NO: 571 - DNA, 42 bp, Artificial Sequence; identical to SEQ ID NO: 203
  Synthetic Sequence

SEQ ID NO: 572 - DNA, 42 bp, Artificial Sequence; identical to SEQ ID NO: 205
  Synthetic Sequence

SEQ ID NO: 573 - DNA, 42 bp, Artificial Sequence; identical to SEQ ID NO: 206
  Synthetic Sequence

SEQ ID NO: 574 - DNA, 44 bp, Artificial Sequence; identical to SEQ ID NO: 208
  Synthetic Sequence

SEQ ID NO: 575 - DNA, 42 bp, Artificial Sequence; identical to SEQ ID NO: 209
  Synthetic Sequence

SEQ ID NO: 576 - DNA, 42 bp, Artificial Sequence; identical to SEQ ID NO: 210
  Synthetic Sequence

SEQ ID NO: 577 - DNA, 42 bp, Artificial Sequence; identical to SEQ ID NO: 214
  Synthetic Sequence

SEQ ID NO: 578 - DNA, 44 bp, Artificial Sequence; identical to SEQ ID NO: 222
  Synthetic Sequence

SEQ ID NO: 579 - DNA, 44 bp, Artificial Sequence; identical to SEQ ID NO: 219
  Synthetic Sequence

SEQ ID NO: 580 - DNA, 44 bp, Artificial Sequence; identical to SEQ ID NO: 221
  Synthetic Sequence

SEQ ID NO: 581 - DNA, 44 bp, Artificial Sequence; identical to SEQ ID NO: 225
  Synthetic Sequence

SEQ ID NO: 582 - DNA, 44 bp, Artificial Sequence; identical to SEQ ID NO: 226
  Synthetic Sequence

SEQ ID NO: 583 - DNA, 42 bp, Artificial Sequence; identical to SEQ ID NO: 227
  Synthetic Sequence

SEQ ID NO: 584 - DNA, 44 bp, Artificial Sequence; identical to SEQ ID NO: 228
  Synthetic Sequence

SEQ ID NO: 585 - DNA, 44 bp, Artificial Sequence; identical to SEQ ID NO: 229
  Synthetic Sequence

SEQ ID NO: 586 - DNA, 42 bp, Artificial Sequence; identical to SEQ ID NO: 233
  Synthetic Sequence

SEQ ID NO: 587 - DNA, 44 bp, Artificial Sequence
  Synthetic Sequence
aggcaaaggatgacaaagggaaaggcaaaggatgacaaagggaa

SEQ ID NO: 588 - DNA, 40 bp, Artificial Sequence; identical to SEQ ID NO: 235
  Synthetic Sequence

SEQ ID NO: 589 - DNA, 42 bp, Artificial Sequence; identical to SEQ ID NO: 236
  Synthetic Sequence

SEQ ID NO: 590 - DNA, 40 bp, Artificial Sequence; identical to SEQ ID NO: 237
  Synthetic Sequence

SEQ ID NO: 591 - DNA, 42 bp, Artificial Sequence; identical to SEQ ID NO: 239
  Synthetic Sequence

SEQ ID NO: 592 - DNA, 42 bp, Artificial Sequence
  Synthetic Sequence
atccagtcagttcctgatgcaatccagtcagttcctgatgca

SEQ ID NO: 593 - DNA, 42 bp, Artificial Sequence; identical to SEQ ID NO: 241
  Synthetic Sequence

SEQ ID NO: 594 - DNA, 42 bp, Artificial Sequence; identical to SEQ ID NO: 242
  Synthetic Sequence

SEQ ID NO: 595 - DNA, 44 bp, Artificial Sequence; identical to SEQ ID NO: 245
  Synthetic Sequence

SEQ ID NO: 596 - DNA, 42 bp, Artificial Sequence; identical to SEQ ID NO: 246
  Synthetic Sequence

SEQ ID NO: 597 - DNA, 42 bp, Artificial Sequence; identical to SEQ ID NO: 247
  Synthetic Sequence

SEQ ID NO: 598 - DNA, 44 bp, Artificial Sequence
  Synthetic Sequence
aaaaagtgcccccatagtttgaaaaaagtgcccccatagtttga

SEQ ID NO: 599 - DNA, 42 bp, Artificial Sequence
  Synthetic Sequence
agagagggcctccactttgatagagagggcctccactttgat

SEQ ID NO: 600 - DNA, 44 bp, Artificial Sequence
  Synthetic Sequence
gcacacaaagtggaagcactttgcacacaaagtggaagcacttt

SEQ ID NO: 601 - DNA, 42 bp, Artificial Sequence
  Synthetic Sequence
caaaagagcccccagtttgagcaaaagagcccccagtttgag

SEQ ID NO: 602 - DNA, 44 bp, Artificial Sequence
  Synthetic Sequence
acactcaaaacctggcggcactacactcaaaacctggcggcact

SEQ ID NO: 603 - DNA, 42 bp, Artificial Sequence; identical to SEQ ID NO: 258
  Synthetic Sequence

SEQ ID NO: 604 - DNA, 44 bp, Artificial Sequence
  Synthetic Sequence
catgcatacatgcacacatacacatgcatacatgcacacataca

SEQ ID NO: 605 - DNA, 38 bp, Artificial Sequence
  Synthetic Sequence
ggaagaacagccctcctctggaagaacagccctcctct

SEQ ID NO: 606 - DNA, 44 bp, Artificial Sequence
  Synthetic Sequence
atgtatgtgggacggtaaaccaatgtatgtgggacggtaaacca

SEQ ID NO: 607 - DNA, 42 bp, Artificial Sequence
  Synthetic Sequence
gaagagagcttgcccttgcatgaagagagcttgcccttgcat

SEQ ID NO: 608 - DNA, 44 bp, Artificial Sequence; identical to SEQ ID NO: 280
  Synthetic Sequence

SEQ ID NO: 609 - DNA, 42 bp, Artificial Sequence; identical to SEQ ID NO: 211
  Synthetic Sequence

SEQ ID NO: 610 - DNA, 42 bp, Artificial Sequence
  Synthetic Sequence
caacaaacatttaatgaggcccaacaaacatttaatgaggcc

SEQ ID NO: 611 - DNA, 44 bp, Artificial Sequence; identical to SEQ ID NO: 342
  Synthetic Sequence

SEQ ID NO: 612 - DNA, 44 bp, Artificial Sequence
  Synthetic Sequence
acggcattaccagacagtattaacggcattaccagacagtatta

SEQ ID NO: 613 - DNA, 44 bp, Artificial Sequence; identical to SEQ ID NO: 343
  Synthetic Sequence

SEQ ID NO: 614 - DNA, 44 bp, Artificial Sequence
  Synthetic Sequence
cattaggaacacatcgcaaaaacattaggaacacatcgcaaaaa

SEQ ID NO: 615 - DNA, 44 bp, Artificial Sequence; identical to SEQ ID NO: 305
  Synthetic Sequence

SEQ ID NO: 616 - DNA, 44 bp, Artificial Sequence
  Synthetic Sequence
tccaaaacatgaattgctgctgtccaaaacatgaattgctgctg

SEQ ID NO: 617 - DNA, 42 bp, Artificial Sequence; identical to SEQ ID NO: 338
  Synthetic Sequence

SEQ ID NO: 618 - DNA, 44 bp, Artificial Sequence; identical to SEQ ID NO: 341
  Synthetic Sequence

SEQ ID NO: 619 - DNA, 44 bp, Artificial Sequence
  Synthetic Sequence
aactcagtaatggtaacggtttaactcagtaatggtaacggttt

SEQ ID NO: 620 - DNA, 42 bp, Artificial Sequence; identical to SEQ ID NO: 128
  Synthetic Sequence

SEQ ID NO: 621 - DNA, 42 bp, Artificial Sequence; identical to SEQ ID NO: 129
  Synthetic Sequence

SEQ ID NO: 622 - DNA, 42 bp, Artificial Sequence; identical to SEQ ID NO: 130
  Synthetic Sequence

SEQ ID NO: 623 - DNA, 44 bp, Artificial Sequence; identical to SEQ ID NO: 183
  Synthetic Sequence

SEQ ID NO: 624 - DNA, 42 bp, Artificial Sequence; identical to SEQ ID NO: 250
  Synthetic Sequence

SEQ ID NO: 625 - DNA, 42 bp, Artificial Sequence; identical to SEQ ID NO: 256
  Synthetic Sequence

SEQ ID NO: 626 - DNA, 44 bp, Artificial Sequence; identical to SEQ ID NO: 261
  Synthetic Sequence

SEQ ID NO: 627 - DNA, 44 bp, Artificial Sequence; identical to SEQ ID NO: 272
  Synthetic Sequence

SEQ ID NO: 628 - DNA, 42 bp, Artificial Sequence; identical to SEQ ID NO: 271
  Synthetic Sequence

SEQ ID NO: 629 - DNA, 44 bp, Artificial Sequence; identical to SEQ ID NO: 273
  Synthetic Sequence

SEQ ID NO: 630 - DNA, 40 bp, Artificial Sequence
  Synthetic Sequence
acaagatcggatctacgggtacaagatcggatctacgggt

SEQ ID NO: 631 - DNA, 38 bp, Artificial Sequence; identical to SEQ ID NO: 433
  Synthetic Sequence

SEQ ID NO: 632 - DNA, 44 bp, Artificial Sequence; identical to SEQ ID NO: 132
  Synthetic Sequence

SEQ ID NO: 633 - DNA, 38 bp, Artificial Sequence
  Synthetic Sequence
gcattcaccgcgtgccttagcattcaccgcgtgcctta

SEQ ID NO: 634 - DNA, 42 bp, Artificial Sequence; identical to SEQ ID NO: 142
  Synthetic Sequence

SEQ ID NO: 635 - DNA, 42 bp, Artificial Sequence; identical to SEQ ID NO: 143
  Synthetic Sequence

SEQ ID NO: 636 - DNA, 42 bp, Artificial Sequence; identical to SEQ ID NO: 145
  Synthetic Sequence

SEQ ID NO: 637 - DNA, 42 bp, Artificial Sequence; identical to SEQ ID NO: 144
  Synthetic Sequence

SEQ ID NO: 638 - DNA, 38 bp, Artificial Sequence
  Synthetic Sequence
caagctcagacggatccgacaagctcagacggatccga

SEQ ID NO: 639 - DNA, 44 bp, Artificial Sequence; identical to SEQ ID NO: 147
  Synthetic Sequence

SEQ ID NO: 640 - DNA, 44 bp, Artificial Sequence; identical to SEQ ID NO: 150
  Synthetic Sequence

SEQ ID NO: 641 - DNA, 44 bp, Artificial Sequence
  Synthetic Sequence
catacagctagataaccaaagacatacagctagataaccaaaga

SEQ ID NO: 642 - DNA, 40 bp, Artificial Sequence; identical to SEQ ID NO: 426
  Synthetic Sequence

SEQ ID NO: 643 - DNA, 42 bp, Artificial Sequence; identical to SEQ ID NO: 152
  Synthetic Sequence

SEQ ID NO: 644 - DNA, 44 bp, Artificial Sequence; identical to SEQ ID NO: 153
  Synthetic Sequence

SEQ ID NO: 645 - DNA, 40 bp, Artificial Sequence; identical to SEQ ID NO: 155
  Synthetic Sequence

SEQ ID NO: 646 - DNA, 44 bp, Artificial Sequence; identical to SEQ ID NO: 156
  Synthetic Sequence

SEQ ID NO: 647 - DNA, 44 bp, Artificial Sequence; identical to SEQ ID NO: 158
  Synthetic Sequence

SEQ ID NO: 648 - DNA, 44 bp, Artificial Sequence; identical to SEQ ID NO: 159
  Synthetic Sequence

SEQ ID NO: 649 - DNA, 34 bp, Artificial Sequence; identical to SEQ ID NO: 160
  Synthetic Sequence

SEQ ID NO: 650 - DNA, 44 bp, +Artificial Sequence
  Synthetic Sequence
ctaccatagggtaaaaccactgctaccatagggtaaaaccactg

SEQ ID NO: 651 - DNA, 44 bp, Artificial Sequence
  Synthetic Sequence
tccgtggttctaccctgtggtatccgtggttctaccctgtggta

SEQ ID NO: 652 - DNA, 44 bp, Artificial Sequence; identical to SEQ ID NO: 163
  Synthetic Sequence

SEQ ID NO: 653 - DNA, 40 bp, Artificial Sequence; identical to SEQ ID NO: 165
  Synthetic Sequence

SEQ ID NO: 654 - DNA, 44 bp, Artificial Sequence
  Synthetic Sequence
ccataaagtaggaaacactacaccataaagtaggaaacactaca

SEQ ID NO: 655 - DNA, 44 bp, Artificial Sequence; identical to SEQ ID NO: 167
  Synthetic Sequence

SEQ ID NO: 656 - DNA, 42 bp, Artificial Sequence; identical to SEQ ID NO: 168
  Synthetic Sequence

SEQ ID NO: 657 - DNA, 44 bp, Artificial Sequence; identical to SEQ ID NO: 169
  Synthetic Sequence

SEQ ID NO: 658 - DNA, 40 bp, Artificial Sequence; identical to SEQ ID NO: 174
  Synthetic Sequence

SEQ ID NO: 659 - DNA, 42 bp, Artificial Sequence; identical to SEQ ID NO: 175
  Synthetic Sequence

SEQ ID NO: 660 - DNA, 40 bp, Artificial Sequence
  Synthetic Sequence
cctcaaggagcctcagtctacctcaaggagcctcagtcta

SEQ ID NO: 661 - DNA, 42 bp, Artificial Sequence; identical to SEQ ID NO: 177
  Synthetic Sequence

SEQ ID NO: 662 - DNA, 44 bp, Artificial Sequence
  Synthetic Sequence
gatcacttttgtgactatgcaagatcacttttgtgactatgcaa

SEQ ID NO: 663 - DNA, 42 bp, Artificial Sequence; identical to SEQ ID NO: 179
  Synthetic Sequence

SEQ ID NO: 664 - DNA, 44 bp, Artificial Sequence
  Synthetic Sequence
cccctatcacaattagcattaacccctatcacaattagcattaa

SEQ ID NO: 665 - DNA, 44 bp, Artificial Sequence; identical to SEQ ID NO: 488
  Synthetic Sequence

SEQ ID NO: 666 - DNA, 42 bp, Artificial Sequence; identical to SEQ ID NO: 466
  Synthetic Sequence

SEQ ID NO: 667 - DNA, 42 bp, Artificial Sequence; identical to SEQ ID NO: 187
  Synthetic Sequence

SEQ ID NO: 668 - DNA, 44 bp, Artificial Sequence; identical to SEQ ID NO: 191
  Synthetic Sequence

SEQ ID NO: 669 - DNA, 44 bp, Artificial Sequence; identical to SEQ ID NO: 193
  Synthetic Sequence

SEQ ID NO: 670 - DNA, 42 bp, Artificial Sequence; identical to SEQ ID NO: 194
  Synthetic Sequence

SEQ ID NO: 671 - DNA, 36 bp, Artificial Sequence; identical to SEQ ID NO: 195
  Synthetic Sequence

SEQ ID NO: 672 - DNA, 44 bp, Artificial Sequence; identical to SEQ ID NO: 196
  Synthetic Sequence

SEQ ID NO: 673 - DNA, 40 bp, Artificial Sequence; identical to SEQ ID NO: 197
  Synthetic Sequence

SEQ ID NO: 674 - DNA, 42 bp, Artificial Sequence; identical to SEQ ID NO: 198
  Synthetic Sequence

SEQ ID NO: 675 - DNA, 44 bp, Artificial Sequence; identical to SEQ ID NO: 199
  Synthetic Sequence

SEQ ID NO: 676 - DNA, 42 bp, Artificial Sequence; identical to SEQ ID NO: 251
  Synthetic Sequence

SEQ ID NO: 677 - DNA, 44 bp, Artificial Sequence; identical to SEQ ID NO: 202
  Synthetic Sequence

SEQ ID NO: 678 - DNA, 42 bp, Artificial Sequence; identical to SEQ ID NO: 203
  Synthetic Sequence

SEQ ID NO: 679 - DNA, 42 bp, Artificial Sequence; identical to SEQ ID NO: 206
  Synthetic Sequence

SEQ ID NO: 680 - DNA, 44 bp, Artificial Sequence; identical to SEQ ID NO: 208
  Synthetic Sequence

SEQ ID NO: 681 - DNA, 42 bp, Artificial Sequence; identical to SEQ ID NO: 209
  Synthetic Sequence

SEQ ID NO: 682 - DNA, 42 bp, Artificial Sequence; identical to SEQ ID NO: 214
  Synthetic Sequence

SEQ ID NO: 683 - DNA, 44 bp, Artificial Sequence; identical to SEQ ID NO: 215
  Synthetic Sequence

SEQ ID NO: 684 - DNA, 44 bp, Artificial Sequence; identical to SEQ ID NO: 221
  Synthetic Sequence

SEQ ID NO: 685 - DNA, 42 bp, Artificial Sequence
  Synthetic Sequence
agaacaatgccttactgagtaagaacaatgccttactgagta

SEQ ID NO: 686 - DNA, 42 bp, Artificial Sequence
  Synthetic Sequence
tcttcccatgcgctatacctctcttcccatgcgctatacctc

SEQ ID NO: 687 - DNA, 42 bp, Artificial Sequence
  Synthetic Sequence
ctagtggtcctaaacatttcactagtggtcctaaacatttca

SEQ ID NO: 688 - DNA, 44 bp, Artificial Sequence; identical to SEQ ID NO: 226
  Synthetic Sequence

SEQ ID NO: 689 - DNA, 42 bp, Artificial Sequence; identical to SEQ ID NO: 227
  Synthetic Sequence

SEQ ID NO: 690 - DNA, 44 bp, Artificial Sequence; identical to SEQ ID NO: 228
  Synthetic Sequence

SEQ ID NO: 691 - DNA, 40 bp, Artificial Sequence
  Synthetic Sequence
agggaggagagccaggagaaagggaggagagccaggagaa

SEQ ID NO: 692 - DNA, 44 bp, Artificial Sequence; identical to SEQ ID NO: 140
  Synthetic Sequence

SEQ ID NO: 693 - DNA, 44 bp, Artificial Sequence; identical to SEQ ID NO: 166
  Synthetic Sequence

SEQ ID NO: 694 - DNA, 40 bp, Artificial Sequence; identical to SEQ ID NO: 277
  Synthetic Sequence

SEQ ID NO: 695 - DNA, 42 bp, Artificial Sequence
  Synthetic Sequence
ctgtaaacatccgactgaaagctgtaaacatccgactgaaag

SEQ ID NO: 696 - DNA, 44 bp, Artificial Sequence; identical to SEQ ID NO: 598
  Synthetic Sequence

SEQ ID NO: 697 - DNA, 42 bp, Artificial Sequence; identical to SEQ ID NO: 599
  Synthetic Sequence

SEQ ID NO: 698 - DNA, 44 bp, Artificial Sequence; identical to SEQ ID NO: 600
  Synthetic Sequence

SEQ ID NO: 699 - DNA, 42 bp, Artificial Sequence; identical to SEQ ID NO: 601
  Synthetic Sequence

SEQ ID NO: 700 - DNA, 44 bp, Artificial Sequence; identical to SEQ ID NO: 602
  Synthetic Sequence

SEQ ID NO: 701 - DNA, 42 bp, Artificial Sequence
  Synthetic Sequence
acactacaaactctgcggcacacactacaaactctgcggcac

SEQ ID NO: 702 - DNA, 44 bp, Artificial Sequence
  Synthetic Sequence
acacacaaaagggaagcactttacacacaaaagggaagcacttt

SEQ ID NO: 703 - DNA, 44 bp, Artificial Sequence
  Synthetic Sequence
agactcaaaagtagtagcacttagactcaaaagtagtagcactt

SEQ ID NO: 704 - DNA, 42 bp, Artificial Sequence; identical to SEQ ID NO: 258
  Synthetic Sequence

SEQ ID NO: 705 - DNA, 42 bp, Artificial Sequence
  Synthetic Sequence
catgcacatgcacacatacatcatgcacatgcacacatacat

SEQ ID NO: 706 - DNA, 38 bp, Artificial Sequence; identical to SEQ ID NO: 605
  Synthetic Sequence

SEQ ID NO: 707 - DNA, 44 bp, Artificial Sequence; identical to SEQ ID NO: 606
  Synthetic Sequence

SEQ ID NO: 708 - DNA, 42 bp, Artificial Sequence; identical to SEQ ID NO: 607
  Synthetic Sequence

SEQ ID NO: 709 - DNA, 44 bp, Artificial Sequence; identical to SEQ ID NO: 263
  Synthetic Sequence

SEQ ID NO: 710 - DNA, 44 bp, Artificial Sequence; identical to SEQ ID NO: 264
  Synthetic Sequence

SEQ ID NO: 711 - DNA, 44 bp, Artificial Sequence; identical to SEQ ID NO: 301
  Synthetic Sequence

SEQ ID NO: 712 - DNA, 44 bp, Artificial Sequence; identical to SEQ ID NO: 518
  Synthetic Sequence

SEQ ID NO: 713 - DNA, 42 bp, .Artificial Sequence; identical to SEQ ID NO: 125
  Synthetic Sequence

SEQ ID NO: 714 - DNA, 42 bp, Artificial Sequence; identical to SEQ ID NO: 443
  Synthetic Sequence

SEQ ID NO: 715 - DNA, 44 bp, Artificial Sequence
  Synthetic Sequence
tacctgcactgttagcactttgtacctgcactgttagcactttg

SEQ ID NO: 716 - DNA, 42 bp, Artificial Sequence; identical to SEQ ID NO: 136
  Synthetic Sequence

SEQ ID NO: 717 - DNA, 44 bp, Artificial Sequence; identical to SEQ ID NO: 151
  Synthetic Sequence

SEQ ID NO: 718 - DNA, 44 bp, Artificial Sequence; identical to SEQ ID NO: 218
  Synthetic Sequence

SEQ ID NO: 719 - DNA, 44 bp, Artificial Sequence; identical to SEQ ID NO: 274
  Synthetic Sequence

SEQ ID NO: 720 - DNA, 42 bp, Artificial Sequence; identical to SEQ ID NO: 275
  Synthetic Sequence

SEQ ID NO: 721 - DNA, 44 bp, Artificial Sequence; identical to SEQ ID NO: 172
  Synthetic Sequence

SEQ ID NO: 722 - DNA, 36 bp, Artificial Sequence
  Synthetic Sequence
tgtcaattcataggtcagtgtcaattcataggtcag

SEQ ID NO: 723 - DNA, 42 bp, Artificial Sequence; identical to SEQ ID NO: 210
  Synthetic Sequence

SEQ ID NO: 724 - DNA, 44 bp, Artificial Sequence; identical to SEQ ID NO: 219
  Synthetic Sequence

SEQ ID NO: 725 - DNA, 44 bp, Artificial Sequence; identical to SEQ ID NO: 229
  Synthetic Sequence

SEQ ID NO: 726 - DNA, 42 bp, Artificial Sequence
  Synthetic Sequence
actatacaacctactacctcaactatacaacctactacctca

SEQ ID NO: 727 - DNA, 44 bp, Artificial Sequence; identical to SEQ ID NO: 123
  Synthetic Sequence

SEQ ID NO: 728 - DNA, 44 bp, Artificial Sequence; identical to SEQ ID NO: 124
  Synthetic Sequence

SEQ ID NO: 729 - DNA, 42 bp, Artificial Sequence; identical to SEQ ID NO: 126
  Synthetic Sequence

SEQ ID NO: 730 - DNA, 40 bp, Artificial Sequence
  Synthetic Sequence
actatacaatctactacctcactatacaatctactacctc

SEQ ID NO: 731 - DNA, 44 bp, Artificial Sequence; identical to SEQ ID NO: 182
  Synthetic Sequence

SEQ ID NO: 732 - DNA, 44 bp, Artificial Sequence; identical to SEQ ID NO: 184
  Synthetic Sequence

SEQ ID NO: 733 - DNA, 44 bp, Artificial Sequence; identical to SEQ ID NO: 200
  Synthetic Sequence

SEQ ID NO: 734 - DNA, 44 bp, Artificial Sequence; identical to SEQ ID NO: 230
  Synthetic Sequence

SEQ ID NO: 735 - DNA, 44 bp, Artificial Sequence; identical to SEQ ID NO: 232
  Synthetic Sequence

SEQ ID NO: 736 - DNA, 44 bp, Artificial Sequence; identical to SEQ ID NO: 243
  Synthetic Sequence

SEQ ID NO: 737 - DNA, 42 bp, Artificial Sequence; identical to SEQ ID NO: 249
  Synthetic Sequence

SEQ ID NO: 738 - DNA, 42 bp, Artificial Sequence; identical to SEQ ID NO: 253
  Synthetic Sequence

SEQ ID NO: 739 - DNA, 44 bp, Artificial Sequence; identical to SEQ ID NO: 254
  Synthetic Sequence

SEQ ID NO: 740 - DNA, 42 bp, Artificial Sequence; identical to SEQ ID NO: 260
  Synthetic Sequence

SEQ ID NO: 741 - DNA, 40 bp, Artificial Sequence; identical to SEQ ID NO: 262
  Synthetic Sequence

SEQ ID NO: 742 - DNA, 42 bp, Artificial Sequence; identical to SEQ ID NO: 255
  Synthetic Sequence

SEQ ID NO: 743 - DNA, 42 bp, Artificial Sequence; identical to SEQ ID NO: 515
  Synthetic Sequence

SEQ ID NO: 744 - DNA, 40 bp, Artificial Sequence; identical to SEQ ID NO: 427
  Synthetic Sequence

SEQ ID NO: 745 - DNA, 42 bp, Artificial Sequence; identical to SEQ ID NO: 428
  Synthetic Sequence

SEQ ID NO: 746 - DNA, 44 bp, Artificial Sequence; identical to SEQ ID NO: 523
  Synthetic Sequence

SEQ ID NO: 747 - DNA, 42 bp, Artificial Sequence; identical to SEQ ID NO: 299
  Synthetic Sequence

SEQ ID NO: 748 - DNA, 44 bp, Artificial Sequence; identical to SEQ ID NO: 467
  Synthetic Sequence

SEQ ID NO: 749 - DNA, 44 bp, Artificial Sequence; identical to SEQ ID NO: 431
  Synthetic Sequence

SEQ ID NO: 750 - DNA, 42 bp, Artificial Sequence; identical to SEQ ID NO: 16
  Synthetic Sequence

SEQ ID NO: 751 - DNA, 44 bp, Artificial Sequence; identical to SEQ ID NO: 616
  Synthetic Sequence

SEQ ID NO: 752 - DNA, 40 bp, Artificial Sequence; identical to SEQ ID NO: 434
  Synthetic Sequence

SEQ ID NO: 753 - DNA, 42 bp, Artificial Sequence; identical to SEQ ID NO: 281
  Synthetic Sequence

SEQ ID NO: 754 - DNA, 38 bp, Artificial Sequence
  Synthetic Sequence
caccaatgccctaggggatcaccaatgccctaggggat

SEQ ID NO: 755 - DNA, 36 bp, Artificial Sequence; identical to SEQ ID NO: 282
  Synthetic Sequence

SEQ ID NO: 756 - DNA, 44 bp, Artificial Sequence; identical to SEQ ID NO: 439
  Synthetic Sequence

SEQ ID NO: 757 - DNA, 38 bp, Artificial Sequence; identical to SEQ ID NO: 440
  Synthetic Sequence

SEQ ID NO: 758 - DNA, 42 bp, Artificial Sequence; identical to SEQ ID NO: 286
  Synthetic Sequence

SEQ ID NO: 759 - DNA, 44 bp, Artificial Sequence; identical to SEQ ID NO: 445
  Synthetic Sequence

SEQ ID NO: 760 - DNA, 42 bp, Artificial Sequence; identical to SEQ ID NO: 446
  Synthetic Sequence

SEQ ID NO: 761 - DNA, 38 bp, Artificial Sequence; identical to SEQ ID NO: 290
  Synthetic Sequence

SEQ ID NO: 762 - DNA, 44 bp, Artificial Sequence; identical to SEQ ID NO: 453
  Synthetic Sequence

SEQ ID NO: 763 - DNA, 44 bp, Artificial Sequence; identical to SEQ ID NO: 173
  Synthetic Sequence

SEQ ID NO: 764 - DNA, 44 bp, Artificial Sequence; identical to SEQ ID NO: 293
  Synthetic Sequence

SEQ ID NO: 765 - DNA, 42 bp, Artificial Sequence; identical to SEQ ID NO: 294
  Synthetic Sequence

SEQ ID NO: 766 - DNA, 44 bp, Artificial Sequence; identical to SEQ ID NO: 295
  Synthetic Sequence

SEQ ID NO: 767 - DNA, 42 bp, Artificial Sequence; identical to SEQ ID NO: 458
  Synthetic Sequence

SEQ ID NO: 768 - DNA, 42 bp, Artificial Sequence; identical to SEQ ID NO: 296
  Synthetic Sequence

SEQ ID NO: 769 - DNA, 42 bp, Artificial Sequence
  Synthetic Sequence
acagtcaggctttggctagatacagtcaggctttggctagat

SEQ ID NO: 770 - DNA, 38 bp, Artificial Sequence; identical to SEQ ID NO: 462
  Synthetic Sequence

SEQ ID NO: 771 - DNA, 42 bp, Artificial Sequence
  Synthetic Sequence
agaggcaggcactcgggcagaagaggcaggcactcgggcaga

SEQ ID NO: 772 - DNA, 44 bp, Artificial Sequence
  Synthetic Sequence
tgaaagtgtatgggctttgtgatgaaagtgtatgggctttgtga

SEQ ID NO: 773 - DNA, 38 bp, Artificial Sequence; identical to SEQ ID NO: 473
  Synthetic Sequence

SEQ ID NO: 774 - DNA, 44 bp, Artificial Sequence; identical to SEQ ID NO: 157
  Synthetic Sequence

SEQ ID NO: 775 - DNA, 44 bp, Artificial Sequence; identical to SEQ ID NO: 478
  Synthetic Sequence

SEQ ID NO: 776 - DNA, 42 bp, Artificial Sequence; identical to SEQ ID NO: 137
  Synthetic Sequence

SEQ ID NO: 777 - DNA, 44 bp, Artificial Sequence; identical to SEQ ID NO: 138
  Synthetic Sequence

SEQ ID NO: 778 - DNA, 44 bp, Artificial Sequence; identical to SEQ ID NO: 186
  Synthetic Sequence

SEQ ID NO: 779 - DNA, 42 bp, Artificial Sequence
  Synthetic Sequence
tacaagtgccctcactgcagttacaagtgccctcactgcagt

SEQ ID NO: 780 - DNA, 44 bp, Artificial Sequence; identical to SEQ ID NO: 217
  Synthetic Sequence

SEQ ID NO: 781 - DNA, 42 bp, Artificial Sequence; identical to SEQ ID NO: 252
  Synthetic Sequence

SEQ ID NO: 782 - DNA, 44 bp, Artificial Sequence; identical to SEQ ID NO: 257
  Synthetic Sequence

SEQ ID NO: 783 - DNA, 40 bp, Artificial Sequence; identical to SEQ ID NO: 279
  Synthetic Sequence

SEQ ID NO: 784 - DNA, 42 bp, Artificial Sequence; identical to SEQ ID NO: 131
  Synthetic Sequence

SEQ ID NO: 785 - DNA, 36 bp, Artificial Sequence; identical to SEQ ID NO: 161
  Synthetic Sequence

SEQ ID NO: 786 - DNA, 44 bp, Artificial Sequence; identical to SEQ ID NO: 222
  Synthetic Sequence

SEQ ID NO: 787 - DNA, 42 bp, Artificial Sequence; identical to SEQ ID NO: 233
  Synthetic Sequence

SEQ ID NO: 788 - DNA, 40 bp, Artificial Sequence; identical to SEQ ID NO: 235
  Synthetic Sequence

SEQ ID NO: 789 - DNA, 42 bp, Artificial Sequence; identical to SEQ ID NO: 236
  Synthetic Sequence

SEQ ID NO: 790 - DNA, 40 bp, Artificial Sequence; identical to SEQ ID NO: 237
  Synthetic Sequence

SEQ ID NO: 791 - DNA, 42 bp, Artificial Sequence; identical to SEQ ID NO: 239
  Synthetic Sequence

SEQ ID NO: 792 - DNA, 42 bp, Artificial Sequence; identical to SEQ ID NO: 241
  Synthetic Sequence

SEQ ID NO: 793 - DNA, 42 bp, Artificial Sequence; identical to SEQ ID NO: 242
  Synthetic Sequence

SEQ ID NO: 794 - DNA, 42 bp, Artificial Sequence; identical to SEQ ID NO: 247
  Synthetic Sequence

SEQ ID NO: 795 - DNA, 44 bp, Artificial Sequence; identical to SEQ ID NO: 280
  Synthetic Sequence

SEQ ID NO: 796 - DNA, 38 bp, Artificial Sequence; identical to SEQ ID NO: 288
  Synthetic Sequence

SEQ ID NO: 797 - DNA, 44 bp, Artificial Sequence; identical to SEQ ID NO: 587
  Synthetic Sequence

SEQ ID NO: 798 - DNA, 44 bp, Artificial Sequence; identical to SEQ ID NO: 245
  Synthetic Sequence

SEQ ID NO: 799 - DNA, 42 bp, Artificial Sequence; identical to SEQ ID NO: 246
  Synthetic Sequence

SEQ ID NO: 800 - DNA, 44 bp, Artificial Sequence; identical to SEQ ID NO: 248
  Synthetic Sequence

SEQ ID NO: 801 - DNA, 44 bp, Artificial Sequence
  Synthetic Sequence
gaacaggtagtctaaacactgggaacaggtagtctaaacactgg

SEQ ID NO: 802 - DNA, 42 bp, Artificial Sequence; identical to SEQ ID NO: 188
  Synthetic Sequence

SEQ ID NO: 803 - DNA, 44 bp, Artificial Sequence; identical to SEQ ID NO: 189
  Synthetic Sequence

SEQ ID NO: 804 - DNA, 42 bp, Artificial Sequence; identical to SEQ ID NO: 148
  Synthetic Sequence

SEQ ID NO: 805 - DNA, 44 bp, Artificial Sequence; identical to SEQ ID NO: 424
  Synthetic Sequence

SEQ ID NO: 806 - DNA, 40 bp, Artificial Sequence; identical to SEQ ID NO: 485
  Synthetic Sequence

SEQ ID NO: 807 - DNA, 42 bp, Artificial Sequence; identical to SEQ ID NO: 592
  Synthetic Sequence

SEQ ID NO: 808 - DNA, 44 bp, Artificial Sequence; identical to SEQ ID NO: 302
  Synthetic Sequence

SEQ ID NO: 809 - DNA, 44 bp, Artificial Sequence; identical to SEQ ID NO: 304
  Synthetic Sequence

SEQ ID NO: 810 - DNA, 44 bp, Artificial Sequence; identical to SEQ ID NO: 305
  Synthetic Sequence

SEQ ID NO: 811 - DNA, 44 bp, Artificial Sequence; identical to SEQ ID NO: 316
  Synthetic Sequence

SEQ ID NO: 812 - DNA, 42 bp, Artificial Sequence
  Synthetic Sequence
acgtggattttcctctacgatacgtggattttcctctacgat

SEQ ID NO: 813 - DNA, 44 bp, Artificial Sequence; identical to SEQ ID NO: 319
  Synthetic Sequence

SEQ ID NO: 814 - DNA, 42 bp, Artificial Sequence; identical to SEQ ID NO: 320
  Synthetic Sequence

SEQ ID NO: 815 - DNA, 42 bp, Artificial Sequence
  Synthetic Sequence
cctacgttccatagtctaccacctacgttccatagtctacca

SEQ ID NO: 816 - DNA, 42 bp, Artificial Sequence; identical to SEQ ID NO: 323
  Synthetic Sequence

SEQ ID NO: 817 - DNA, 44 bp, Artificial Sequence
  Synthetic Sequence
aagatgtggaccatactacataaagatgtggaccatactacata

SEQ ID NO: 818 - DNA, 44 bp, Artificial Sequence; identical to SEQ ID NO: 324
  Synthetic Sequence

SEQ ID NO: 819 - DNA, 42 bp, Artificial Sequence; identical to SEQ ID NO: 325
  Synthetic Sequence

SEQ ID NO: 820 - DNA, 42 bp, Artificial Sequence
  Synthetic Sequence
agccacagtcaccttctgatcagccacagtcaccttctgatc

SEQ ID NO: 821 - DNA, 44 bp, Artificial Sequence; identical to SEQ ID NO: 291
  Synthetic Sequence

SEQ ID NO: 822 - DNA, 42 bp, Artificial Sequence; identical to SEQ ID NO: 154
  Synthetic Sequence

SEQ ID NO: 823 - DNA, 42 bp, Artificial Sequence
  Synthetic Sequence
gtctgtcaaatcataggtcatgtctgtcaaatcataggtcat

SEQ ID NO: 824 - DNA, 40 bp, Artificial Sequence
  Synthetic Sequence
tgtgaacaatttctaggaattgtgaacaatttctaggaat

SEQ ID NO: 825 - DNA, 42 bp, Artificial Sequence; identical to SEQ ID NO: 211
  Synthetic Sequence

SEQ ID NO: 826 - DNA, 42 bp, Artificial Sequence
  Synthetic Sequence
aaggggttcaccgagcaacataaggggttcaccgagcaacat

SEQ ID NO: 827 - DNA, 44 bp, Artificial Sequence; identical to SEQ ID NO: 330
  Synthetic Sequence

SEQ ID NO: 828 - DNA, 44 bp, Artificial Sequence
  Synthetic Sequence
aaagtggatgttcctctatgataaagtggatgttcctctatgat

SEQ ID NO: 829 - DNA, 44 bp, Artificial Sequence
  Synthetic Sequence
actgagggttagtggaccgtgtactgagggttagtggaccgtgt

SEQ ID NO: 830 - DNA, 42 bp, Artificial Sequence; identical to SEQ ID NO: 331
  Synthetic Sequence

SEQ ID NO: 831 - DNA, 40 bp, Artificial Sequence
  Synthetic Sequence
aaccaggttccaccccagcaaaccaggttccaccccagca

SEQ ID NO: 832 - DNA, 38 bp, Artificial Sequence; identical to SEQ ID NO: 336
  Synthetic Sequence

SEQ ID NO: 833 - DNA, 42 bp, Artificial Sequence; identical to SEQ ID NO: 338
  Synthetic Sequence

SEQ ID NO: 834 - DNA, 44 bp, Artificial Sequence
  Synthetic Sequence
gaataatgacaggctcaccgtagaataatgacaggctcaccgta

SEQ ID NO: 835 - DNA, 44 bp, Artificial Sequence; identical to SEQ ID NO: 341
  Synthetic Sequence

SEQ ID NO: 836 - DNA, 42 bp, Artificial Sequence
  Synthetic Sequence
ggttcaaaccatgagtcgagcggttcaaaccatgagtcgagc

SEQ ID NO: 837 - DNA, 42 bp, Artificial Sequence
  Synthetic Sequence
ggagtcgagtgatggttcaaaggagtcgagtgatggttcaaa

SEQ ID NO: 838 - DNA, 44 bp, Artificial Sequence; identical to SEQ ID NO: 342
  Synthetic Sequence

SEQ ID NO: 839 - DNA, 44 bp, Artificial Sequence; identical to SEQ ID NO: 612
  Synthetic Sequence

SEQ ID NO: 840 - DNA, 44 bp, Artificial Sequence; identical to SEQ ID NO: 343
  Synthetic Sequence

SEQ ID NO: 841 - DNA, 44 bp, Artificial Sequence; identical to SEQ ID NO: 344
  Synthetic Sequence

SEQ ID NO: 842 - DNA, 44 bp, Artificial Sequence; identical to SEQ ID NO: 619
  Synthetic Sequence

SEQ ID NO: 843 - DNA, 44 bp, Artificial Sequence; identical to SEQ ID NO: 346
  Synthetic Sequence

SEQ ID NO: 844 - DNA, 40 bp, Artificial Sequence
  Synthetic Sequence
tgatggacaacaaattaggttgatggacaacaaattaggt

SEQ ID NO: 845 - DNA, 44 bp, Artificial Sequence
  Synthetic Sequence
tatctcacagaataaacttggttatctcacagaataaacttggt

SEQ ID NO: 846 - DNA, 44 bp, Artificial Sequence
  Synthetic Sequence
tcacatcagtgccattctaaattcacatcagtgccattctaaat

SEQ ID NO: 847 - DNA, 44 bp, Artificial Sequence
  Synthetic Sequence
gtcttatgtgtgcgtgtatgtagtcttatgtgtgcgtgtatgta

SEQ ID NO: 848 - DNA, 44 bp, Artificial Sequence
  Synthetic Sequence
gtgtaggtgtgtgtatgtatatgtgtaggtgtgtgtatgtatat

SEQ ID NO: 849 - DNA, 44 bp, Artificial Sequence
  Synthetic Sequence
agacacacgcacatcagtcataagacacacgcacatcagtcata

SEQ ID NO: 850 - DNA, 44 bp, Artificial Sequence
  Synthetic Sequence
acaccaagatcaatgaaagaggacaccaagatcaatgaaagagg

SEQ ID NO: 851 - DNA, 42 bp, Artificial Sequence
  Synthetic Sequence
tcaccagtgccagtccaagaatcaccagtgccagtccaagaa

SEQ ID NO: 852 - DNA, 44 bp, Artificial Sequence
  Synthetic Sequence
tgtgaaaagcactatactacgttgtgaaaagcactatactacgt

SEQ ID NO: 853 - DNA, 44 bp, Artificial Sequence
  Synthetic Sequence
ctccatacttctttacattccactccatacttctttacattcca

SEQ ID NO: 854 - DNA, 44 bp, Artificial Sequence
  Synthetic Sequence
ctcatcaaagctggctgtgatactcatcaaagctggctgtgata

SEQ ID NO: 855 - DNA, 42 bp, Artificial Sequence
  Synthetic Sequence
ctcctcaaagctggctgtgatctcctcaaagctggctgtgat

SEQ ID NO: 856 - DNA, 42 bp, Artificial Sequence
  Synthetic Sequence
tgagacacactttgcccagtgtgagacacactttgcccagtg

SEQ ID NO: 857 - DNA, 40 bp, Artificial Sequence
  Synthetic Sequence
tcaatggttgtctagctttatcaatggttgtctagcttta

SEQ ID NO: 858 - DNA, 44 bp, Artificial Sequence
  Synthetic Sequence
catatcacaacgatcgttccttcatatcacaacgatcgttcctt

SEQ ID NO: 859 - DNA, 44 bp, Artificial Sequence
  Synthetic Sequence
aaaaagaacagccactgtgataaaaaagaacagccactgtgata

SEQ ID NO: 860 - DNA, 44 bp, Artificial Sequence
  Synthetic Sequence
acaacaaaatcactagtcttccacaacaaaatcactagtcttcc

SEQ ID NO: 861 - DNA, 44 bp, Artificial Sequence
  Synthetic Sequence
gacatctttacctgacagtattgacatctttacctgacagtatt

SEQ ID NO: 862 - DNA, 44 bp, Artificial Sequence; identical to SEQ ID NO: 8
  Synthetic Sequence

SEQ ID NO: 863 - DNA, 42 bp, Artificial Sequence
  Synthetic Sequence
acaaattcggatctacagggtacaaattcggatctacagggt

SEQ ID NO: 864 - DNA, 42 bp, Artificial Sequence
  Synthetic Sequence
gcaagaactcagactgtgatggcaagaactcagactgtgatg

SEQ ID NO: 865 - DNA, 44 bp, Artificial Sequence
  Synthetic Sequence
accagtacctgatgtaatactcaccagtacctgatgtaatactc

SEQ ID NO: 866 - DNA, 44 bp, Artificial Sequence
  Synthetic Sequence
actcatcaaaatggctgtgataactcatcaaaatggctgtgata

SEQ ID NO: 867 - DNA, 44 bp, Artificial Sequence
  Synthetic Sequence
actcgtcaaaatggctgtgataactcgtcaaaatggctgtgata

SEQ ID NO: 868 - DNA, 42 bp, Artificial Sequence
  Synthetic Sequence
taggagagagaaaaagactgataggagagagaaaaagactga

SEQ ID NO: 869 - DNA, 44 bp, Artificial Sequence
  Synthetic Sequence
gtgaattcttccagtgccattagtgaattcttccagtgccatta

SEQ ID NO: 870 - DNA, 40 bp, Artificial Sequence
  Synthetic Sequence
cggggcgagagaatgataagcggggcgagagaatgataag

SEQ ID NO: 871 - DNA, 42 bp, Artificial Sequence
  Synthetic Sequence
cccttatcagttctccgtccacccttatcagttctccgtcca

SEQ ID NO: 872 - DNA, 42 bp, Artificial Sequence
  Synthetic Sequence
attacccgttagtgtcggtcaattacccgttagtgtcggtca

SEQ ID NO: 873 - DNA, 40 bp, Artificial Sequence
  Synthetic Sequence
gcgctacttcaggtacctgagcgctacttcaggtacctga

SEQ ID NO: 874 - DNA, 42 bp, Artificial Sequence
  Synthetic Sequence
ataggccgggacaagtgcaatataggccgggacaagtgcaat

SEQ ID NO: 875 - DNA, 44 bp, Artificial Sequence
  Synthetic Sequence
caagaattgcgtttggacaatccaagaattgcgtttggacaatc

SEQ ID NO: 876 - DNA, 42 bp, Artificial Sequence
  Synthetic Sequence
cgtaggaactctatacctcgccgtaggaactctatacctcgc

SEQ ID NO: 877 - DNA, 44 bp, Artificial Sequence
  Synthetic Sequence
agagcacggtatgaagttcctaagagcacggtatgaagttccta

SEQ ID NO: 878 - DNA, 44 bp, Artificial Sequence
  Synthetic Sequence
tgtcgtaccagatagtgcattttgtcgtaccagatagtgcattt

SEQ ID NO: 879 - DNA, 44 bp, Artificial Sequence
  Synthetic Sequence
aaacggacgaaagtcccaccgaaaacggacgaaagtcccaccga

SEQ ID NO: 880 - DNA, 44 bp, Artificial Sequence; identical to SEQ ID NO: 153
  Synthetic Sequence

SEQ ID NO: 881 - DNA, 44 bp, Artificial Sequence
  Synthetic Sequence
ttaatgagtgtggatctagtcattaatgagtgtggatctagtca

SEQ ID NO: 882 - DNA, 42 bp, Artificial Sequence
  Synthetic Sequence
caatgcgactacaatgcacctcaatgcgactacaatgcacct

SEQ ID NO: 883 - DNA, 44 bp, Artificial Sequence
  Synthetic Sequence
catttcatatgcaacgtaaatacatttcatatgcaacgtaaata

SEQ ID NO: 884 - DNA, 44 bp, Artificial Sequence
  Synthetic Sequence
actgtcgacggacagctctcttactgtcgacggacagctctctt

SEQ ID NO: 885 - DNA, 44 bp, Artificial Sequence
  Synthetic Sequence
acaaagagagcaattccatgacacaaagagagcaattccatgac

SEQ ID NO: 886 - DNA, 42 bp, Artificial Sequence
  Synthetic Sequence
acagacaaagcctagtagaggacagacaaagcctagtagagg

SEQ ID NO: 887 - DNA, 42 bp, Artificial Sequence
  Synthetic Sequence
agaattaccagctgatatttaagaattaccagctgatattta

SEQ ID NO: 888 - DNA, 44 bp, Artificial Sequence
  Synthetic Sequence
aattgctggaatcaagttgctgaattgctggaatcaagttgctg

SEQ ID NO: 889 - DNA, 42 bp, Artificial Sequence
  Synthetic Sequence
actgtcgacggatagctctctactgtcgacggatagctctct

SEQ ID NO: 890 - DNA, 42 bp, Artificial Sequence
  Synthetic Sequence
aaccagctaaccacactgccaaaccagctaaccacactgcca

SEQ ID NO: 891 - DNA, 44 bp, Artificial Sequence
  Synthetic Sequence
ttggcattcaccgcgtgccttattggcattcaccgcgtgcctta

SEQ ID NO: 892 - DNA, 44 bp, Artificial Sequence
  Synthetic Sequence
atgctttggtaatctagctttaatgctttggtaatctagcttta

SEQ ID NO: 893 - DNA, 44 bp, Artificial Sequence
  Synthetic Sequence
agagcacggtattaagttcctaagagcacggtattaagttccta

SEQ ID NO: 894 - DNA, 42 bp, Artificial Sequence
  Synthetic Sequence
tagccgctgtcacacgcacaatagccgctgtcacacgcacaa

SEQ ID NO: 895 - DNA, 44 bp, Artificial Sequence
  Synthetic Sequence
gcactgatttcgaatggtgctagcactgatttcgaatggtgcta

SEQ ID NO: 896 - DNA, 44 bp, Artificial Sequence
  Synthetic Sequence
cacaagttcggatttacgggttcacaagttcggatttacgggtt

SEQ ID NO: 897 - DNA, 40 bp, Artificial Sequence
  Synthetic Sequence
aggccgggactagtgcaattaggccgggactagtgcaatt

SEQ ID NO: 898 - DNA, 42 bp, Artificial Sequence
  Synthetic Sequence
agcacgagtgttcggtctagtagcacgagtgttcggtctagt

SEQ ID NO: 899 - DNA, 42 bp, Artificial Sequence
  Synthetic Sequence
gtgcaaacgattttcaacacagtgcaaacgattttcaacaca

SEQ ID NO: 900 - DNA, 42 bp, Artificial Sequence
  Synthetic Sequence
cacacctgaaattttgctcaacacacctgaaattttgctcaa

SEQ ID NO: 901 - DNA, 42 bp, Artificial Sequence
  Synthetic Sequence
gtgaattctcccagtgccaaggtgaattctcccagtgccaag

SEQ ID NO: 902 - DNA, 44 bp, Artificial Sequence
  Synthetic Sequence
catgaaatgaaatcgacatgaacatgaaatgaaatcgacatgaa

SEQ ID NO: 903 - DNA, 44 bp, Artificial Sequence
  Synthetic Sequence
agtcgcaggctccacttaaataagtcgcaggctccacttaaata

SEQ ID NO: 904 - DNA, 44 bp, Artificial Sequence
  Synthetic Sequence
aatcagctttcaaaatgatctcaatcagctttcaaaatgatctc

SEQ ID NO: 905 - DNA, 44 bp, Artificial Sequence
  Synthetic Sequence
accagtttcctgtgaaacctaaaccagtttcctgtgaaacctaa

SEQ ID NO: 906 - DNA, 42 bp, Artificial Sequence
  Synthetic Sequence
cagctattccgacatcttgcccagctattccgacatcttgcc

SEQ ID NO: 907 - DNA, 44 bp, Artificial Sequence
  Synthetic Sequence
ctcacatttacaaattgagattctcacatttacaaattgagatt

SEQ ID NO: 908 - DNA, 44 bp, Artificial Sequence
  Synthetic Sequence
cagagcacctgatgaagtacaacagagcacctgatgaagtacaa

SEQ ID NO: 909 - DNA, 44 bp, Artificial Sequence
  Synthetic Sequence
tctacagctagaataccaaagatctacagctagaataccaaaga

SEQ ID NO: 910 - DNA, 44 bp, Artificial Sequence
  Synthetic Sequence
ttgagagtcactaagtacctgattgagagtcactaagtacctga

SEQ ID NO: 911 - DNA, 36 bp, Artificial Sequence
  Synthetic Sequence
gcacaggcacagagtgacgcacaggcacagagtgac

SEQ ID NO: 912 - DNA, 44 bp, Artificial Sequence
  Synthetic Sequence
catacagctaaaatcaccaaagcatacagctaaaatcaccaaag

SEQ ID NO: 913 - DNA, 42 bp, Artificial Sequence; identical to SEQ ID NO: 726
  Synthetic Sequence

SEQ ID NO: 914 - DNA, 42 bp, Artificial Sequence; identical to SEQ ID NO: 143
  Synthetic Sequence

SEQ ID NO: 915 - DNA, 40 bp, Artificial Sequence
  Synthetic Sequence
ctcactcaaggaggttgtgactcactcaaggaggttgtga

SEQ ID NO: 916 - DNA, 44 bp, Artificial Sequence
  Synthetic Sequence
ctcacagtataatcctgtgattctcacagtataatcctgtgatt

SEQ ID NO: 917 - DNA, 42 bp, Artificial Sequence
  Synthetic Sequence
tcagctatgccgacatcttgctcagctatgccgacatcttgc

SEQ ID NO: 918 - DNA, 44 bp, Artificial Sequence
  Synthetic Sequence
taggacaaactttacccagtgctaggacaaactttacccagtgc

SEQ ID NO: 919 - DNA, 42 bp, Artificial Sequence
  Synthetic Sequence
aaaggccgggaagtgtgcaataaaggccgggaagtgtgcaat

SEQ ID NO: 920 - DNA, 42 bp, Artificial Sequence
  Synthetic Sequence
tcaggccggtgaatgtgcaattcaggccggtgaatgtgcaat

SEQ ID NO: 921 - DNA, 42 bp, Artificial Sequence
  Synthetic Sequence
tcaggccgtctcaagtgcaattcaggccgtctcaagtgcaat

SEQ ID NO: 922 - DNA, 44 bp, Artificial Sequence
  Synthetic Sequence
tcgggctgtgaaaagtgcaatatcgggctgtgaaaagtgcaata

SEQ ID NO: 923 - DNA, 42 bp, Artificial Sequence
  Synthetic Sequence
ccgaacttattggctcgaataccgaacttattggctcgaata

SEQ ID NO: 924 - DNA, 44 bp, Artificial Sequence
  Synthetic Sequence
gctttctgagcaacaatcaaaagctttctgagcaacaatcaaaa

SEQ ID NO: 925 - DNA, 42 bp, Artificial Sequence
  Synthetic Sequence
cgccagtaagcggaaaaagaccgccagtaagcggaaaaagac

SEQ ID NO: 926 - DNA, 42 bp, Artificial Sequence
  Synthetic Sequence
actggataccaccagctgtgtactggataccaccagctgtgt

SEQ ID NO: 927 - DNA, 44 bp, Artificial Sequence
  Synthetic Sequence
tgagataaacaaagcccagtgatgagataaacaaagcccagtga

SEQ ID NO: 928 - DNA, 42 bp, Artificial Sequence
  Synthetic Sequence
cccatcaaagctggctgtgatcccatcaaagctggctgtgat

SEQ ID NO: 929 - DNA, 44 bp, Artificial Sequence
  Synthetic Sequence
tcaggatacattcagtatacgttcaggatacattcagtatacgt

SEQ ID NO: 930 - DNA, 44 bp, Artificial Sequence
  Synthetic Sequence
gttacgtatactgaaggtatacgttacgtatactgaaggtatac

SEQ ID NO: 931 - DNA, 44 bp, Artificial Sequence; identical to SEQ ID NO: 122
  Synthetic Sequence

SEQ ID NO: 932 - DNA, 42 bp, Artificial Sequence
  Synthetic Sequence
tcacacttgaggtctcagggatcacacttgaggtctcaggga

SEQ ID NO: 933 - DNA, 42 bp, Artificial Sequence; identical to SEQ ID NO: 130
  Synthetic Sequence

SEQ ID NO: 934 - DNA, 44 bp, Artificial Sequence
  Synthetic Sequence
cacatcaaagctggctgtgatacacatcaaagctggctgtgata

SEQ ID NO: 935 - DNA, 42 bp, Artificial Sequence
  Synthetic Sequence
aaccagctaaccacactgcctaaccagctaaccacactgcct

SEQ ID NO: 936 - DNA, 44 bp, Artificial Sequence
  Synthetic Sequence
actgctagtttccacccggtgaactgctagtttccacccggtga

SEQ ID NO: 937 - DNA, 42 bp, Artificial Sequence
  Synthetic Sequence
catgcgaattttcacccggtgcatgcgaattttcacccggtg

SEQ ID NO: 938 - DNA, 44 bp, Artificial Sequence
  Synthetic Sequence
actgcaagtgttcacccggtgaactgcaagtgttcacccggtga

SEQ ID NO: 939 - DNA, 42 bp, Artificial Sequence
  Synthetic Sequence
actccagtttttctcccggtgactccagtttttctcccggtg

SEQ ID NO: 940 - DNA, 42 bp, Artificial Sequence
  Synthetic Sequence
caagctgatttacacccggtgcaagctgatttacacccggtg

SEQ ID NO: 941 - DNA, 42 bp, Artificial Sequence
  Synthetic Sequence
ttagctgatgtacacccggtgttagctgatgtacacccggtg

SEQ ID NO: 942 - DNA, 44 bp, Artificial Sequence
  Synthetic Sequence
taggtgatttttcacccggtgataggtgatttttcacccggtga

SEQ ID NO: 943 - DNA, 40 bp, Artificial Sequence
  Synthetic Sequence
ctgtagatgttaacccggtgctgtagatgttaacccggtg

SEQ ID NO: 944 - DNA, 44 bp, Artificial Sequence
  Synthetic Sequence
gcgacagcaagtaaactgtgatgcgacagcaagtaaactgtgat

SEQ ID NO: 945 - DNA, 42 bp, Artificial Sequence
  Synthetic Sequence
agctgaatgtgtctctagtcaagctgaatgtgtctctagtca

SEQ ID NO: 946 - DNA, 42 bp, Artificial Sequence; identical to SEQ ID NO: 945
  Synthetic Sequence

SEQ ID NO: 947 - DNA, 42 bp, Artificial Sequence
  Synthetic Sequence
tgaagagagcgactccatgactgaagagagcgactccatgac

SEQ ID NO: 948 - DNA, 44 bp, Artificial Sequence
  Synthetic Sequence
tgaagagagcgcctccatgacatgaagagagcgcctccatgaca

SEQ ID NO: 949 - DNA, 42 bp, Artificial Sequence
  Synthetic Sequence
tcgcatctactgagcctaccttcgcatctactgagcctacct

SEQ ID NO: 950 - DNA, 44 bp, Artificial Sequence
  Synthetic Sequence
tctgcagcttctcgtggtgctttctgcagcttctcgtggtgctt

SEQ ID NO: 951 - DNA, 44 bp, Artificial Sequence
  Synthetic Sequence
acccaagaataccagacatatcacccaagaataccagacatatc

SEQ ID NO: 952 - DNA, 42 bp, Artificial Sequence
  Synthetic Sequence
aacatggataggagctacgggaacatggataggagctacggg

SEQ ID NO: 953 - DNA, 42 bp, Artificial Sequence
  Synthetic Sequence
agcacggaaacatatgtacggagcacggaaacatatgtacgg

SEQ ID NO: 954 - DNA, 42 bp, Artificial Sequence
  Synthetic Sequence
agcacggaaacaaatgtacggagcacggaaacaaatgtacgg

SEQ ID NO: 955 - DNA, 44 bp, Artificial Sequence
  Synthetic Sequence
ctcggattatgaagattacgggctcggattatgaagattacggg

SEQ ID NO: 956 - DNA, 44 bp, Artificial Sequence
  Synthetic Sequence
ctcagcagaaacttatacgggtctcagcagaaacttatacgggt

SEQ ID NO: 957 - DNA, 42 bp, Artificial Sequence
  Synthetic Sequence
tacaacccaaaatggatccgctacaacccaaaatggatccgc

SEQ ID NO: 958 - DNA, 42 bp, Artificial Sequence
  Synthetic Sequence
ctcagcggaaacattacgggtctcagcggaaacattacgggt

SEQ ID NO: 959 - DNA, 42 bp, Artificial Sequence
  Synthetic Sequence
acacacagctcgatctacaggacacacagctcgatctacagg

SEQ ID NO: 960 - DNA, 44 bp, Artificial Sequence
  Synthetic Sequence
attgccgtactgaacgatctcaattgccgtactgaacgatctca

SEQ ID NO: 961 - DNA, 44 bp, Artificial Sequence
  Synthetic Sequence
catcatcctgataaacgattcgcatcatcctgataaacgattcg

SEQ ID NO: 962 - DNA, 44 bp, Artificial Sequence
  Synthetic Sequence
tgaactagaaaatgtgcataattgaactagaaaatgtgcataat

SEQ ID NO: 963 - DNA, 44 bp, Artificial Sequence
  Synthetic Sequence
gagatgagtaacggttctagtcgagatgagtaacggttctagtc

SEQ ID NO: 964 - DNA, 44 bp, Artificial Sequence
  Synthetic Sequence
ctgtaagctagattacatatcactgtaagctagattacatatca

SEQ ID NO: 965 - DNA, 44 bp, Artificial Sequence
  Synthetic Sequence
tttccaactcgcttcagtgtcatttccaactcgcttcagtgtca

SEQ ID NO: 966 - DNA, 44 bp, Artificial Sequence
  Synthetic Sequence
ttcggtaacgcttcagtgtcatttcggtaacgcttcagtgtcat

SEQ ID NO: 967 - DNA, 44 bp, Artificial Sequence
  Synthetic Sequence
ttcggttacgcttcagtgtcatttcggttacgcttcagtgtcat

SEQ ID NO: 968 - DNA, 44 bp, Artificial Sequence
  Synthetic Sequence
tcacatccctaatcagtgtcattcacatccctaatcagtgtcat

SEQ ID NO: 969 - DNA, 44 bp, Artificial Sequence
  Synthetic Sequence
tctactctttctaggaggttgttctactctttctaggaggttgt

SEQ ID NO: 970 - DNA, 44 bp, Artificial Sequence
  Synthetic Sequence
atggaaacaccaacgacgtattatggaaacaccaacgacgtatt

SEQ ID NO: 971 - DNA, 38 bp, Artificial Sequence
  Synthetic Sequence
tcactacccatgtctttcatcactacccatgtctttca

SEQ ID NO: 972 - DNA, 40 bp, Artificial Sequence
  Synthetic Sequence
gctatgccaacatcttgcctgctatgccaacatcttgcct

SEQ ID NO: 973 - DNA, 42 bp, Artificial Sequence
  Synthetic Sequence
actgaactgcctacatcttgcactgaactgcctacatcttgc

SEQ ID NO: 974 - DNA, 44 bp, Artificial Sequence
  Synthetic Sequence
tgtagactgccatttcttgccatgtagactgccatttcttgcca

SEQ ID NO: 975 - DNA, 44 bp, Artificial Sequence
  Synthetic Sequence
tgaagccggttggtagctttaatgaagccggttggtagctttaa

SEQ ID NO: 976 - DNA, 44 bp, Artificial Sequence
  Synthetic Sequence
tcaaggcttcatcaacaacgaatcaaggcttcatcaacaacgaa

SEQ ID NO: 977 - DNA, 42 bp, Artificial Sequence
  Synthetic Sequence
tggacagctatggcctgatgatggacagctatggcctgatga

SEQ ID NO: 978 - DNA, 40 bp, Artificial Sequence
  Synthetic Sequence
cacaaacaaccaggcctccacacaaacaaccaggcctcca

SEQ ID NO: 979 - DNA, 44 bp, Artificial Sequence
  Synthetic Sequence
agctttggtaacctagctttatagctttggtaacctagctttat

SEQ ID NO: 980 - DNA, 44 bp, Artificial Sequence
  Synthetic Sequence
gttcagaatcatgtcgaaagctgttcagaatcatgtcgaaagct

SEQ ID NO: 981 - DNA, 44 bp, Artificial Sequence
  Synthetic Sequence
tcggctttcaactaatgatctctcggctttcaactaatgatctc

SEQ ID NO: 982 - DNA, 44 bp, Artificial Sequence
  Synthetic Sequence
actagctttcacgatgatctcaactagctttcacgatgatctca

SEQ ID NO: 983 - DNA, 44 bp, Artificial Sequence
  Synthetic Sequence
actggctttcacgatgatctcaactggctttcacgatgatctca

SEQ ID NO: 984 - DNA, 44 bp, Artificial Sequence
  Synthetic Sequence
ttactgaatttatatggtgctattactgaatttatatggtgcta

SEQ ID NO: 985 - DNA, 44 bp, Artificial Sequence
  Synthetic Sequence
tacaatattacatactacctcatacaatattacatactacctca

SEQ ID NO: 986 - DNA, 44 bp, Artificial Sequence
  Synthetic Sequence
gcacgacttttcaaatactttggcacgacttttcaaatactttg

SEQ ID NO: 987 - DNA, 44 bp, Artificial Sequence
  Synthetic Sequence
gactgtggcaaagcattcacttgactgtggcaaagcattcactt

SEQ ID NO: 988 - DNA, 40 bp, Artificial Sequence
  Synthetic Sequence
acacctgaaactttgctcacacacctgaaactttgctcac

SEQ ID NO: 989 - DNA, 44 bp, Artificial Sequence
  Synthetic Sequence
ggggcattcaaacaacatatcaggggcattcaaacaacatatca

SEQ ID NO: 990 - DNA, 42 bp, Artificial Sequence
  Synthetic Sequence
tggcattcaccgcgtgccttatggcattcaccgcgtgcctta

SEQ ID NO: 991 - DNA, 44 bp, Artificial Sequence
  Synthetic Sequence
cgtgaattcatgcagtgccattcgtgaattcatgcagtgccatt

SEQ ID NO: 992 - DNA, 44 bp, Artificial Sequence
  Synthetic Sequence
acgatggaaaagataaccagtgacgatggaaaagataaccagtg

SEQ ID NO: 993 - DNA, 44 bp, Artificial Sequence
  Synthetic Sequence
tctcctggtcgcacaactaatatctcctggtcgcacaactaata

SEQ ID NO: 994 - DNA, 42 bp, Artificial Sequence
  Synthetic Sequence
ttctgcctgttgatcacgagcttctgcctgttgatcacgagc

SEQ ID NO: 995 - DNA, 44 bp, Artificial Sequence
  Synthetic Sequence
tcaccgcagttaagatgcattttcaccgcagttaagatgcattt

SEQ ID NO: 996 - DNA, 44 bp, Artificial Sequence
  Synthetic Sequence
tcccgcacatgcgcattgctcatcccgcacatgcgcattgctca

SEQ ID NO: 997 - DNA, 42 bp, Artificial Sequence
  Synthetic Sequence
aagggtattctcgagcaataaaagggtattctcgagcaataa

SEQ ID NO: 998 - DNA, 44 bp, Artificial Sequence
  Synthetic Sequence
tcaggccggggagagtgcaatatcaggccggggagagtgcaata

SEQ ID NO: 999 - DNA, 44 bp, Artificial Sequence
  Synthetic Sequence
agcgtcattacctgacagtattagcgtcattacctgacagtatt

SEQ ID NO: 1000 - DNA, 44 bp, Artificial Sequence
  Synthetic Sequence
aagctgttcgagaattctcaggaagctgttcgagaattctcagg

SEQ ID NO: 1001 - DNA, 44 bp, Artificial Sequence
  Synthetic Sequence
tctgaatggcatcggagtacaatctgaatggcatcggagtacaa

SEQ ID NO: 1002 - DNA, 44 bp, Artificial Sequence
  Synthetic Sequence
ccagtacctatgtgtagtacaaccagtacctatgtgtagtacaa

SEQ ID NO: 1003 - DNA, 40 bp, Artificial Sequence
  Synthetic Sequence
cagtacttttgtgtagtacacagtacttttgtgtagtaca

SEQ ID NO: 1004 - DNA, 44 bp, Artificial Sequence
  Synthetic Sequence
agcgaagatttgggggccagtaagcgaagatttgggggccagta

SEQ ID NO: 1005 - DNA, 42 bp, Artificial Sequence
  Synthetic Sequence
tcatttctcgcacctacctcatcatttctcgcacctacctca

SEQ ID NO: 1006 - DNA, 42 bp, Artificial Sequence
  Synthetic Sequence
tcgaagcaaaggcctacgcaatcgaagcaaaggcctacgcaa

SEQ ID NO: 1007 - DNA, 38 bp, Artificial Sequence
  Synthetic Sequence
atatcccgccgcgatcgtaatatcccgccgcgatcgta

SEQ ID NO: 1008 - DNA, 44 bp, Artificial Sequence
  Synthetic Sequence
cataccactttgtacaaccaaacataccactttgtacaaccaaa

SEQ ID NO: 1009 - DNA, 42 bp, Artificial Sequence
  Synthetic Sequence
agctacttggaggggaccaatagctacttggaggggaccaat

SEQ ID NO: 1010 - DNA, 44 bp, Artificial Sequence
  Synthetic Sequence
agctcctacccgaaacatgtaaagctcctacccgaaacatgtaa

SEQ ID NO: 1011 - DNA, 44 bp, Artificial Sequence
  Synthetic Sequence
aagaagagaataggctctagtcaagaagagaataggctctagtc

SEQ ID NO: 1012 - DNA, 44 bp, Artificial Sequence
  Synthetic Sequence
tgagcgttatccgtgcacgtgttgagcgttatccgtgcacgtgt

SEQ ID NO: 1013 - DNA, 42 bp, Artificial Sequence
  Synthetic Sequence
gcaacgctcaaaagtcctgtggcaacgctcaaaagtcctgtg

SEQ ID NO: 1014 - DNA, 42 bp, Artificial Sequence
  Synthetic Sequence
ccatgccaacagttgactgtgccatgccaacagttgactgtg

SEQ ID NO: 1015 - DNA, 44 bp, Artificial Sequence
  Synthetic Sequence
aataagagcggcaccactacttaataagagcggcaccactactt

SEQ ID NO: 1016 - DNA, 44 bp, Artificial Sequence
  Synthetic Sequence
ttacctgcggcactactacttattacctgcggcactactactta

SEQ ID NO: 1017 - DNA, 42 bp, Artificial Sequence
  Synthetic Sequence
ggtcagtgttagtgaggtgtgggtcagtgttagtgaggtgtg

SEQ ID NO: 1018 - DNA, 44 bp, Artificial Sequence
  Synthetic Sequence
ctacagtcgcgaaagatttgcactacagtcgcgaaagatttgca

SEQ ID NO: 1019 - DNA, 42 bp, Artificial Sequence
  Synthetic Sequence
tacagtcttctatgcattccatacagtcttctatgcattcca

SEQ ID NO: 1020 - DNA, 42 bp, Artificial Sequence
  Synthetic Sequence
tcactgggtactcctgatacttcactgggtactcctgatact

SEQ ID NO: 1021 - DNA, 42 bp, Artificial Sequence
  Synthetic Sequence
aaaaggattcctctcaaaaccaaaaggattcctctcaaaacc

SEQ ID NO: 1022 - DNA, 40 bp, Artificial Sequence
  Synthetic Sequence
taccagattaggatgagatttaccagattaggatgagatt

SEQ ID NO: 1023 - DNA, 40 bp, Artificial Sequence
  Synthetic Sequence
ctacaagagttcgacatcacctacaagagttcgacatcac

SEQ ID NO: 1024 - DNA, 38 bp, Artificial Sequence
  Synthetic Sequence
cgtgaaaactaaaaagctacgtgaaaactaaaaagcta

SEQ ID NO: 1025 - DNA, 40 bp, Artificial Sequence
  Synthetic Sequence
atcagaaaacatcgagaaacatcagaaaacatcgagaaac

SEQ ID NO: 1026 - DNA, 40 bp, Artificial Sequence
  Synthetic Sequence
cataacaacaaccacccgcccataacaacaaccacccgcc

SEQ ID NO: 1027 - DNA, 40 bp, Artificial Sequence
  Synthetic Sequence
ataccacccttcctccctcaataccacccttcctccctca

SEQ ID NO: 1028 - DNA, 40 bp, Artificial Sequence
  Synthetic Sequence
gctttgccaaagtcttgcctgctttgccaaagtcttgcct

SEQ ID NO: 1029 - DNA, 38 bp, Artificial Sequence
  Synthetic Sequence
tgcagcagacacttcacggtgcagcagacacttcacgg

SEQ ID NO: 1030 - DNA, 44 bp, Artificial Sequence
  Synthetic Sequence
ccaaactgcttctaattcttgcccaaactgcttctaattcttgc

SEQ ID NO: 1031 - DNA, 40 bp, Artificial Sequence
  Synthetic Sequence
agttttgccagagtcttgccagttttgccagagtcttgcc

SEQ ID NO: 1032 - DNA, 40 bp, Artificial Sequence
  Synthetic Sequence
ctccactgctacatcatgccctccactgctacatcatgcc

SEQ ID NO: 1033 - DNA, 40 bp, Artificial Sequence
  Synthetic Sequence
aatgctttcccacccggcgaaatgctttcccacccggcga

SEQ ID NO: 1034 - DNA, 36 bp, Artificial Sequence
  Synthetic Sequence
caaacacccatgcctacacaaacacccatgcctaca

SEQ ID NO: 1035 - DNA, 38 bp, Artificial Sequence
  Synthetic Sequence
agccgacacagtacgggcaagccgacacagtacgggca

SEQ ID NO: 1036 - DNA, 42 bp, Artificial Sequence
  Synthetic Sequence
aataccaacacatggcaattgaataccaacacatggcaattg

SEQ ID NO: 1037 - DNA, 42 bp, Artificial Sequence
  Synthetic Sequence
aggagcagcaacaaacaaggtaggagcagcaacaaacaaggt

SEQ ID NO: 1038 - DNA, 42 bp, Artificial Sequence
  Synthetic Sequence
catagctcaggctaaaacaaacatagctcaggctaaaacaaa

SEQ ID NO: 1039 - DNA, 42 bp, Artificial Sequence
  Synthetic Sequence
tgatttgttcgcgttgctcaatgatttgttcgcgttgctcaa

SEQ ID NO: 1040 - DNA, 42 bp, Artificial Sequence
  Synthetic Sequence
tcctgcaacgactggcatttatcctgcaacgactggcattta

SEQ ID NO: 1041 - DNA, 42 bp, Artificial Sequence
  Synthetic Sequence
ccttgacagggataccaattgccttgacagggataccaattg

SEQ ID NO: 1042 - DNA, 42 bp, Artificial Sequence
  Synthetic Sequence
tcgtcagagaaagaccagtgatcgtcagagaaagaccagtga

SEQ ID NO: 1043 - DNA, 42 bp, Artificial Sequence
  Synthetic Sequence
ttgtgaacgggattacggtcattgtgaacgggattacggtca

SEQ ID NO: 1044 - DNA, 42 bp, Artificial Sequence
  Synthetic Sequence
cgaaatgcgtctcatacaaaacgaaatgcgtctcatacaaaa

SEQ ID NO: 1045 - DNA, 44 bp, Artificial Sequence
  Synthetic Sequence
tcatcacacgtgatcgatgatatcatcacacgtgatcgatgata

SEQ ID NO: 1046 - DNA, 40 bp, Artificial Sequence; identical to SEQ ID NO: 485
  Synthetic Sequence

SEQ ID NO: 1047 - DNA, 44 bp, Artificial Sequence; identical to SEQ ID NO: 860
  Synthetic Sequence

SEQ ID NO: 1048 - DNA, 44 bp, Artificial Sequence
  Synthetic Sequence
acaaattcggatctacagggtaacaaattcggatctacagggta

SEQ ID NO: 1049 - DNA, 44 bp, Artificial Sequence
  Synthetic Sequence
cacaaattcggttctacagggtcacaaattcggttctacagggt

SEQ ID NO: 1050 - DNA, 42 bp, Artificial Sequence
  Synthetic Sequence
acaaccagctaagacactgccacaaccagctaagacactgcc

SEQ ID NO: 1051 - DNA, 42 bp, Artificial Sequence; identical to SEQ ID NO: 188
  Synthetic Sequence

SEQ ID NO: 1052 - DNA, 44 bp, Artificial Sequence; identical to SEQ ID NO: 191
  Synthetic Sequence

SEQ ID NO: 1053 - DNA, 42 bp, Artificial Sequence; identical to SEQ ID NO: 192
  Synthetic Sequence

SEQ ID NO: 1054 - DNA, 44 bp, Artificial Sequence; identical to SEQ ID NO: 193
  Synthetic Sequence

SEQ ID NO: 1055 - DNA, 40 bp, Artificial Sequence; identical to SEQ ID NO: 197
  Synthetic Sequence

SEQ ID NO: 1056 - DNA, 42 bp, Artificial Sequence
  Synthetic Sequence
ggctgtcaattcataggtcatggctgtcaattcataggtcat

SEQ ID NO: 1057 - DNA, 44 bp, Artificial Sequence
  Synthetic Sequence
cccaacaacatgaaactacctacccaacaacatgaaactaccta

SEQ ID NO: 1058 - DNA, 42 bp, Artificial Sequence; identical to SEQ ID NO: 214
  Synthetic Sequence

SEQ ID NO: 1059 - DNA, 44 bp, Artificial Sequence
  Synthetic Sequence
caagtggtcctaaacatttcaccaagtggtcctaaacatttcac

SEQ ID NO: 1060 - DNA, 44 bp, Artificial Sequence; identical to SEQ ID NO: 226
  Synthetic Sequence

SEQ ID NO: 1061 - DNA, 42 bp, Artificial Sequence; identical to SEQ ID NO: 227
  Synthetic Sequence

SEQ ID NO: 1062 - DNA, 42 bp, Artificial Sequence
  Synthetic Sequence
ttagccgctgtcacacgcacattagccgctgtcacacgcaca

SEQ ID NO: 1063 - DNA, 44 bp, Artificial Sequence
  Synthetic Sequence
ggtacaatcaacggtcaatggtggtacaatcaacggtcaatggt

SEQ ID NO: 1064 - DNA, 40 bp, Artificial Sequence; identical to SEQ ID NO: 237
  Synthetic Sequence

SEQ ID NO: 1065 - DNA, 44 bp, Artificial Sequence
  Synthetic Sequence
tcacagttgccagctgagattatcacagttgccagctgagatta

SEQ ID NO: 1066 - DNA, 44 bp, Artificial Sequence
  Synthetic Sequence
ccaatcagttcctgatgcagtaccaatcagttcctgatgcagta

SEQ ID NO: 1067 - DNA, 44 bp, Artificial Sequence
  Synthetic Sequence
aagaattgcgtttggacaatcaaagaattgcgtttggacaatca

SEQ ID NO: 1068 - DNA, 42 bp, Artificial Sequence
  Synthetic Sequence
aagtgtccgatacggttgtggaagtgtccgatacggttgtgg

SEQ ID NO: 1069 - DNA, 44 bp, Artificial Sequence; identical to SEQ ID NO: 245
  Synthetic Sequence

SEQ ID NO: 1070 - DNA, 42 bp, Artificial Sequence; identical to SEQ ID NO: 246
  Synthetic Sequence

SEQ ID NO: 1071 - DNA, 42 bp, Artificial Sequence; identical to SEQ ID NO: 247
  Synthetic Sequence

SEQ ID NO: 1072 - DNA, 44 bp, Artificial Sequence
  Synthetic Sequence
ctaccccaacaaatagcacttactaccccaacaaatagcactta

SEQ ID NO: 1073 - DNA, 44 bp, Artificial Sequence
  Synthetic Sequence
ctaccccaacttgatagcacttctaccccaacttgatagcactt

SEQ ID NO: 1074 - DNA, 44 bp, Artificial Sequence
  Synthetic Sequence
ctaccccaaagagaagcacttactaccccaaagagaagcactta

SEQ ID NO: 1075 - DNA, 42 bp, Artificial Sequence; identical to SEQ ID NO: 187
  Synthetic Sequence

SEQ ID NO: 1076 - DNA, 44 bp, Artificial Sequence; identical to SEQ ID NO: 612
  Synthetic Sequence

SEQ ID NO: 1077 - DNA, 44 bp, Artificial Sequence; identical to SEQ ID NO: 619
  Synthetic Sequence

SEQ ID NO: 1078 - DNA, 44 bp, Artificial Sequence; identical to SEQ ID NO: 122
  Synthetic Sequence

SEQ ID NO: 1079 - DNA, 44 bp, Artificial Sequence; identical to SEQ ID NO: 123
  Synthetic Sequence

SEQ ID NO: 1080 - DNA, 44 bp, Artificial Sequence; identical to SEQ ID NO: 124
  Synthetic Sequence

SEQ ID NO: 1081 - DNA, 44 bp, Artificial Sequence
  Synthetic Sequence
aaccatacaaccaactacctcaaaccatacaaccaactacctca

SEQ ID NO: 1082 - DNA, 44 bp, Artificial Sequence
  Synthetic Sequence
aactattcaatctactacctcaaactattcaatctactacctca

SEQ ID NO: 1083 - DNA, 44 bp, Artificial Sequence; identical to SEQ ID NO: 127
  Synthetic Sequence

SEQ ID NO: 1084 - DNA, 44 bp, Artificial Sequence
  Synthetic Sequence
aactatacaaactactacctcaaactatacaaactactacctca

SEQ ID NO: 1085 - DNA, 44 bp, Artificial Sequence
  Synthetic Sequence
aacaacacaacttactacctcaaacaacacaacttactacctca

SEQ ID NO: 1086 - DNA, 44 bp, Artificial Sequence
  Synthetic Sequence
aacagcacaaactactacctcaaacagcacaaactactacctca

SEQ ID NO: 1087 - DNA, 44 bp, Artificial Sequence
  Synthetic Sequence
atacatacttctttacattccaatacatacttctttacattcca

SEQ ID NO: 1088 - DNA, 44 bp, Artificial Sequence; identical to SEQ ID NO: 8
  Synthetic Sequence

SEQ ID NO: 1089 - DNA, 44 bp, Artificial Sequence
  Synthetic Sequence
acaaatccggatctacagggtaacaaatccggatctacagggta

SEQ ID NO: 1090 - DNA, 44 bp, Artificial Sequence
  Synthetic Sequence
acacattcggttctacagggtaacacattcggttctacagggta

SEQ ID NO: 1091 - DNA, 44 bp, Artificial Sequence
  Synthetic Sequence
cacaaaccattctgtgctgctacacaaaccattctgtgctgcta

SEQ ID NO: 1092 - DNA, 44 bp, Artificial Sequence
  Synthetic Sequence
tacaaaccatgatgtgctgctatacaaaccatgatgtgctgcta

SEQ ID NO: 1093 - DNA, 44 bp, Artificial Sequence
  Synthetic Sequence
caccaatatttacgtgctgctacaccaatatttacgtgctgcta

SEQ ID NO: 1094 - DNA, 44 bp, Artificial Sequence
  Synthetic Sequence
ctccaatatttacgtgctgctactccaatatttacgtgctgcta

SEQ ID NO: 1095 - DNA, 44 bp, Artificial Sequence
  Synthetic Sequence
ctccaatatttacatgctgctactccaatatttacatgctgcta

SEQ ID NO: 1096 - DNA, 44 bp, Artificial Sequence
  Synthetic Sequence
tacctgcactgtaagcactttgtacctgcactgtaagcactttg

SEQ ID NO: 1097 - DNA, 42 bp, Artificial Sequence
  Synthetic Sequence
ctacctgcactgtgagcacttctacctgcactgtgagcactt

SEQ ID NO: 1098 - DNA, 44 bp, Artificial Sequence; identical to SEQ ID NO: 200
  Synthetic Sequence

SEQ ID NO: 1099 - DNA, 44 bp, Artificial Sequence
  Synthetic Sequence
tatctgcactaaatgcaccttatatctgcactaaatgcacctta

SEQ ID NO: 1100 - DNA, 44 bp, Artificial Sequence
  Synthetic Sequence
taactacacaagatgcaccttataactacacaagatgcacctta

SEQ ID NO: 1101 - DNA, 44 bp, Artificial Sequence; identical to SEQ ID NO: 217
  Synthetic Sequence

SEQ ID NO: 1102 - DNA, 44 bp, Artificial Sequence; identical to SEQ ID NO: 218
  Synthetic Sequence

SEQ ID NO: 1103 - DNA, 44 bp, Artificial Sequence
  Synthetic Sequence
cgagttttgcatggatttgcaccgagttttgcatggatttgcac

SEQ ID NO: 1104 - DNA, 44 bp, Artificial Sequence
  Synthetic Sequence
tcagttttgcatgggtttgcactcagttttgcatgggtttgcac

SEQ ID NO: 1105 - DNA, 44 bp, Artificial Sequence; identical to SEQ ID NO: 230
  Synthetic Sequence

SEQ ID NO: 1106 - DNA, 44 bp, Artificial Sequence
  Synthetic Sequence
ccaacaccagtctgataagctaccaacaccagtctgataagcta

SEQ ID NO: 1107 - DNA, 42 bp, Artificial Sequence
  Synthetic Sequence
acagttcttcagctggcagctacagttcttcagctggcagct

SEQ ID NO: 1108 - DNA, 42 bp, Artificial Sequence
  Synthetic Sequence
acagctcttcaactggcagctacagctcttcaactggcagct

SEQ ID NO: 1109 - DNA, 44 bp, Artificial Sequence
  Synthetic Sequence
tggaaatccctggcaatgtgattggaaatccctggcaatgtgat

SEQ ID NO: 1110 - DNA, 44 bp, Artificial Sequence
  Synthetic Sequence
tggtaatccctggcaatgtgattggtaatccctggcaatgtgat

SEQ ID NO: 1111 - DNA, 42 bp, Artificial Sequence; identical to SEQ ID NO: 251
  Synthetic Sequence

SEQ ID NO: 1112 - DNA, 42 bp, Artificial Sequence; identical to SEQ ID NO: 252
  Synthetic Sequence

SEQ ID NO: 1113 - DNA, 44 bp, Artificial Sequence
  Synthetic Sequence
agcctatcctggattacttgaaagcctatcctggattacttgaa

SEQ ID NO: 1114 - DNA, 44 bp, Artificial Sequence
  Synthetic Sequence
aacctatcctggattacttgaaaacctatcctggattacttgaa

SEQ ID NO: 1115 - DNA, 42 bp, Artificial Sequence
  Synthetic Sequence
agcggaacttagccactgtgaagcggaacttagccactgtga

SEQ ID NO: 1116 - DNA, 44 bp, Artificial Sequence
  Synthetic Sequence
tgcagaacttagccactgtgaatgcagaacttagccactgtgaa

SEQ ID NO: 1117 - DNA, 42 bp, Artificial Sequence
  Synthetic Sequence
gcagaacttaaccactgtgaagcagaacttaaccactgtgaa

SEQ ID NO: 1118 - DNA, 44 bp, Artificial Sequence
  Synthetic Sequence
tgaagaacttagccactgtgaatgaagaacttagccactgtgaa

SEQ ID NO: 1119 - DNA, 44 bp, Artificial Sequence
  Synthetic Sequence
cactgaacttagccactgtgaacactgaacttagccactgtgaa

SEQ ID NO: 1120 - DNA, 44 bp, Artificial Sequence
  Synthetic Sequence
acactgatttcaaatggtgctaacactgatttcaaatggtgcta

SEQ ID NO: 1121 - DNA, 44 bp, Artificial Sequence
  Synthetic Sequence
taaccgatttcaaatggtgctataaccgatttcaaatggtgcta

SEQ ID NO: 1122 - DNA, 44 bp, Artificial Sequence
  Synthetic Sequence
cttccagtcgggaatgtttacacttccagtcgggaatgtttaca

SEQ ID NO: 1123 - DNA, 44 bp, Artificial Sequence; identical to SEQ ID NO: 273
  Synthetic Sequence

SEQ ID NO: 1124 - DNA, 44 bp, Artificial Sequence
  Synthetic Sequence
ctgagagtgtaggatgtttacactgagagtgtaggatgtttaca

SEQ ID NO: 1125 - DNA, 42 bp, Artificial Sequence; identical to SEQ ID NO: 275
  Synthetic Sequence

SEQ ID NO: 1126 - DNA, 44 bp, Artificial Sequence
  Synthetic Sequence
cttccagtcaaggatgtttacacttccagtcaaggatgtttaca

SEQ ID NO: 1127 - DNA, 44 bp, Artificial Sequence
  Synthetic Sequence
acaggccgggacaagtgcaataacaggccgggacaagtgcaata

SEQ ID NO: 1128 - DNA, 40 bp, Artificial Sequence
  Synthetic Sequence
aggccgggacgagtgcaataaggccgggacgagtgcaata

SEQ ID NO: 1129 - DNA, 44 bp, Artificial Sequence
  Synthetic Sequence
tacctgcacaaacagcactttttacctgcacaaacagcactttt

SEQ ID NO: 1130 - DNA, 44 bp, Artificial Sequence; identical to SEQ ID NO: 523
  Synthetic Sequence

SEQ ID NO: 1131 - DNA, 42 bp, Artificial Sequence; identical to SEQ ID NO: 432
  Synthetic Sequence

SEQ ID NO: 1132 - DNA, 42 bp, Artificial Sequence; identical to SEQ ID NO: 131
  Synthetic Sequence

SEQ ID NO: 1133 - DNA, 44 bp, Artificial Sequence; identical to SEQ ID NO: 132
  Synthetic Sequence

SEQ ID NO: 1134 - DNA, 44 bp, Artificial Sequence
  Synthetic Sequence
cttcagttatcatagtactgtacttcagttatcatagtactgta

SEQ ID NO: 1135 - DNA, 42 bp, Artificial Sequence; identical to SEQ ID NO: 16
  Synthetic Sequence

SEQ ID NO: 1136 - DNA, 42 bp, Artificial Sequence; identical to SEQ ID NO: 137
  Synthetic Sequence

SEQ ID NO: 1137 - DNA, 44 bp, Artificial Sequence
  Synthetic Sequence
caaacaccattgtcacactccacaaacaccattgtcacactcca

SEQ ID NO: 1138 - DNA, 44 bp, Artificial Sequence; identical to SEQ ID NO: 891
  Synthetic Sequence

SEQ ID NO: 1139 - DNA, 42 bp, Artificial Sequence
  Synthetic Sequence
acaggttaagggtctcagggaacaggttaagggtctcaggga

SEQ ID NO: 1140 - DNA, 42 bp, Artificial Sequence; identical to SEQ ID NO: 143
  Synthetic Sequence

SEQ ID NO: 1141 - DNA, 44 bp, Artificial Sequence
  Synthetic Sequence
tcacgagttagggtctcagggatcacgagttagggtctcaggga

SEQ ID NO: 1142 - DNA, 42 bp, Artificial Sequence; identical to SEQ ID NO: 144
  Synthetic Sequence

SEQ ID NO: 1143 - DNA, 44 bp, Artificial Sequence; identical to SEQ ID NO: 147
  Synthetic Sequence

SEQ ID NO: 1144 - DNA, 42 bp, Artificial Sequence; identical to SEQ ID NO: 466
  Synthetic Sequence

SEQ ID NO: 1145 - DNA, 44 bp, Artificial Sequence; identical to SEQ ID NO: 150
  Synthetic Sequence

SEQ ID NO: 1146 - DNA, 44 bp, Artificial Sequence
  Synthetic Sequence
atgccctttcattattgcactgatgccctttcattattgcactg

SEQ ID NO: 1147 - DNA, 44 bp, Artificial Sequence
  Synthetic Sequence
atgcccttttaatattgcactgatgcccttttaatattgcactg

SEQ ID NO: 1148 - DNA, 42 bp, Artificial Sequence; identical to SEQ ID NO: 152
  Synthetic Sequence

SEQ ID NO: 1149 - DNA, 42 bp, Artificial Sequence
  Synthetic Sequence
agctggttgaaggggaccaaaagctggttgaaggggaccaaa

SEQ ID NO: 1150 - DNA, 42 bp, Artificial Sequence; identical to SEQ ID NO: 154
  Synthetic Sequence

SEQ ID NO: 1151 - DNA, 44 bp, Artificial Sequence
  Synthetic Sequence
tagctggttgaaagggaccaaatagctggttgaaagggaccaaa

SEQ ID NO: 1152 - DNA, 44 bp, Artificial Sequence
  Synthetic Sequence
cacataggaatagaaagccatacacataggaatagaaagccata

SEQ ID NO: 1153 - DNA, 44 bp, Artificial Sequence
  Synthetic Sequence
tacgcgtattcttaagcaataatacgcgtattcttaagcaataa

SEQ ID NO: 1154 - DNA, 42 bp, Artificial Sequence
  Synthetic Sequence
gcctgattcacaacaccagctgcctgattcacaacaccagct

SEQ ID NO: 1155 - DNA, 44 bp, Artificial Sequence; identical to SEQ ID NO: 650
  Synthetic Sequence

SEQ ID NO: 1156 - DNA, 44 bp, Artificial Sequence
  Synthetic Sequence
gcatcgttaccagacagtgttagcatcgttaccagacagtgtta

SEQ ID NO: 1157 - DNA, 40 bp, Artificial Sequence; identical to SEQ ID NO: 165
  Synthetic Sequence

SEQ ID NO: 1158 - DNA, 44 bp, Artificial Sequence
  Synthetic Sequence
tagtagtgctgtctactttatgtagtagtgctgtctactttatg

SEQ ID NO: 1159 - DNA, 42 bp, Artificial Sequence
  Synthetic Sequence
gagctacagtgcttcatctcagagctacagtgcttcatctca

SEQ ID NO: 1160 - DNA, 40 bp, Artificial Sequence
  Synthetic Sequence
agtacatcatctatactgtaagtacatcatctatactgta

SEQ ID NO: 1161 - DNA, 42 bp, Artificial Sequence
  Synthetic Sequence
gggattcctgggaaaactggagggattcctgggaaaactgga

SEQ ID NO: 1162 - DNA, 44 bp, Artificial Sequence
  Synthetic Sequence
ccatctatggaattcagttctcccatctatggaattcagttctc

SEQ ID NO: 1163 - DNA, 44 bp, Artificial Sequence
  Synthetic Sequence
cacccttggaattcagttctcacacccttggaattcagttctca

SEQ ID NO: 1164 - DNA, 44 bp, Artificial Sequence
  Synthetic Sequence
acaaagttctgtaatgcactgaacaaagttctgtaatgcactga

SEQ ID NO: 1165 - DNA, 42 bp, Artificial Sequence
  Synthetic Sequence
cactggtacaaggattgggagcactggtacaaggattgggag

SEQ ID NO: 1166 - DNA, 44 bp, Artificial Sequence
  Synthetic Sequence
ccaaagttctgtcatgcactgaccaaagttctgtcatgcactga

SEQ ID NO: 1167 - DNA, 44 bp, Artificial Sequence
  Synthetic Sequence
gctcatttttgtgactatgcaagctcatttttgtgactatgcaa

SEQ ID NO: 1168 - DNA, 44 bp, Artificial Sequence; identical to SEQ ID NO: 662
  Synthetic Sequence

SEQ ID NO: 1169 - DNA, 44 bp, Artificial Sequence
  Synthetic Sequence
gatcatttttgtgactatgcaagatcatttttgtgactatgcaa

SEQ ID NO: 1170 - DNA, 44 bp, Artificial Sequence; identical to SEQ ID NO: 181
  Synthetic Sequence

SEQ ID NO: 1171 - DNA, 42 bp, Artificial Sequence; identical to SEQ ID NO: 188
  Synthetic Sequence

SEQ ID NO: 1172 - DNA, 42 bp, Artificial Sequence; identical to SEQ ID NO: 871
  Synthetic Sequence

SEQ ID NO: 1173 - DNA, 44 bp, Artificial Sequence; identical to SEQ ID NO: 202
  Synthetic Sequence

SEQ ID NO: 1174 - DNA, 44 bp, Artificial Sequence
  Synthetic Sequence
agctgcattatgggttccgttaagctgcattatgggttccgtta

SEQ ID NO: 1175 - DNA, 42 bp, Artificial Sequence
  Synthetic Sequence
actgggactttgtaggccagtactgggactttgtaggccagt

SEQ ID NO: 1176 - DNA, 44 bp, Artificial Sequence
  Synthetic Sequence
agcgggactttgcgggccagttagcgggactttgcgggccagtt

SEQ ID NO: 1177 - DNA, 42 bp, Artificial Sequence
  Synthetic Sequence
ccacatggagttgctgttacaccacatggagttgctgttaca

SEQ ID NO: 1178 - DNA, 44 bp, Artificial Sequence
  Synthetic Sequence
tccacatggagcggctgttacatccacatggagcggctgttaca

SEQ ID NO: 1179 - DNA, 44 bp, Artificial Sequence
  Synthetic Sequence
cccaacaacttgaaactacctacccaacaacttgaaactaccta

SEQ ID NO: 1180 - DNA, 44 bp, Artificial Sequence; identical to SEQ ID NO: 219
  Synthetic Sequence

SEQ ID NO: 1181 - DNA, 44 bp, Artificial Sequence
  Synthetic Sequence
tcatcattaccaggcagtattatcatcattaccaggcagtatta

SEQ ID NO: 1182 - DNA, 44 bp, Artificial Sequence
  Synthetic Sequence
gcatcattaccaggcagtattagcatcattaccaggcagtatta

SEQ ID NO: 1183 - DNA, 42 bp, Artificial Sequence
  Synthetic Sequence
ttttcccatgccctatgcctcttttcccatgccctatgcctc

SEQ ID NO: 1184 - DNA, 44 bp, Artificial Sequence
  Synthetic Sequence
caagtggtcctgaacatttcaccaagtggtcctgaacatttcac

SEQ ID NO: 1185 - DNA, 44 bp, Artificial Sequence; identical to SEQ ID NO: 228
  Synthetic Sequence

SEQ ID NO: 1186 - DNA, 44 bp, Artificial Sequence
  Synthetic Sequence
tcacagttgcctgcagagattatcacagttgcctgcagagatta

SEQ ID NO: 1187 - DNA, 44 bp, Artificial Sequence
  Synthetic Sequence
cacatggttagatcaagcacaacacatggttagatcaagcacaa

SEQ ID NO: 1188 - DNA, 44 bp, Artificial Sequence
  Synthetic Sequence
tgcatggttagatcaagcacaatgcatggttagatcaagcacaa

SEQ ID NO: 1189 - DNA, 44 bp, Artificial Sequence
  Synthetic Sequence
ctttgacaatactattgcactgctttgacaatactattgcactg

SEQ ID NO: 1190 - DNA, 44 bp, Artificial Sequence
  Synthetic Sequence
caatgacaatactattgcactgcaatgacaatactattgcactg

SEQ ID NO: 1191 - DNA, 44 bp, Artificial Sequence
  Synthetic Sequence
ctatgacaatactattgcactgctatgacaatactattgcactg

SEQ ID NO: 1192 - DNA, 44 bp, Artificial Sequence
  Synthetic Sequence
caacaaaatcactgatgctggacaacaaaatcactgatgctgga

SEQ ID NO: 1193 - DNA, 44 bp, Artificial Sequence
  Synthetic Sequence
tacagatggataccgtgcaatttacagatggataccgtgcaatt

SEQ ID NO: 1194 - DNA, 44 bp, Artificial Sequence; identical to SEQ ID NO: 305
  Synthetic Sequence

SEQ ID NO: 1195 - DNA, 42 bp, Artificial Sequence
  Synthetic Sequence
taacgcgagccgaacgaacaataacgcgagccgaacgaacaa

SEQ ID NO: 1196 - DNA, 44 bp, Artificial Sequence
  Synthetic Sequence
ccctattagcaatattgcactaccctattagcaatattgcacta

SEQ ID NO: 1197 - DNA, 44 bp, Artificial Sequence
  Synthetic Sequence
ccctataagcaatattgcactaccctataagcaatattgcacta

SEQ ID NO: 1198 - DNA, 42 bp, Artificial Sequence
  Synthetic Sequence
cgatgtagtccaagggcacatcgatgtagtccaagggcacat

SEQ ID NO: 1199 - DNA, 44 bp, Artificial Sequence
  Synthetic Sequence
ctacgccaacaaatagcacttactacgccaacaaatagcactta

SEQ ID NO: 1200 - DNA, 44 bp, Artificial Sequence
  Synthetic Sequence
taccccaatttgatagcacttttaccccaatttgatagcacttt

SEQ ID NO: 1201 - DNA, 42 bp, Artificial Sequence
  Synthetic Sequence
tgacaaccatctaaccagccttgacaaccatctaaccagcct

SEQ ID NO: 1202 - DNA, 44 bp, Artificial Sequence
  Synthetic Sequence
tgccaatattgatgtgctgctttgccaatattgatgtgctgctt

SEQ ID NO: 1203 - DNA, 44 bp, Artificial Sequence
  Synthetic Sequence
ctccagtatttatgtgctgcttctccagtatttatgtgctgctt

SEQ ID NO: 1204 - DNA, 44 bp, Artificial Sequence
  Synthetic Sequence
gcagtaccattcaaagagctatgcagtaccattcaaagagctat

SEQ ID NO: 1205 - DNA, 44 bp, Artificial Sequence
  Synthetic Sequence
caggatgaatccttgttactgacaggatgaatccttgttactga

SEQ ID NO: 1206 - DNA, 42 bp, Artificial Sequence
  Synthetic Sequence
cgcacagtgtgtacaatgcagcgcacagtgtgtacaatgcag

SEQ ID NO: 1207 - DNA, 42 bp, Artificial Sequence
  Synthetic Sequence
catccacattgtatgcgctgtcatccacattgtatgcgctgt

SEQ ID NO: 1208 - DNA, 44 bp, Artificial Sequence
  Synthetic Sequence
ttggcatttagcccattcctgattggcatttagcccattcctga

SEQ ID NO: 1209 - DNA, 44 bp, Artificial Sequence
  Synthetic Sequence
aaacatcactgcaagtcttaacaaacatcactgcaagtcttaac

SEQ ID NO: 1210 - DNA, 42 bp, Artificial Sequence
  Synthetic Sequence
agaggagagccgtgtatgactagaggagagccgtgtatgact

SEQ ID NO: 1211 - DNA, 44 bp, Artificial Sequence
  Synthetic Sequence
acaggccatctgtgttatattcacaggccatctgtgttatattc

SEQ ID NO: 1212 - DNA, 38 bp, Artificial Sequence
  Synthetic Sequence
aggccgggacgagtgcaataggccgggacgagtgcaat

SEQ ID NO: 1213 - DNA, 42 bp, Artificial Sequence
  Synthetic Sequence
gtacaaaccacagtgtgctgcgtacaaaccacagtgtgctgc

SEQ ID NO: 1214 - DNA, 44 bp, Artificial Sequence; identical to SEQ ID NO: 356
  Synthetic Sequence

SEQ ID NO: 1215 - DNA, 44 bp, Artificial Sequence; identical to SEQ ID NO: 343
  Synthetic Sequence

SEQ ID NO: 1216 - DNA, 44 bp, Artificial Sequence
  Synthetic Sequence
aaaatctctgcaggcaaatgtgaaaatctctgcaggcaaatgtg

SEQ ID NO: 1217 - DNA, 44 bp, Artificial Sequence; identical to SEQ ID NO: 357
  Synthetic Sequence

SEQ ID NO: 1218 - DNA, 44 bp, Artificial Sequence; identical to SEQ ID NO: 342
  Synthetic Sequence

SEQ ID NO: 1219 - DNA, 44 bp, Artificial Sequence
  Synthetic Sequence
agagaaccattaccattactaaagagaaccattaccattactaa

SEQ ID NO: 1220 - DNA, 44 bp, Artificial Sequence
  Synthetic Sequence
cccaccgacaacaatgaatgttcccaccgacaacaatgaatgtt

SEQ ID NO: 1221 - DNA, 34 bp, Artificial Sequence
  Synthetic Sequence
gctccaggcagcccaaagctccaggcagcccaaa

SEQ ID NO: 1222 - DNA, 34 bp, Artificial Sequence
  Synthetic Sequence
cccacgcaccagggtaacccacgcaccagggtaa

SEQ ID NO: 1223 - DNA, 44 bp, Artificial Sequence
  Synthetic Sequence
atgttcaaataagcttttgtaaatgttcaaataagcttttgtaa

SEQ ID NO: 1224 - DNA, 44 bp, Artificial Sequence
  Synthetic Sequence
ttttttttcaacttgttacagcttttttttcaacttgttacagc

SEQ ID NO: 1225 - DNA, 44 bp, Artificial Sequence
  Synthetic Sequence
aaacaaagcacctctccaaaaaaaacaaagcacctctccaaaaa

SEQ ID NO: 1226 - DNA, 44 bp, Artificial Sequence
  Synthetic Sequence
gctaacaaggaatgctgccaaagctaacaaggaatgctgccaaa

SEQ ID NO: 1227 - DNA, 44 bp, Artificial Sequence
  Synthetic Sequence
gagaaattttcagggctactgagagaaattttcagggctactga

SEQ ID NO: 1228 - DNA, 42 bp, Artificial Sequence
  Synthetic Sequence
tgaatccttgcccaggtgcattgaatccttgcccaggtgcat

SEQ ID NO: 1229 - DNA, 42 bp, Artificial Sequence
  Synthetic Sequence
gagctgagtggagcacaaacagagctgagtggagcacaaaca

SEQ ID NO: 1230 - DNA, 44 bp, Artificial Sequence
  Synthetic Sequence
ttgttcaaccagttactaatctttgttcaaccagttactaatct

SEQ ID NO: 1231 - DNA, 44 bp, Artificial Sequence
  Synthetic Sequence
agctgccggcattaaagggctaagctgccggcattaaagggcta

SEQ ID NO: 1232 - DNA, 44 bp, Artificial Sequence
  Synthetic Sequence
ccaaattagctttttaaatagaccaaattagctttttaaataga

SEQ ID NO: 1233 - DNA, 44 bp, Artificial Sequence
  Synthetic Sequence
aacccaatatcaaacatatcacaacccaatatcaaacatatcac

SEQ ID NO: 1234 - DNA, 44 bp, Artificial Sequence
  Synthetic Sequence
ccaagaaatagcctttcaaacaccaagaaatagcctttcaaaca

SEQ ID NO: 1235 - DNA, 34 bp, Artificial Sequence
  Synthetic Sequence
accccgtgccactgtgtaccccgtgccactgtgt

SEQ ID NO: 1236 - DNA, 44 bp, Artificial Sequence
  Synthetic Sequence
catgtcataagccatttatttccatgtcataagccatttatttc

SEQ ID NO: 1237 - DNA, 44 bp, Artificial Sequence
  Synthetic Sequence
ttgggagaccctggtctgcactttgggagaccctggtctgcact

SEQ ID NO: 1238 - DNA, 44 bp, Artificial Sequence
  Synthetic Sequence
ctaatgaccgcagaaagccattctaatgaccgcagaaagccatt

SEQ ID NO: 1239 - DNA, 44 bp, Artificial Sequence
  Synthetic Sequence
cattcaacaaacatttaatgagcattcaacaaacatttaatgag

SEQ ID NO: 1240 - DNA, 44 bp, Artificial Sequence; identical to SEQ ID NO: 170
  Synthetic Sequence

SEQ ID NO: 1241 - DNA, 44 bp, Artificial Sequence
  Synthetic Sequence
aagaagtgcaccatgtttgtttaagaagtgcaccatgtttgttt

SEQ ID NO: 1242 - DNA, 42 bp, Artificial Sequence
  Synthetic Sequence
tgcctggcacctacacactaatgcctggcacctacacactaa

SEQ ID NO: 1243 - DNA, 42 bp, Artificial Sequence
  Synthetic Sequence
tgctaaatgatcccctggtgctgctaaatgatcccctggtgc

SEQ ID NO: 1244 - DNA, 44 bp, Artificial Sequence
  Synthetic Sequence
ccaattaagtcttttaaataaaccaattaagtcttttaaataaa

SEQ ID NO: 1245 - DNA, 42 bp, Artificial Sequence
  Synthetic Sequence
cacttcactgcctgcagacaacacttcactgcctgcagacaa

SEQ ID NO: 1246 - DNA, 44 bp, Artificial Sequence
  Synthetic Sequence
cgttcctgataagtgaataaaacgttcctgataagtgaataaaa

SEQ ID NO: 1247 - DNA, 44 bp, Artificial Sequence
  Synthetic Sequence
gcagttcagaaaattaaatagagcagttcagaaaattaaataga

SEQ ID NO: 1248 - DNA, 44 bp, Artificial Sequence
  Synthetic Sequence
gttctccaatacctaggcacaagttctccaatacctaggcacaa

SEQ ID NO: 1249 - DNA, 44 bp, Artificial Sequence; identical to SEQ ID NO: 344
  Synthetic Sequence

SEQ ID NO: 1250 - DNA, 44 bp, Artificial Sequence
  Synthetic Sequence
tagggtcacacaggatgtgaattagggtcacacaggatgtgaat

SEQ ID NO: 1251 - DNA, 44 bp, Artificial Sequence
  Synthetic Sequence
acaaggatgaatctttgttactacaaggatgaatctttgttact

SEQ ID NO: 1252 - DNA, 40 bp, Artificial Sequence
  Synthetic Sequence
cagaactgttcccgctgctacagaactgttcccgctgcta

SEQ ID NO: 1253 - DNA, 42 bp, Artificial Sequence
  Synthetic Sequence
aggttacccgagcaactttgcaggttacccgagcaactttgc

SEQ ID NO: 1254 - DNA, 44 bp, Artificial Sequence
  Synthetic Sequence
gaggggagttttctttcaaaaggaggggagttttctttcaaaag

SEQ ID NO: 1255 - DNA, 44 bp, Artificial Sequence
  Synthetic Sequence
atccttgaataggtgtgttgcaatccttgaataggtgtgttgca

SEQ ID NO: 1256 - DNA, 44 bp, Artificial Sequence
  Synthetic Sequence
tttacagggtggcccatttaaatttacagggtggcccatttaaa

SEQ ID NO: 1257 - DNA, 44 bp, Artificial Sequence
  Synthetic Sequence
caaagagcatgatatttgacagcaaagagcatgatatttgacag

SEQ ID NO: 1258 - DNA, 44 bp, Artificial Sequence
  Synthetic Sequence
ggtcaatatttacctctcaggtggtcaatatttacctctcaggt

SEQ ID NO: 1259 - DNA, 44 bp, Artificial Sequence
  Synthetic Sequence
tcaggccatcagcagctgctattcaggccatcagcagctgctat

SEQ ID NO: 1260 - DNA, 42 bp, Artificial Sequence
  Synthetic Sequence
ccaggaattgatgaccagctgccaggaattgatgaccagctg

SEQ ID NO: 1261 - DNA, 42 bp, Artificial Sequence
  Synthetic Sequence
aggacccagagaacaactcagaggacccagagaacaactcag

SEQ ID NO: 1262 - DNA, 42 bp, Artificial Sequence
  Synthetic Sequence
acctagggatcgtcaaagggaacctagggatcgtcaaaggga

SEQ ID NO: 1263 - DNA, 44 bp, Artificial Sequence
  Synthetic Sequence
tttcctctgcaaacagttgtaatttcctctgcaaacagttgtaa

SEQ ID NO: 1264 - DNA, 44 bp, Artificial Sequence
  Synthetic Sequence
tttagtcaatatcaagatttattttagtcaatatcaagatttat

SEQ ID NO: 1265 - DNA, 34 bp, Artificial Sequence
  Synthetic Sequence
aagcttcccgggcagctaagcttcccgggcagct

SEQ ID NO: 1266 - DNA, 44 bp, Artificial Sequence
  Synthetic Sequence
tgcccatggactgcatggtgcttgcccatggactgcatggtgct

SEQ ID NO: 1267 - DNA, 42 bp, Artificial Sequence
  Synthetic Sequence
gctgattgcctctgtgccaatgctgattgcctctgtgccaat

SEQ ID NO: 1268 - DNA, 44 bp, Artificial Sequence
  Synthetic Sequence
aacgccggggccacgttgctaaaacgccggggccacgttgctaa

SEQ ID NO: 1269 - DNA, 44 bp, Artificial Sequence
  Synthetic Sequence
cgaaaggagattggccatgtaacgaaaggagattggccatgtaa

SEQ ID NO: 1270 - DNA, 44 bp, Artificial Sequence
  Synthetic Sequence
ttcctactgaaatctgacaatcttcctactgaaatctgacaatc

SEQ ID NO: 1271 - DNA, 44 bp, Artificial Sequence
  Synthetic Sequence
gaaagaccccatttaacttgaagaaagaccccatttaacttgaa

SEQ ID NO: 1272 - DNA, 44 bp, Artificial Sequence
  Synthetic Sequence
tgaacaatccagataattgctttgaacaatccagataattgctt

SEQ ID NO: 1273 - DNA, 36 bp, Artificial Sequence
  Synthetic Sequence
tcccctgcaagtggtgcttcccctgcaagtggtgct

SEQ ID NO: 1274 - DNA, 40 bp, Artificial Sequence
  Synthetic Sequence
tcccacacccaaggcttgcatcccacacccaaggcttgca

SEQ ID NO: 1275 - DNA, 42 bp, Artificial Sequence
  Synthetic Sequence
gaaaccaagtatgggtcgcctgaaaccaagtatgggtcgcct

SEQ ID NO: 1276 - DNA, 44 bp, Artificial Sequence
  Synthetic Sequence
tgtgtgcaattacccattttattgtgtgcaattacccattttat

SEQ ID NO: 1277 - DNA, 44 bp, Artificial Sequence
  Synthetic Sequence
atttaaaaggcttttaaatgatatttaaaaggcttttaaatgat

SEQ ID NO: 1278 - DNA, 44 bp, Artificial Sequence
  Synthetic Sequence
atagtagaccgtatagcgtacgatagtagaccgtatagcgtacg

SEQ ID NO: 1279 - DNA, 42 bp, Artificial Sequence
  Synthetic Sequence
actggggctgcatgctgctcaactggggctgcatgctgctca

SEQ ID NO: 1280 - DNA, 44 bp, Artificial Sequence
  Synthetic Sequence
ctactgttaatgacctatttctctactgttaatgacctatttct

SEQ ID NO: 1281 - DNA, 44 bp, Artificial Sequence
  Synthetic Sequence
cctaaatacctggtatttgagacctaaatacctggtatttgaga

SEQ ID NO: 1282 - DNA, 44 bp, Artificial Sequence
  Synthetic Sequence
ctttgacagcattttaattatactttgacagcattttaattata

SEQ ID NO: 1283 - DNA, 44 bp, Artificial Sequence
  Synthetic Sequence
gaacacaccaaggataatttctgaacacaccaaggataatttct

SEQ ID NO: 1284 - DNA, 44 bp, Artificial Sequence
  Synthetic Sequence
agttatgaaatgtcatcaataaagttatgaaatgtcatcaataa

SEQ ID NO: 1285 - DNA, 42 bp, Artificial Sequence
  Synthetic Sequence
cacaggaagtggccttcaatacacaggaagtggccttcaata

SEQ ID NO: 1286 - DNA, 44 bp, Artificial Sequence
  Synthetic Sequence
attgtttgcactctgccagtttattgtttgcactctgccagttt

SEQ ID NO: 1287 - DNA, 44 bp, Artificial Sequence
  Synthetic Sequence
gagctgaactcaaaaccaaatggagctgaactcaaaaccaaatg

SEQ ID NO: 1288 - DNA, 44 bp, Artificial Sequence
  Synthetic Sequence
tctttattgcaaagtcagtatgtctttattgcaaagtcagtatg

SEQ ID NO: 1289 - DNA, 44 bp, Artificial Sequence
  Synthetic Sequence
aaccctaggagagggtgccattaaccctaggagagggtgccatt

SEQ ID NO: 1290 - DNA, 42 bp, Artificial Sequence
  Synthetic Sequence
attctgcccctggatatgcatattctgcccctggatatgcat

SEQ ID NO: 1291 - DNA, 38 bp, Artificial Sequence
  Synthetic Sequence
aaccaagcagccgggcagtaaccaagcagccgggcagt

SEQ ID NO: 1292 - DNA, 42 bp, Artificial Sequence
  Synthetic Sequence
agcagggctccctcaccagcaagcagggctccctcaccagca

SEQ ID NO: 1293 - DNA, 44 bp, Artificial Sequence; identical to SEQ ID NO: 305
  Synthetic Sequence

SEQ ID NO: 1294 - DNA, 40 bp, Artificial Sequence
  Synthetic Sequence
cgccgccccgcacctgctgccgccgccccgcacctgctgc

SEQ ID NO: 1295 - DNA, 42 bp, Artificial Sequence
  Synthetic Sequence
acatctcggggatcatcatgtacatctcggggatcatcatgt

SEQ ID NO: 1296 - DNA, 44 bp, Artificial Sequence
  Synthetic Sequence
gggccctatattaatggaccaagggccctatattaatggaccaa

SEQ ID NO: 1297 - DNA, 44 bp, Artificial Sequence
  Synthetic Sequence
agtaaagccaagtagtgcatgaagtaaagccaagtagtgcatga

SEQ ID NO: 1298 - DNA, 44 bp, Artificial Sequence
  Synthetic Sequence
aagaaggaccttgtaataaataaagaaggaccttgtaataaata

SEQ ID NO: 1299 - DNA, 42 bp, Artificial Sequence
  Synthetic Sequence
ccagatgctaagcactggaagccagatgctaagcactggaag

SEQ ID NO: 1300 - DNA, 44 bp, Artificial Sequence
  Synthetic Sequence
taaccactctccaagtaccaaataaccactctccaagtaccaaa

SEQ ID NO: 1301 - DNA, 44 bp, Artificial Sequence
  Synthetic Sequence
ttaacaggcagttctgctgctattaacaggcagttctgctgcta

SEQ ID NO: 1302 - DNA, 44 bp, Artificial Sequence; identical to SEQ ID NO: 337
  Synthetic Sequence

SEQ ID NO: 1303 - DNA, 42 bp, Artificial Sequence
  Synthetic Sequence
agaagtgcaccgcgaatgtttagaagtgcaccgcgaatgttt

SEQ ID NO: 1304 - DNA, 40 bp, Artificial Sequence
  Synthetic Sequence
ttaagagcccggctttgcctttaagagcccggctttgcct

SEQ ID NO: 1305 - DNA, 44 bp, Artificial Sequence
  Synthetic Sequence
atccacgttttaaataccaaagatccacgttttaaataccaaag

SEQ ID NO: 1306 - DNA, 42 bp, Artificial Sequence
  Synthetic Sequence
tgcctcccacacacagctttatgcctcccacacacagcttta

SEQ ID NO: 1307 - DNA, 44 bp, Artificial Sequence
  Synthetic Sequence
ttccccggcaccagcacaaagtttccccggcaccagcacaaagt

SEQ ID NO: 1308 - DNA, 44 bp, Artificial Sequence
  Synthetic Sequence
caatcagaggcaatcaagcacacaatcagaggcaatcaagcaca

SEQ ID NO: 1309 - DNA, 44 bp, Artificial Sequence
  Synthetic Sequence
taattctaaagacaaagcacaataattctaaagacaaagcacaa

SEQ ID NO: 1310 - DNA, 42 bp, Artificial Sequence
  Synthetic Sequence
ggttgtcaggaacagaagtgcggttgtcaggaacagaagtgc

SEQ ID NO: 1311 - DNA, 44 bp, Artificial Sequence; identical to SEQ ID NO: 1193
  Synthetic Sequence

SEQ ID NO: 1312 - DNA, 44 bp, Artificial Sequence
  Synthetic Sequence
acttgatcaaacagagcacaacacttgatcaaacagagcacaac

SEQ ID NO: 1313 - DNA, 44 bp, Artificial Sequence
  Synthetic Sequence
ttttctcctgactgattgcactttttctcctgactgattgcact

SEQ ID NO: 1314 - DNA, 44 bp, Artificial Sequence
  Synthetic Sequence
ttaaaatgacatggataatgcattaaaatgacatggataatgca

SEQ ID NO: 1315 - DNA, 40 bp, Artificial Sequence
  Synthetic Sequence
agaagcgcctttggcagctaagaagcgcctttggcagcta

SEQ ID NO: 1316 - DNA, 44 bp, Artificial Sequence
  Synthetic Sequence
tacctgcactatgagcactttgtacctgcactatgagcactttg

SEQ ID NO: 1317 - DNA, 44 bp, Artificial Sequence
  Synthetic Sequence
gtcatgatcatcccacactaatgtcatgatcatcccacactaat

SEQ ID NO: 1318 - DNA, 42 bp, Artificial Sequence
  Synthetic Sequence
tggcacctatgcccaccagcatggcacctatgcccaccagca

SEQ ID NO: 1319 - DNA, 44 bp, Artificial Sequence
  Synthetic Sequence
gctttgacaatatcattgcactgctttgacaatatcattgcact

SEQ ID NO: 1320 - DNA, 42 bp, Artificial Sequence
  Synthetic Sequence
gtcggcatctacacttgcactgtcggcatctacacttgcact

SEQ ID NO: 1321 - DNA, 42 bp, Artificial Sequence
  Synthetic Sequence
acctgctgccactggcacttaacctgctgccactggcactta

SEQ ID NO: 1322 - DNA, 44 bp, Artificial Sequence
  Synthetic Sequence
ggcatgaatttattgtgcaataggcatgaatttattgtgcaata

SEQ ID NO: 1323 - DNA, 36 bp, Artificial Sequence
  Synthetic Sequence
gctggcagggaagtagtggctggcagggaagtagtg

SEQ ID NO: 1324 - DNA, 42 bp, Artificial Sequence
  Synthetic Sequence
ataacacctacgagcactgccataacacctacgagcactgcc

SEQ ID NO: 1325 - DNA, 44 bp, Artificial Sequence
  Synthetic Sequence
agtcacagcatccattaataaaagtcacagcatccattaataaa

SEQ ID NO: 1326 - DNA, 44 bp, Artificial Sequence
  Synthetic Sequence
atgagaagactgtcacaatcaaatgagaagactgtcacaatcaa

SEQ ID NO: 1327 - DNA, 44 bp, Artificial Sequence
  Synthetic Sequence
ctgccaaaccaattaatacctcctgccaaaccaattaatacctc

SEQ ID NO: 1328 - DNA, 44 bp, Artificial Sequence
  Synthetic Sequence
tcatattttagttctgcactgatcatattttagttctgcactga

SEQ ID NO: 1329 - DNA, 44 bp, Artificial Sequence
  Synthetic Sequence
cacataacaggtgctcaaataacacataacaggtgctcaaataa

SEQ ID NO: 1330 - DNA, 44 bp, Artificial Sequence
  Synthetic Sequence
tagagattgtttcaacactgaatagagattgtttcaacactgaa

SEQ ID NO: 1331 - DNA, 44 bp, Artificial Sequence
  Synthetic Sequence
gtctccacagaaacttttgtccgtctccacagaaacttttgtcc

SEQ ID NO: 1332 - DNA, 44 bp, Artificial Sequence
  Synthetic Sequence
acccggtctgccagaagctgctacccggtctgccagaagctgct

SEQ ID NO: 1333 - DNA, 44 bp, Artificial Sequence
  Synthetic Sequence
ttcaatagggcataggtgccaattcaatagggcataggtgccaa

SEQ ID NO: 1334 - DNA, 44 bp, Artificial Sequence
  Synthetic Sequence
ctccaaagaacattactgtgatctccaaagaacattactgtgat

SEQ ID NO: 1335 - DNA, 44 bp, Artificial Sequence; identical to SEQ ID NO: 344
  Synthetic Sequence

SEQ ID NO: 1336 - DNA, 44 bp, Artificial Sequence
  Synthetic Sequence
atcaatgctatgtgatctgcatatcaatgctatgtgatctgcat

SEQ ID NO: 1337 - DNA, 44 bp, Artificial Sequence
  Synthetic Sequence
tcaccccaaagttgtggcaatatcaccccaaagttgtggcaata

SEQ ID NO: 1338 - DNA, 44 bp, Artificial Sequence
  Synthetic Sequence
atgtgacagagccaagcacaaaatgtgacagagccaagcacaaa

SEQ ID NO: 1339 - DNA, 44 bp, Artificial Sequence
  Synthetic Sequence
acctacactgaaactgccaaaaacctacactgaaactgccaaaa

SEQ ID NO: 1340 - DNA, 40 bp, Artificial Sequence
  Synthetic Sequence
ttaccaagggcgactcgcatttaccaagggcgactcgcat

SEQ ID NO: 1341 - DNA, 44 bp, Artificial Sequence; identical to SEQ ID NO: 305
  Synthetic Sequence

SEQ ID NO: 1342 - DNA, 44 bp, Artificial Sequence
  Synthetic Sequence
cccgtatgtaataaatgtgctacccgtatgtaataaatgtgcta

SEQ ID NO: 1343 - DNA, 44 bp, Artificial Sequence
  Synthetic Sequence
ttaagttttgaaaagtacatagttaagttttgaaaagtacatag

SEQ ID NO: 1344 - DNA, 44 bp, Artificial Sequence
  Synthetic Sequence
aaagcataccagctgaaccaaaaaagcataccagctgaaccaaa

SEQ ID NO: 1345 - DNA, 44 bp, Artificial Sequence
  Synthetic Sequence
cacaagttcctgcaaatgcacacacaagttcctgcaaatgcaca

SEQ ID NO: 1346 - DNA, 44 bp, Artificial Sequence
  Synthetic Sequence
aaaagagaccttcatatgcaaaaaaagagaccttcatatgcaaa

SEQ ID NO: 1347 - DNA, 44 bp, Artificial Sequence; identical to SEQ ID NO: 201
  Synthetic Sequence

SEQ ID NO: 1348 - DNA, 44 bp, Artificial Sequence
  Synthetic Sequence
aagcatatttctcccactgtgaaagcatatttctcccactgtga

SEQ ID NO: 1349 - DNA, 42 bp, Artificial Sequence
  Synthetic Sequence
tcctgatggtcgaagtgccaatcctgatggtcgaagtgccaa

SEQ ID NO: 1350 - DNA, 44 bp, Artificial Sequence
  Synthetic Sequence
cataattacagaaaattgcactcataattacagaaaattgcact

SEQ ID NO: 1351 - DNA, 44 bp, Artificial Sequence
  Synthetic Sequence
acacttagcaggttgtattataacacttagcaggttgtattata

SEQ ID NO: 1352 - DNA, 36 bp, Artificial Sequence
  Synthetic Sequence
tcacccgaggcgcacttatcacccgaggcgcactta